(19) United States
(12) Patent Application Publication
(10) Pub. No.: US 2015/0315557 A1
(43) Pub. Date: Nov. 5, 2015

(54) MEGANUCLEASE VARIANTS CLEAVING THE GENOME OF A PATHOGENIC NON-INTEGRATING VIRUS AND USES THEREOF

(71) Applicant: CELLECTIS, Paris (FR)

(72) Inventors: Andre Choulika, Paris (FR); Frederic Cedrone, Paris (FR); Julianne Smith, Le Plessis Robinson (FR)

(73) Assignee: CELLECTIS, Paris (FR)

(21) Appl. No.: 14/744,668

(22) Filed: Jun. 19, 2015

Related U.S. Application Data
(63) Continuation of application No. 13/322,266, filed on Mar. 19, 2012, now abandoned.

(30) Foreign Application Priority Data
May 26, 2009 (IB) PCT/IB2009/006039
Sep. 24, 2009 (IB) PCT/IB2009/007171
Mar. 5, 2010 (IB) PCT/IB2010/050968

(51) Int. Cl. C12N 9/22

(52) U.S. Cl. CPC C12N 9/22

(57) ABSTRACT

An I-CreI variant, wherein at least one of the two I-Cre1 monomers has at least two substitutions, one in each of the two functional subdomains of the LAGLIDADG core domain situated from positions 26 to 40 and 44 to 77 of I-CreI, said variant being able to cleave a DNA target sequence from the genome of a non-integrating virus, in particular herpes simplex virus (HSV) or Hepatitis B virus (HBV) for use in genome engineering and for in vivo and ex vivo (gene cell therapy) genome therapy as well as the treatment of a virus infection.

The invention relates to a meganuclease variant cleaving the genome of a non-integrating virus and in particular the genome of a Herpes Simplex Virus or Hepatitis B virus. The present invention also relates to a vector encoding said variant, as well as to a cell, animal or plant modified by this vector and to the use of these meganuclease variants and derived products for genome engineering and for in vivo and ex vivo (gene cell therapy) genome therapy as well as the treatment of a Herpesviridae infection or Hepadnaviridae infection.

Viral infections of various sorts are a serious and continuing health, agricultural and economic problem worldwide. In particular viruses present specific treatment and control problems as they always comprise an intracellular stage to their life cycle, in which the nucleic acid genome of the virus is inserted into a host cell and normally transported to the nucleus. During this stage of the virus life cycle, the virus genome can enter into a dormant state whilst inside a host cell, in which the production of new virus particles/proteins/copies of the viral genome ceases. These characteristics present a significant problem as most medicaments and treatments for viral infection consist of compounds which affect aspects of virus biology involved in the active stages of the virus life cycle, such as compounds which target a viral enzyme or structural protein. Therefore whilst in a dormant state the viral genome resident in the cytoplasm or nucleus of a host cell cannot be affected by most conventional anti-virus medicaments and therefore persists.

The present invention relates to viruses which do not integrate into the host genome following insertion of the viral genomic/genetic material into the host cell. That is the viral genetic material exists as an episomal/separate DNA molecule. Most important viruses exhibit such a life cycle, for example DNA ds (double stranded) viruses like Herpesviridae, Adenoviridae, Papovaviridae and Poxviridae; DNA ss (single stranded) viruses like Parvoviridae and DNA ds viruses that replicate through a single stranded RNA intermediate such as Hepadnaviridae.

To illustrate the utility of the meganuclease variants according to the present invention, the inventors have worked to establish the effects of their invention upon two important pathogenic viruses Hepatitis B and Herpes Simplex Virus.

Hepatitis B, a virus of the family Hepadnaviridae, is an example of an epidemiologically important virus which following insertion of the virus genome into a host cell, then exists as an episomal DNA molecule separate from the host cell genome in the nucleus.

Infection with hepatitis B virus (HBV) is a world health problem, leading to more than 1 million deaths per year according to the World Health Organization. HBV is transmitted through infected blood, body fluids and by sexual intercourse.

HBV exhibits genetic variability with an estimated rate of 1.4 to 3.2×10⁻⁵ nucleotide substitutions per site per year. A large number of virus variants arise during replication as a result of nucleotide misincorporations, due to the absence of any proof reading capacity by the viral polymerase. This variability has resulted in well recognized subtypes of the virus (Schaefer, World J. Gastroenterol., 2007, 13:14-21).

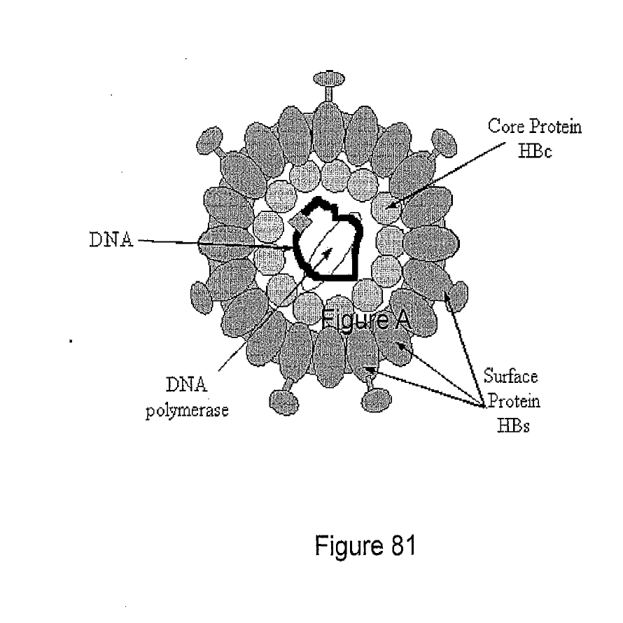

HBV is an enveloped DNA-containing virus that replicates through an RNA intermediate. The infectious (“Dane”) particle consists of an inner core plus an outer surface coat (FIG. 81). The virus is a spherical particle with a diameter of 42 nm and is composed of an outer shell (or envelope) composed of several proteins known collectively as HBs which surrounds an inner protein shell, composed of HBc protein. Finally the HBc protein surrounds the viral DNA and the viral DNA polymerase.

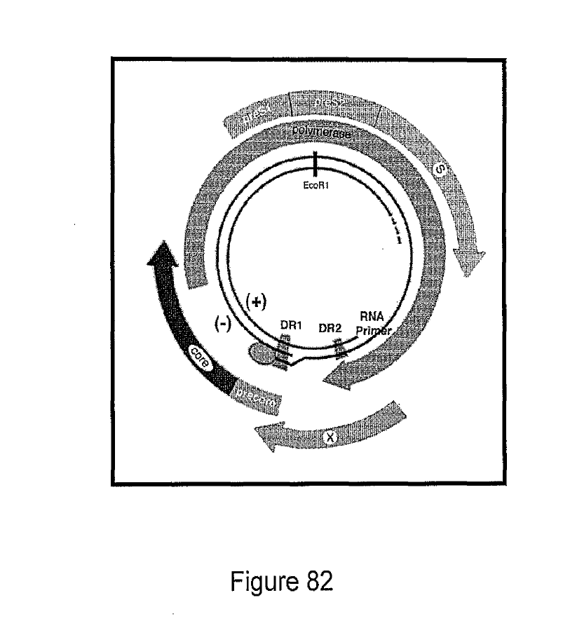

The HBV virion genome is circular and approximately 3.2 kb in size and consists of DNA that is mostly double stranded. It comprises four overlapping open reading frames running in one direction and no non-coding regions. The four overlapping open reading frames (ORFs) in the genome are responsible for the transcription and expression of seven different HBV proteins. The four ORFs are known as C, S, P and X. The C ORF codes for the viral core protein and the e-antigen, the S ORF codes for three related viral envelope proteins, the P ORF codes for viral DNA polymerase and the X ORF codes for a 16.5 kDa protein whose function is not well defined (FIG. 82).

The C ORF is divided into the precore region and the core region by two in-frame initiating ATG codons. The hepatitis B virus core antigen (HBcAg) is initiated from the second ATG and thus contains only the core region. The virus core antigens associate to form the hepatitis B core that encapsulates HBV DNA and DNA polymerase. This protein has been shown to be essential for viral DNA replication. A second protein, the hepatitis B e-antigen (HBeAg) is initiated from the first ATG in the C ORF and thus consists of the pre-core and core region. This protein is targeted to the endoplasmic reticulum where it is cleaved at the N and C terminus and then secreted as a non-particulate HBeAg. This protein is not essential for viral replication and its function remains unknown. The S ORF encodes for three envelope proteins known as small (S), medium (M), and large (L) hepatitis B surface antigen. All three proteins contain the structural domain. The extra domain in M is known as pre-S2 while L contains the pre-S2 and pre-S1 domains. The pre-S1 domain is thought to be the substrate for the viral receptor on hepatocytes and thus essential for viral attachment and entry. All three envelope proteins are components of the infectious viral particles also referred to as Dane particles. However, the S protein by itself or associated with the larger envelope proteins have been shown to form spheres and filaments that are secreted from infected cells in at least 100-fold excess over infectious viral particles. It is thought that these spheres and filaments may serve to titrate out antibodies that are produced by the immune system and thus aid the infectious viral particles to escape the immune system. The P ORF codes for the viral DNA polymerase. This protein consists of two major domains tethered by an intervening spacer region. The amino-terminal domain plays a critical role in the packaging of pre-genomic RNA and in the priming of minus strand DNA while the carboxyterminal domain is a reverse transcriptase that also has RNase H activity. This protein is essential for viral DNA replication. The X ORF encodes a protein that has been shown to be essential for virus replication in animals but dispensable for viral DNA synthesis in transfected tissue culture cells. It has been suggested that the X protein may play a role in transcriptional activation as well as stimulation of signal transduction pathways and regulation of apoptosis (Seeger and Mason, Microbiol. Mol. Biol. Rev., 2000, 51-68).

The viral genome consists of two partially overlapping DNA strands, called the − and + strands. The − strand is the larger of the two strands and is approximately 3.02 kb-3.32 kb in length and has a protein covalently attached to its 5′ end. The + strand, is approximately 1.7-2.8 kb in length and has an RNA oligonucleotide attached at its 5′ end.

The viral DNA is found in the nucleus soon after infection of the cell. The partially double-stranded DNA is rendered fully double-stranded by completion of the (+) sense strand and removal of the protein molecule from the (−) sense strand and a short sequence of RNA from the (+) sense strand and the ends are rejoined. This fully double-stranded DNA, a closed and circular DNA structure is known as cccDNA (covalently closed circular DNA).

HBV is a vaccine-preventable disease. Current vaccines are composed of the surface antigen of HBV and are produced by two different methods: plasma derived or recombinant DNA (Maupas et al, Lancet, 1976, 7974: 1367-1370; Mahoney, Clin. Microbiol. Rev., 1999, 12:351-366). However HBV vaccines are not available to all at risk individuals and/or are not always administered in the correct form and so cases of HBV infection persist throughout the world.

HBV infection can result in two distinct disease states, acute and chronic HBV infection. Acute HBV is the initial, rapid onset, short duration illness that results from infection with HBV. About 70% of adults with acute hepatitis B have few or no symptoms, while the remaining 30% develop significant symptoms (Seeger and Mason, Microbiol. Mol. Biol. Rev., 2000, 51-68). Rarely (in less than 1% of adults), individuals with acute hepatitis B can develop acute liver failure (fulminant hepatitis).

Chronic hepatitis B infection may take one of two forms: chronic persistent hepatitis, a condition characterized by persistence of HBV but in which liver damage is minimal; and chronic active hepatitis, in which there is aggressive destruction of liver tissue leading to cirrhosis and/or cancer such as hepatocellular carcinoma.

The prevalence of chronic HBV infection varies greatly in different parts of the world. Chronic HBV infection is highly endemic in developing regions with large populations such as South East Asia, China, sub-Saharan Africa and the Amazon Basin; moderately endemic in parts of Eastern and Southern Europe, the Middle East, Japan, and part of South America and low in most developed areas, such as North America, Northern and Western Europe and Australia.

When HBV infection results in a chronic disease, this cannot currently be cured. Therefore the goal of therapy is the long-term suppression of viral replication, as this is associated with a reduced risk of the development of advanced liver disease including liver cirrhosis and cancer. There are currently two major families of drugs that have been approved for the treatment of chronic Hepatitis B infections, interferons, which boost the immune system in order to eliminate or diminish the virus, and nucleoside/nucleotide analogues, which inhibit viral replication. As all treatments for Hepatitis B infections are administered overlong periods of time, one of the major problems is the development of drug resistance. This is particularly the case for nucleoside/nucleotide analogues for which there are a growing number of documented viral polymerase mutants that result in drug resistance (Tillman, World J. Gastroenterol., 2007, 13:125-140).

Liver transplantation is the only long term treatment available for patients with liver failure. However, liver transplantation is complicated by the risk of recurrent hepatitis B infection in patients where the initial liver failure was due to hepatitis B infection or who have a chronic HBV infection or a high risk of HBV reinfection; this problem significantly impairs graft and patient survival. In the absence of treatment, HBV reinfection occurs in 75%-80% of persons who undergo liver transplantation.

Therefore in the prior art significant problems exist with treating patients who are chronically infected with HBV and more specifically with reducing the HBV viral titer as far as possible in a patient who requires a liver transplant.

A promising target for the development of treatments for HBV infection and more generally non-integrating viruses is the intracellular episomal HBV/NIV (Non Integrating Virus) genome. The intracellular HBV genome is the molecular basis of HBV persistence. It has been found in both animal models and clinical investigations that cccDNA persists even after years of antiviral therapy and is responsible for the rapid increases in viral titer following withdrawal of treatment or the development of resistance.

It has been proposed (WO 2008/119000, Iowa University) that the intracellular genome of HBV could be targeted and potentially inactivated using a variety of methods such as via RNA interference (RNAi), short interfering RNA (siRNA) and engineered polydactyl zinc finger protein domains in combination with cleavage domains generated against a target(s) in the HBV genome. To date however none of these methods have been shown able to specifically target and/or affect the HBV virus.

Potential problems exist with all of these proposed mechanisms, for instance although it appears that RNAi can suppress virtually all classes of DNA and RNA virus against which they have been tested (Dykxhoom, D M and Lieberman, J (2006). PLoS Med 3: e242 and Leonard, J N and Schaffer, D V (2006). Gene Ther 13: 532-540), clinical studies are increasingly showing that viruses are able to elude the effects of RNAi (Gitlin, L, Karelsky, S and Andino, R (2002). Nature 418: 430-434 and Gitlin, L, Stone, J K and Andino, R (2005). J Virol 79: 1027-1035) with apparent ease.

Likewise in theory zinc finger domains could be generated which are specific to targets in the HBV genome and therefore Zinc finger nucleases (ZFNs), which are chimeric proteins composed of a ‘specific’ zinc finger DNA-binding domain linked to a non-specific DNA-cleavage domain, could be generated to a target in the HBV genome. Such ZFNs would not be useful as in general ZFNs are known to be highly cytotoxic (Porteus M H, Baltimore D (2003) Science 300: 763 and Beumer K, Bhattacharyya G, Bibikova M, Trautman J K, Carroll D (2006) Genetics 172: 2391-2403.) due to their cleavage of non-target sequences, leading to genome degradation. Although various steps have been attempted to attenuate these cytotoxic effects thus far ZFNs remain simply too toxic for routine use. The generation of such ZFNs is also a laborsome endeavour as the combination of a given zinc finger domain, following its generation, with a nuclease domain requires a substantial amount of work to ensure firstly that the combination is functional and secondly specific.

Another important group of non-integrating pathogenic viruses are from the family Herpesviridae. Of the more than 100 known Herpesviridae viruses, only 8 routinely infect humans: herpes simplex virus types 1 and 2, varicella-zoster virus, cytomegalovirus, Epstein-Barr virus, human herpes virus 6 (variants A and B), human herpes virus 7, Kaposi's sarcoma virus and human herpes virus 8. A simian virus, called B virus, occasionally infects humans. All herpes viruses can establish latent infection within specific tissues, which are characteristic for each virus (Medical Microbiology, 4^{th} Edition, Virology, Herpes viruses, Whitley R J, 1996).

Herpes viruses infect members of all groups of vertebrates, as well as some invertebrates. Herpes viruses have been typically classified into three groups based upon details of tissue tropism, pathogenicity and viral behaviour under conditions of culture in the laboratory. The three types include: the alpha-herpes viruses which are neurotropic, have a rapid replication cycle and a broad host and cell range; and the beta- and gamma-herpes viruses which differ in genome size and structure but which both replicate more slowly and in a much more restricted range of cells of glandular and/or lymphatic origin. To date, eight discrete human herpes viruses have been described; each causing a characteristic disease (Norberg et al, J Clin Microbiol, 2006, 44, 4511-4514).

Herpes simplex virus types 1 and 2 (HSV-1 and -2) will be used to illustrate the problems presented by Herpesviridae viruses. In the present patent application references to Herpes Simplex Virus and/or HSV refer to both HSV-1 and HSV-2. HSV-1 and HSV-2 are the primary agents of recurrent facial and genital herpetic lesions. Infections although mild in terms of the severity of symptoms, can lead to significant psychological trauma. They are also a major cause of encephalitis. Herpes simplex virus-1/-2 are highly adapted human pathogens with a rapid lytic replication cycle and also exhibit the ability to invade sensory neurons without showing any cytopathology. Latent infections are subject to reactivation whereby infectious virus can be recovered in peripheral tissue enervated by the latently infected neurons following a specific physiological stress. A major factor in these “switches” from lytic to latent infection and back involves changes in transcription patterns, mainly as a result of the interaction between viral promoters, the viral genome and cellular transcriptional machinery.

HSV is a nuclear replicating DNA virus. The HSV envelope contains at least 8 glycoproteins. The capsid itself is made up of 6 proteins. The major one is the capsid protein U_{L}19. The matrix which contacts both the envelope and the capsid contains at least 15-20 proteins.

The HSV-1 genome is a linear, double stranded DNA duplex 152,261 base pairs (bp) in length, and with a base composition of 68% G+C which circularizes upon infection. The virus encodes nearly 100 transcripts and more than 70 open translational reading frames (ORFs). Most ORFs are expressed by a single transcript. About 40 genes are considered as essential for virus replication in culture and these are listed in Table I below.

| | | Required for | |
|---|---|---|---|
| | | replication in | |
| Name | kinetics | culture? | Function |
| “a” | — | Yes | cis genome cleavage, packaging signal |
| TR_{L} | — | Yes | Terminal Long Repeat |
| gL (U_{L}1) | early | Yes | viral entry, associates with gH - polyadenylation usage changes with time |
| Helicase | early | Yes | DNA replication |
| (U_{L}5) | | | |
| U_{L}6 | late | Yes | capsid protein, capsid maturation, DNA packaging |
| Helicase/Primase | early | Yes | DNA replication |
| (UL8) | | | |
| a0 | IE | Yes | immediate-early transcription regulator (mRNA spliced) |
| Ori binding | early | Yes | DNA replication |
| protein | | | |
| (U_{L}9) | | | |
| U_{L}11 | late | Yes | tegument protein, capsid egress and envelopement |
| Alkaline | early | Yes | DNA packaging, capsid egress |
| exonuclease | | | |
| (U_{L}12) | | | |
| U_{L}15 | late | Yes | DNA packaging, cleavage of replicating DNA, spliced mRNA |
| U_{L}17 | late | Yes | cleavage and packaging of DNA |
| Capsid | late | Yes | Triplex |
| (U_{L}18) | | | |
| Capsid | late | Yes | major capsid protein, hexon |
| (U_{L}19) | | | |
| U_{L}20 | late | Yes | membrane associated, virion egress |
| gH (U_{L}22) | late | Yes | viral entry, functions with gL |
| U_{L}25 | late | Yes | tegument protein, capsid maturation, DNA packaging |
| U_{L}26 | early | Yes | Maturational protease |
| U_{L}26.5 | early | Yes | Scaffolding protein |
| gB (U_{L}27) | early | Yes | Glycoprotein required for virus entry |
| U_{L}28 | early | Yes | capsid maturation, DNA packaging |
| U_{L}29 | early | Yes | single-stranded DNA binding protein, DNA replication |
| DNA pol | early | Yes | DNA replication |
| (UL30) | | | |
| U_{L}32 | late | Yes | capsid maturation, DNA packaging |
| U_{L}33 | late | Yes | capsid maturation, DNA packaging |
| U_{L}35 | late | Yes | capsid protein, capsomer tips |
| U_{L}38 | late | Yes | Capsid protein, triplex |
| U_{L}39 | early | Yes | Large subunit ribonucleotide reductase |
| U_{L}40 | early | Yes | Small subunit ribonucleotide reductase |
| U_{L}42 | late | Yes | Polymerase accessory protein DNA replication |
| a-TIF | — | Yes | virion-associated transcriptional activator, enhances immediate-early |
| (UL48) | | | transcription through cellular Oct-1 and CTF binding at “TATGARAT” sites |
| Helicase/primase | early | Yes | DNA replication |
| (UL52) | | | |
| a27 (UL54) | immediate- | Yes | immediate-early regulatory protein, inhibits splicing |
| IR_{L} | — | Yes | Internal Long Repeat |
| R_{L}/Rₛ | — | Yes | Joint region, contains “a” sequences |
| Junction | | | |
| IRₛ | — | Yes | Internal Short Repeat |
| a4 | Immediate- | Yes | immediate-early transcriptional activator |
| Oriₛ | — | Yes | Origin of replication |
| gD (Uₛ6) | late | Yes | virus entry, binds HSV4EM |
| TRₛ | — | Yes | Terminal Short Repeat |
| “a” | — | Yes | cis genome cleavage, packaging signal |

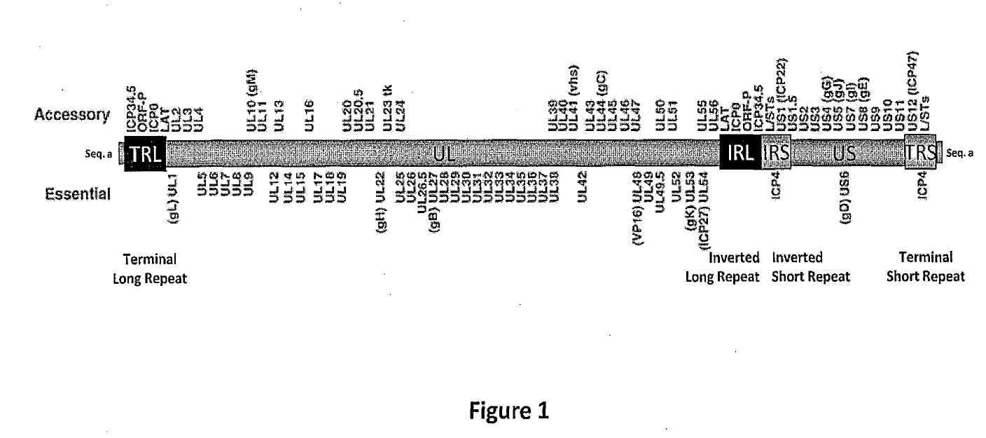

The HSV-1 genome is divided into six important regions (FIG. 1): 1) the ends of the linear molecules, the “a” sequences: these are important in both circularization of the viral DNA, and in packaging the DNA in the virion; 2) the 9,000 bp long repeat (R_{L}), which encode both an important immediate early regulatory protein (a0) and the promoter of most of the “gene” for the latency associated transcript (LAT); (3) the long unique region (U_{L}), which is 108,000 bp long, encodes at least 56 distinct proteins (actually more because some ORFs are spliced and expressed in redundant ways); it contains genes for the DNA replication enzymes and the capsid proteins, as well as many other proteins; 4) the 6,600 bp short repeats (R_{S}) encode the very important “a” immediate early protein; this is a very powerful transcriptional activator which acts along with a0/ICP0 and a27 (ICP27/UL54) (in the U_{L}) to stimulate the infected cell for all viral gene expression that leads to viral DNA replication; 5) the origins of replication: the ori_{L} is in the middle of the U_{L} region; the ori_{S} is in the R_{S} and thus, is present in two copies. All sets of ori's operate during infection to give a very complicated replication complex, very similar to that seen in the replication of phage T4; 6) the 13,000 bp unique short region (U_{S}) encodes 12 ORFs, a number of which are glycoproteins important in viral host range and response to host defence.

Five HSV-1 genes (a4 or ICP4, a0 or ICP0, a27 or ICP27/U_{L} 54, a22 or ICP22/U_{S}1, and a47 or ICP47/U_{S}12) are expressed and function at the earliest stages of the productive infection cycle. The “immediate-early” or “a” phase of gene expression is mediated by the action of α-TIF through its interaction with cellular transcription factors at specific enhancer elements associated with the individual a-transcript promoters. Activation of the host cell transcriptional machinery by the action of “a” gene products, results in the expression of the “early” or “b” genes. Seven of these are necessary and sufficient for viral DNA replication under all conditions DNA polymerase (U_{L}30), DNA binding proteins (U_{L}42 and U_{L}29 or ICP8), ORI binding protein (U_{L}9), and the helicase/primase complex (U_{L}5, 8, and 52). When sufficient levels of these proteins have accumulated within the infected cell, viral DNA replication ensues. Accessory or “non-essential” proteins for virus replication can be substituted for their function by one or another proteins.

HSV can adopt two different post-infection phenotypes: (i) productive infection or (ii) latent infection. The most recent models posit that when viral DNA migrates to nuclear pods, which are PML-associated subnuclear structures, it is either circularized by cellular DNA repair enzymes acting on the “a” sequences or remains linear through the action of the immediate-early ICP0 protein, which inhibits cellular DNA repair. In the former case, latent infection ensues while in the latter, productive replication takes place.

The vegetative replication of viral DNA which occurs during productive infection, represents a critical and central event in the viral replication cycle. High level of DNA replication irreversibly drives a cell to producing virus, which eventually results in its destruction. DNA replication also has a significant influence on viral gene expression. Early expression is significantly reduced or shut off following the start of DNA replication, while late genes begin to be expressed at high levels.

In a latent infection the viral genome is maintained intact in specific sensory neurons where it is genetically equivalent to that present in the viral particle, but the highly regulated productive cycle cascade of gene expression, so characteristic of herpes virus infections, does not occur. As a consequence, any transcription during latent infection with most herpes viruses is from a very restricted portion of the viral genome, and this transcription is important in some aspect of the process itself. During the latent phase, productive cycle genes are generally transcriptionally and functionally quiescent and only the latency associated transcript (LAT) is expressed. The promoter for the LAT contains neuron-specific cis-acting elements. The maintenance of the HSV genome in latently infected neurons requires no viral gene expression. HSV DNA is maintained as a nucleosomal, circular episome in latent infections and low levels of genome replication may occur or be necessary for the establishment or maintenance of a latent infection from which virus can be efficiently reactivated. The process of reactivation from latency is triggered by stress as well as other signals which are thought to transiently lead to increased transcriptional activity in the harboring neuron. The sensory nerve ganglia survive repeated reactivation without losing function. It appears to also occur without either extensive cytopathology associated with normal vegetative viral replication or with the death of only a very few cells. This process may be augmented by viral genes known to interfere with apoptosis, such as ICP34.5, which act to prevent neuronal death during reactivation where limited replication occurs (Maryam Ahmed et al., J Virol. 2002 January; 76(2): 717-729. doi: 10.1128/JVI.76.2.717-729.2002; Guey-Chuen Perng et al., J Virol. 2002 February; 76(3): 1224-1235. doi: 10.1128/JVI.76.3.1224-1235.2002; Ling Jin et al., J Virol. 2005 October; 79(19): 12286-12295. doi: 10.1128/JVI.79.19.12286-12295.2005).

To date, HSV treatments have been limited to antiviral substances that can reduce the level of infection by reducing the level of virus proliferation during vegetative infection. However, such antiviral substances have no effect on quiescent virus during the latency phase.

Other possible treatments which have been investigated include improving the immune response so as to keep the number of viral particles below the proliferation limit and so make episodes of virus replication of less severity, duration or asymptomatic. Some clinical trials are currently running for vaccines but the question of their efficiency on quiescent HSV is still uncertain. (Clin Vaccine Immunol. 2008 November; 15 (11):1638-43; Pediatric Research, 2001, 49:4; Curr Pharm Des. 2007; 13(19):1975-88)

The inventors seeing these problems with prior art approaches to treating virus infections, in particular the persistence of dormant copies of the virus genome, have now developed a new set of materials which target the otherwise stable episomal virus genome in situ within the nucleus or cytoplasm of an infected cell. The inventors have validated their work using the important diseases hepatitis B and Herpes Simplex Virus and have generated several meganuclease variants which can effectively recognize and cleave different targets in the HBV/HSV episomal genome leading to the cleavage and elimination or inactivation of the copies of the virus genome that allow the virus to persist.

In the wild, meganucleases are essentially represented by homing endonucleases. Homing Endonucleases (HEs) are a widespread family of natural meganucleases including hundreds of proteins families (Chevalier, B. S. and B. L. Stoddard, Nucleic Acids Res., 2001, 29, 3757-3774). These proteins are encoded by mobile genetic elements which propagate by a process called “homing”: the endonuclease cleaves a cognate allele from which the mobile element is absent, thereby stimulating a homologous recombination event that duplicates the mobile DNA into the recipient locus. Given their exceptional cleavage properties in terms of efficacy and specificity, they could represent ideal scaffolds to derive novel, highly specific endonucleases.

HEs belong to four major families. The LAGLIDADG family, named after a conserved peptidic motif involved in the catalytic center, is the most widespread and the best characterized group. Seven structures are now available. Whereas most proteins from this family are monomeric and display two LAGLIDADG motifs, a few have only one motif, and thus dimerize to cleave palindromic or pseudo-palindromic target sequences.

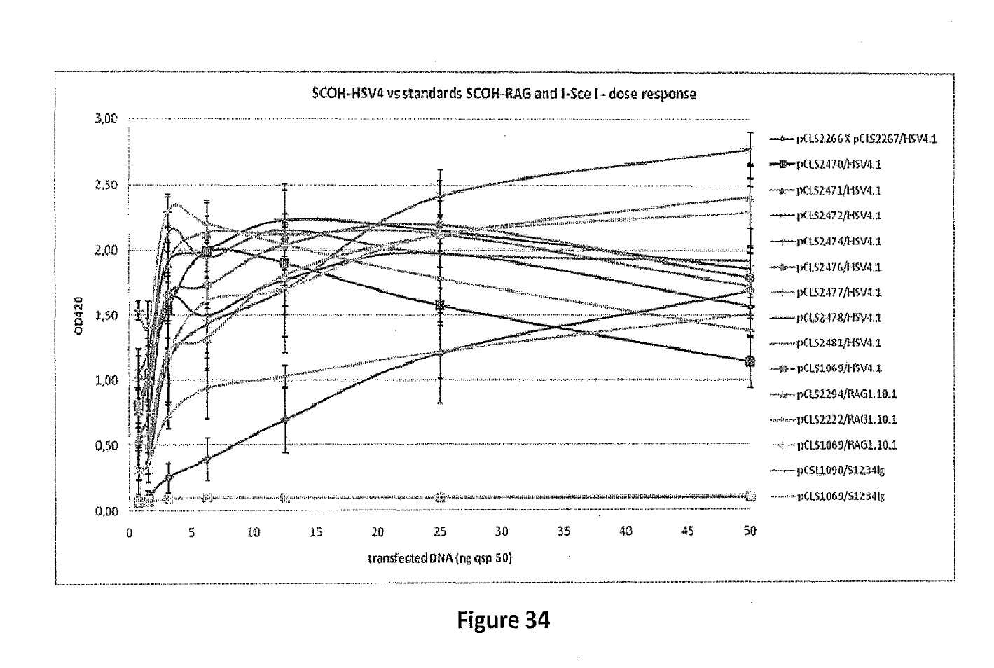

Although the LAGLIDADG peptide is the only conserved region among members of the family, these proteins share a very similar architecture (FIG. 34). The catalytic core is flanked by two DNA-binding domains with a perfect twofold symmetry for homodimers such as I-CreI (Chevalier, et al., Nat. Struct. Biol., 2001, 8, 312-316), I-MsoI (Chevalier et al., J. Mol. Biol., 2003, 329, 253-269) and I-CeuI (Spiegel et al., Structure, 2006, 14, 869-880) and with a pseudo symmetry for monomers such as I-SceI (Moure et al., J. Mol. Biol., 2003, 334, 685-69, I-DmoI (Silva et al., J. Mol. Biol., 1999, 286, 1123-1136) or I-AniI (Bolduc et al., Genes Dev., 2003, 17, 2875-2888). Both monomers and both domains (for monomeric proteins) contribute to the catalytic core, organized around divalent cations. Just above the catalytic core, the two LAGLIDADG peptides also play an essential role in the dimerization interface. DNA binding depends on two typical saddle-shaped αββαββα folds, sitting on the DNA major groove. Other domains can be found, for example in inteins such as PI-PfuI (Ichiyanagi et al., J. Mol. Biol., 2000, 300, 889-901) and PI-SceI (Moure et al., Nat. Struct. Biol., 2002, 9, 764-770), whose protein splicing domain is also involved in DNA binding.

The making of functional chimeric meganucleases, by fusing the N-terminal l-DmoI domain with an I-CreI monomer (Chevalier et al., Mol. Cell., 2002, 10, 895-905; Epinat et al., Nucleic Acids Res, 2003, 31, 2952-62; International PCT Application WO 03/078619 (Cellectis) and WO 2004/031346 (Fred Hutchinson Cancer Research Center, Stoddard et al)) have demonstrated the plasticity of LAGLIDADG proteins.

Different groups have also used a semi-rational approach to locally alter the specificity of the I-CreI (Seligman et al., Genetics, 1997, 147, 1653-1664; Sussman et al., J. Mol. Biol., 2004, 342, 31-41; International PCT Applications WO 2006/097784, WO 2006/097853, WO 2007/060495 and WO 2007/049156 (Cellectis); Arnould et al., J. Mol. Biol., 2006, 355, 443-458; Rosen et al., Nucleic Acids Res., 2006, 34, 4791-4800; Smith et al., Nucleic Acids Res., 2006, 34, e149), I-SceI (Doyon et al., J. Am. Chem. Soc., 2006, 128, 2477-2484), PI-SceI (Gimble et al., J. Mol. Biol., 2003, 334, 993-1008) and 1-MsoI (Ashworth et al., Nature, 2006, 441, 656-659).

In addition, hundreds of I-CreI derivatives with locally altered specificity were engineered by combining the semi-rational approach and High Throughput Screening:
Residues Q44, R68 and R70 or Q44, R68, D75 and I77 of I-CreI were mutagenized and a collection of variants with altered specificity at positions ±3 to 5 of the DNA target (5NNN DNA target) were identified by screening (International PCT Applications WO 2006/097784 and WO 2006/097853 (Cellectis); Arnould et al., J. Mol. Biol., 2006, 355, 443-458; Smith et al., Nucleic Acids Res., 2006, 34, e149).
Residues K28, N30 and Q38 or N30, Y33 and Q38 or K28, Y33, Q38 and S40 of I-CreI were mutagenized and a collection of variants with altered specificity at positions ±8 to 10 of the DNA target (10NNN DNA target) were identified by screening (Smith et al., Nucleic Acids Res., 2006, 34, e149; International PCT Applications WO 2007/060495 and WO 2007/049156 (Cellectis)).

Two different variants were combined and assembled in a functional heterodimeric endonuclease able to cleave a chimeric target resulting from the fusion of two different halves of each variant DNA target sequence (Arnould et al., precited; International PCT Applications WO 2006/097854 and WO 2007/034262).

Furthermore, residues 28 to 40 and 44 to 77 of I-CreI were shown to form two separable functional subdomains, able to bind distinct parts of a homing endonuclease target half-site (Smith et al. Nucleic Acids Res., 2006, 34, e149; International PCT Applications WO 2007/049095 and WO 2007/057781 (Cellectis)).

The combination of mutations from the two subdomains of I-CreI within the same monomer allowed the design of novel chimeric molecules (homodimers) able to cleave a palindromic combined DNA target sequence comprising the nucleotides at positions ±3 to 5 and ±8 to 10 which are bound by each subdomain (Smith et al., Nucleic Acids Res., 2006, 34, e149; International PCT Applications WO 2007/049095 and WO 2007/057781 (Cellectis)).

The method for producing meganuclease variants and the assays based on cleavage-induced recombination in mammal or yeast cells, which are used for screening variants with altered specificity are described in the International PCT Application WO 2004/067736; Epinat et al., Nucleic Acids Res., 2003, 31, 2952-2962; Chames et al., Nucleic Acids Res., 2005, 33, e178, and Arnould et al., J. Mol. Biol., 2006, 355, 443-458. These assays result in a functional LacZ reporter gene which can be monitored by standard methods.

The combination of the two former steps allows a larger combinatorial approach, involving four different subdomains. The different subdomains can be modified separately and combined to obtain an entirely redesigned meganuclease variant (heterodimer or single-chain molecule) with chosen specificity. In a first step, couples of novel meganucleases are combined in new molecules (“half-meganucleases”) cleaving palindromic targets derived from the target one wants to cleave. Then, the combination of such “half-meganucleases” can result in a heterodimeric species cleaving the target of interest. The assembly of four sets of mutations into heterodimeric endonucleases cleaving a model target sequence or a sequence from different genes has been described in the following Cellectis International patent applications: XPC gene (WO2007/093918), RAG gene (WO2008/010093), HPRT gene (WO2008/059382), beta-2 microglobulin gene (WO2008/102274), Rosa26 gene (WO2008/152523), Human hemoglobin beta gene (WO2009/13622) and Human interleukin-2 receptor gamma chain gene (WO2009019614).

These variants can be used to cleave genuine chromosomal sequences and have paved the way for novel perspectives in several fields, including gene therapy.

Even though the base-pairs ±1 and ±2 do not display any contact with the protein, it has been shown that these positions are not devoid of content information (Chevalier et al., J. Mol. Biol., 2003, 329, 253-269), especially for the base-pair ±1 and could be a source of additional substrate specificity (Argast et al., J. Mol. Biol., 1998, 280, 345-353; Jurica et al., Mol. Cell., 1998, 2, 469-476; Chevalier et al., Nucleic Acids Res., 2001, 29, 3757-3774). In vitro selection of cleavable I-CreI targets (Argast et al., precited) randomly mutagenized, revealed the importance of these four base-pairs on protein binding and cleavage activity. It has been suggested that the network of ordered water molecules found in the active site was important for positioning the DNA target (Chevalier et al., Biochemistry, 2004, 43, 14015-14026). In addition, the extensive conformational changes that appear in this region upon I-CreI binding suggest that the four central nucleotides could contribute to the substrate specificity, possibly by sequence dependent conformational preferences (Chevalier et al., 2003, precited).

The inventors of the present invention have developed a new approach and have created a new type of non-integrating virus agent which can target and eliminate the virus whilst it is inside a target cell by targeting the viral genome with one or more highly specific DNA restriction enzyme. Such highly specific DNA restriction enzymes recognizing specific viral sequences could act on proliferating virus as well as on latent viral DNA.

These materials can be used to manipulate the virus genome so as to elucidate aspects of virus biology and/or as a medicament to directly target and eliminate virus genomic material from the nuclei of infected cells.

According to a first aspect of the present invention there is provided an I-CreI variant which cleaves a DNA target in the genome of a pathogenic non-integrating virus (NIV), for use in treating an infection of said NIV.

The inventors have therefore created a new class of meganuclease based reagents which are useful for studying a NIV in vitro and in vivo; this class of reagents also represent a potential new class of anti-NIV medicament, which instead of acting upon the virion or any component thereof, acts upon the intracellular genomic of the virus.

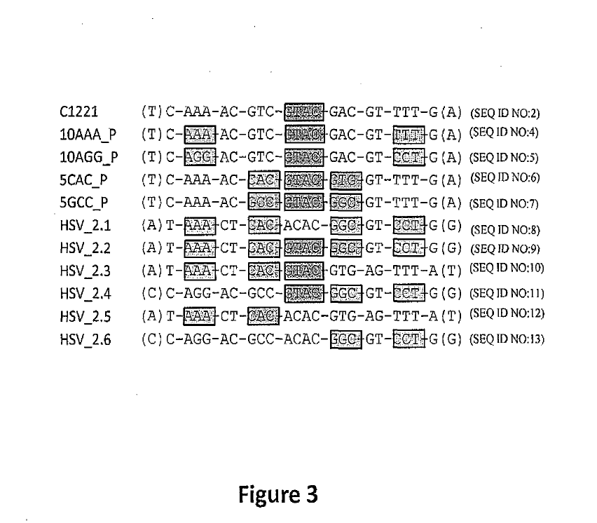

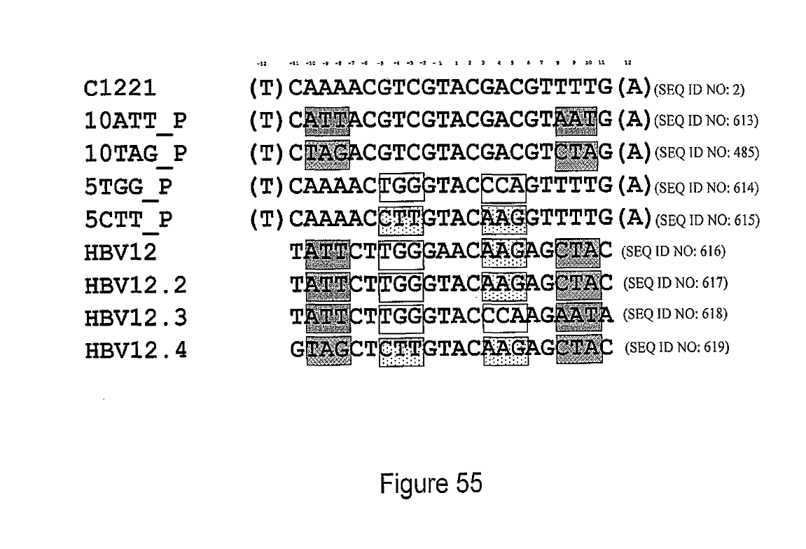

To validate their invention, the Inventors have identified a series of DNA targets in the genome of the Herpesviridae Virus Herpes Simplex Virus (HSV), that are cleaved by I-CreI variants (Table II to VIII and FIGS. 3, 24 and 49-52) and in the genome of the NIV hepatitis B virus (HBV), that are cleaved by I-CreI variants (FIGS. 55, 62, 70 and 84).

Target sequences can be chosen from one or more regions of the virus genome, for instance in the coding sequence of a virus gene and in particular in a gene (s) which is essential for the virus. In the present patent application essential genes are those genes which must remain active in order for the virus to be able to direct the manufacture and assembly of further virus particles which are able to exit the host cell and infect further cells. In addition to essential genes, other types of essential genetic elements can exist such as the regulatory elements of essential genes and/or structural sequence elements of the virus genome that are necessary for its packaging. For instance if the structure of the virus genetic material can be disrupted for instance by linearization or a strand break, this could make the viral genome susceptible to degradation by the innate anti-viral in vivo systems such as nuclease digestion.

For most viruses the majority of genes encoded by the virus are essential and hence inactivation of one or more of these viral genes either directly for instance by a truncation event or indirectly by for instance interrupting a regulatory sequence prevents this virus genome from producing further infective virus particles.

In particular the NIV is a virus from a family selected from the group comprising: Herpesviridae, Adenoviridae, Papovaviridae, Poxviridae, Parvoviridae, Hepadnaviridae.

In particular the NIV is selected from the group comprising: herpes simplex virus 1, herpes simplex virus 2, herpes simplex virus 3, Varicella zoster virus, Epstein-Barr virus, Cytomegalovirus, Herpes lymphotropic virus, Roseolovirus, Rhadinovirus, Adenovirus, Papillomavirus, Polyomavirus, variola virus, vaccinia virus, cowpox virus, monkeypox virus, camel pox, variola virus, vaccinia virus, cowpox virus, monkeypox virus, tanapox virus, yaba monkey tumor virus, molluscum contagiosum virus, Parvovirus B19, hepatitis B.

Multiple examples of genomic sequences for all these viruses are available from public databases such as the National Center for Biotechnology Information (http://www.ncbi.nlm.nih.gov/) or the virus genomics and bioinformatics resources centre at University College London (http//www.biochem.ucl.ac.uk/bsm/virus_database/VIDA.html).

According to a preferred aspect of the present invention a combinatorial approach was used to entirely redesign the DNA binding domain of the I-CreI protein and thereby engineer novel meganucleases with fully engineered specificity.

In particular therefore the I-CreI variant is characterized in that at least one of the two I-CreI monomers has at least two substitutions, one in each of the two functional subdomains of the LAGLIDADG core domain comprises mutations at two or more of positions 26, 28, 30, 32, 33, 38 and/or 40 and 44, 68, 70, 75 and/or 77 of I-CreI, said variant being able to cleave a DNA target sequence from the genome of a non-integrating virus (NIV), and being obtainable by a method comprising at least the steps of:

(a) constructing a first series of I-CreI variants having at least one substitution in a first functional subdomain of the LAGLIDADG core domain in at least one of positions 26, 28, 30, 32, 33, 38 of I-CreI,

(b) constructing a second series of I-CreI variants having at least one substitution in a second functional subdomain of the LAGLIDADG core domain in at least one of positions 44, 68, 70, 75 and/or 77 of I-CreI,

(c) selecting and/or screening the variants from the first series of step (a) which are able to cleave a mutant I-CreI site wherein at least one of (i) the nucleotide triplet in positions −10 to −8 of the I−CreI site has been replaced with the nucleotide triplet which is present in positions −10 to −8 of said DNA target sequence from the NIV genome and (ii) the nucleotide triplet in positions +8 to +10 has been replaced with the reverse complementary sequence of the nucleotide triplet which is present in positions −10 to −8 of said DNA target sequence from the NIV genome,

(d) selecting and/or screening the variants from the second series of step (b) which are able to cleave a mutant I-CreI site wherein at least one of (i) the nucleotide triplet in positions −5 to −3 of the I−CreI site has been replaced with the nucleotide triplet which is present in positions −5 to −3 of said DNA target sequence from the NIV genome and (ii) the nucleotide triplet in positions +3 to +5 has been replaced with the reverse complementary sequence of the nucleotide triplet which is present in position −5 to −3 of said DNA target sequence from the NIV genome,

(e) selecting and/or screening the variants from the first series of step (a) which are able to cleave a mutant I-CreI site wherein at least one of (i) the nucleotide triplet in positions +8 to +10 of the I-CreI site has been replaced with the nucleotide triplet which is present in positions +8 to +10 of said DNA target sequence from the NIV genome and (ii) the nucleotide triplet in positions −10 to −8 has been replaced with the reverse complementary sequence of the nucleotide triplet which is present in positions +8 to +10 of said DNA target sequence from the NIV genome,

(f) selecting and/or screening the variants from the second series of step (b) which are able to cleave a mutant I-CreI site wherein at least one of (i) the nucleotide triplet in positions +3 to +5 of the I-CreI site has been replaced with the nucleotide triplet which is present in positions +3 to +5 of said DNA target sequence from the NIV genome and (ii) the nucleotide triplet in positions −5 to −3 has been replaced with the reverse complementary sequence of the nucleotide triplet which is present in positions +3 to +5 of said DNA target sequence from the NIV genome,

(g) combining in a single variant, the mutation(s) in positions 26, 28, 30, 32, 33, 38 and/or 40 and 44, 68, 70, 75 and/or 77 of two variants from step (c) and step (d), to obtain a novel homodimeric I-CreI variant which cleaves a sequence wherein (i) the nucleotide triplet in positions −10 to −8 is identical to the nucleotide triplet which is present in positions −10 to −8 of said DNA target sequence from the NIV genome, (ii) the nucleotide triplet in positions +8 to +10 is identical to the reverse complementary sequence of the nucleotide triplet which is present in positions −10 to −8 of said DNA target sequence from the NIV genome, (iii) the nucleotide triplet in positions −5 to −3 is identical to the nucleotide triplet which is present in positions −5 to −3 of said DNA target sequence from the NIV genome and (iv) the nucleotide triplet in positions +3 to +5 is identical to the reverse complementary sequence of the nucleotide triplet which is present in positions −5 to −3 of said DNA target sequence from the NIV genome, and/or

(h) combining in a single variant, the mutation(s) in positions 26, 28, 30, 32, 33, 38 and 44, 68, 70, 75 and/or 77 of two variants from step (e) and step (f), to obtain a novel homodimeric I-CreI variant which cleaves a sequence wherein (i) the nucleotide triplet in positions +8 to +10 of the I-CreI site has been replaced with the nucleotide triplet which is present in positions +8 to +10 of said DNA target sequence from the NIV genome and (ii) the nucleotide triplet in positions −10 to −8 is identical to the reverse complementary sequence of the nucleotide triplet in positions +8 to +10 of said DNA target sequence from the NIV genome, (iii) the nucleotide triplet in positions +3 to +5 is identical to the nucleotide triplet which is present in positions +3 to +5 of said DNA target sequence from the NIV genome, (iv) the nucleotide triplet in positions −5 to −3 is identical to the reverse complementary sequence of the nucleotide triplet which is present in positions +3 to +5 of said DNA target sequence from the NIV genome,
(i) combining the variants obtained in steps (g) and (h) to form heterodimers, and

(j) selecting and/or screening the heterodimers from step (i) which are able to cleave said DNA target sequence from the NIV genome.

In particular the heterodimer of step (i) may comprise monomers obtained in steps (g) and (h), with the same DNA target recognition and cleavage activity properties.

Alternatively the heterodimer of step (i) may comprise monomers obtained in steps (g) and (h), with different DNA target recognition and cleavage activity properties.

In particular the first series of I-CreI variants of step (a) are derived from a first parent meganuclease.

In particular the second series of variants of step (b) are derived from a second parent meganuclease.

In particular the first and second parent meganucleases are identical.

Alternatively the first and second parent meganucleases are different.

In particular the variant may be obtained by a method comprising the additional steps of:

(k) selecting heterodimers from step (j) and constructing a third series of variants having at least one substitution in at least one of the monomers of said selected heterodimers,

(l) combining said third series variants of step (k) and screening the resulting heterodimers for enhanced cleavage activity against said DNA target from the NIV genome.

The inventors have found that although specific meganucleases can be generated to a particular target in the Non-integrating Virus genome using the above method, that such meganucleases can be improved further by additional rounds of substitution and selection against the intended target.

In particular in step (k) the substitutions in the third series of variants are introduced by site directed mutagenesis in a DNA molecule encoding said third series of variants, and/or by random mutagenesis in a DNA molecule encoding said third series of variants.

In the additional rounds of substitution and selection, the substitution of residues in the meganucleases can be performed randomly, that is wherein the chances of a substitution event occurring are of equal chance across all the residues of the meganuclease. Or on a site directed basis wherein the chances of certain residues being subject to a substitution is higher than other residues.

In particular steps (k) and (l) are repeated at least two times and wherein the heterodimers selected in step (k) of each further iteration are selected from heterodimers screened in step (l) of the previous iteration which showed increased cleavage activity against said DNA target from the NIV genome.

The inventors have found that the meganucleases can be further improved by using multiple iterations of the additional steps (k) and (l).

In particular the variant comprises one or more substitutions in positions 137 to 143 of I-CreI that modify the specificity of the variant towards the nucleotide in positions ±1 to 2, ±6 to 7 and/or ±11 to 12 of the target site in the NIV genome.

In particular the variant comprises one or more substitutions on the entire I-CreI sequence that improve the binding and/or the cleavage properties of the variant towards said DNA target sequence from the NIV genome.

As well as specific mutations at the residue identified above, the present invention also encompasses the substitution of any of the residues present in the I-CreI enzyme.

In particular wherein said substitutions are replacement of the initial amino acids with amino acids selected in the group consisting of A, D, E, F, G, H, I, K, M, N, P, Q, R, S, T, Y, C, W, L and V.

In particular the variant is a heterodimer, resulting from the association of a first and a second monomer having different mutations in positions 26, 28, 30, 32, 33, 38 and/or 40 and 44, 68, 70, 75 and/or 77 of I-CreI, said heterodimer being able to cleave a non-palindromic DNA target sequence from the N1V genome.

In particular the variant may be characterized in that it recognizes and cleaves a target sequence which comprises a specific nucleotide or group(s) of nucleotide(s) at one or more of positions ±1 to 12 which differs from the C1221 target (SEQ ID NO: 2) at least by one nucleotide.

In particular in the positions ±3 to 5, ±8 to 10 or ±1 to 2.

In particular wherein the sequence of nucleotides at the specified position is selected from the following groups:

±3 to 5 - CAC, GCC, GTG, GGC, GGT, ACC, CTG, TAC,
GTC, GTT, ATG, TGG;
±8 to 10 - AAA, AGG, TTT, CCT, AAG, ACT, CTT, AGT,
TGC, GGG, GCT, TGG, ATT;
±1 to 2 - GTAC, GTAA, TTAC, ACAC, GAGA, GAAC,
TTTT, ATAA.

As explained above the I-CreI enzyme acts as a dimer, by ensuring that the variant is a heterodimer this allows a specific combination of two different I-CreI monomers which increases the possible targets cleaved by the variant.

In particular the heterodimeric variant is an obligate heterodimer variant having at least one pair of mutations in corresponding residues of the first and the second monomers which mediate an intermolecular interaction between the two I-CreI monomers, wherein the first mutation of said pair(s) is in the first monomer and the second mutation of said pair(s) is in the second monomer and said pair(s) of mutations impairs the formation of functional homodimers from each monomer without preventing the formation of a functional heterodimer, able to cleave the genomic DNA target from the NIV genome.

The inventors have previously established a number of residue changes which can ensure an I-CreI monomer is an obligate heterodimer (WO2008/093249, CELLECTIS).

In particular the monomers have at least one of the following pairs of mutations, respectively for the first and the second monomer:

a) the substitution of the glutamic acid in position 8 with a basic amino acid, preferably an arginine (first monomer) and the substitution of the lysine in position 7 with an acidic amino acid, preferably a glutamic acid (second monomer); the first monomer may further comprise the substitution of at least one of the lysine residues in positions 7 and 96, by an arginine.

b) the substitution of the glutamic acid in position 61 with a basic amino acid, preferably an arginine (first monomer) and the substitution of the lysine in position 96 with an acidic amino acid, preferably a glutamic acid (second monomer); the first monomer may further comprise the substitution of at least one of the lysine residues in positions 7 and 96, by an arginine

c) the substitution of the leucine in position 97 with an aromatic amino acid, preferably a phenylalanine (first monomer) and the substitution of the phenylalanine in position 54 with a small amino acid, preferably a glycine (second monomer); the first monomer may further comprise the substitution of the phenylalanine in position 54 by a tryptophane and the second monomer may further comprise the substitution of the leucine in position 58 or lysine in position 57, by a methionine, and

d) the substitution of the aspartic acid in position 137 with a basic amino acid, preferably an arginine (first monomer) and the substitution of the arginine in position 51 with an acidic amino acid, preferably a glutamic acid (second monomer).

In particular the variant, which is an obligate heterodimer, wherein the first and the second monomer, respectively, further comprises the D137R mutation and the R51D mutation.

In particular the variant, which is an obligate heterodimer, wherein the first monomer further comprises the K7R, E8R, E61R, K96R and L97F or K7R, E8R, F54W, E61R, K96R and L97F mutations and the second monomer further comprises the K7E, F54G, L58M and K96E or K7E, F54G, K57M and K96E mutations.

Alternatively there is provided a single-chain chimeric meganuclease which comprises two monomers or core domains of one or two variant(s) according to the first aspect of the present invention, or a combination of both as previously described in WO03078619 and WO2009095742, from CELLECTIS relating to single-chain meganucleases.

The single chain meganuclease of the present invention further comprises obligate heterodimer mutations as described above so as to obtain single chain obligate heterodimer meganuclease variants.

An alternative approach to ensuring that the variant consists of a specific combination of monomers is to link the selected monomers for instance using a peptide linker.

In particular the single-chain meganuclease comprises a first and a second monomer according to the first aspect of the present invention, connected by a peptidic linker.

### (i) Herpesviridae Viruses

In particular the DNA target is within an essential gene or regulatory element or structural element of the Herpesviridae Virus genome.

Most particularly the Herpesviridae Virus is a virus which causes a disease in higher animals and in particular mammals.

In particular the Herpesviridae Virus is a virus selected from the group comprising: herpes simplex virus type 1, herpes simplex virus type 2, varicella-zoster virus, cytomegalovirus, Epstein-Barr virus, human herpes virus 6 (variants A and B), human herpes virus 7, Kaposi's sarcoma virus and human herpes virus 8.

Multiple examples of genomic sequences for all these viruses are available from public databases such as the National Center for Biotechnology Information (http://www.ncbi.nim.nih.gov/) or the virus genomics and bioinformatics resources centre at University College London (http://www.biochem.ucl.ac.uk/bsm/virus_database/VIDA.html).

These publicly available resources together with the detailed materials and methods described in the present patent application mean that meganuclease variants cleaving appropriate targets in their genomes can be generated and that in turn these variants can be used to cleave the viral genomic material in vivo for therapeutic and/or research purposes in accordance with the various aspects of the present invention.

In particular the herpes simplex virus is Herpes Simplex Virus (HSV) Type 1 or Type 2.

In particular the DNA target sequence is from a Herpes Simplex Virus Type 1 or Type 2.

In particular the variants may be selected from the group consisting of SEQ ID NO: 25 to 36, 40 to 90, 93 to 151, 153 to 168, 171 to 246, 249 to 252, 267 to 273, 275 to 288, 290 to 433, 436 to 445, 455 to 463, 470 to 471, 511 to 521, 522 to 531, 541 to 554 and 592 to 605.

In particular the single chain variants may be selected from the group consisting of SEQ ID NO: 253 to 261, 446 to 454, 465-466, 532 to 534, 535, 556 to 568, 571 to 580, 583 to 590 and 607 to 612.

In particular said DNA target is selected from the group consisting of the sequences SEQ ID NO: 8 to 13, 17 to 24, 472, 477 to 482, 487 to 492, 497 to 502, 507 to 510.

In particular said DNA target is within a DNA sequence essential for HSV replication, viability, packaging or virulence.

In particular the DNA target is within an open reading frame of the HSV genome, selected from the group: RL2, RS1, US2, UL19, UL30 or UL5.

In the present patent application the inventors provide meganuclease variants which can cleave targets in the RL2/ICP0 gene (targets HSV 12 and 4, SEQ ID NO: 20 and 17 respectively); in the RS1 gene (targets HSV 13 and 14, SEQ ID NO: 21 and 22 respectively); in the US2 gene (target HSV 1, SEQ ID NO: 23), in the UL19 gene (target HSV 2, SEQ ID NO: 24), in the UL30 gene (target HSV8) and in the UL5 gene (target HSV9). The cleavage of these sites in the HSV genome in vivo would therefore disrupt the sequence encoding the corresponding gene and thereby following a disruption and/or alteration of these gene sequences inactivate the HSV genome.

The RL2 gene encodes an important immediate early transcription factor acting as a regulatory protein (a0). This gene is considered as non essential due to its possible replacement by cellular transcription factors. However, it has been considered of major interest due to its localization in TRL, which is essential for HSV-1. Moreover, the central role of a0 during acute infection, latency establishment and virus reactivation has lead us to consider ICP0 as an integrator of essential signals. ICP0 gene is located in the 9 kb RL region repeated twice in HSV genome. This RL region encodes most of the gene for the latency associated transcript. This region is the unique active region during latency phase. Thus, targeting ICP0 gene would allow targeting an “opened” genomic sequence of quiescent virus and an important immediate early protein during virus infection and vegetative production. Many meganucleases can be built to recognize sequences in ICP0 gene. HSV4 described latter is one of them.

HSV12 is an example of a target from within the RL2 gene for which meganuclease variants can be generated. The HSV12 target sequence (cctggacatggagacggggaacat, SEQ ID NO: 501) is located at positions 5168-5191 bp and 121180-121203 bp in exon 3 of the RL2 gene repeated from positions 2086 to 5698 and from positions 120673 to 124285. Shown in Table II are two heterodimeric I-CreI variants which recognize and cleave the HSV12 target. Throughout the present patent application the sequence of the I-CreI variants described herein may be made using the following notation 24V33C etc. In this notation the numeral refers to the amino acid number in the I-CreI monomer and the letter refers to the amino acid present in this variant. If a residue is not explicitly listed this means this residue is identical to the residue in the wild type or parent 1-CreI monomer as appropriate.

TABLE II
Example of heterodimeric meganuclease variants
cleaving the HSV12
(cctggacatggagacggggaacat) target

HSV12.3-M1 (SEQ ID NO: 25)
24V33C38S44I50R70S75N77R132V
+
HSV12.4-ME-132V (SEQ ID NO: 26)
19S8K30R33S44K66H68Y70S77T87I132V139R163S
HSV12.3-M1-80K (SEQ ID NO: 27)
24V33C38S44I50R70S75N77R80K132V
+
HSV12.4-ME-132V (SEQ ID NO: 26)
19S8K30R33S44K66H68Y70S77T87I132V139R163S

ICP4 (RS1) gene is located in the RS region (6.6 kb) repeated twice in HSV-1 genome. IRS and TRS are located from positions 125974 to 132604 and from 145585 to 152259. The ICP4 virus essential gene, functions at the earliest stages of the productive infection cycle. RS1 encodes the immediate early transcription activator (a4) which, upon infection, directs cellular machinery to viral gene expression. This protein functions in association with ICP0 (a0) and ICP27 (a27) to improve viral gene expression and viral mRNA translation. Thus targeting ICP4 gene is of major interest in a meganuclease mediated antiviral approach.

The ICP4 gene can be targeted by many meganucleases. For example, sequences aggggacggggaacagcgggtggt (SEQ ID NO: 21) and ctcttcttcgtcttcgggggtcgc (SEQ ID NO: 22) are recognized and efficiently cleaved by I-Cre I variants HSV13 and HSV14. HSV13 target sequence is located from positions 128569 to 128592 and from 149641 to 149664 (NC_{—}001806). An example of I-CreI variant targeting HSV13 is shown in Table III.

TABLE III
Example of heterodimeric meganuclease variants
cleaving the HSV13
(atgttccccgtctccatgtccagg) target

HSV13-3-M15-19S (SEQ ID NO: 28)
6S 19S 28E 33R 38R 40K 43L 44N 68H 70S 75Y 77N
79G 80K
+
HSV13-4-MD (SEQ ID NO: 29)
30R 44R 60E 68Y 70S 75N 77D 80G
HSV13-3-M16-19S (SEQ ID NO: 30)
6S 19S 28E 33R 38R 40K 44N 68H 70S 75Y 77N 79G
80K 105A
+
HSV13-4-MD (SEQ ID NO: 29)
30R 44R 60E 68Y 70S 75N 77D 80G

HSV14 target sequence is located from positions 128569 to 128592 and from 149641 to 149664 (NC 001806). An example of I-CreI variant targeting HSV 14 is shown in Table IV.

TABLE IV
Example of heterodimeric meganuclease variants
cleaving the HSV14
(ctcttcttcgtcttcgggggtcgc) sequence

HSV14.3-MA-19S (SEQ ID NO: 31)
19S33G38C44K66H68 Y70S77T
+
HSV14.4-MB (SEQ ID NO: 32)
33H40R43L44K54I68A70S115V129A

The US2 gene is located in the US region of the HSV-1 genome. The 12 open reading frames contained in this 13 kb region are implicated in virus defense against host response, most of gene products are glycoproteins. The US2 gene is located from positions 134053 to 134928, less than 2 kb downstream the IRS region coding a4. This gene encodes a possible envelope-associated protein which interacts with cytokeratin 18. By targeting this gene the inventors of the present invention wanted to evaluate the accessibility of this locus as well as have an evaluation of the cleavage effect of this non essential viral gene toward HSV infection.

Among the multiple sequences recognized by I-CreI variants, the HSV 1 target sequence atgggacgtcgtaagggggcctgg, (SEQ ID NO: 23) (134215-134238) is targeted by meganuclease as detailed in Table V below.

TABLE V
Example of heterodimeric meganuclease variants
cleaving the HSV1
(atgggacgtcgtaagggggcctgg) target

HSV1.3-M5 (SEQ ID NO: 470)
30R33G38T106P
+
HSV1.4-MF (SEQ ID NO: 471)
30G38R44K57E70E75N108V

HSV2 is a 24 bp (non-palindromic) target present in the UL19 gene encoding the HSV-1 major capsid protein. This 5.7 kb gene in present in one copy in the locus 35023 to 40768 of the UL region. The HSV1-major capsid protein is expressed without maturation from an ORF located from 36404 to 40528. The target HSV2 is located from nucleotide 36966 to 36989 (accession number NC_{—}001806. The HSV2 target is recognized and cleaved by the meganuclease shown in Table VI below.

TABLE VI
Example of heterodimeric meganuclease variants
cleaving the HSV2
(ataaactcacacacggcgtcctgg) target

HSV2.3-M1 (SEQ ID NO: 33)
44D68T70S75R77R80K
+
HSV2.4-MC (SEQ ID NO: 34)
28E38R40K44K54I70S75N

HSV4 is a 24 bp (non-palindromic) target present in the RL2 gene encoding the ICP0 or a0 protein. This 3.6 kb gene repeated twice in TRL (2086 to 5698) and IRL (120673 to 124285) regions is formed of three exons: position 2261 to 2317, 3083 to 3749, 3886 to 5489 and 120882 to 122485, 122622 to 123288, 124054 to 124110. The target sequence present in exon 2 corresponds to positions 3498 to 3521 and 122850 to 122873 in the two copies of the HSV-1 ICP₀ gene (accession number NC_{—}001806). The HSV4 target is recognized and cleaved by the meganuclease shown in Table VII below.

TABLE VII
Example of heterodimeric meganuclease variants
cleaving the HSV4
(ccaagctggtgtacctgatagtgg) target

HSV4.3 optimised variant (SEQ ID NO: 35)44M70A80K1
32V146K156G
+
HSV4.4 optimised variant (SEQ ID NO: 36)
32E38Y44A68Y70S75Y77K105A

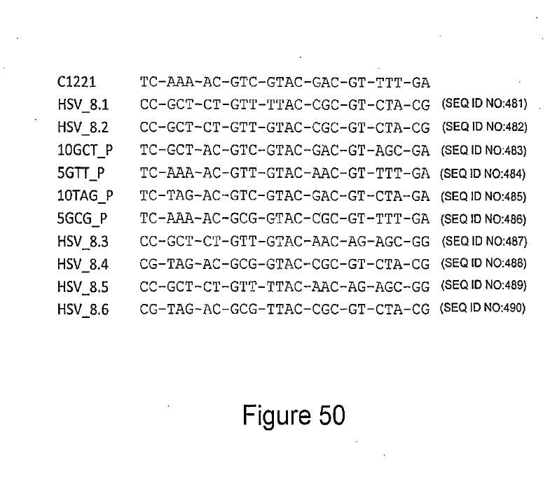

HSV8 is a 24 bp (non-palindromic) target (HSV8: CC-GCT-CT-GTT-TTAC-CGC-GT-CTA-CG, SEQ ID NO:481, FIG. 50) present in the UL30 gene encoding the DNA polymerase catalytic subunit of HSV-1. The herpes simplex virus DNA polymerase (HSV pol) holoenzyme consists of a large catalytic (UL30) and a small auxiliary subunit (UL42) (Franz C et al., Virology. 1999 Jan. 5; 253(1):55-64).

This 4 kb gene is present in one copy at position 62606 to 66553 of the UL region. The UL30 gene is required during viral genome multiplication. For optimal DNA synthesis HSV-1 needs replication proteins, ICP8, DNA polymerase (UL30/UL42), and helicase-primase (UL5/UL52/UL8) (Nimonkar A V & Boehmer P E., J Biol Chem. 2004 May 21; 279(21):21957-65). This gene is expressed at the early stage of acute infection and is considered as essential for virus replication in cell culture. The target HSV8 is located from nucleotide 63600 to 63623 (accession number NC_{—}001806; FIG. 1).

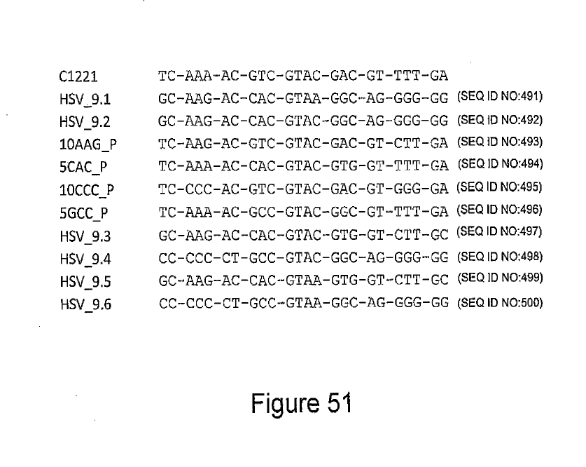

HSV9 is a 24 bp (non-palindromic) target (HSV9: GC-AAG-AC-CAC-GTAA-GGC-AG-GGG-GG (SEQ ID NO: 491), FIG. 51) present in the UL5 gene encoding a subunit of helicase-primase of HSV-1.

This 3.4 kb gene is present in one copy at position 11753 to 15131 of the UL region. The UL5 gene is one of the genes required during viral genome multiplication (Nimonkar A V & Boehmer P E., J Biol Chem. 2004 May 21; 279(21):21957-65). This gene is expressed at the early stage of acute infection and is considered as essential for virus replication in cell culture (Zhu L, Weller S K. Virology. 1988 October; 166(2):366-78). The target HSV9 is located from nucleotide 12833 to 12856 (accession number NC_{—}001806; FIG. 1).

### (ii) Hepadnaviridae Viruses

Alternatively the DNA target is within an essential gene or regulatory element or structural element of the Hepadnaviridae Virus genome.

In particular the Hepadnaviridae Virus is a virus which causes a disease in higher animals and in particular mammals.

Most particularly the DNA target is from the genome of hepatitis B.

In particular the DNA target is from a hepatitis B virus of genotype A.

As indicated above HBV exhibits genetic variability with an estimated rate of 1.4-3.2×10⁻⁵ nucleotide substitutions per site per year. A large number of virus variants arise during replication as a result of nucleotide misincorporations in the absence of any proof reading capacity by the viral polymerase. This variability has resulted in well recognized subtypes of the virus. HBV has been classified into 8 well defined genotypes on the basis of an inter-group divergence of 8% or more in the complete genomic sequence, each having a distinct geographical distribution. Genotype A is most commonly found in Northern Europe, North America and Central Africa, while genotype B predominates in Asia (China, Indonesia and Vietnam). Genotype C is found in the Far East in Korea, China, Japan and Vietnam as well as the Pacific and Island Countries, while genotype D is found in the Mediterranean countries, the Middle East extending to India, North America and parts of the Asia-Pacific region. Genotype E is related to Africa while genotype F is found predominately in South America, including among Amerindian populations, and also Polynesia. Genotype G has been found in North America and Europe while the most recently identified genotype H has been reported from America (Schaefer, World J. Gastroenterol., 2007, 13:14-21).

In the present patent application the inventors have also generated meganuclease variants to targets present in the genome of hepatitis B virus either in genotype A subtype adw2 (Preisler-Adams et al., Nucleic Acids Research, 1993, Vol. 21, No. 9), which corresponds to Genbank accession number X70185 or in subtype adr, which corresponds to Genbank accession number M38636.

In particular said DNA target is within a DNA sequence essential for HBV replication, viability, packaging or virulence.

In particular the DNA target is within an open reading frame of the HBV genome, selected from the group: C ORF, S ORF, P ORF and X ORF.

The HBV virion genome contains four overlapping open reading frames (ORFs) in the genome which are responsible for the transcription and expression of seven different hepatitis B proteins. The transcription and translation of these proteins is through the use of multiple in-frame start codons. The HBV genome also contains parts that regulate transcription, determine the site of polyadenylation and a specific transcript for encapsidation into the capsid.

Details concerning the four overlapping open reading frames of the HBV genome are detailed in the introduction above. In particular the DNA target is located in one of the HBV genomic genes selected from the group: viral core protein, e-antigen, small (S) hepatitis B surface antigen, medium (M) hepatitis B surface antigen, large (L) hepatitis B surface antigen, viral DNA polymerase, X protein.

In the present patent application the inventors provide meganuclease variants which can cleave targets in the S ORF and P ORF (target HBV12, SEQ ID NO: 616) and two independent targets in the C ORF (target HBV8, SEQ ID NO: 685 and target HBV3, SEQ ID NO: 723). The cleavage of these sites in the HBV genome in vivo would therefore disrupt the sequence encoding the small (S) hepatitis B surface antigen, medium (M) hepatitis B surface antigen, large (L) hepatitis B surface antigen, viral DNA polymerase, viral core protein and e-antigen of the virus and thereby following a disruption and/or alteration of these gene sequences inactivate the HBV genome.

In particular the variants may be selected from the group consisting of SEQ ID NO: 621 to 626; 628 to 633; 635 to 647; 665 to 678; 690 to 697; 699 to 702; 705 to 715; 730 to 734; 736 to 740; 743 to 750; 752 to 759; 761 to 765; 767 to 771; 780 to 798.

In particular the single chain variants may be selected from the group consisting of SEQ ID NO: 788, 799 to 800, 804 to 805.

In particular said DNA target is selected from the group consisting of the sequences SEQ ID NO: 616 to 619; 685 to 688; 723 to 728.

According to a second aspect of the present invention there is provided a polynucleotide fragment encoding a variant according to the first aspect of the present invention. The polynucleotide fragment can be either cDNA or mRNA encoding a variant according the first aspect of the present invention.

According to a third aspect of the present invention there is provided an expression vector comprising at least one polynucleotide fragment according to the second aspect of the present invention.

In particular the expression vector, includes a targeting construct comprising a sequence to be introduced flanked by sequences sharing homologies with the regions surrounding said DNA target sequence from the non-integrating Virus genome.

One important use of a variant according to the present invention is in increasing the incidence of homologous recombination events at or around the site where the variant cleaves its target. The present invention therefore also relates to a unified genetic construct which encodes the variant under the control of suitable regulatory sequences as well as sequences homologous to portions of the Non-integrating Virus genome surrounding the variant DNA target site. Following cleavage of the target site by the variant these homologous portions can act as a complementary sequences in a homologous recombination reactions with the Non-integrating Virus genome replacing the existing Non-integrating Virus genome sequence with a new sequence engineered between the two homologous portions in the unified genetic construct.

Preferably, homologous sequences of at least 50 bp, preferably more than 100 bp and more preferably more than 200 bp are used. Shared DNA homologies are located in regions flanking upstream and downstream the site of the break and the DNA sequence to be introduced should be located between the two arms.

Therefore, the targeting construct is preferably from 200 bp to 6000 bp, more preferably from 1000 bp to 2000 bp; it comprises: a sequence which has at least 200 bp of homologous sequence flanking the target site, for repairing the cleavage and a sequence for inactivating the Non-integrating Virus genome and/or a sequence of an exogeneous gene of interest.

For the insertion of a sequence, DNA homologies are generally located in regions directly upstream and downstream to the site of the break (sequences immediately adjacent to the break; minimal repair matrix). However, when the insertion is associated with a deletion of ORF sequences flanking the cleavage site, shared DNA homologies are located in regions upstream and downstream the region of the deletion.

A vector which can be used in the present invention includes, but is not limited to, a viral vector, a plasmid, a RNA vector or a linear or circular DNA or RNA molecule which may consist of a chromosomal, non chromosomal, semisynthetic or synthetic nucleic acids. Preferred vectors are those capable of autonomous replication (episomal vector) and/or expression of nucleic acids to which they are linked (expression vectors). Large numbers of suitable vectors are known to those of skill in the art and commercially available.

Viral vectors include retrovirus, adenovirus, parvovirus (e. g. adenoassociated viruses), coronavirus, negative strand RNA viruses such as orthomyxovirus (e.g., influenza virus), rhabdovirus (e. g., rabies and vesicular stomatitis virus), paramyxovirus (e. g. measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox and canarypox). Other viruses include Norwalk virus, togavirus, flavivirus, reoviruses, papovavirus, hepadnavirus, and hepatitis virus, for example. Examples of retroviruses include: avian leukosissarcoma, mammalian C-type, B-type viruses, D type viruses, HTLV-BLV group, lentivirus, spumavirus (Coffin, J. M., Retroviridae: The viruses and their replication, In Fundamental Virology, Third Edition, B. N. Fields, et al., Eds., Lippincott-Raven Publishers, Philadelphia, 1996).

Vectors can comprise selectable markers, for example: neomycin phosphotransferase, histidinol dehydrogenase, dihydrofolate reductase, hygromycin phosphotransferase, herpes simplex virus thymidine kinase, adenosine deaminase, glutamine synthetase, and hypoxanthine-guanine phosphoribosyl transferase (HRPT) for eukaryotic cell culture; TRP1 for S. cerevisiae; tetracycline, rifampicin or ampicillin resistance in E. coli.

In particular for the purposes of gene therapy and in accordance with a preferred embodiment of the present invention, the viral vector is selected from the group comprising lentiviruses, Adeno-associated viruses (AAV) and Adenoviruses.

A particular advantage of using virus vectors to deliver a variant which cleaves a virus target for a therapeutic purpose, is that the administration of the virus vector per se will illicit an immune response from the treated organism which in turn will impede the virus infection.

In accordance with another aspect of the present invention the variant and targeting construct may be on different nucleic acid constructs.

In accordance with another aspect of the present invention the variant in a peptide form and the targeting construct as a nucleic acid molecule may be used in combination.

In particular, wherein the sequence to be introduced is a sequence which inactivates the Non-integrating Virus genome.

In particular, wherein the sequence which inactivates the Non-integrating Virus genome comprises in the 5′ to 3′ orientation: a first transcription termination sequence and a marker cassette including a promoter, the marker open reading frame and a second transcription termination sequence, and said sequence interrupts the transcription of the coding sequence.

In particular, wherein said sequence sharing homologics with the regions surrounding DNA target sequence is from the Non-integrating Virus genome is a fragment of the Non-integrating Virus genome comprising sequences upstream and downstream of the cleavage site, so as to allow the deletion of coding sequences flanking the cleavage site.

According to a fourth aspect of the present invention there is provided a host cell which is modified by a polynucleotide according to a second aspect of the present invention or a vector according to a third aspect of the present invention.

A cell according to the present invention may be made according to a method, comprising at least the step of:

(a) introducing into a cell, a meganuclease, as defined above, so as to induce a double stranded cleavage at a site of interest of the Non-integrating Virus genome comprising a DNA recognition and cleavage site of said meganuclease, and thereby generate a cell comprising at least one modified Non-integrating Virus genome, in particular having repaired the double-strands break, by non-homologous end joining, and

(b) isolating the cell of step (a), by any appropriate mean.

The cell which is modified may be any cell of interest. For making transgenic/knock-out animals, the cells are pluripotent precursor cells such as embryo-derived stem (ES) cells, which are well-known in the art. For making recombinant cell lines, the cells may advantageously be human cells, for example HSV infecting cell lines such as human hepatoblastoma cell lines, hepatocellular carcinoma (Fellig et al., (2004) Biochemical and Biophysical Research Communications, Volume 321, Issue 2, Pages 269-274) or a more general cell line such as CHO or HEK293 (ATCC # CRL-1573) cells. The meganuclease can be provided directly to the cell or through an expression vector comprising the polynucleotide sequence encoding said meganuclease linked to regulatory sequences suitable for directing its expression in the cell used.

In addition to generating cells comprising modified Non-integrating Virus genomes, the present invention also relates to modifying a copy(ies) of the Non-integrating Virus genome which have been genomically integrated into the host cell genome. Such modified cell lines are useful for elucidating aspects of virus biology amongst many other potential uses.

Such a modified cell line would have a number of potential uses including the elucidation of aspects of the biology of the modified NIV genome as well as a model for screening compounds and other substances for therapeutic effects against cells comprising the modified NIV genome.

The present invention therefore also relates to meganuclease variants which can recognise and cleave targets comprised in genomic insertions of viruses which do not normally insert into the host cell genome. The non-specific insertion of viral genetic material into the host cell genome as a disease causing mechanism is currently being investigated. For example in hepatitis B, chronic infection with this virus is associated with a greatly elevated risk of hepatocellular carcinoma. In the past this association has been explained as a side effect of the episomal hepatitis B genome upon the hepatocyte host cells. Although this is doubtless true, recently the random genomic insertion of copies of the hepatitis B genome into the host cell genome has also been shown to be a causative factor in hepatocyte carcinoma (Goodarzi et al., 2008, Hep. Mon; 8 (2): 129-133).

Hepatocellular carcinoma is one of the most common cancers in the world and hence a treatment for this condition, using a meganuclease variant which can cleave the randomly integrated hepatitis B genome and have a therapeutic affect upon hepatocytes via one or more of mechanisms detailed herein is therefore also within the scope of the present invention as are other meganuclease variants to genomically integrated copies of virus genetic material which cause a disease phenotype.

Such a modified cell line would have a number of potential uses including the elucidation of aspects of the biology of the modified Non-integrating Virus genome as well as a model for screening compounds and other substances for therapeutic effects against cells comprising the modified Non-integrating Virus genome.

The present invention therefore also relates to meganuclease variants which can recognise and cleave targets comprised in genomic insertions of viruses which do not normally insert into the host cell genome. The non-specific insertion of viral genetic material into the host cell genome as a disease causing mechanism is currently being investigated.

According to a fifth aspect of the present invention there is provided a non-human transgenic animal or plant which is modified by a polynucleotide according to a second aspect of the present invention or a vector according to a third aspect of the present invention. In particular these non-human transgenic animals or transgenic plants comprise a copy of the Non-integrating Virus genome integrated into the genome of the host organism.

The subject-matter of the present invention is also a method for making a transgenic animal comprising an integrated Non-integrating Virus genome, comprising at least the step of:

(a) introducing into a pluripotent precursor cell or an embryo of an animal, a meganuclease, as defined above, so as to induce a double stranded cleavage at a site of interest of the integrated Non-integrating Virus genome comprising a DNA recognition and cleavage site of said meganuclease, and thereby generate a genomically modified precursor cell or embryo having repaired the double-strands break by non-homologous end joining,

(b) developing the genomically modified animal precursor cell or embryo of step (a) into a chimeric animal, and

(c) deriving a transgenic animal from a chimeric animal of step (b).

Alternatively, the Non-integrating Virus genome may be inactivated by insertion of a sequence of interest by homologous recombination between the genome of the animal and a targeting DNA construct according to the present invention.

Such transgenic animals/plants therefore can be used as model organisms to study the effects of genomically integrated virus genetic material which has been either introduced using a meganuclease based homologous recombination system or alternatively has been altered using a specific meganuclease variant.

In particular the targeting DNA is introduced into the cell under conditions appropriate for introduction of the targeting DNA into the site of interest.

In particular, step (b) comprises the introduction of the genomically modified precursor cell obtained in step (a), into blastocysts, so as to generate chimeric animals.

Such a transgenic animal could be used as a multicellular animal model to elucidate aspects of HSV biology by means of engineering the provirus present in the progenitor cell line. Such transgenic animals also could be used to screen and characterise the effects of novel anti-HSV medicaments.

In particular the targeting DNA construct is inserted in a vector.

For making transgenic animals/recombinant cell lines, including human cell lines expressing an heterologous protein of interest, the targeting DNA comprises the sequence of the exogenous gene encoding the protein of interest, and eventually a marker gene, flanked by sequences upstream and downstream of and essential gene in the Non-integrating Virus genome, as defined above, so as to generate genomically modified cells (animal precursor cell or embryo/animal or human cell) having replaced the HSV gene by the exogenous gene of interest, by homologous recombination.

The exogenous gene and the marker gene are inserted in an appropriate expression cassette, as defined above, in order to allow expression of the heterologous protein/marker in the transgenic animal/recombinant cell line.

The meganuclease can be used either as a polypeptide or as a polynucleotide construct encoding said polypeptide. It is introduced into somatic cells of an individual, by any convenient means well-known to those in the art, which are appropriate for the particular cell type, alone or in association with either at least an appropriate vehicle or carrier and/or with the targeting DNA.

According to the present invention, the meganuclease (polypeptide) can be associated with:
liposomes, polyethyleneimine (PEI); in such a case said association is administered and therefore introduced into somatic target cells.
membrane translocating peptides (Bonetta, The Scientist, 2002, 16, 38; Ford et al., Gene Ther., 2001, 8, 1-4; Wadia and Dowdy, Curr. Opin. Biotechnol., 2002, 13, 52-56); in such a case, the sequence of the variant/single-chain meganuclease is fused with the sequence of a membrane translocating peptide (fusion protein).

Alternatively, the meganuclease (polynucleotide encoding said meganuclease) and/or the targeting DNA is inserted in a vector. Vectors comprising targeting DNA and/or nucleic acid encoding a meganuclease can be introduced into a cell by a variety of methods (e.g., injection, direct uptake, projectile bombardment, liposomes, electroporation). Meganucleases can be stably or transiently expressed into cells using expression vectors. Techniques of expression in eukaryotic cells are well known to those in the art. (See Current Protocols in Human Genetics: Chapter 12 “Vectors For Gene Therapy” & Chapter 13 “Delivery Systems for Gene Therapy”). Optionally, it may be preferable to incorporate a nuclear localization signal into the recombinant protein to be sure that it is expressed within the nucleus.

Once in a cell, the meganuclease and if present, the vector comprising targeting DNA and/or nucleic acid encoding a meganuclease are imported or translocated by the cell from the cytoplasm to the site of action in the nucleus or the cytoplasm.

According to this aspect of the present invention there is provided a kit for carrying out the treatment of a NIV infection using an I-CreI variant according to the first aspect of the present invention, or a nucleotide molecule according to the second or third aspects of the present invention, characterized by a container with a solution comprising the following reactants:

an I-CreI variant which can recognise and cleave a DNA target sequence in the genome of the NIV; or

a nucleotide molecule which encodes an I-CreI variant which can recognise and cleave a DNA target sequence in the genome of the NIV;

any necessary preservative.

Such a kit may in particular also comprise further materials such as those necessary to allow intracellular, intranuclear entry of the active ingredient or increase its efficacy such as other anti-viral medicaments.

According to a further aspect of the present invention there is provided the use of at least one variant or at least one single-chain chimeric meganuclease according to the first aspect of the present invention, or at least one vector according to the third aspect of the present invention, for Non-integrating Virus genome engineering, for non-therapeutic or purposes.

In particular the variant or single-chain chimeric meganuclease, or vector is associated with a targeting DNA construct.

In particular the use of the variant is for inducing a double-strand break in a site of interest of the Non-integrating Virus genome comprising a Non-integrating Virus genomic DNA target sequence, thereby inducing a DNA recombination event, a DNA loss or DNA degradation.

According to the invention, said double-strand break is for: modifying a specific sequence in the Non-integrating Virus genome, so as to induce cessation of a Non-integrating Virus genome function such as replication, attenuating or activating the Non-integrating Virus genome or a gene therein, introducing a mutation into a site of interest of a Non-integrating Virus gene, introducing an exogenous gene or a part thereof, inactivating or deleting the Non-integrating Virus genome or a part thereof or leaving the DNA unrepaired and degraded.

According to this aspect of the present invention the use of the meganuclease according to the present invention, comprises at least the following steps: 1) introducing a double-strand break at a site of interest of the Non-integrating Virus genome comprising at least one recognition and cleavage site of said meganuclease, by contacting said cleavage site with said meganuclease; 2) providing a targeting DNA construct comprising the sequence to be introduced flanked by sequences sharing homologies to the targeted locus. Said meganuclease can be provided directly to the cell or through an expression vector comprising the polynucleotide sequence encoding said meganuclease and suitable for its expression in the used cell. This strategy is used to introduce a DNA sequence at the target site, for example to generate knock-in or knock-out animal models or cell lines that can be used for drug testing.

According to a further aspect of the present invention the use of the meganuclease, comprises at least the following steps: 1) introducing a double-strand break at a site of interest of the Non-integrating Virus genome comprising at least one recognition and cleavage site of said meganuclease, by contacting said cleavage site with said meganuclease; 2) maintaining said broken genomic locus under conditions appropriate for homologous recombination with chromosomal DNA sharing homologies to regions surrounding the cleavage site.

According to still further aspect of the present invention the use of the meganuclease, comprises at least the following steps: 1) introducing a double-strand break at a site of interest of the Non-integrating Virus genome comprising at least one recognition and cleavage site of said meganuclease, by contacting said cleavage site with said meganuclease; 2) maintaining said broken genomic locus under conditions appropriate for repair of the double-strands break by non-homologous end joining.

According to a further aspect of the present invention the variant is used for genome therapy or the making of knock-out Non-integrating Virus genomes, the sequence to be introduced is a sequence which inactivates the Non-integrating Virus genome. All Non-integrating Virus genomes present in the cell have to be targeted in order to totally inactivate the pathogenicity of the virus. In addition, the sequence may also delete the Non-integrating Virus genome or part thereof, and introduce an exogenous gene or part thereof (knock-in/gene replacement). For making knock-in Non-integrating Virus genomes the DNA which repairs the site of interest may comprise the sequence of an exogenous gene of interest, and a selection marker, such as the G418 resistance gene. Alternatively, the sequence to be introduced can be any other sequence used to alter the DNA in some specific way including a sequence used to modify a specific sequence, to attenuate or activate the endogenous gene of interest in the Non-integrating Virus genome or to introduce a mutation into a site of interest in the Non-integrating Virus genome.

Inactivation of the Non-integrating Virus genome may occur by insertion of a transcription termination signal that will interrupt the transcription of an essential gene such as a viral DNA polymerase and result in a truncated protein. In this case, the sequence to be introduced comprises, in the 5′ to 3′ orientation: at least a transcription termination sequence (polyA1), preferably said sequence further comprises a marker cassette including a promoter and the marker open reading frame (ORF) and a second transcription termination sequence for the marker gene ORF (polyA2). This strategy can be used with any variant cleaving a target downstream of the relevant gene promoter and upstream of the stop codon.

Inactivation of the Non-integrating Virus genome may also occur by insertion of a marker gene within an essential gene of Non-integrating Virus, which would disrupt the coding sequence. The insertion can in addition be associated with deletions of ORF sequences flanking the cleavage site and eventually, the insertion of an exogenous gene of interest (gene replacement).

In addition, inactivation of Non-integrating Virus may also occur by insertion of a sequence that would destabilize the mRNA transcript of an essential gene.

The present invention also provides a composition characterized in that it comprises at least one variant as defined above (variant or single-chain derived chimeric meganuclease) and/or at least one expression vector encoding the variant, as defined above.

In particular the composition comprises a targeting DNA construct comprising a sequence which inactivates the Non-integrating Virus genome, flanked by sequences sharing homologies with the Non-integrating Virus genomic DNA cleavage site of said variant, as defined above.

Preferably, said targeting DNA construct is either included in a recombinant vector or it is included in an expression vector comprising the polynucleotide(s) encoding the variant according to the invention.

The subject-matter of the present invention is also the use of at least one meganuclease and/or one expression vector, as defined above, for the preparation of a medicament for preventing, improving or curing a Non-integrating Virus and in particular a HSV infection in an individual in need thereof.

The subject-matter of the present invention is also the use of at least one variant and/or one expression vector, as defined above, for the preparation of a medicament for preventing, improving or curing a pathological condition associated with a Non-integrating Virus infection in an individual in need thereof.

In particular compositions according to the present invention may comprise more than one variant. The genome of a virus is subject to more changes than the genome of a higher organism such as a prokaryotic or eukaryotic cell. Therefore in a population of viruses in an infected individual it is possible that the DNA target recognized by the variant will be altered and hence the variant will not cut this target. To lessen the potential effects of such mutants, compositions according to the present invention may comprise variants which recognize and cleave different targets in the Non-integrating Virus genome. The chances of a particular virus having mutations in all the various targets cleaved by the variants contained in the composition are very low and hence the virus will be recognized and acted upon by at least one of the variants present in the composition.

The use of the meganuclease may comprise at least the step of (a) inducing in at least one Non-integrating Virus genome contained in an at least one cell of infected individual a double stranded cleavage at a site of interest of the Non-integrating Virus genome comprising at least one recognition and cleavage site of said meganuclease by contacting said cleavage site with said meganuclease, and (b) introducing into said at least one cell a targeting DNA, wherein said targeting DNA comprises (1) DNA sharing homologies to the region surrounding the cleavage site and (2) DNA which inactivates the Non-integrating Virus genome upon recombination between the targeting DNA and the Non-integrating Virus genome, as defined above. The targeting DNA is introduced into the Non-integrating Virus genome under conditions appropriate for introduction of the targeting DNA into the site of interest. The targeting construct may comprise sequences for deleting the Non-integrating Virus genome or a portion thereof and introducing the sequence of an exogenous gene of interest (gene replacement).

Alternatively, the Non-integrating Virus genome may be inactivated by the mutagenesis of an open reading frame therein, by the repair of the double-strands break by non-homologous end joining. In the absence of a repair matrix, the DNA double-strand break in an exon will be repaired essentially by the error-prone Non Homologous End Joining pathway NHEJ, resulting in small deletions (a few nucleotides) or small insertions (a few nucleotides), that will inactivate the cleavage site, and result in frame shift mutation.

In this case the use of the meganuclease comprises at least the step of: inducing in virus infected tissue(s) of the an individual a double stranded cleavage at a site of interest of in the Non-integrating Virus genome comprising at least one recognition and cleavage site of the meganuclease by contacting the cleavage site with the meganuclease, and thereby inducing mutagenesis of an open reading frame in the Non-integrating Virus genome by repair of the double-strands break by non-homologous end joining.

According to the present invention, said double-stranded cleavage may be induced, ex vivo by introduction of said meganuclease into infected cells isolated for instance from the circulatory system of the donor/individual and then transplantation of the modified cells back into the diseased individual.

The subject-matter of the present invention is also a method for preventing, improving or curing NIV infection and in particular a Herpes Simplex Virus Type 1 or Type 2 infection or Hepatitis B virus infection, in an individual in need thereof, said method comprising at least the step of administering to said individual a composition as defined above, by any means.

For purposes of therapy, the meganucleases and a pharmaceutically acceptable excipient are administered in a therapeutically effective amount. Such a combination is said to be administered in a “therapeutically effective amount” if the amount administered is physiologically significant. An agent is physiologically significant if its presence results in a detectable change in the physiology of the recipient. In the present context, an agent is physiologically significant if its presence results in a decrease in the severity of one or more symptoms of the targeted Non-integrating Virus and in particular Herpes Simplex Virus Type 1 or 2 infection.

In particular as far as possible the meganuclease comprising compositions should be non-immunogenic, i.e., engender little or no adverse immunological response. A variety of methods for ameliorating or eliminating deleterious immunological reactions of this sort can be used in accordance with the invention. One means of achieving this is to ensure that the meganuclease is substantially free of N-formyl methionine. Another way to avoid unwanted immunological reactions is to conjugate meganucleases to polyethylene glycol (“PEG”) or polypropylene glycol (“PPG”) (preferably of 500 to 20,000 Daltons average molecular weight (MW)). Conjugation with PEG or PPG, as described by Davis et al. (U.S. Pat. No. 4,179,337) for example, can provide non-immunogenic, physiologically active, water soluble endonuclease conjugates with anti-viral activity. Similar methods also using a polyethylene-polypropylene glycol copolymer are described in Saifer et al. (U.S. Pat. No. 5,006,333).

### DEFINITIONS

Throughout the present patent application a number of terms and features are used to present and describe the present invention, to clarify the meaning of these terms a number of definitions are set out below and wherein a feature or term is not otherwise specifically defined or obvious from its context the following definitions apply.
Amino acid residues in a polypeptide sequence are designated herein according to the one-letter code, in which, for example, Q means Gin or Glutamine residue, R means Arg or Arginine residue and D means Asp or Aspartic acid residue.
Amino acid substitution means the replacement of one amino acid residue with another, for instance the replacement of an Arginine residue with a Glutamine residue in a peptide sequence is an amino acid substitution.
Altered/enhanced/increased/improved cleavage activity, refers to an increase in the detected level of meganuclease cleavage activity, see below, against a target DNA sequence by a second meganuclease in comparison to the activity of a first meganuclease against the target DNA sequence. Normally the second meganuclease is a variant of the first and comprise one or more substituted amino acid residues in comparison to the first meganuclease.
by “beta-hairpin” it is intended two consecutive beta-strands of the antiparallel beta-sheet of a LAGLIDADG homing endonuclease core domain (β₁β₂ or β₃β₄) which are connected by a loop or a turn,
by “chimeric DNA target” or “hybrid DNA target” it is intended the fusion of a different half of two parent meganuclease target sequences. In addition at least one half of said target may comprise the combination of nucleotides which are bound by at least two separate subdomains (combined DNA target).
Cleavage activity: the cleavage activity of the variant according to the invention may be measured by any well-known, in vitro or in vivo cleavage assay, such as those described in the International PCT Application WO 2004/067736; Epinat et al., Nucleic Acids Res., 2003, 31, 2952-2962; Chames et al., Nucleic Acids Res., 2005, 33, e178; Arnould et al., J. Mol. Biol., 2006, 355, 443-458, and Arnould et al., J. Mol. Biol., 2007, 371, 49-65. For example, the cleavage activity of the variant of the invention may be measured by a direct repeat recombination assay, in yeast or mammalian cells, using a reporter vector. The reporter vector comprises two truncated, non-functional copies of a reporter gene (direct repeats) and the genomic (non-palindromic) DNA target sequence within the intervening sequence, cloned in a yeast or a mammalian expression vector. Usually, the genomic DNA target sequence comprises one different half of each (palindromic or pseudo-palindromic) parent homodimeric meganuclease target sequence. Expression of the heterodimeric variant results in a functional endonuclease which is able to cleave the genomic DNA target sequence. This cleavage induces homologous recombination between the direct repeats, resulting in a functional reporter gene (LacZ, for example), whose expression can be monitored by an appropriate assay. The specificity of the cleavage by the variant may be assessed by comparing the cleavage of the (non-palindromic) DNA target sequence with that of the two palindromic sequences cleaved by the parent homodimeric meganucleases or compared with wild type meganuclease.
by “selection or selecting” it is intended to mean the isolation of one or more meganuclease variants based upon an observed specified phenotype, for instance altered cleavage activity. This selection can be of the variant in a peptide form upon which the observation is made or alternatively the selection can be of a nucleotide coding for selected meganuclease variant.
by “screening” it is intended to mean the sequential or simultaneous selection of one or more meganuclease variant (s) which exhibits a specified phenotype such as altered cleavage activity.
by “derived from” it is intended to mean a meganuclease variant which is created from a parent meganuclease and hence the peptide sequence of the meganuclease variant is related to (primary sequence level) but derived from (mutations) the sequence peptide sequence of the parent meganuclease.
by “domain” or “core domain” it is intended the “LAGLIDADG homing endonuclease core domain” which is the characteristic α₁β₁β₂α₂β₃β₄α₃ fold of the homing endonucleases of the LAGLIDADG family, corresponding to a sequence of about one hundred amino acid residues. Said domain comprises four beta-strands (β₁β₂β₃β₄) folded in an antiparallel beta-sheet which interacts with one half of the DNA target. This domain is able to associate with another LAGLIDADG homing endonuclease core domain which interacts with the other half of the DNA target to form a functional endonuclease able to cleave said DNA target. For example, in the case of the dimeric homing endonuclease I-CreI (163 amino acids), the LAGLIDADG homing endonuclease core domain corresponds to the residues 6 to 94.
by “DNA target”, “DNA target sequence”, “target sequence”, “target-site”, “target”, “site”; “site of interest”; “recognition site”, “recognition sequence”, “homing recognition site”, “homing site”, “cleavage site” it is intended a 20 to 24 bp double-stranded palindromic, partially palindromic (pseudo-palindromic) or non-palindromic polynucleotide sequence that is recognized and cleaved by a LAGLIDADG homing endonuclease such as I-CreI, or a variant, or a single-chain chimeric meganuclease derived from I-CreI. These terms refer to a distinct DNA location, preferably a genomic location, at which a double stranded break (cleavage) is to be induced by the meganuclease. The DNA target is defined by the 5′ to 3′ sequence of one strand of the double-stranded polynucleotide, as indicated for C1221 (see FIG. 3, SEQ ID NO: 2). Cleavage of the DNA target occurs at the nucleotides at positions +2 and −2, respectively for the sense and the antisense strand. Unless otherwise indicated, the position at which cleavage of the DNA target by an I-CreI meganuclease variant occurs, corresponds to the cleavage site on the sense strand of the DNA target.
by “DNA target half-site”, “half cleavage site” or half-site” it is intended the portion of the DNA target which is bound by each LAGLIDADG homing endonuclease core domain.
by “DNA target sequence from the HBV genome” it is intended a 20 to 24 bp sequence of the HBV genome which is recognized and cleaved by a meganuclease variant. In particular the DNA target sequence from then HBV genome is in an essential gene sequence and/or within an essential regulatory sequence and/or within an essential structural sequence of the HBV genome.
by “DNA target sequence from the HSV genome” it is intended a 20 to 24 bp sequence of the HSV genome which is recognized and cleaved by a meganuclease variant. In particular the DNA target sequence from then HSV genome is in an essential gene sequence and/or within an essential regulatory sequence and/or within an essential structural sequence of the HSV genome.
by “first/second/third/n^{th} series of variants” it is intended a collection of variant meganucleases, each of which comprises one or more amino acid substitution in comparison to a parent meganuclease from which all the variants in the series are derived.
by “functional variant” it is intended a variant which is able to cleave a DNA target sequence, preferably said target is a new target which is not cleaved by the parent meganuclease. For example, such variants have amino acid variation at positions contacting the DNA target sequence or interacting directly or indirectly with said DNA target.
by “heterodimer” it is intended to mean a meganuclease comprising two non-identical monomers. In particular the monomers may differ from each other in their peptide sequence and/or in the DNA target half-site which they recognise and cleave.
by “homologous” is intended a sequence with enough identity to another one to lead to a homologous recombination between sequences, more particularly having at least 95% identity, preferably 97% identity and more preferably 99%.
by “I-CreI” it is intended the wild-type I-CreI having the sequence of pdb accession code 1 g9y, corresponding to the sequence SEQ ID NO: 1 in the sequence listing.
by “I-CreI variant with novel specificity” it is intended a variant having a pattern of cleaved targets different from that of the parent meganuclease. The terms “novel specificity”, “modified specificity”, “novel cleavage specificity”, “novel substrate specificity” which are equivalent and used indifferently, refer to the specificity of the variant towards the nucleotides of the DNA target sequence. In the present patent application the I-CreI variants described comprise an additional Alanine after the first Methionine of the wild type I-CreI sequence and three additional amino acid residues (SEQ ID NO: 3). In the present application, I-CreI variants may be homodimers (meganuclease comprising two identical monomers) or heterodimers (meganuclease comprising two non-identical monomers).

These variants also comprise two additional Alanine residues and an Aspartic Acid residue after the final Proline of the wild type I-CreI sequence. These additional residues do not affect the properties of the enzyme and to avoid confusion these additional residues do not affect the numeration of the residues in I-CreI or a variant referred in the present patent application, as these references exclusively refer to residues of the wild type I-CreI enzyme (SEQ ID NO: 1) as present in the variant, so for instance residue 2 of I-CreI is in fact residue 3 of a variant which comprises an additional Alanine after the first Methionine.
by “I-CreI site” it is intended a 22 to 24 bp double-stranded DNA sequence which is cleaved by I-CreI. I-CreI sites include the wild-type non-palindromic I-CreI homing site and the derived palindromic sequences such as the sequence 5′-t₋₁₂c₋₁₁a₋₁₀a₋₉a₋₈a₋₇c₋₆g₋₂t₋₁a₊₁c₊₂g₊₃a₊₄c₊₅g₊₆t₊₇t₊₈t₊₉t₊₁₀g₊₁₁a₊₁₂ (SEQ ID NO: 2), also called C1221.
“identity” refers to sequence identity between two nucleic acid molecules or polypeptides. Identity can be determined by comparing a position in each sequence which may be aligned for purposes of comparison. When a position in the compared sequence is occupied by the same base, then the molecules are identical at that position. A degree of similarity or identity between nucleic acid or amino acid sequences is a function of the number of identical or matching nucleotides at positions shared by the nucleic acid sequences. Various alignment algorithms and/or programs may be used to calculate the identity between two sequences, including FASTA, or BLAST which are available as a part of the GCG sequence analysis package (University of Wisconsin, Madison, Wis.), and can be used with, e.g., default settings.
by “meganuclease”, it is intended an endonuclease having a double-stranded DNA target sequence of 12 to 45 bp. The meganuclease is either a dimeric enzyme, wherein each domain is on a monomer or a monomeric enzyme comprising the two domains on a single polypeptide.
by “meganuclease domain”, it is intended the region which interacts with one half of the DNA target of a meganuclease and is able to associate with the other domain of the same meganuclease which interacts with the other half of the DNA target to form a functional meganuclease able to cleave said DNA target.
by “meganuclease variant” or “variant” it is intended a meganuclease obtained by replacement of at least one residue in the amino acid sequence of the parent meganuclease with a different amino acid.
by “monomer” it is intended to mean a peptide encoded by the open reading frame of the I-CreI gene or a variant thereof, which when allowed to dimerise forms a functional I-CreI enzyme. In particular the monomers dimerise via interactions mediated by the LAGLIDADG motif.
by “mutation” is intended the substitution, deletion, insertion of one or more nucleotides/amino acids in a polynucleotide (cDNA, gene) or a polypeptide sequence. Said mutation can affect the coding sequence of a gene or its regulatory sequence. It may also affect the structure of the genomic sequence or the structure/stability of the encoded mRNA.
Nucleotides are designated as follows: one-letter code is used for designating the base of a nucleoside: a is adenine, t is thymine, u is uracile, c is cytosine, and g is guanine. For the degenerated nucleotides, r represents g or a (purine nucleotides), k represents g or t, s represents g or c, w represents a or t, m represents a or c, y represents t or c (pyrimidine nucleotides), d represents g, a or t, v represents g, a or c, b represents g, t or c, h represents a, t or c, and n represents g, a, t or c.
by “parent meganuclease” it is intended to mean a wild type meganuclease or a variant of such a wild type meganuclease with identical properties or alternatively a meganuclease with some altered characteristic in comparison to a wild type version of the same meganuclease. In the present invention the parent meganuclease can refer to the initial meganuclease from which the first series of variants are derived in step a, or the meganuclease from which the second series of variants are derived in step b., or the meganuclease from which the third series of variants are derived in step k.
by “peptide linker” it is intended to mean a peptide sequence of at least 10 and preferably at least 17 amino acids which links the C-terminal amino acid residue of the first monomer to the N-terminal residue of the second monomer and which allows the two variant monomers to adopt the correct conformation for activity and which does not alter the specificity of either of the monomers for their targets.
by “subdomain” it is intended the region of a LAGLIDADG homing endonuclease core domain which interacts with a distinct part of a homing endonuclease DNA target half-site.
by “single-chain meganuclease”, “single-chain chimeric meganuclease”, “single-chain meganuclease derivative”, “single-chain chimeric meganuclease derivative” or “single-chain derivative” it is intended a meganuclease comprising two LAGLIDADG homing endonuclease domains or core domains linked by a peptidic spacer. The single-chain meganuclease is able to cleave a chimeric DNA target sequence comprising one different half of each parent meganuclease target sequence.
by “single-chain obligate heterodimer”, it is intended a single-chain derived from an obligate heterodimer, as defined above.
by “targeting DNA construct/minimal repair matrix/repair matrix” it is intended to mean a DNA construct comprising a first and second portions which are homologous to regions 5′ and 3′ of the DNA target in situ. The DNA construct also comprises a third portion positioned between the first and second portion which comprise some homology with the corresponding DNA sequence in situ or alternatively comprise no homology with the regions 5′ and 3′ of the DNA target in situ. Following cleavage of the DNA target, a homologous recombination event is stimulated between the genome containing the Non-integrating Virus genome and the repair matrix, wherein the genomic sequence containing the DNA target is replaced by the third portion of the repair matrix and a variable part of the first and second portions of the repair matrix.
by “vector” is intended a nucleic acid molecule capable of transporting another nucleic acid to which it has been linked into a host cell in vitro, in vivo or ex vivo.

For a better understanding of the invention and to show how the same may be carried into effect, there will now be shown by way of example only, specific embodiments, methods and processes according to the present invention with reference to the accompanying drawings in which:

FIG. 1: HSV-1 genome schematic representation. Gene considered as accessory (upper) and essential (down) are represented from both parts of linear form of virus DNA.

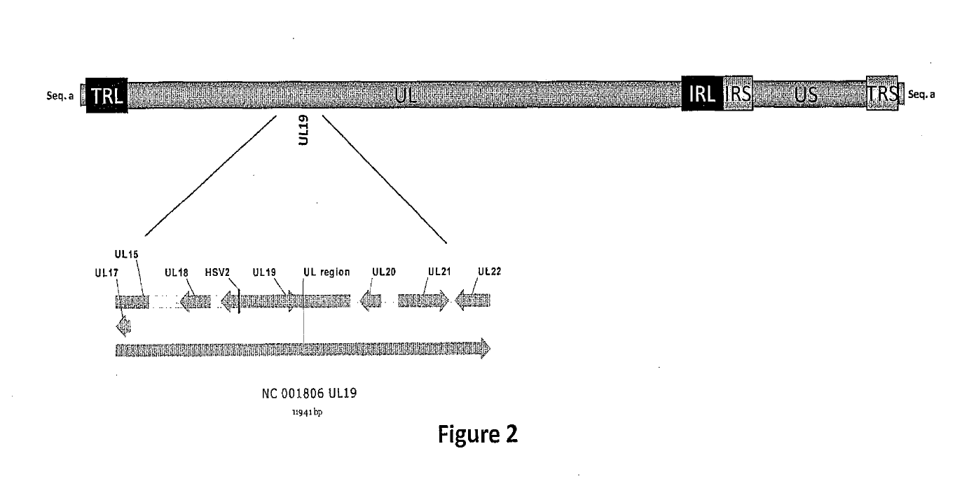

FIG. 2: HSV-1 genome schematic representation with HSV2 and UL19 localization

FIG. 3: The HSV2 and C1221 I-CreI target sequences and their derivatives. 10AAA_P, 5CAC_P, 10AGG_P, 5GCC_P are close derivatives found to be cleaved by previously obtained I-CreI mutants. They differ from C1221 by the boxed motives. C1221, 10AAAP_P, 5CAC_P, 10AGG_P, 5GCC_P were first described as 24 bp sequences, but structural data suggest that only the 22 bp are relevant for protein/DNA interaction. However, positions 12 are indicated in parenthesis. In the HSV2.2 target, the ACAC sequence in the middle of the target is replaced with GTAC, the bases found in C1221. HSV2.3 is the palindromic sequence derived from the left part of HSV2.2, and HSV2.4 is the palindromic sequence derived from the right part of HSV2.2. As shown in the Figure, the boxed motives from 10AAA_P, 5CAC_P, 10AGG_P, 5GCC_P are found in the HSV2 series of targets

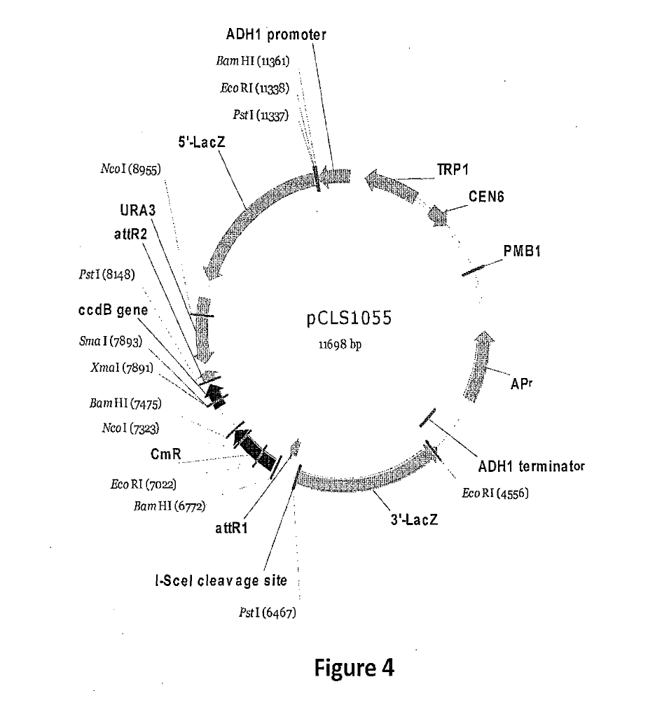

FIG. 4: pCLS1055

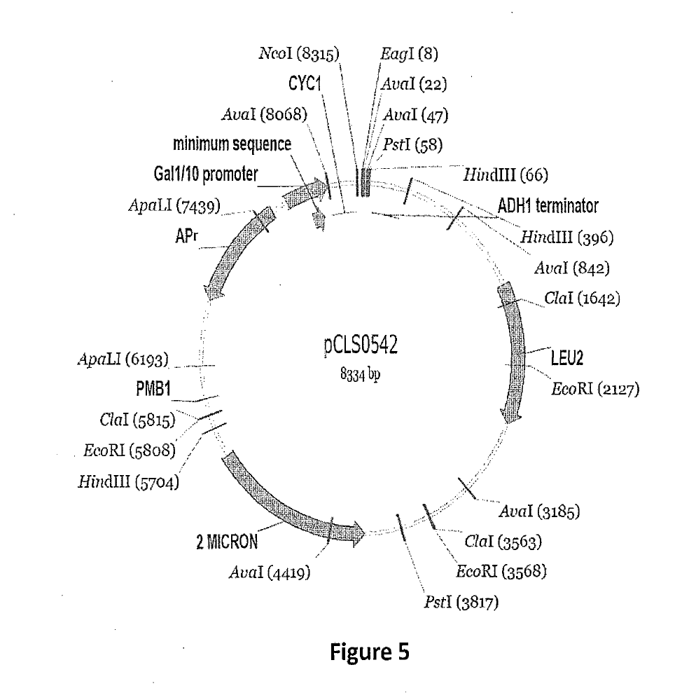

FIG. 5: pCLS0542

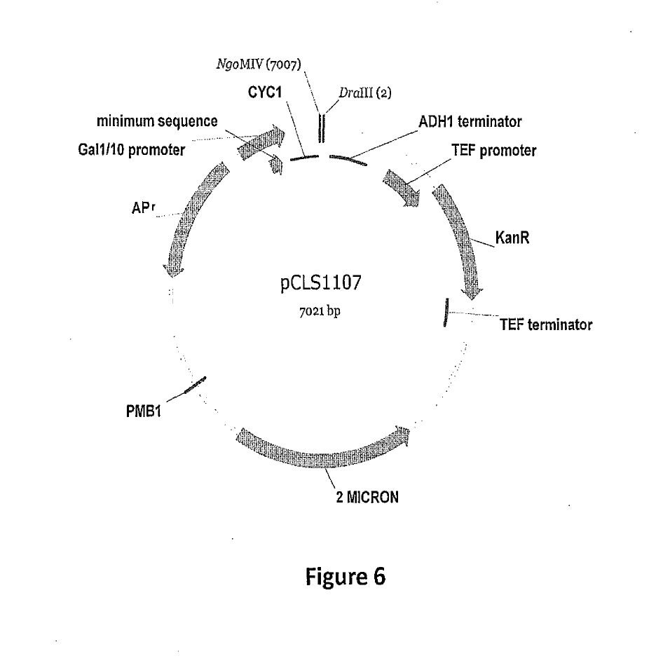

FIG. 6: pCLS1107

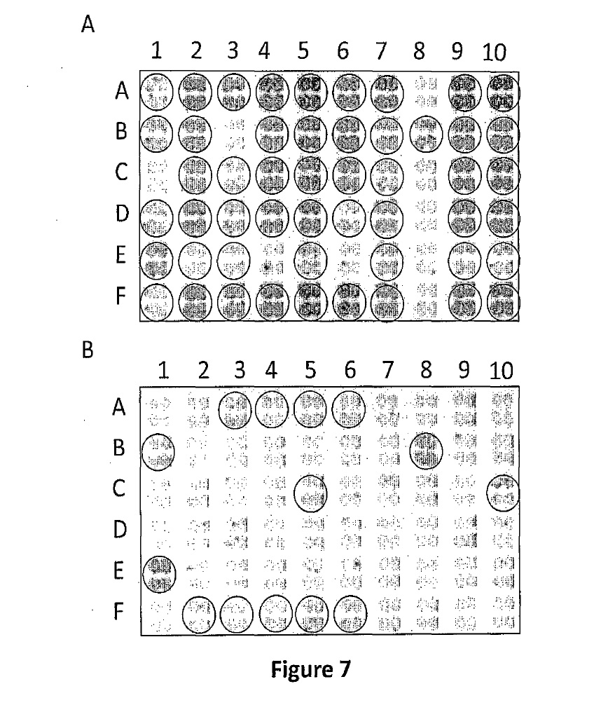

FIG. 7: Cleavage of HSV2.2 and HSV2 by heterodimeric mutants from database. A. Secondary screening of combinations of I-CreI mutants with the HSV2.2. target. B. Secondary screening of the same combinations of I-CreI mutants with the HSV2 target.

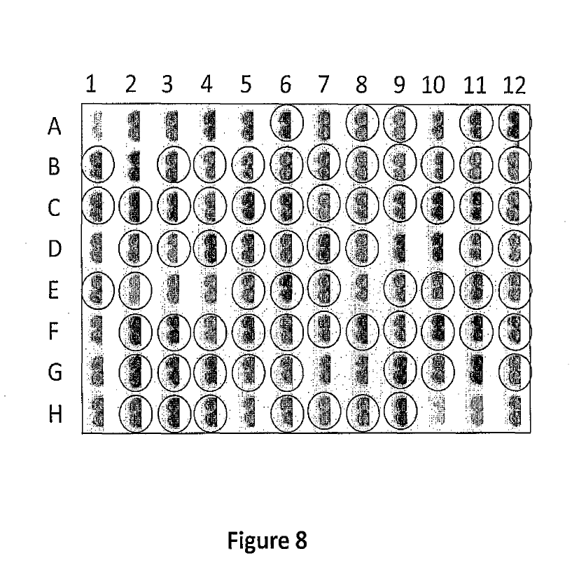

FIG. 8: Improvement of HSV2.5 cleavage: A series of I-CreI N75 mutants cutting HSV2.3 and HSV2.5 were optimized by random mutagenesis. Cleavage is tested with the HSV2.5 target. Mutants displaying high specific cleavage activity of HSV2.5 (and HSV2.3) are circled. H10 is a negative control. H11 and H12 are positive controls.

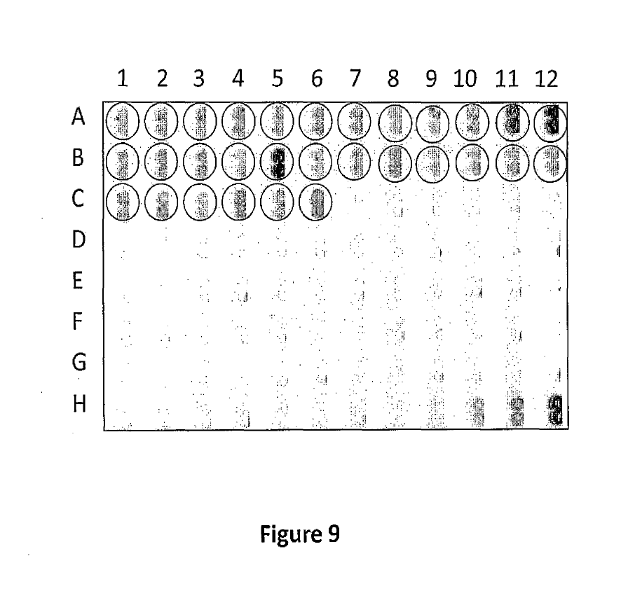

FIG. 9: Improvement of HSV2.6 cleavage: A series of I-CreI N75 mutants cutting HSV2.4 and HSV2.6 were optimized by random mutagenesis. Cleavage is tested with the HSV2.6 target (panel A) and HSV2.4 (panel B). Mutants displaying specific cleavage activity of HSV2.6 (and HSV2.4) are circled. D10 is a negative control. D11 and D12 are positive controls.

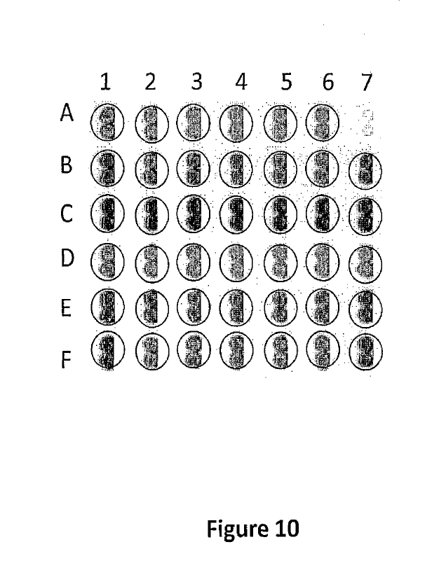

FIG. 10: Cleavage of HSV2 by optimized heterodimeric mutants from random mutagenesis. Combinations displaying high cleavage activity of HSV2 are circled.

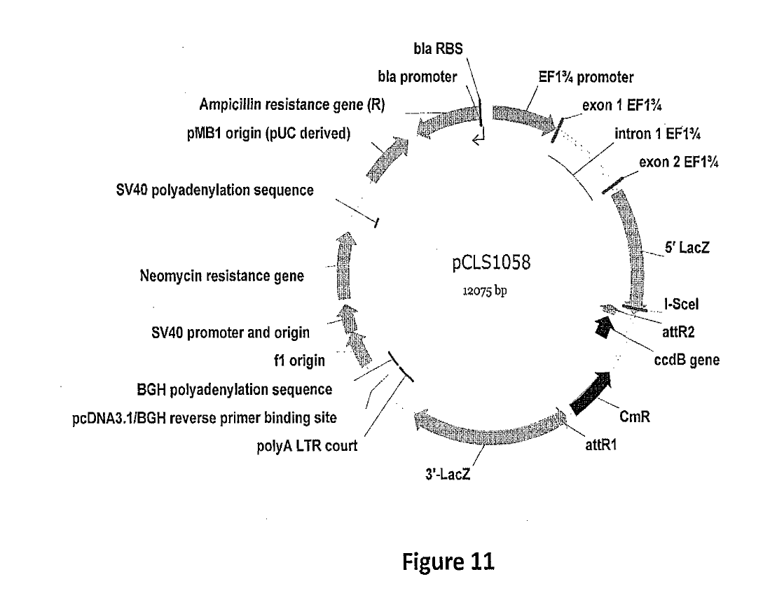

FIG. 11: pCLS1058

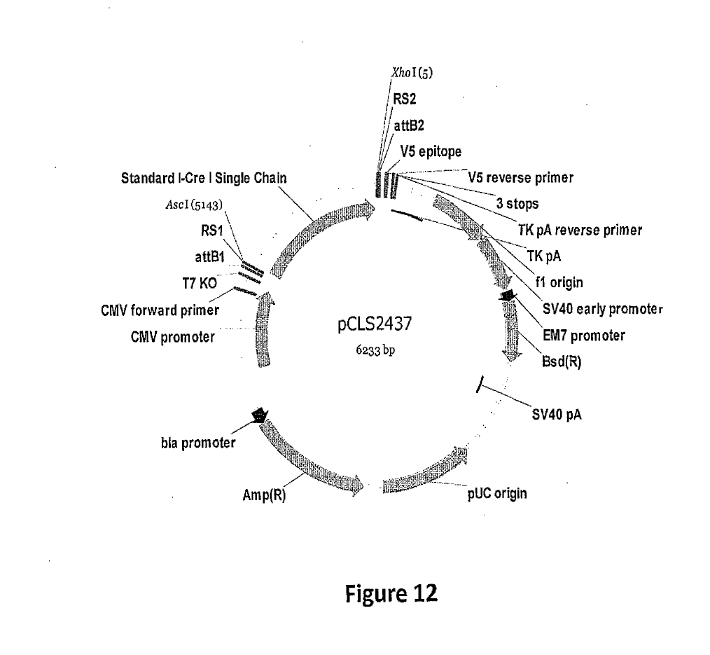

FIG. 12: pCLS2437

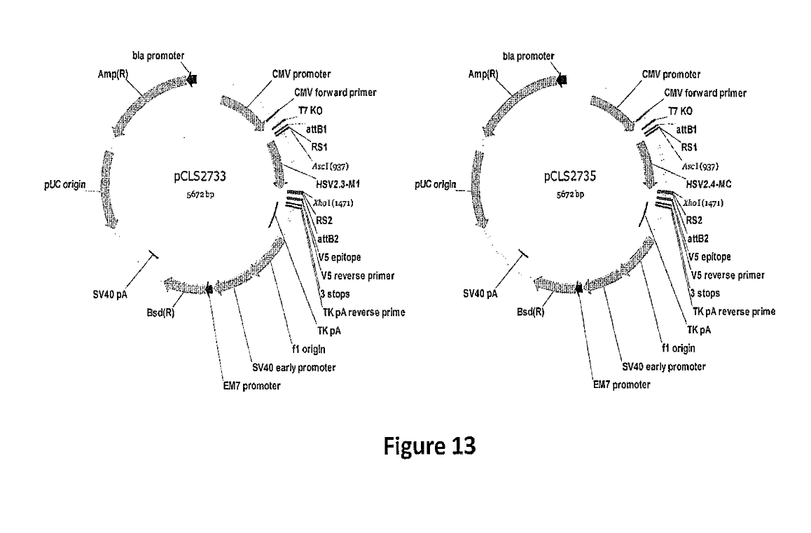

FIG. 13: pCLS2733 and pCLS2735

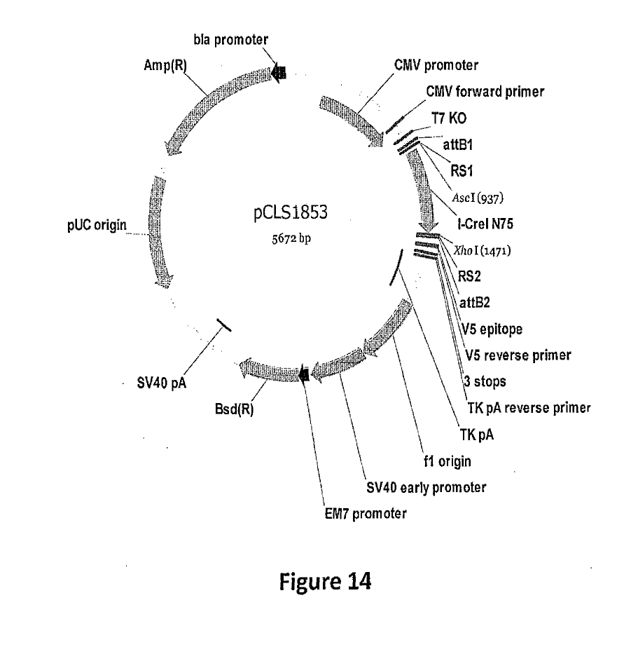

FIG. 14: pCLS1853

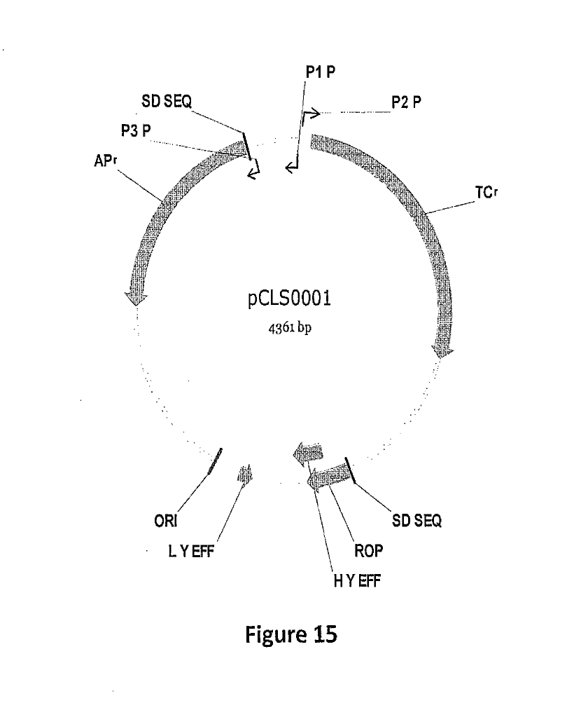

FIG. 15: pCLS0001

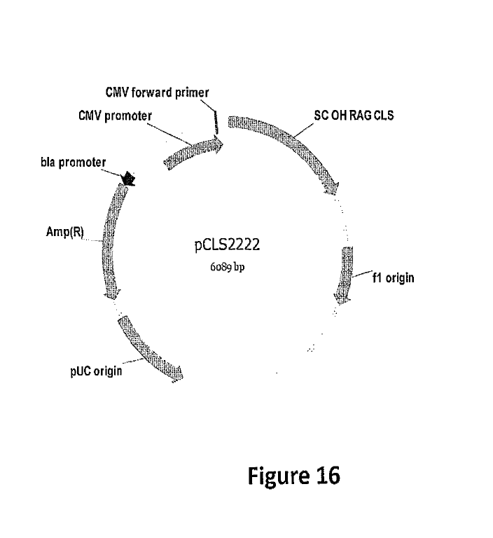

FIG. 16: pCLS2222 positive control expressing SCOH-RAG1.10 meganuclease.

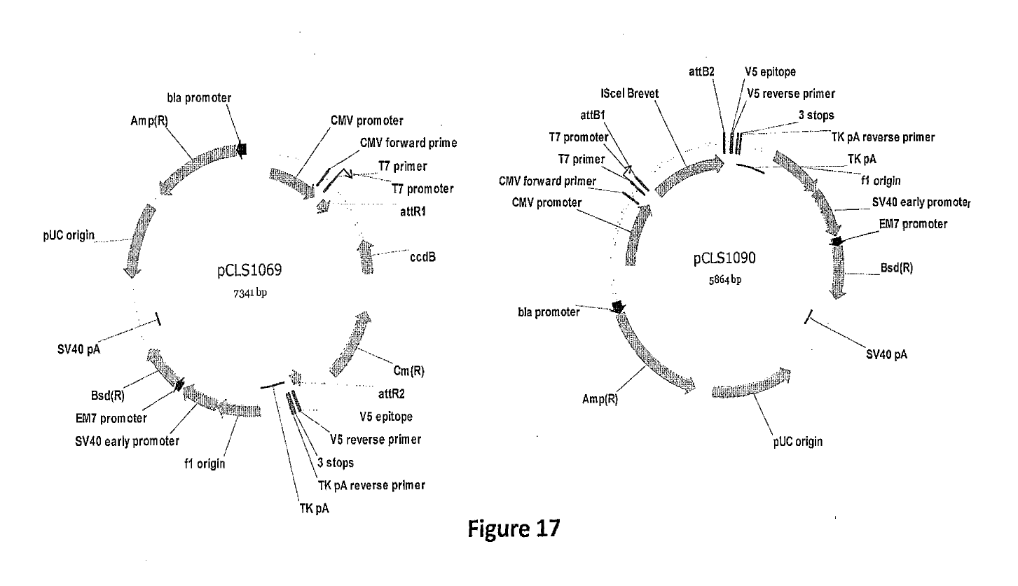

FIG. 17: pCLS1069 (empty vector) and pCLS1090 (positive control expressing I-SceI)

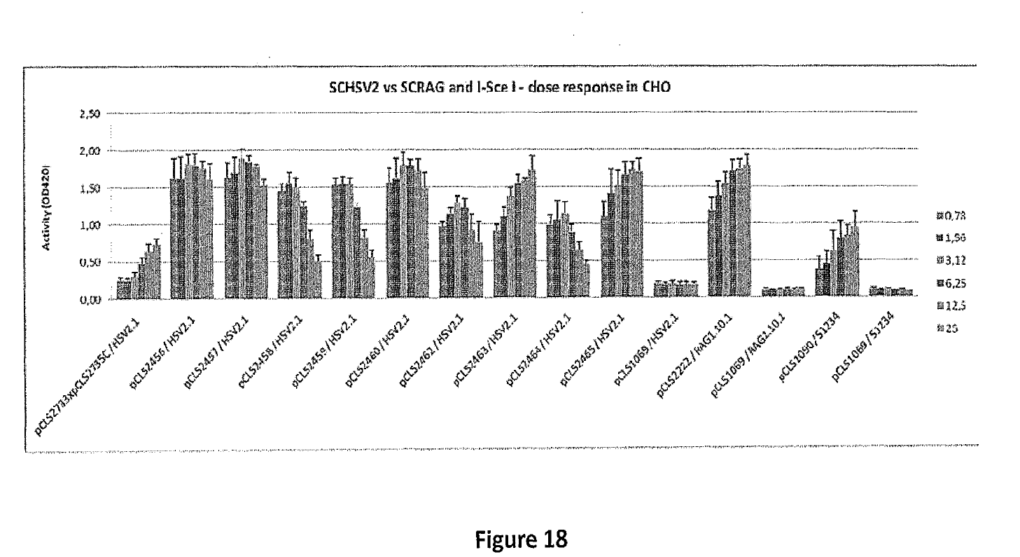

FIG. 18: Example of activity cleavage in CHO cells of designed single chain SCOH-HSV2 variants compared to initial heterodimer, I-SceI and SCOH-RAG1.10 meganucleases as positive controls.

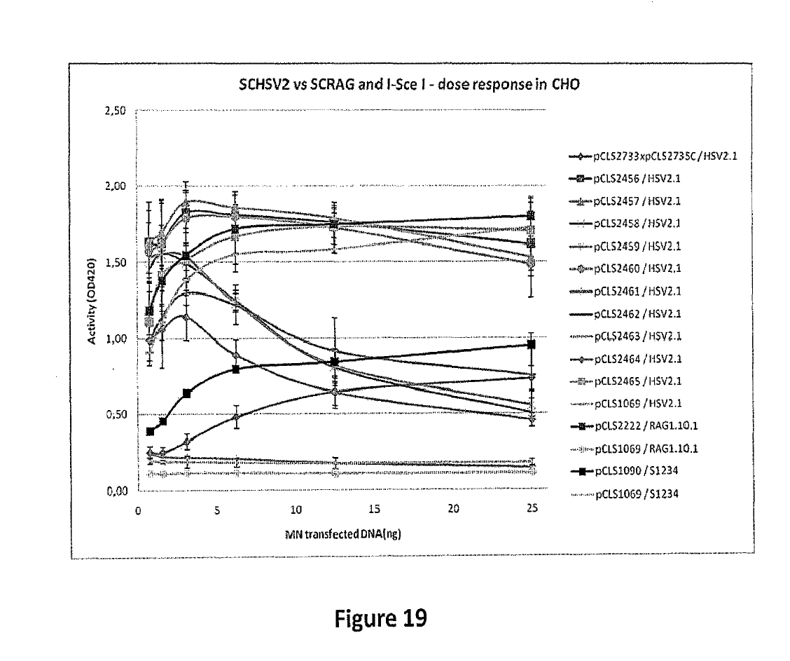

FIG. 19: Example of activity cleavage in CHO cells of single chain SCOH-HSV2 variants compared to initial heterodimer, I-SceI and SCOH-RAG1.10 meganucleases as positive controls.

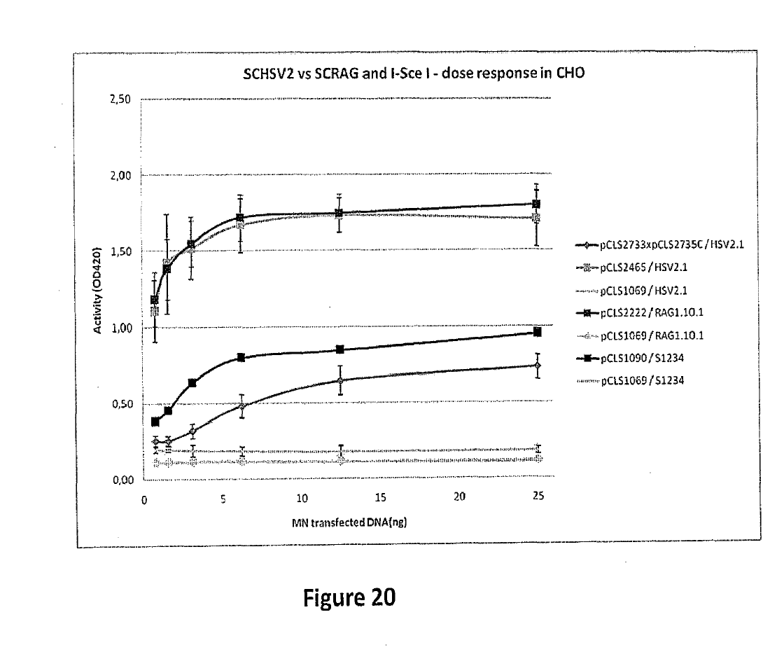

FIG. 20: Example of activity cleavage in CHO cells of single chain SCOH-HSV2-M1-105A132V-MC132V compared to initial heterodimer, I-SceI and SCOH-RAG1.10 meganucleases as positive controls.

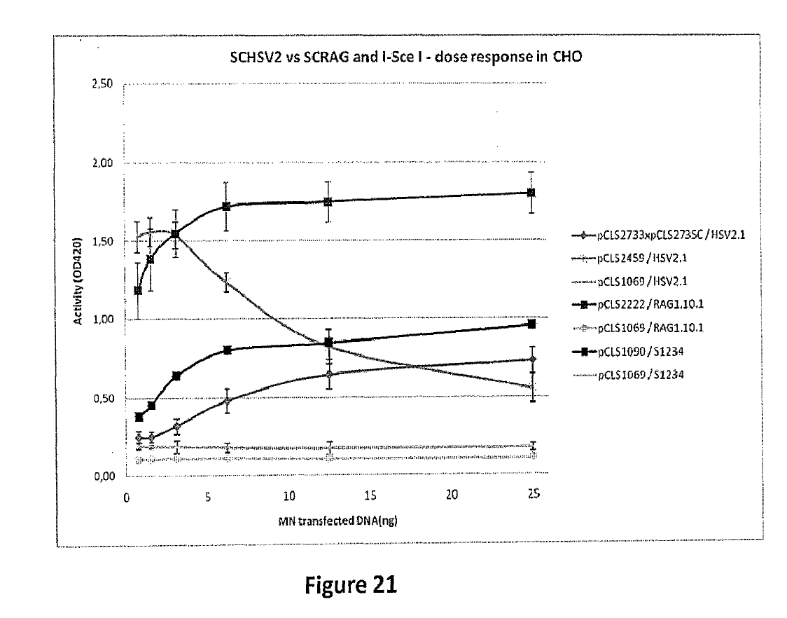

FIG. 21: Example of activity cleavage in CHO cells of single chain SCOH-HSV2-M1-MC-80K105A132V (pCLS2459) compared to initial heterodimer, I-SceI and SCOH-RAG1.10 meganucleases as positive controls.

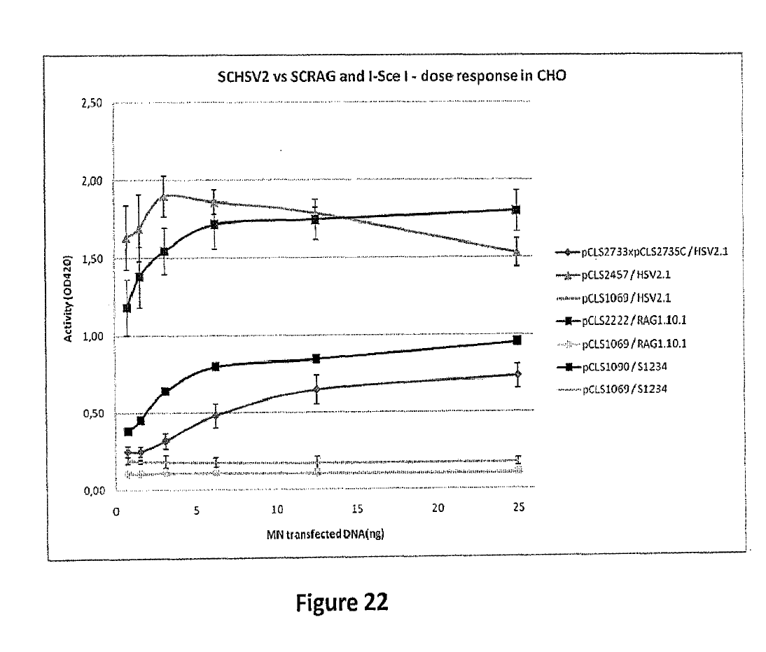

FIG. 22: Example of activity cleavage in CHO cells of single chain SCOH-HSV2-M1-MC-132V (pCLS2457) compared to initial heterodimer, I-SceI and SCOH-RAG1.10 meganucleases as positive controls.

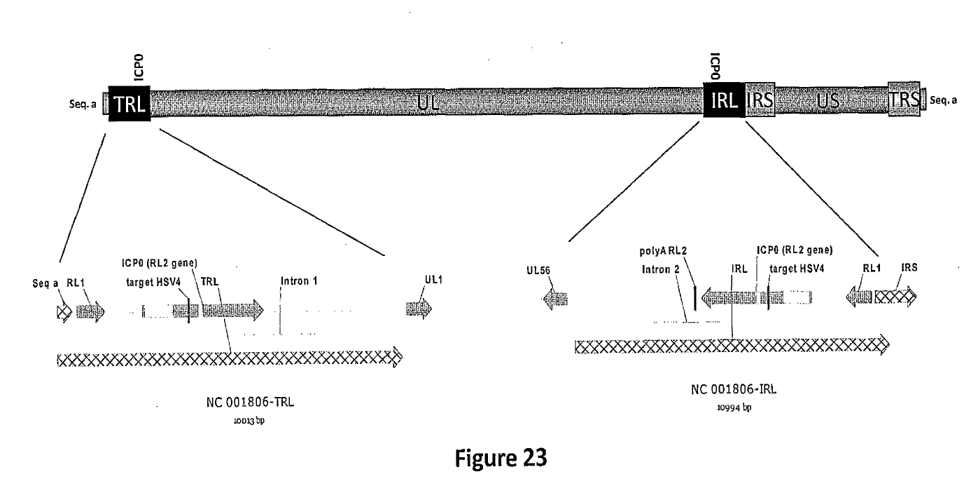

FIG. 23: HSV-1 genome schematic representation with HSV4 and ICP0 (or RL2) genes localization

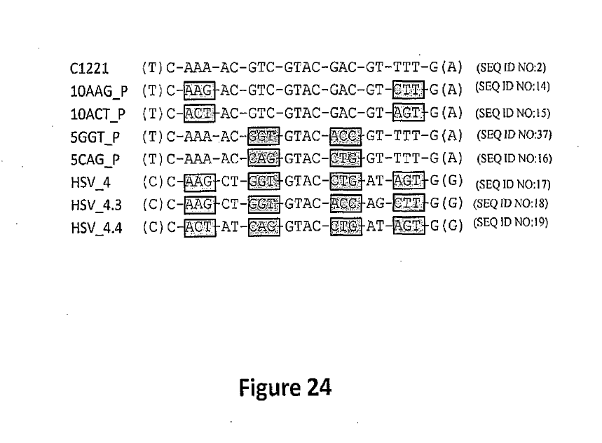

FIG. 24: The HSV4 and C1221 I-Cre I target sequences and their derivatives. 10AAG_P, 5GGT_P, 5CAG_P, 10ACT_P are close derivatives found to be cleaved by previously obtained I-CreI mutants. They differ from C1221 by the boxed motives. C1221, 10AAG_P, 5GGT_P, 5CAG_P, 10ACT_P were first described as 24 bp sequences, but structural data suggest that only the 22 bp are relevant for protein/DNA interaction. However, positions ±12 are indicated in parenthesis. In the HSV4 target, the GTAC sequence in the middle of the target is found in C1221. HSV4.3 is the palindromic sequence derived from the left part of HSV4, and HSV4.4 is the palindromic sequence derived from the right part of HSV4. As shown in the figure, the boxed motives from 10AAG_P, 5GGT_P, 5CAG_P, 10ACT_P are found in the HSV4 series of targets

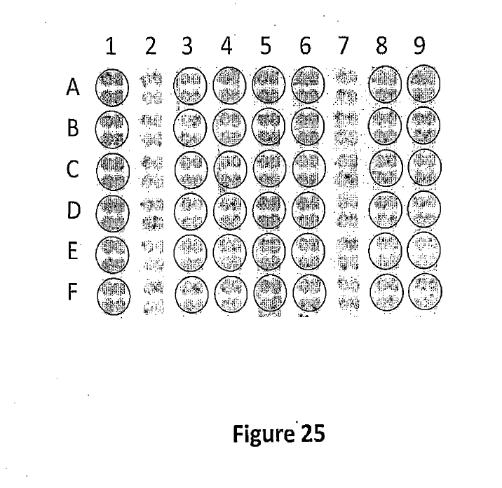

FIG. 25: Cleavage of HSV4 by heterodimeric combinations of mutants obtained after combinatorial process.

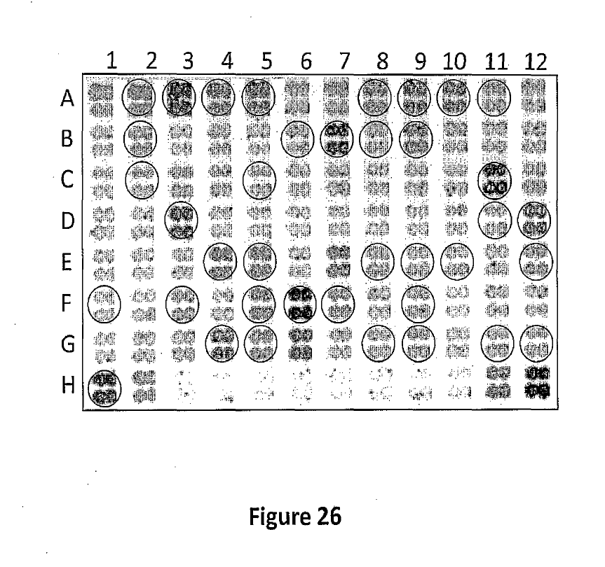

FIG. 26: Improvement of HSV4.3 cleavage: A series of I-CreI N75 mutants cutting HSV4.3 were optimized by random mutagenesis. Cleavage is tested with the HSV4.3 target. Mutants displaying high specific cleavage activity of HSV4.3 are circled. H10 is a negative control. H11 and H12 are positive controls.

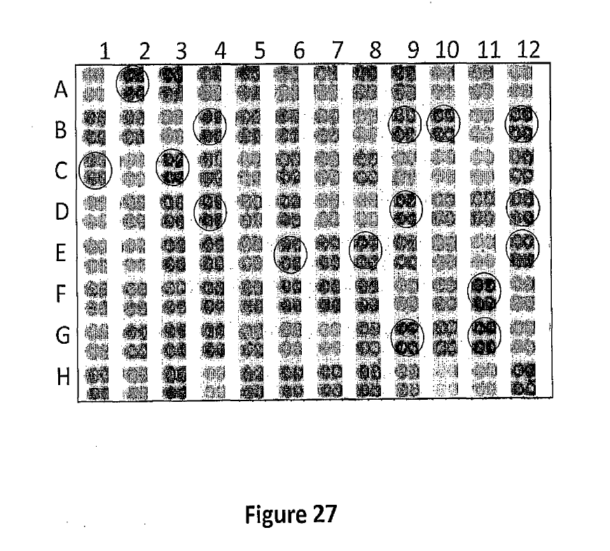

FIG. 27: Improvement of HSV4.4 cleavage: A series of I-CreI N75 mutants cutting HSV4.4 were optimized by random mutagenesis. Cleavage is tested with the HSV4.4 target. 14 mutants displaying higher specific cleavage activity of HSV4.4 than best starting one are circled. H10 is a negative control. H11 and H12 are positive controls.

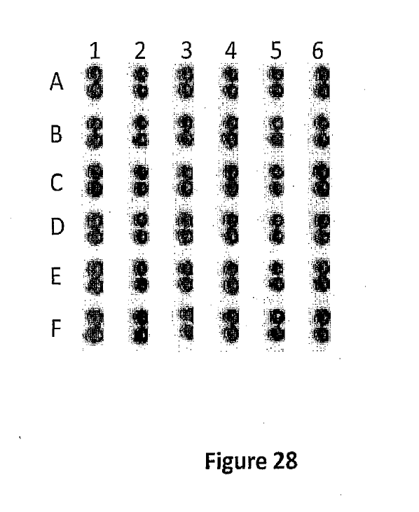

FIG. 28: Cleavage of HSV4 by optimized heterodimeric mutants from random mutagenesis. All combinations are displaying high cleavage activity of HSV4.

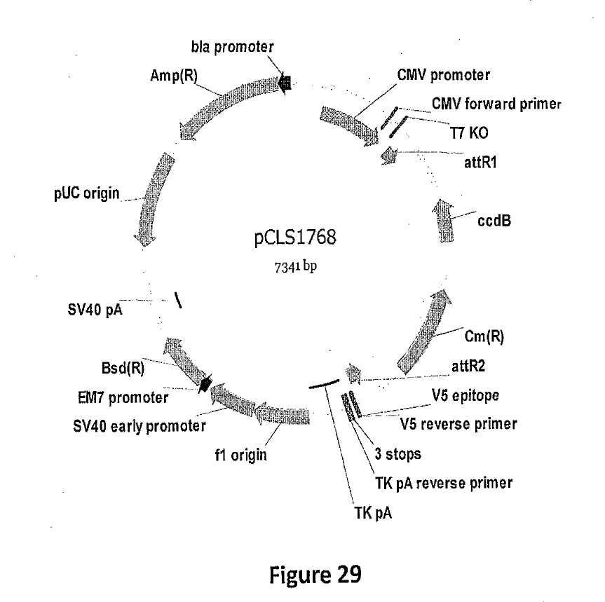

FIG. 29: pCLS1768

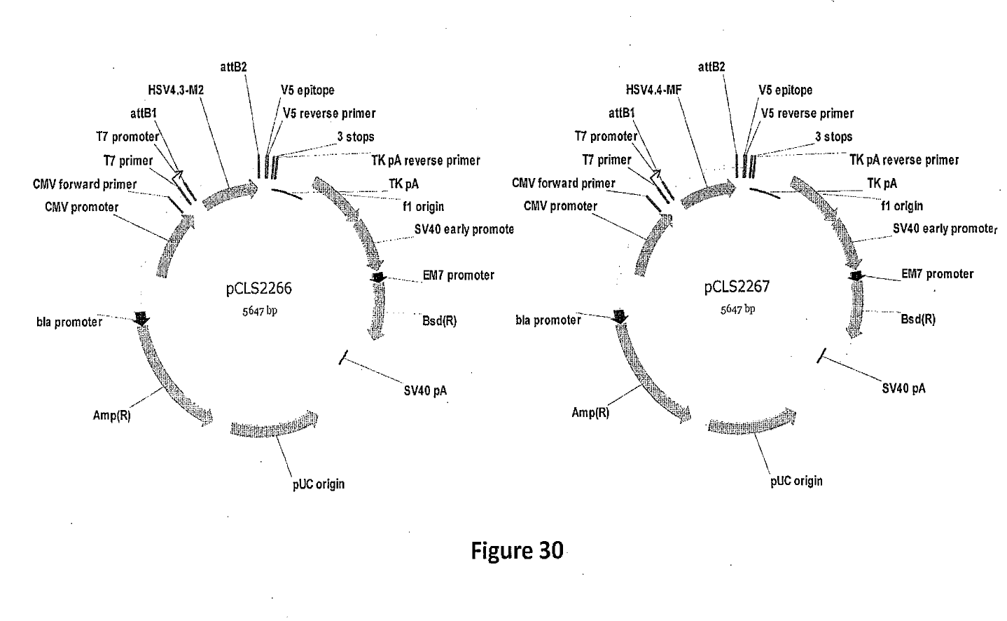

FIG. 30: pCLS2266 and pCLS2267

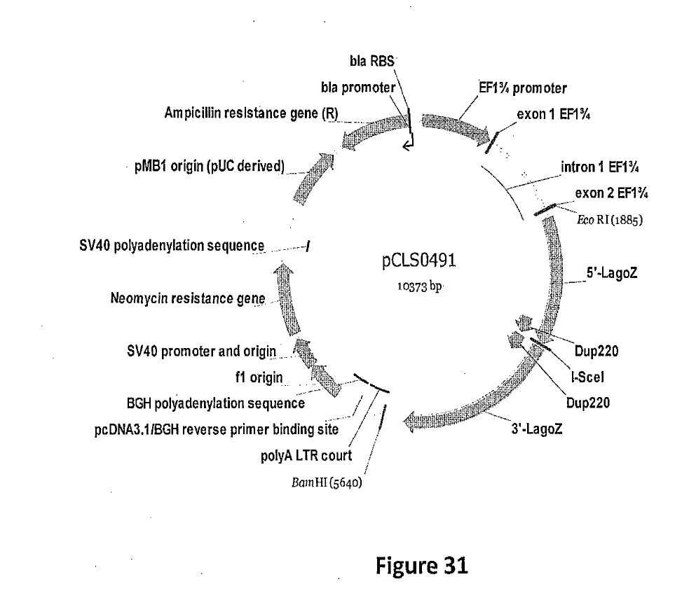

FIG. 31: pCLS0491

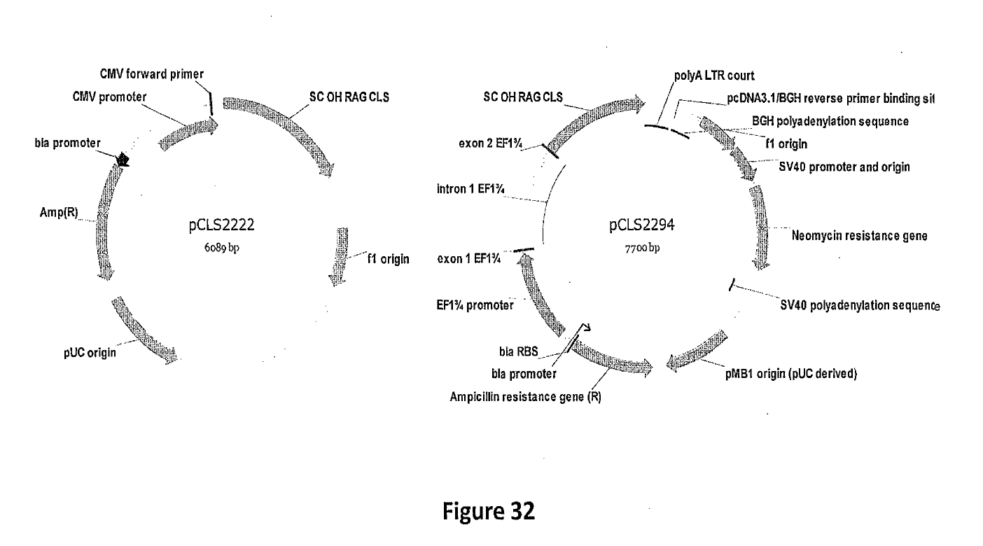

FIG. 32: pCLS2222, positive control expressing SCOH-RAG-CLS meganuclease under pCMV promoter, and pCLS2294, positive control expressing SCOH-RAG-CLS meganuclease under pEF1alpha promoter.

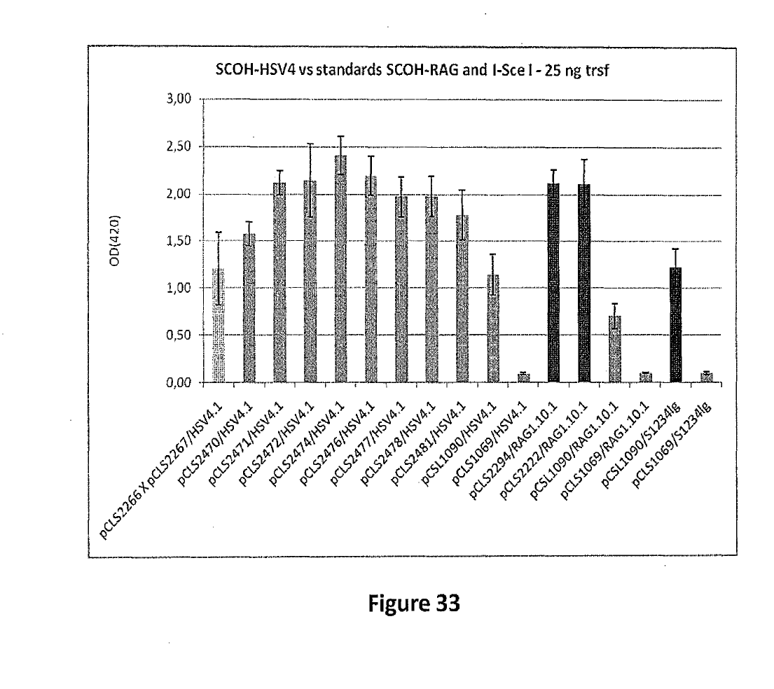

FIG. 33: Example of activity cleavage in CHO cells of designed single chain SCOH-HSV4 variants compared to initial heterodimer, I-Sce I and SCOH-RAG-CLS meganucleases as positive controls.

FIG. 34: Example of activity cleavage in CHO cells of single chain SCOH-HSV4 variants compared to initial heterodimer, I-Sce I and SCOH-RAG1.10 meganucleases as positive controls.

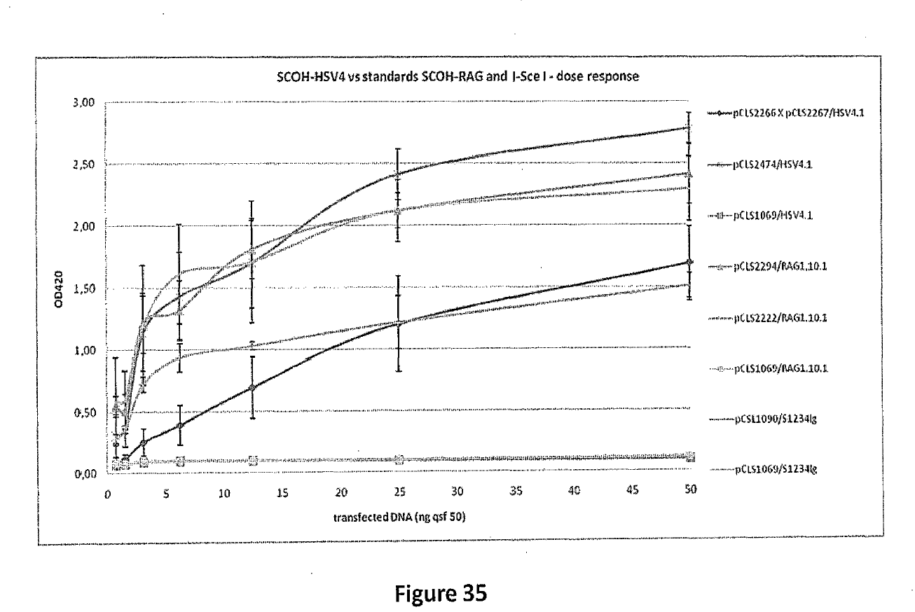

FIG. 35: Example of activity cleavage in CHO cells of single chain SCOH-HSV4-M2-54L-MF (pCLS2474) compared to initial heterodimer, I-SceI and SCOH-RAG-CLS meganucleases as positive controls.

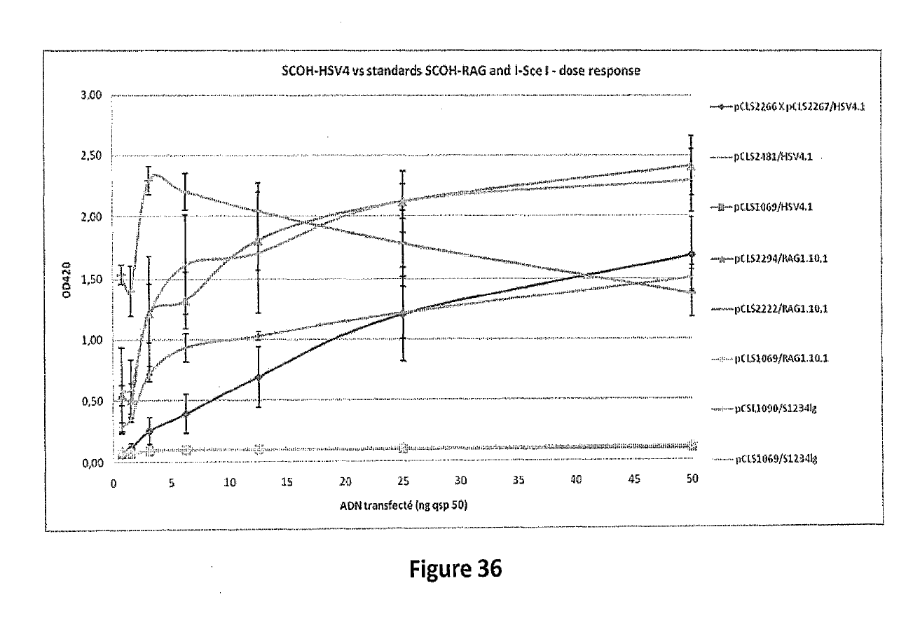

FIG. 36: Example of activity cleavage in CHO cells of single chain SCOH-HSV4-M2-105A-MF-80K 132V (pCLS2481) compared to initial heterodimer, I-SceI and SCOH-RAG-CLS meganucleases as positive controls.

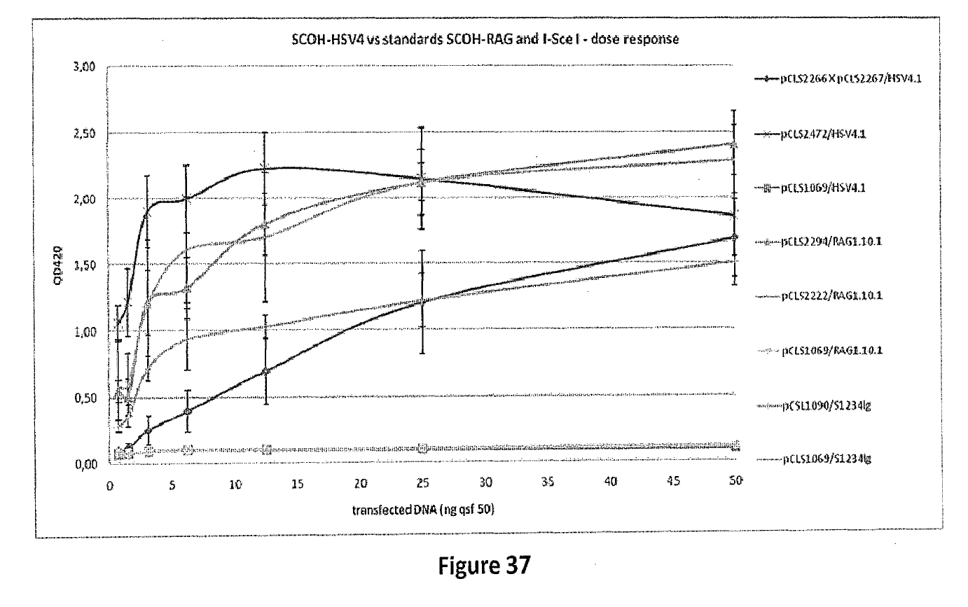

FIG. 37: Example of activity cleavage in CHO cells of single chain SCOH-HSV4-M2-MF-132V (pCLS2472) compared to initial heterodimer, I-SceI and SCOH-RAG-CLS meganucleases as positive controls.

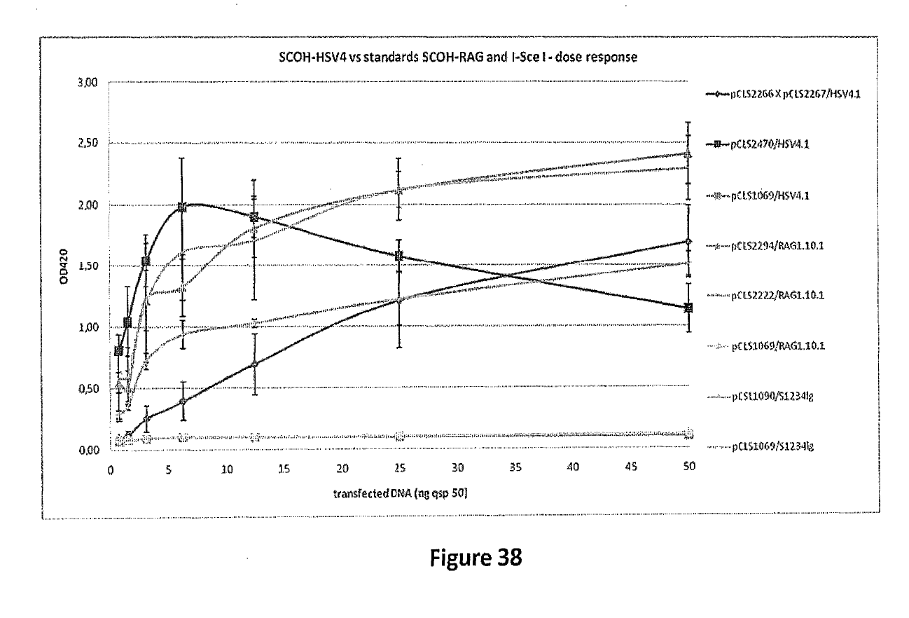

FIG. 38: Example of activity cleavage in CHO cells of single chain SCOH-HSV4-M2-MF (pCLS2470) compared to initial heterodimer, I-SceI and SCOH-RAG-CLS meganucleases as positive controls.

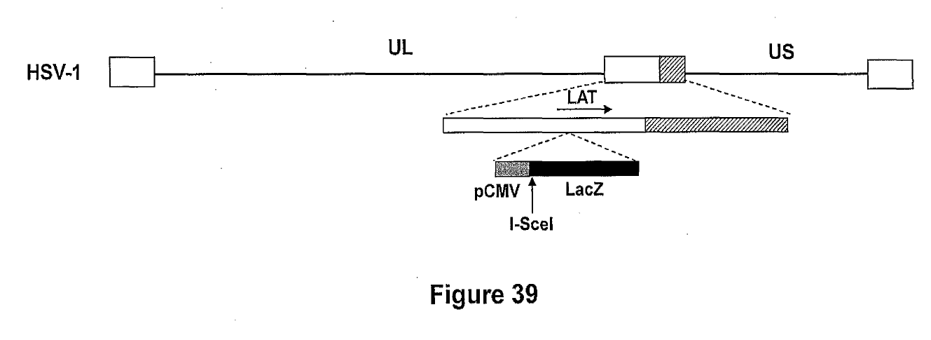

FIG. 39: Genomic structure of recombinant virus. The overall structure of the HSV-1 genome is shown with unique long (U_{L}) and unique short (U_{S}) regions flanked by inverted terminal repeats. The LAT region located in the terminal repeats has been expanded and the location of the LAT transcript are shown. An expression cassette containing the CMV promoter and the LacZ coding sequence was inserted in the major LAT gene. I-SceI target site was cloned between the CMV promoter and the LacZ gene.

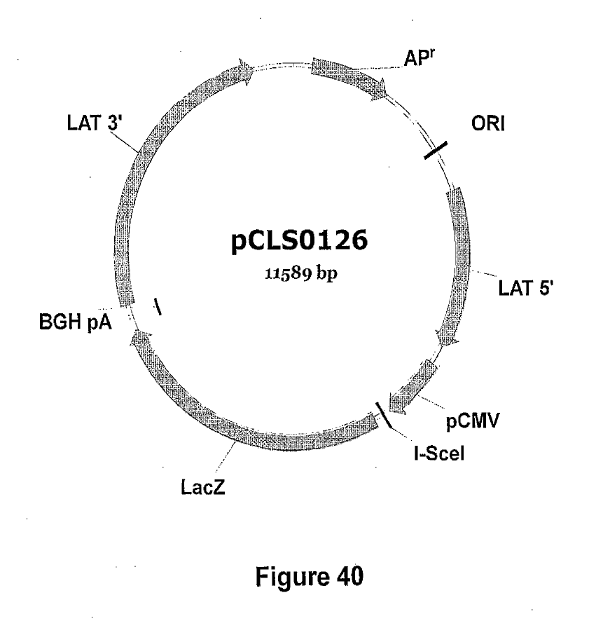

FIG. 40: pCLS0126

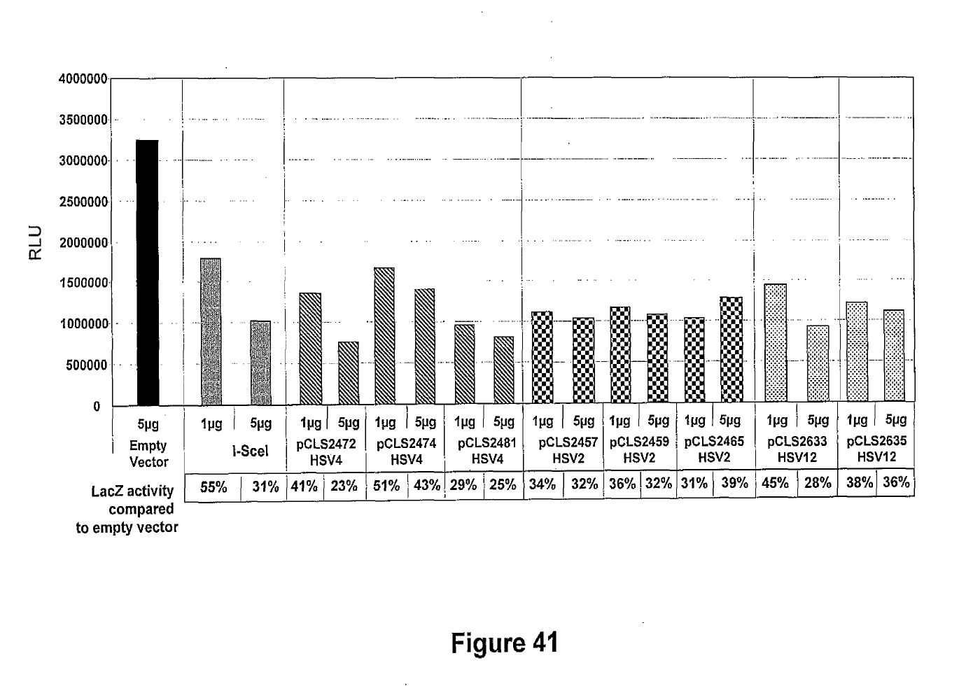

FIG. 41: Example of inhibition of viral replication by I-CreI single chain obligate heterodimer variants cleaving HSV2, HSV4 or HSV12 target sequences. COS-7 cells were transfected with empty vector, plasmid expressing I-SceI or plasmid expressing I-CreI variants cleaving HSV2, HSV4 or HSV12 target sequences. Twenty-four hours later the transfected cells were infected with rHSV-1 which expresses the LacZ gene. Beta-galactosidase activity levels, indicative of LacZ gene expression, was assayed twenty-four hours after infection. The detected activity levels are depicted in the histogram with the percent activity compared to empty vector indicated below the histogram.

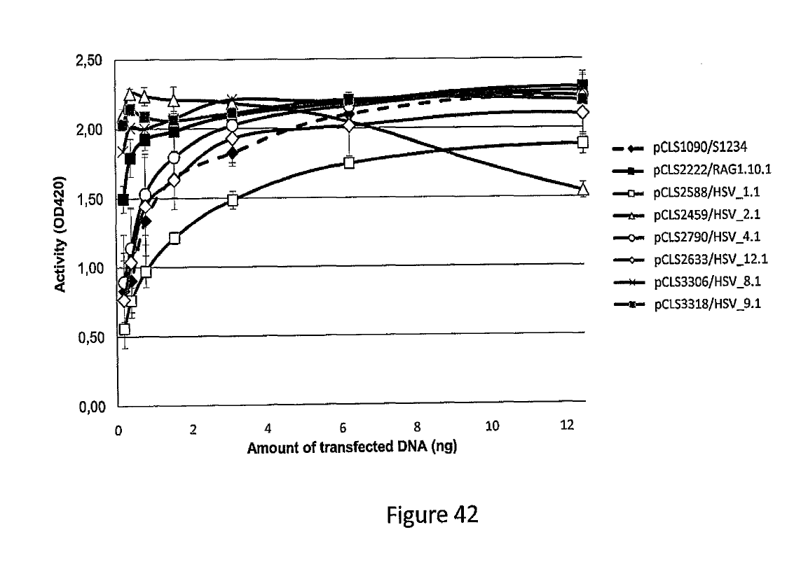

FIG. 42: Activity cleavage in CHO cells of single chain obligate heterodimer SCOH-HSV1-M5-132V-MF (pCLS2588), SCOH-HSV2-M1-MC80K105A132V (pCLS2459), SCOH-HSV4-M2-105A-MF-80K132V (pCLS2790), SCOH-HSV8b562-B (pCLS3306), SCOH-HSV9-bu-F (pCLS3318) and SCOH-HSV12-M1-ME-132V (pCLS2633), I-SceI (pCLS1090) and SCOH-RAG-CLS (pCLS2222) meganucleases as positive controls.

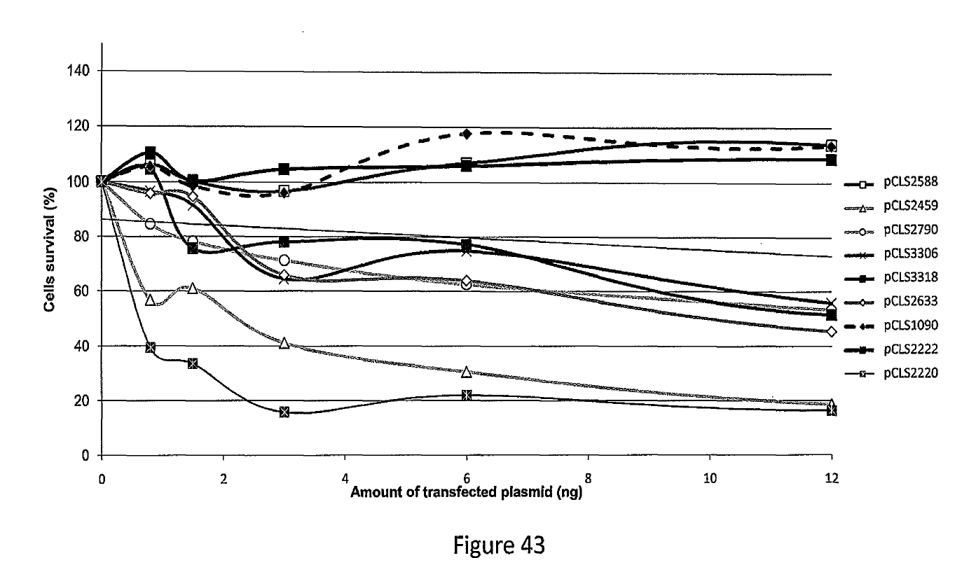

FIG. 43: Evaluation of the toxicity of SCOH-HSV meganucleases by a cell survival assay in CHO cells. Various amounts of plasmid expressing I-Cre I variants cleaving HSV1, HSV2, HSV4, HSV8, HSV9 or HSV12 target sequences and a constant amount of plasmid encoding GFP were used to co-transfect CHO cells. Cell survival is expressed as the percentage of cells expressing GFP 6 days after transfection, as described in the ‘Materials and Methods’ section. I-SceI (pCLS1090) and mRag1 (pCLS2222) meganucleases are shown as a control for non-toxicity and I-Cre I (pCLS2220) is shown as a control for toxicity.

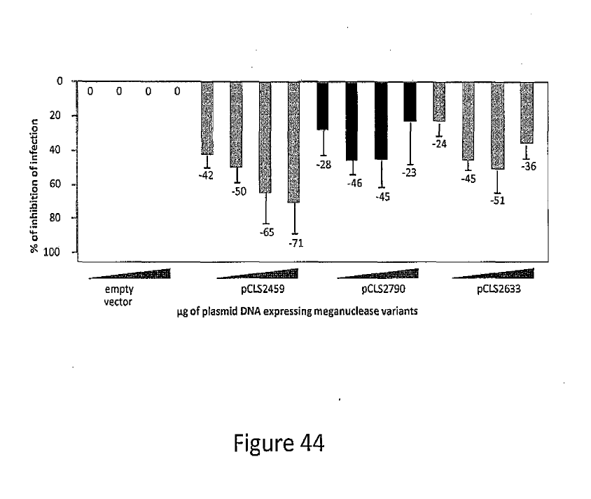

FIG. 44: Inhibition of viral replication by I-Cre I single chain obligate heterodimer variants cleaving HSV2, HSV4 or HSV12 target sequences. COS-7 cells were transfected with various amounts (0.3, 1, 5 and 10 μg) of empty vector or plasmid expressing I-Cre I variants. Twenty-four hours later the transfected cells were infected with rHSV-1 which expresses the LacZ gene at a MOI of 10⁻³. Beta-galactosidase activity levels, indicative of LacZ gene expression, was assayed twenty-four hours after infection. Results are expressed as the reduction of signal (in %) compared to the samples transfected with the same amount of empty vector.

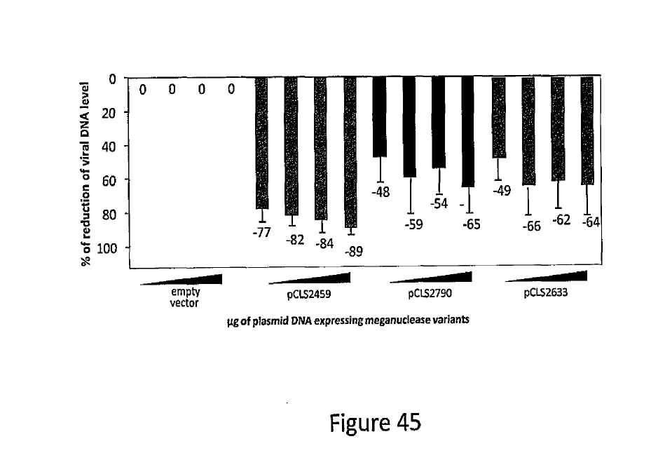

FIG. 45: Inhibition of viral replication by I-Cre I single chain obligate heterodimer variants cleaving HSV2, HSV4 or HSV12 target sequences. COS-7 cells were transfected with various amounts (0.3, 1, 5 and 10 μg) of empty vector or plasmid expressing I-Cre I variants. Twenty-four hours later the transfected cells were infected with rHSV-1 at a MOI of 10⁻³. The percentage of reduction of viral DNA level was assessed 24 hours later by Q-PCR compared to the samples transfected with the same amount of empty vector.

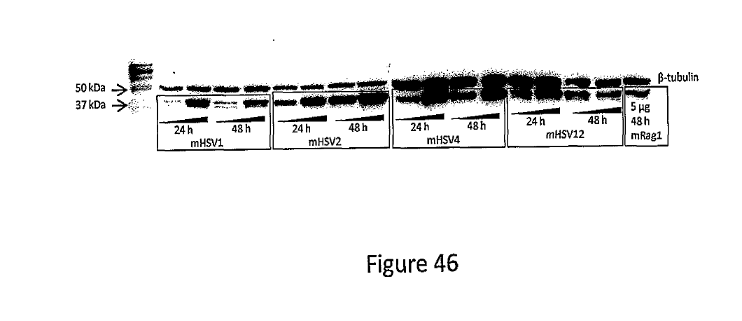

FIG. 46: Meganuclease expression levels were analysed in COS-7 cells by western blotting at different times after transfection with various amounts (1 and 5 μg) of plasmid expressing I-Cre I variants using a rabbit polyclonal antibody against I-Cre I. Antibody against β-tubulin was used for the loading control.

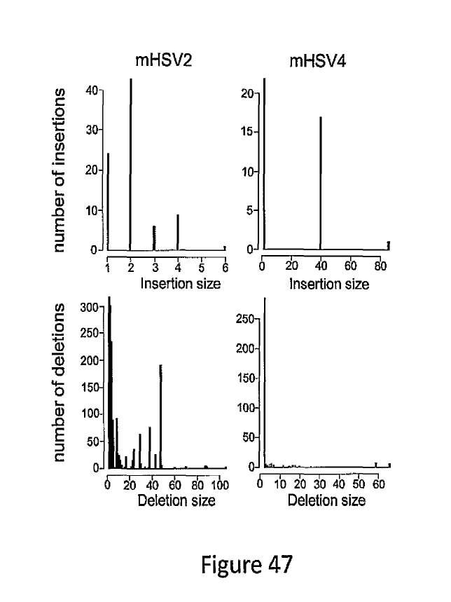

FIG. 47: Distribution and frequencies of meganuclease-induced deletions and insertions (indels) in the rHSV-1 genome after treatment with HSV2 and HSV4 meganucleases. 10356 PCR products were sequenced for HSV2, and 12228 for HSV4. The total number (and frequency) of observed deletions or insertions is indicated in the Table XXXVI. We also sequenced 23527 PCR products for HSV2 and 16961 for HSV4, in the absence of meganuclease treatments and found 12 events for HSV2, and no indel for HSV4.

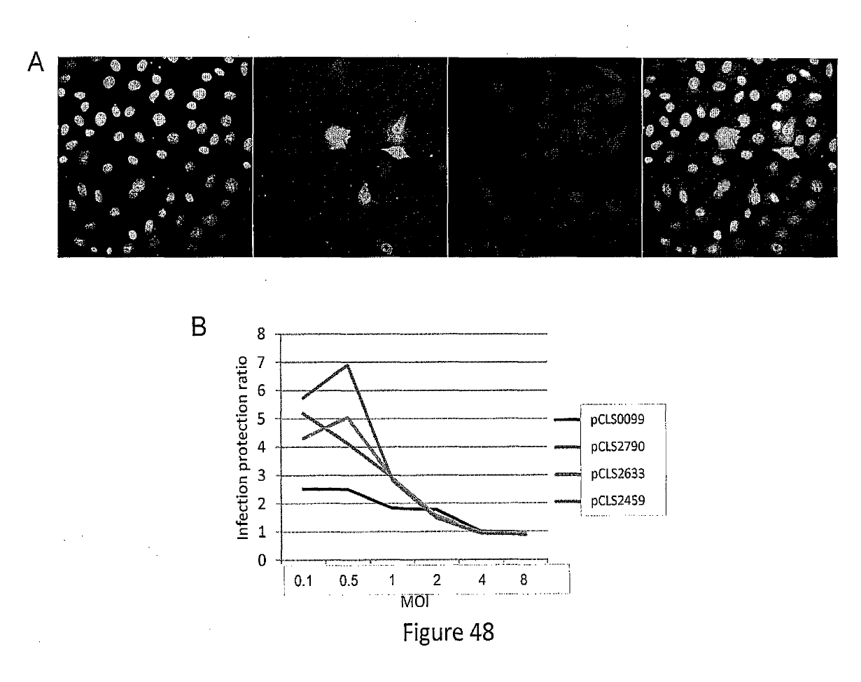

FIG. 48: Meganuclease-mediated inhibition of infection by a wild type HSV-1 virus. BSR cells were co-transfected with 1.5 μg of meganuclease expressing plasmid and 1.5 μg of a GFP expressing plasmid, and infected 48 hours later with various MOI (0.1, 0.5, 1, 2, 4, 8) of wild type HSV1 virus. Infection was assessed 8 hours later by immunocytochemistry with an antibody recognizing the gC viral glycoprotein, and we monitored the number of GFP+ HSV1+, GFP+ HSV1−, GFP− HSV1+ and GFP− HSV1− cells. A representative panel of these four categories is featured on (A). For each transfection, we quantified the level of infection inhibition as the following ratio: (number of HSV 1+GFP− cells/number of GFP− cells)/number of HSV1+ GFP+ cells/number of GFP+ cells). The levels plotted on panel are the average of three independent experiments (B).

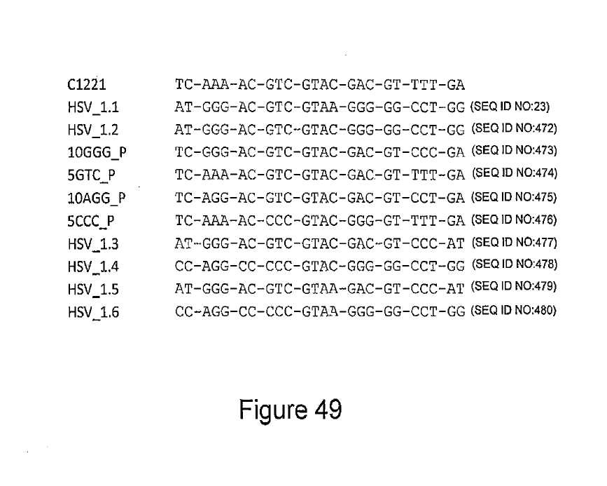

FIG. 49: represents target sequences of meganucleases described in Example 4.

FIG. 50: represents target sequences of meganucleases described in Example 5.

FIG. 51: represents target sequences of meganucleases described in Example 6.

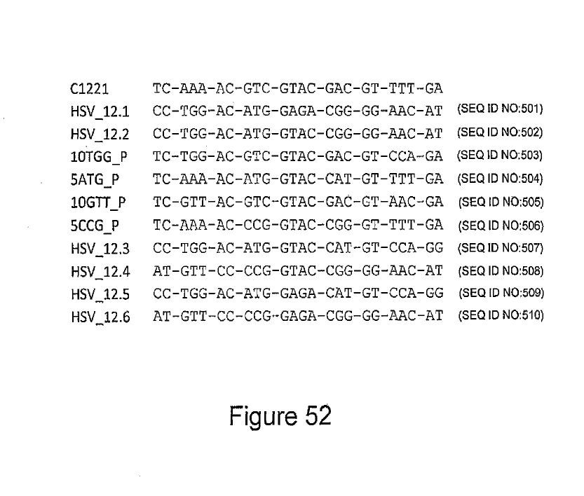

FIG. 52: represents target sequences of meganucleases described in Example 7.

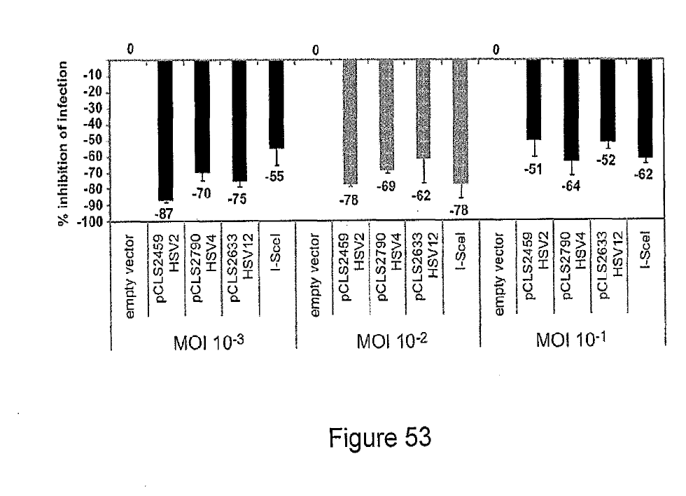

FIG. 53: Inhibition of viral replication by I-Cre I single chain obligate heterodimer variants cleaving HSV2, HSV4 or HSV 12 target sequences at increased MOIs. A single concentration (5 μg) of meganuclease expression plasmid was introduced in COS-7 cells and infected 24 hours later with rHSV1 at a MOI of 10⁻³, 10⁻² or 10⁻¹. Viral load was monitored by Q-PCR.

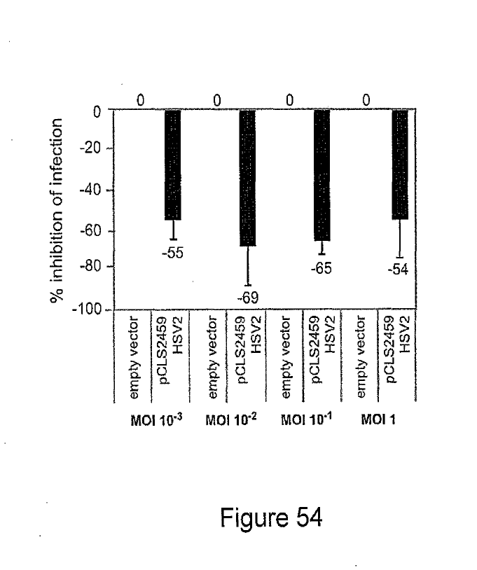

FIG. 54: Meganuclease-mediated inhibition of infection by a wild type HSV1 virus in COS-7 cells at increased MOIs. A single concentration (5 μg) of plasmid DNA expressing the I-Cre I variant cleaving HSV2 was introduced in COS-7 cells and infected 24 hours later with wt HSV 1 at a MOI of 10⁻³ to 1. Viral load was monitored by Q-PCR.

FIG. 55: The HBV12 target sequences and its derivatives. 10ATT_P, 10TAG_P, 5TGG_P and 5CTT_P are close derivatives cleaved by previously obtained I-CreI variants. They differ from C1221 by the boxed motives. C1221, 10ATT_P, 10TAG_P, 5TGG_P and 5CTT_P were first described as 24 bp sequences, but structural data suggest that only the 22 bp are relevant for protein/DNA interaction. However, positions +12 are indicated in parenthesis. HBV12 is the DNA sequence located at positions 2828-2850 of the Hepatitis B genome (accession number X70185). In the HBV12.2 target, the GAAC sequence in the middle of the target is replaced with GTAC, the bases found in C1221. HBV12.3 is the palindromic sequence derived from the left part of HBV12.2, and HBV12.4 is the palindromic sequence derived from the right part of HBV12.2. As shown in the figure, the boxed motives from 10ATT_P, 10TAG_P, 5TGG_P and 5CTT_P are found in the HBV12 series of targets.

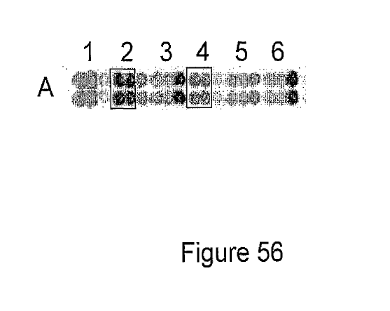

FIG. 56: Cleavage of HBV12.3 target by combinatorial variants. The figure displays an example of screening of I-CreI combinatorial variants with the HBV12.3 target. Each cluster contains 6 spots: In the 4 left spots, the yeast strain containing the HBV12.3 target mated with a variant from the combinatorial library described in Example 10. The right 2 spots are an internal control. On the filter, the sequence of the positive variants at positions A2 and A4 are KSRSQS/DYSSR and KSSNQS/DYSSR +66H, respectively, (according to the nomenclature of Table XXXXVIIII).

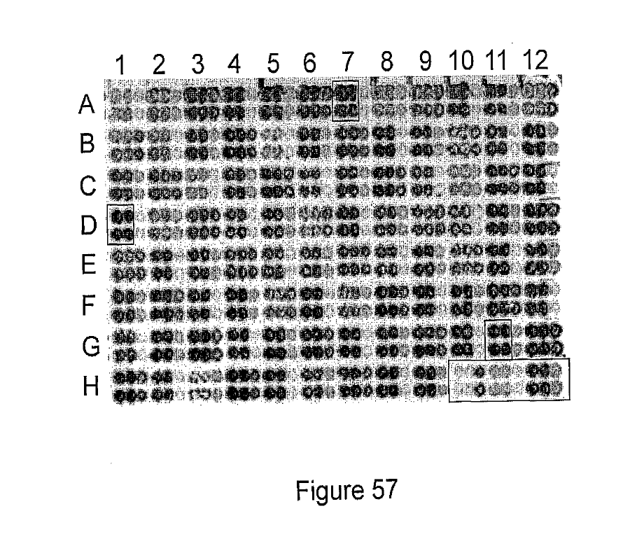

FIG. 57: Cleavage of HBV12.4 target by combinatorial variants. The Figure displays an example of screening of I-CreI combinatorial variants with the HBV12.4 target. Each cluster contains 6 spots: In the 4 left spots, the yeast strain containing the HBV12.4 target mated with a variant from the combinatorial library described in Example 11. The right 2 spots are an internal control. H10, H11 and H12 are negative and positive controls of different strength. On the filter, the sequence of the positive variants at positions A7, D1 and G11 are KNNCQS/RYSDN, KNHCQS/RYSNQ and KNHCQS/RYSYN, respectively, (according to the nomenclature of Table LI and Table LII).

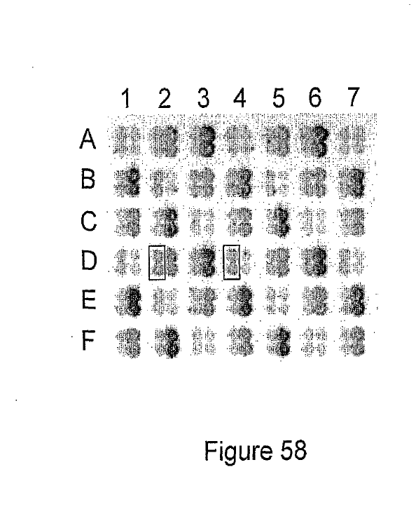

FIG. 58: Cleavage of the HBV12 target sequences by heterodimeric combinatorial variants. The figure displays an example of screening of combinations of I-CreI variants against the HBV12 target. Each cluster contains 4 spots: In the 2 left spots, a yeast strain co-expressing an HBV12.3 and an HBV12.4 variant mated with a yeast strain containing the HBV12 target. The right 2 spots are an internal control. The heterodimers displaying the strongest signal with the HBV12 target are observed at positions D2 and D4, corresponding to yeast co-expressing the HBV12.3 variant KSSNQS/DYSSR +66H with the HBV12.4 variants KNHCQS/RYSYN or KNHCQS/RYSNQ, respectively (according to the nomenclature of Table LIII).

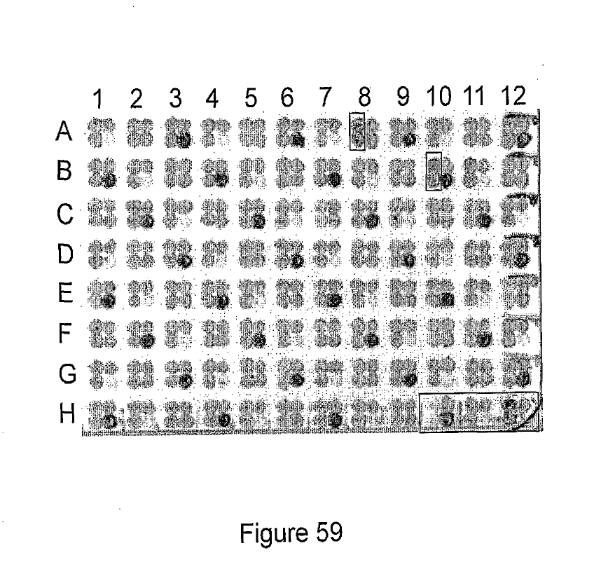

FIG. 59: Cleavage of the HBV12 target Example of screening against the HBV12 target of I-CreI refined variants obtained by random mutagenesis of initial variants cleaving HBV12.3 and co-expressed with a variant cutting HBV12.4. Each cluster contains 4 spots: In the 2 left spots, the yeast strain containing the HBV12 target and the HBV12.4 variant KNHCQS/RYSNQ mated with a different clone from the random mutagenesis library described in Example 13 (except for H10, H11 and H12: negative and positive controls of different strength). The top right spot is the HBV12.4 variant/HBV12 target strain mated with one of the initial HBV12.3 variants KSSNQS/DYSSR +66H (according to the nomenclature of Table L); the lower right spot is an internal control. On the filter, the sequence of the positive variants at positions A8 and B10 are 32Q, 38C, 44D, 68Y, 70S, 75S, 77R, 80A and 24F, 32Q, 38C, 44D, 68Y, 70S, 75S, 77R respectively.

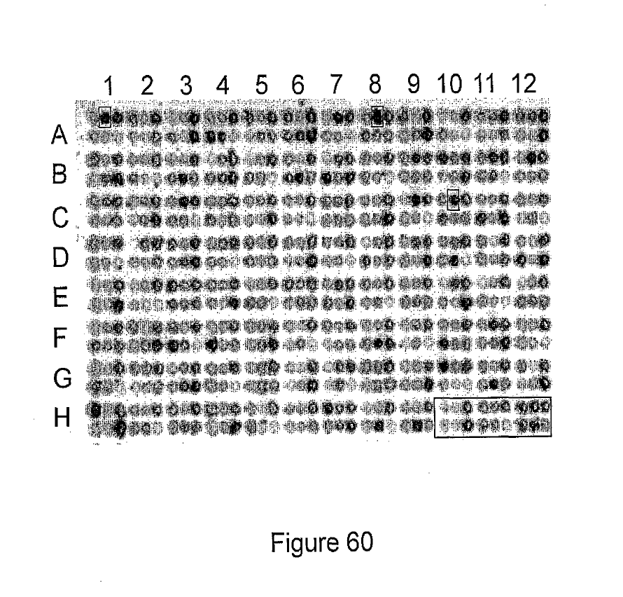

FIG. 60: Cleavage of the HBV12 target. Example of screening against the HBV12 target of I-CreI refined variants obtained by site-directed mutagenesis of variants cleaving the HBV12.3 target and co-expressed with a variant cutting HBV12.4. Each cluster contains 6 spots: For the 4 left spots, each spot represents the yeast strain containing the HBV12 target and the HBV12.4 variant KNHCQS/RYSNQ mated with a different clone from the site-directed mutagenesis library described in Example 14. The top right spot is the HBV12.4 variant/HBV12 target strain mated with one of the HBV12.3 optimized variants 32Q, 38C, 44D, 68Y, 70S, 75S, 77R, 80A (Table LIV); the lower right spot is an internal control. H10, H11 and H112 are negative and positive controls of different strength. The sequence of the positive variants at positions A1, A8 and C10 are 24F, 32Q, 38C, 44D, 68Y, 70S, 75S, 77R, 80K 24F, 32Q, 38C, 44D, 68Y, 70S, 75S, 77R, 87L, 153G and 24F, 32Q, 38C, 44D, 68Y, 70S, 75S, 77R, 105A, 132V, respectively.

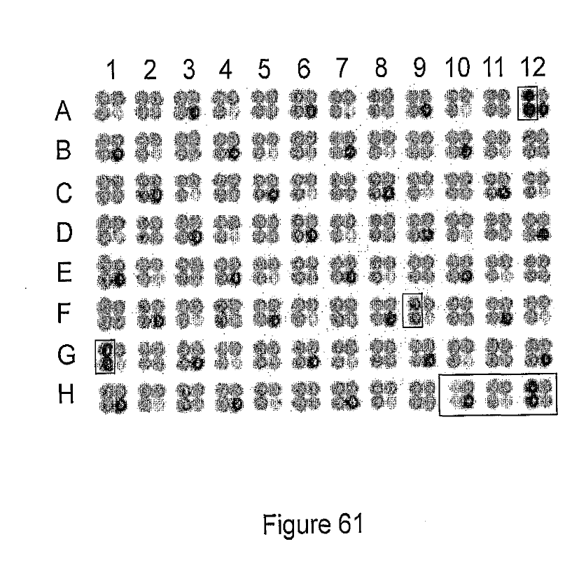

FIG. 61: Cleavage of the HBV12 target. Example of screening against the HBV12 target of I-CreI refined variants obtained by site-directed mutagenesis of variants cleaving the HBV12.4 target and co-expressed with a variant cutting HBV12.3. Each cluster contains 4 spots: In the 2 left spots, the yeast strain containing the HBV12 target and the HBV12.3 variant KSRSQS/DYSSR mated with a different clone from the site-directed mutagenesis library described in Example 13. The top right spot is the HBV12.3 variant/HBV12 target strain mated with one of the initial HBV12.4 variants KNHCQS/RYSNQ (according to the nomenclature of Table LII); the lower right spot is an internal control. H10, H11 and H12 are negative and positive controls of different strength. The sequence of the positive variants at positions A12, F9, and G1 are 32H, 33C, 40R, 44R, 68Y, 70S, 75N, 77Q; 32H, 33C, 44R, 68Y, 70S, 75Y, 77Q, 87L and 19S, 32H, 33C, 44R, 68Y, 70S, 75D77R, respectively.

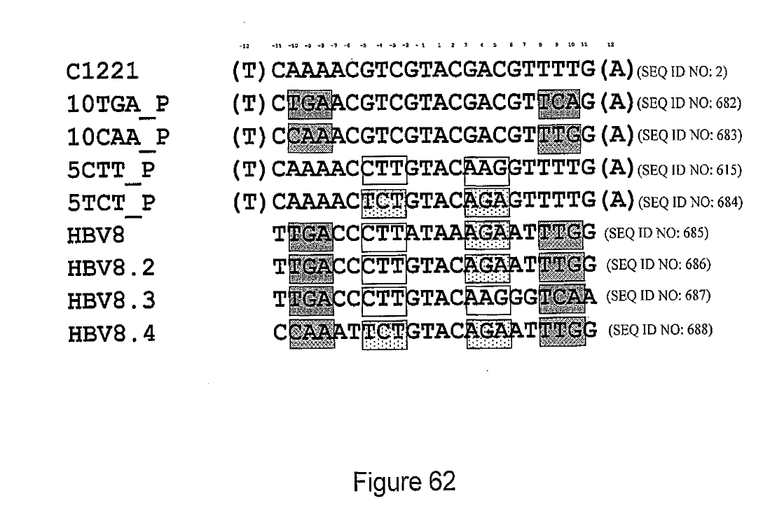

FIG. 62: The HBV8 target sequences and its derivatives. 10TGA_P, 10CAA_P, 5CTT_P and 5TCT_P are close derivatives cleaved by previously obtained I-CreI variants. They differ from C1221 by the boxed motives. C1221, 10TGA_P, 10CAA_P, 5CTT_P and 5TCT_P were first described as 24 bp sequences, but structural data suggest that only the 22 bp are relevant for protein/DNA interaction. However, positions ±12 are indicated in parenthesis. HBV8 is the DNA sequence located at positions 1908-1929 of the Hepatitis B genome (accession number X70185). In the HBV8.2 target, the ATAA sequence in the middle of the target is replaced with GTAC, the bases found in C1221. HBV8.3 is the palindromic sequence derived from the left part of HBV8.2, and HBV8.4 is the palindromic sequence derived from the right part of HBV8.2. As shown in the Figure, the boxed motives from 10TGA_P, 10CAA_P, 5CTT_P and 5TCT_P are found in the HBV8 series of targets.

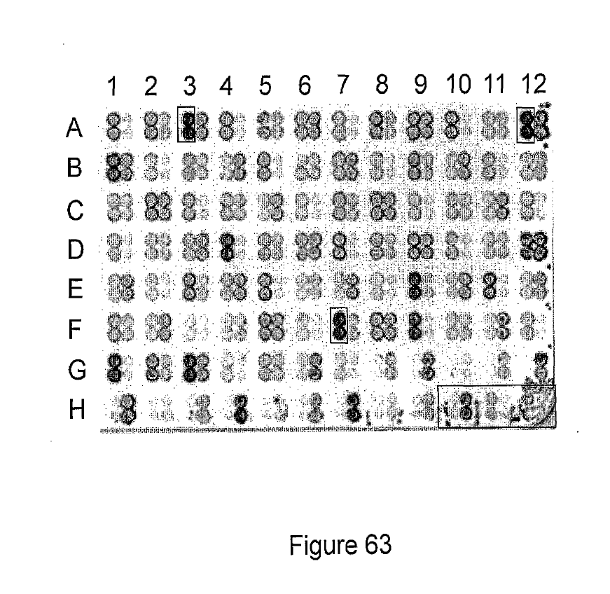

FIG. 63: Cleavage of HBV8.3 target by combinatorial variants. The Figure displays an Example of screening of I-CreI combinatorial variants with the HBV8.3 target. Each cluster contains 4 spots: In the 2 left spots, the yeast strain containing the HBV8.3 target mated with a variant from the combinatorial library described in Example 17. The right 2 spots are an internal control. On the filter, the sequence of the positive variants at positions A3, A12 and F9 are KNSCRS/RYSDN, KHSCHS/RYSYN and KNSARS/RYSDN, respectively, (according to the nomenclature of Table LXIII).

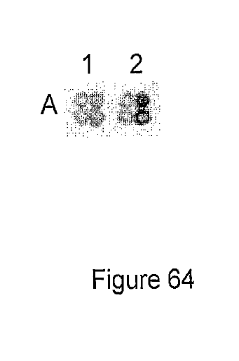

FIG. 64: Cleavage of HBV8.4 target by combinatorial variants. The Figure displays an Example of screening of I-CreI combinatorial variants with the HBV8.4 target. Each cluster contains 4 spots: In the 2 left spots, the yeast strain containing the HBV8.4 target mated with a variant from the combinatorial library described in Example 18. The right 2 spots are an internal control. On the filter, the sequence of the positive variants at positions A1, A2 are KNSHQQ/QRSNK and KNSHQQ/QRSNK+163Q, respectively, (according to the nomenclature of Table LIX and Table LX).

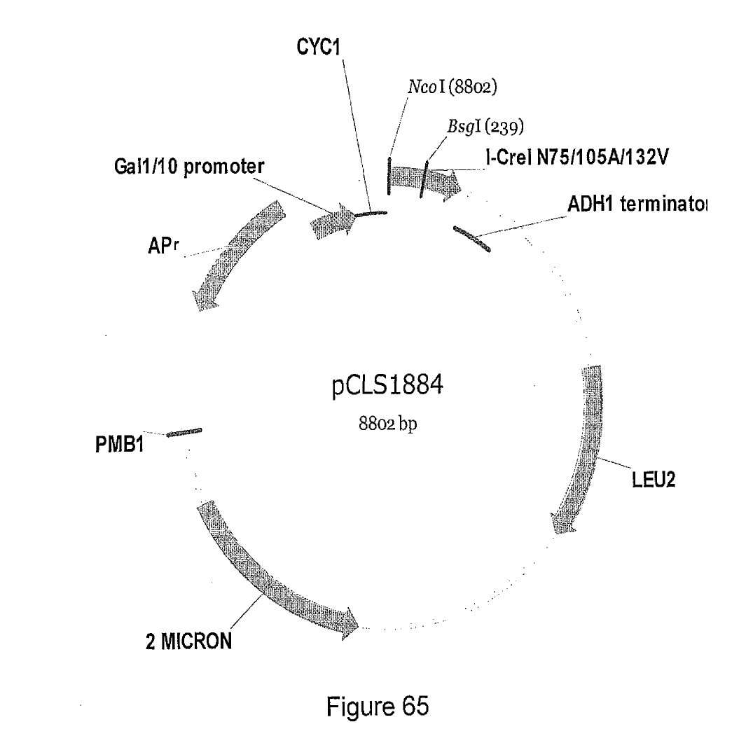

FIG. 65: pCLS1884 plasmid map.

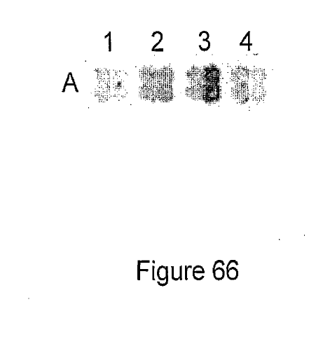

FIG. 66: Cleavage of HBV8.4 target by combinatorial variants containing 105A and 132V mutations. The figure displays an example of screening of I-CreI combinatorial variants with the HBV8.4 target. Each cluster contains 4 spots: In the 2 left spots, the yeast strain containing the HBV8.4 target mated with a variant from the combinatorial library containing the 105A and 132V substitutions described in Example 19. The right 2 spots are an internal control. On the filter, the sequence of the positive variants at positions A1, A2, A3 and A4 are KNSHQQ/KASNI +105A132V, KNEYQS/QSSNR+105A132V, KNEYQS/QASNR+105A132V and KNSHQQ/KNANI +105A32V respectively, (according to the nomenclature of Table LXI).

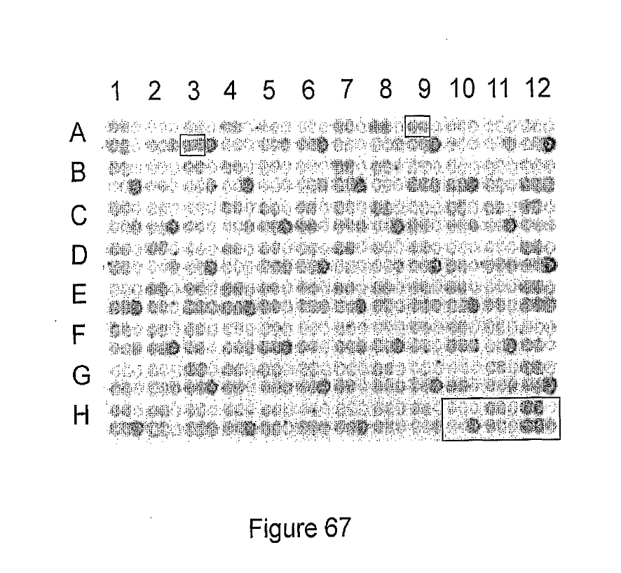

FIG. 67: Cleavage of the HBV8 target. Example of screening against the HBV8 target of I-CreI refined variants obtained by random mutagenesis of initial variants cleaving HBV8.4 and co-expressed with a variant cutting HBV8.3. Each cluster contains 6 spots: In the 4 left spots, the yeast strain containing the HBV8 target and the HBV8.3 variant KNSCRS/RYSDN mated with two different clones from the random mutagenesis library (clone 1, upper left and middle spots; clone 2, lower left and middle spots) described in Example 20. H10, H11 and H12: negative and positive controls of different strength. The 2 right spots are an internal control. On the filter, the sequence of the positive variants at positions A3 and A9 are 33H, 40Q, 70S, 75N, 77K, 105A, 132V and 33H, 40Q, 68A, 70S, 75N, 77R, 105A, 132V, respectively.

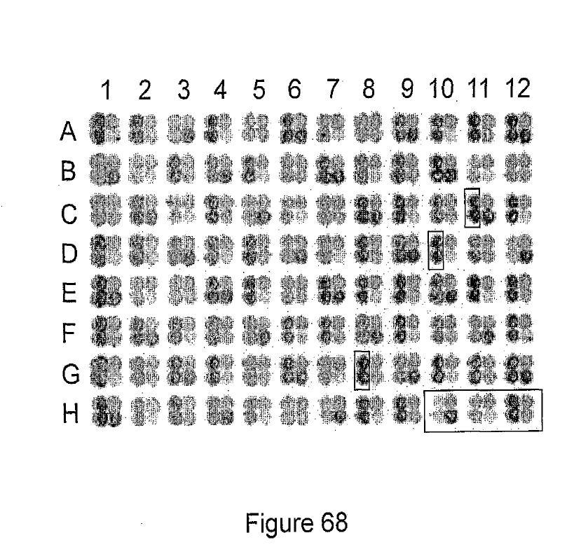

FIG. 68: Cleavage of the HBV8 target. Example of screening against the HBV8 target of I-CreI refined variants obtained by site-directed mutagenesis of variants cleaving the HBV8.4 target and co-expressed with a variant cutting HBV8.3. Each cluster contains 4 spots: In the 2 left spots, the yeast strain containing the HBV8 target and the HBV8.3 variant KNSCRS/RYSDN mated with a different clone from the site-directed mutagenesis library described in Example 21. The top right spot is the HBV8.3 variant/HBV8 target strain mated with one of the optimized HBV8.4 variants 33H, 40Q, 70S, 75N, 77K, 105A, 132V (according to the nomenclature of Table LXIV); the lower right spot is an internal control. H10, H11 and H12 are negative and positive controls of different strength. The sequence of the positive variants at positions C11, D10, and G8 are 19S, 33H, 40Q, 70S, 75N, 77K, 105A, 132V; 19S, 33H, 40Q, 70S, 75N, 77K, 105A and 19S, 33H, 40Q, 43I, 70S, 75N, 77K, 105A, 132V, respectively.

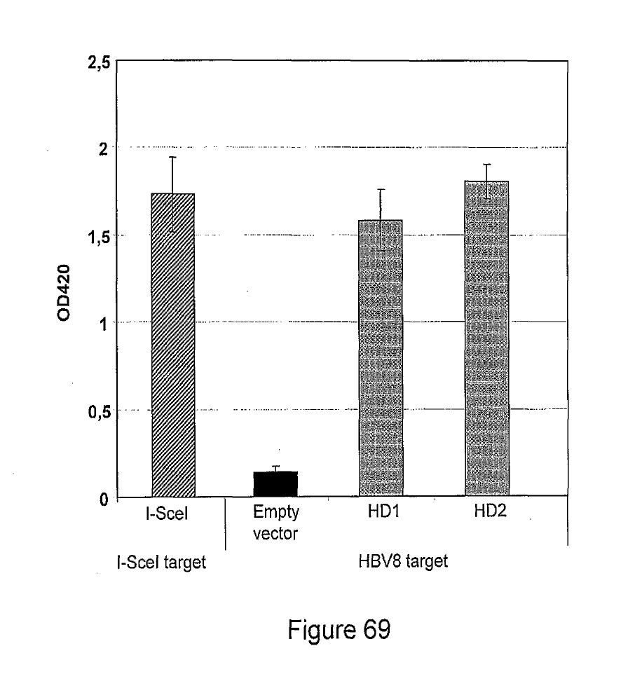

FIG. 69: HBV8 target cleavage in CHO cells. Extrachromosomal assay in CHO cells for heterodimers displaying strong cleavage activity against the HBV8 target as described in Example 20. OD values indicated were observed 3 hours after lysis/revelation buffer addition. HD1 represents the results obtained with co-expression of the HBV8.3 variant 33C, 38R, 44R, 68Y, 70S, 77N with HBV8.4 variant 19S, 33H, 40Q, 43I, 70S, 75N, 77K, 105A, 132V. HD2 represents the results obtained with co-expression of the HBV8.3 variant 33C, 38R, 44R, 68Y, 70S, 77N and HBV8.4 variant 19S, 33H, 40Q, 70S, 75N, 77K, 105A, 132V. I-SceI and empty vector are presented as positive and negative controls, respectively.

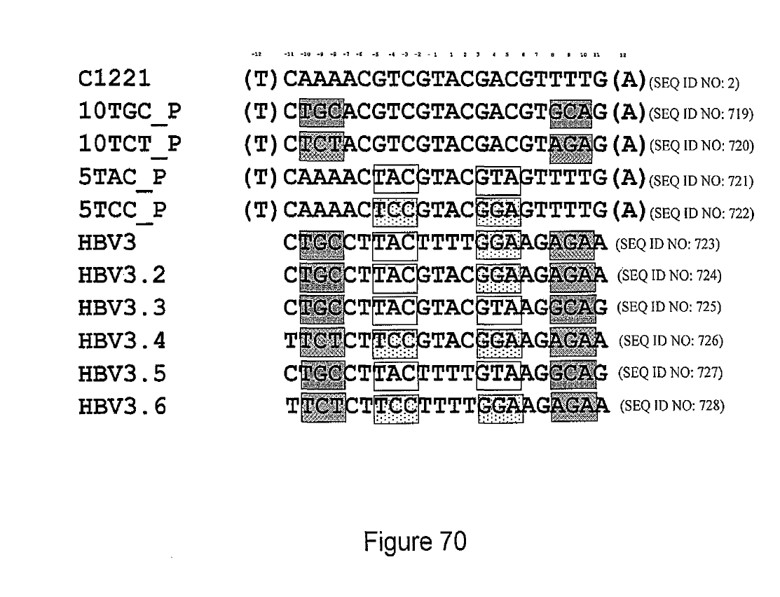

FIG. 70: The HBV3 target sequences and its derivatives. 10TGC_P, 10TCT_P, 5TAC_P and 5_TCCP are close derivatives cleaved by previously obtained I-CreI variants. They differ from C1221 by the boxed motives. C1221, 10TGC_P, 10TCT_P, 5TAC_P and 5TCC_P were first described as 24 bp sequences, but structural data suggest that only the 22 bp are relevant for protein/DNA interaction. However, positions ±12 are indicated in parenthesis. HBV3 is the DNA sequence located at positions 2216-2237 of the Hepatitis B genome (accession number M38636). In the HBV3.2 target, the TTTT sequence in the middle of the target is replaced with GTAC, the bases found in C1221. HBV3.3 is the palindromic sequence derived from the left part of HBV3.2, and HBV3.4 is the palindromic sequence derived from the right part of HBV3.2. HBV3.5 and HBV3.6 are pseudo-palindromic targets similar to HBHV3.3 and HBV3.4 except that they contain the tttt sequence at positions −2 to 2. As shown in the figure, the boxed motives from 10TGC_P, 10TCT_P, 5TAC_P and 5TCC_P are found in the HBV3 series of targets.

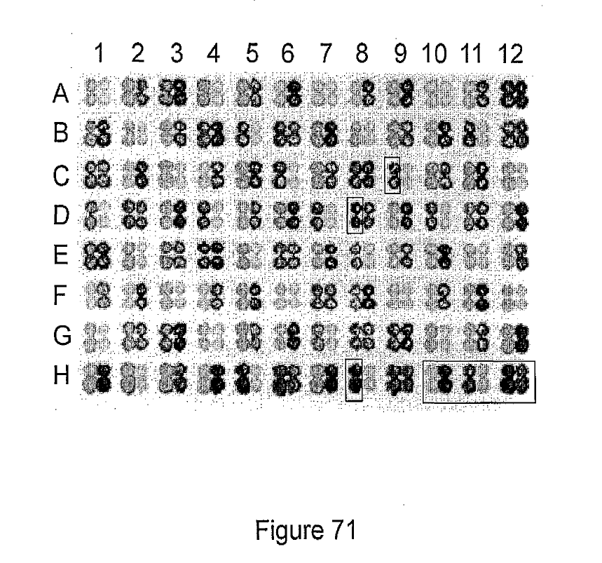

FIG. 71: Cleavage of HBV3.3 target by combinatorial variants. The figure displays an example of screening of I-CreI combinatorial variants with the HBV3.3 target. Each cluster contains 4 spots: In the 2 left spots, the yeast strain containing the HBV3.3 target mated with a variant from the combinatorial library described in Example 24. The right 2 spots are an internal control. On the filter, the sequence of the positive variants at positions C9, D8 and H8 are KNSCRS/AYSRT, KNSSRQ/AYSRI and KNSCSS/NYSRY, respectively, (according to the nomenclature of Table LXIV and LXV).

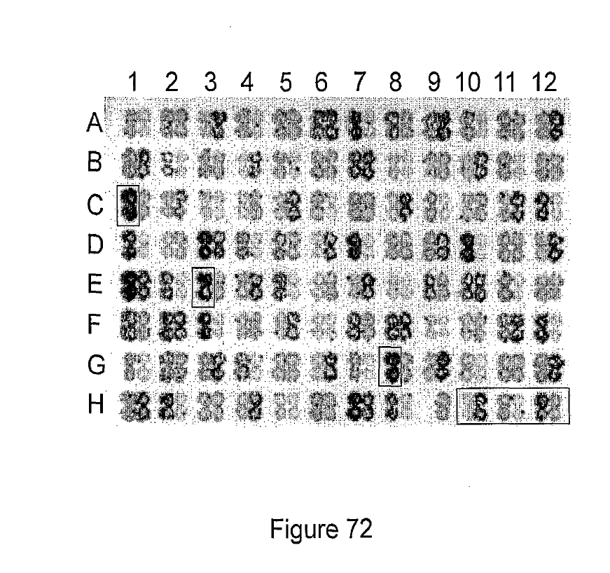

FIG. 72: Cleavage of HBV3.4 target by combinatorial variants. The figure displays an example of screening of I-CreI combinatorial variants with the HBV3.4 target. Each cluster contains 4 spots: In the 2 left spots, the yeast strain containing the HBV3.4 target mated with a variant from the combinatorial library described in Example 25. The right 2 spots are an internal control. On the filter, the sequence of the positive variants at positions C1, E3 and G8 are KNSCYS/KYSNV +45M, KNSSYS/KHNNI and KNSGYS/KYSNV +45M, respectively, (according to the nomenclature of Table LXVI and Table LXVII).

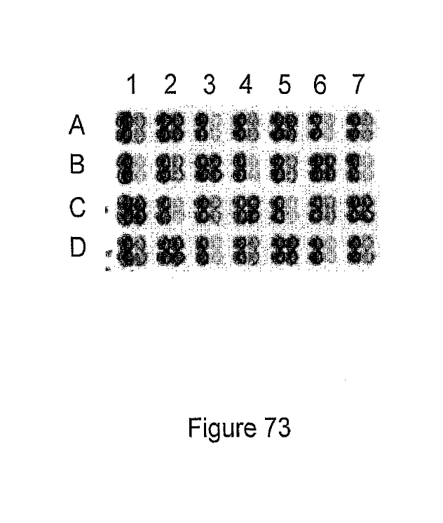

FIG. 73: Cleavage of the HBV3.2 target sequences by heterodimeric combinatorial variants. The figure displays an example of screening of combinations of I-CreI variants against the HBV3.2 target. Each cluster contains 4 spots: In the 2 left spots; a yeast strain co-expressing the HBV3.3 and HBV3.4 combinatorial variants was mated with a yeast strain containing the HBV3 target as described in Example 26. The right 2 spots are an internal control. All heterodimers tested resulted in strong cleavage of the HBV3.2 target.

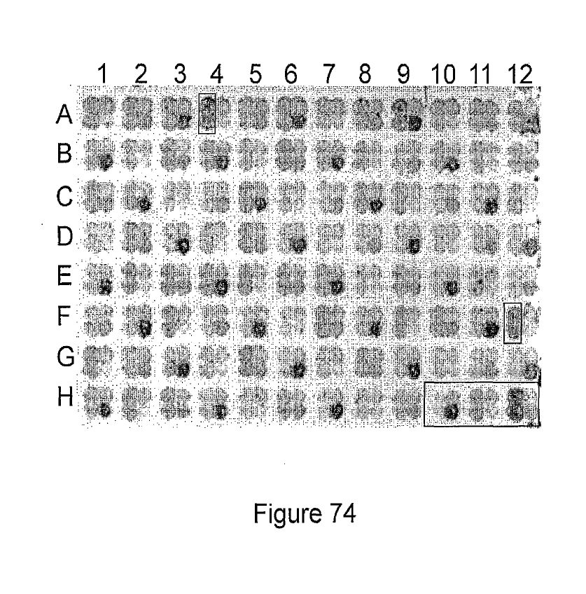

FIG. 74: Cleavage of the HBV3.5 target. Example of screening against the HBV3.5 target of I-CreI refined variants obtained by random mutagenesis of initial variants cleaving HBV8.3. Each cluster contains 4 spots: In the 2 left spots, the yeast strain containing the HBV8.5 target mated with a clone from the random mutagenesis library described in Example 27. H10, H11 and H12: negative and positive controls of different strength. The top right spot is the HBV3.5 target strain mated with one of the initial HBV3.3 variants KNSCRS/AYSRT (according to the nomenclature of Table LXV). The right lower spot is an internal control. On the filter, the sequence of the positive variants at positions A4 and F12 are 26R, 33C, 38S, 44N, 68Y, 70S, 75R, 77Y, 81T and 33C, 38R, 44A, 68Y, 70S, 75R, 77T, 132V, respectively.

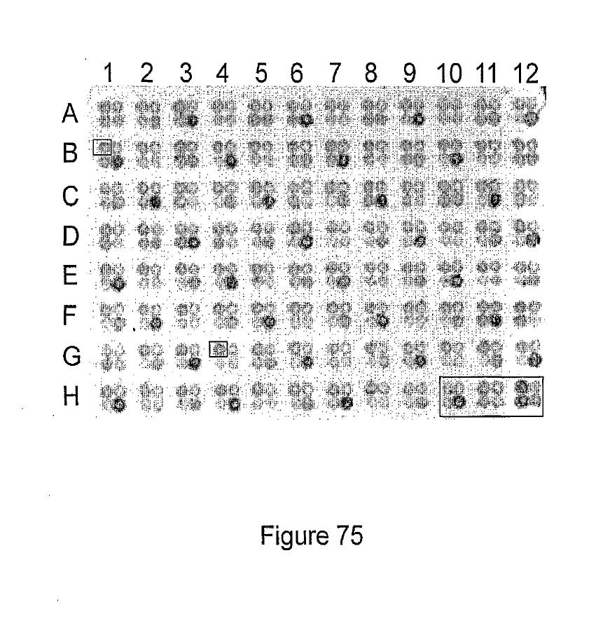

FIG. 75: Cleavage of the HBV3.6 target. Example of screening against the HBV3.6 target of I-CreI refined variants obtained by random mutagenesis of initial variants cleaving HBV8.4. Each cluster contains 4 spots: In the 2 left spots, the yeast strain containing the HBV8.6 target mated with two different clones from the random mutagenesis library described in Example 28. H10, H11 and H12: negative and positive controls of different strength. The top right spot is the HBV3.6 target strain mated with one of the initial HBV3.4 variants KNSGYS/KYSNY (according to the nomenclature of Table LXVI). The right lower spot is an internal control. On the filter, the sequence of the positive variants at positions B1 and G4 are 33C, 38Y, 44K, 64I, 68Y, 70S, 75N, 77Y, 85R and 33S, 38Y, 44K, 45M, 68Y, 70S, 75N, 77V, 86T, respectively.

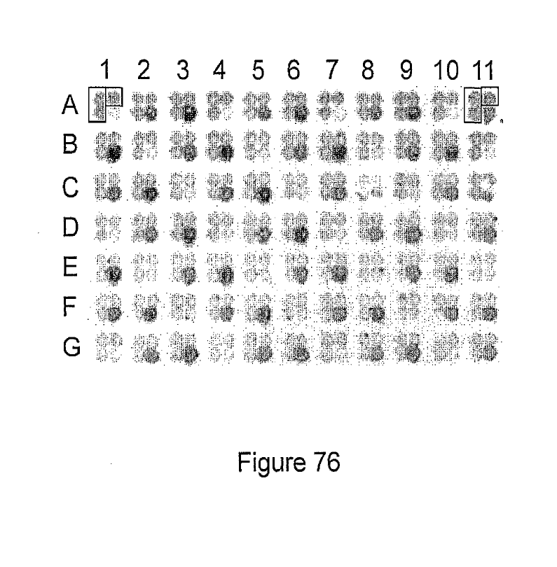

FIG. 76: Cleavage of the HBV3 target sequences by optimized heterodimeric variants. The figure displays an example of screening of I-CreI variants against the HBV3 target. Each cluster contains 4 spots: In the 2 left spots and the upper right spot, a yeast strain co-expressing an HBV3.3 and an HBV3.4 variant mated with a yeast strain containing the HBV3 target. The lower right spot is an internal control. The heterodimers displaying the strongest signal with the HBV3 target are observed at positions A1 and A11, corresponding to yeast co-expressing the HBV3.3 variant 26R, 33C, 38S, 44N, 68Y, 70S, 75R, 77Y, 81T with the HBV3.4 variants 33S, 38Y, 44K, 68Y, 70S, 75N, 77L and 2D, 33S, 38Y, 44K, 68Y, 70S, 75N, 77Y, 140M, respectively.

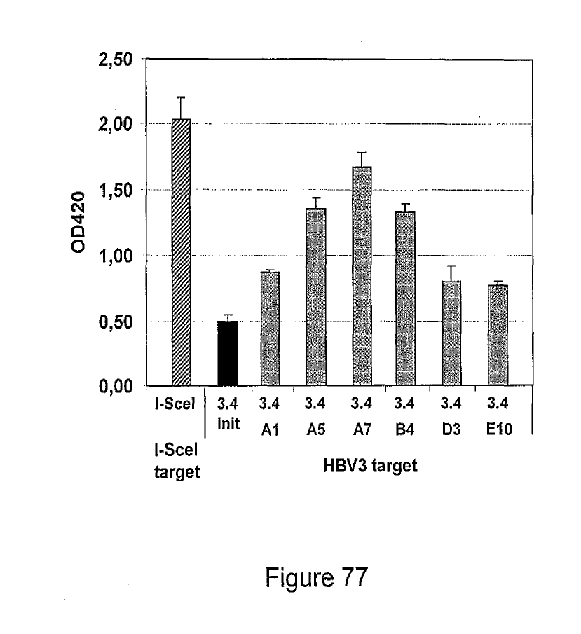

FIG. 77: Cleavage of the HBV3 target. Example of secondary screening against the HBV3 target of I-CreI refined variants obtained by random mutagenesis of variants cleaving the HBV3.4 target and co-expressed with a variant cutting HBV3.3. Extrachromosomal assay in CHO cells for heterodimers displaying cleavage activity against the HBV3 target as described in Example 30. HBV3.4 variants, both the initial (33S, 38Y, 44K, 68Y, 70S, 75N, 77L) and optimized (see Table LXXII) variants, were co-expressed with the HBV3.3 variant 26R, 33C, 38S, 44N, 68Y, 70S, 75R, 77Y, 81T and examined for their ability to cleave the HBV3 target. OD values indicated were observed 3 hours after lysis/revelation buffer addition. I-SceI is presented as a positive control.

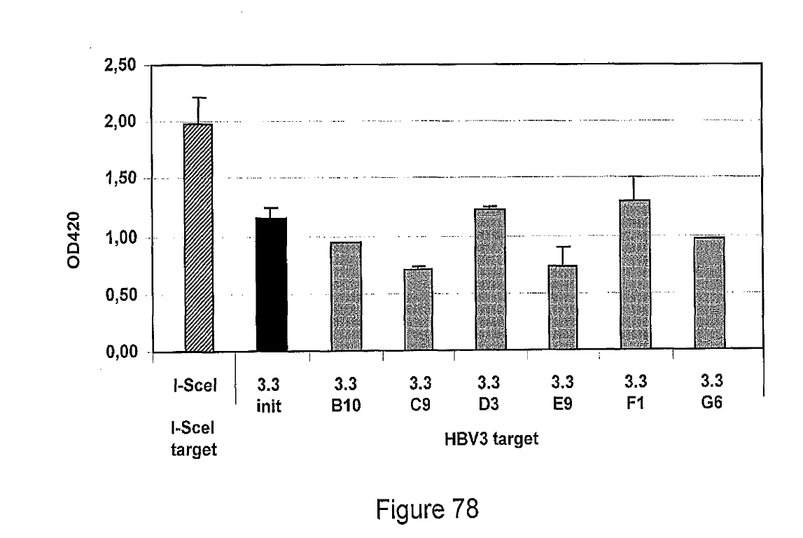

FIG. 78: Cleavage of the HBV3 target. Example of secondary screening against the HBV3 target of I-CreI refined variants obtained by random mutagenesis of variants cleaving the HBV3.3 target and co-expressed with a variant cutting HBV3.4. Extrachromosomal assay in CHO cells for heterodimers displaying cleavage activity against the HBV3 target as described in Example 31. HBV3.3 variants, both the initial (26R, 33C, 38S, 44N, 68Y, 70S, 75R, 77Q, 81T) and optimized (see Table LXXIII) variants, were co-expressed with the HBV3.4 variant 19S, 33C, 38Y, 44K, 68Y, 70S, 75N, 77Q and examined for their ability to cleave the HBV3 target. OD values indicated were observed 3 hours after lysis/revelation buffer addition. I-SceI is presented as a positive control.

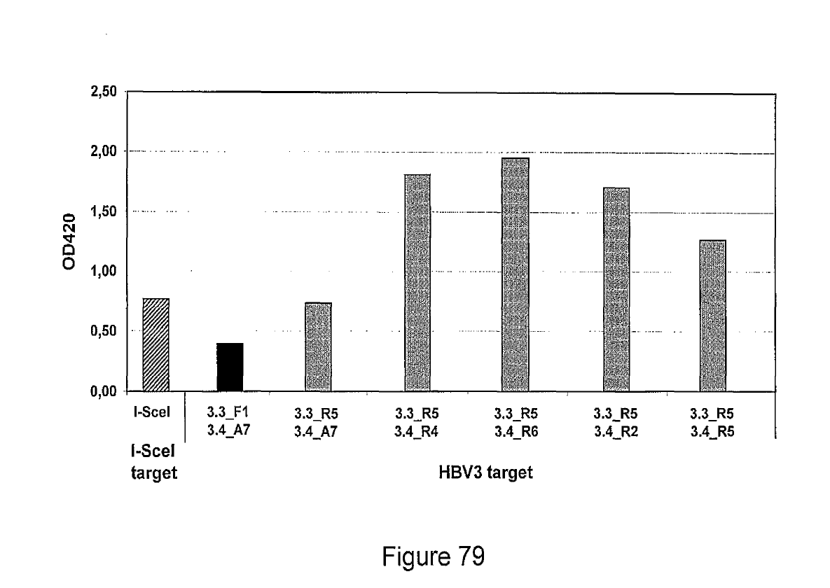

FIG. 79: Cleavage of the HBV3 target. Example of secondary screening against the HBV3 target of I-CreI refined variants obtained by site-directed mutagenesis. Extrachromosomal assay in CHO cells for heterodimers displaying cleavage activity against the HBV3 target as described in Example 32. HBV3.3 variant containing site-directed mutations (3.3_R5) was co-expressed with either the initial HBV3.4 variant (3.4_A7) or one of four HBV3.4 variants containing site-directed mutations (3.4_R2, 3.4_R4, 3.4_R5, 3.4_R6; see Table LXXIV) variants, and examined for their ability to cleave the HBV3 target in comparison to the original HBV3 heterodimer (3.3_F1/3.4_A7). OD values indicated were observed 3 hours after lysis/revelation buffer addition. I-SceI is presented as a positive control.

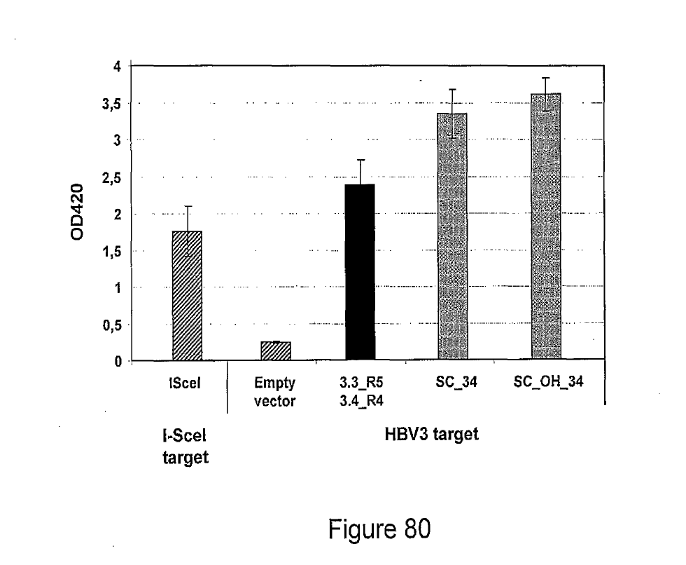

FIG. 80: Cleavage of the HBV3 target. Example of screening of I-CreI single chain molecules for cleavage activity against the HBV3 target. Extrachromosomal assay in CHO cells for single chain molecules displaying cleavage activity against the HBV3 target as described in Example 33. Two single-chain molecules (SC_{—}34 and SC_OH_{—}34) were examined for their ability to cleave the HBV3 target in comparison to the HBV3 heterodimer (3.3_R5/3.4_R4). OD values indicated were observed 3 hours after lysis/revelation buffer addition. I-SceI and empty vector are presented as positive and negative controls, respectively.

FIG. 81: shows schematic representation of HBV as an enveloped DNA-containing virus. The viral particle consists of an inner core plus an outer surface coat.

FIG. 82: shows a schematic representation of the HBV genome.

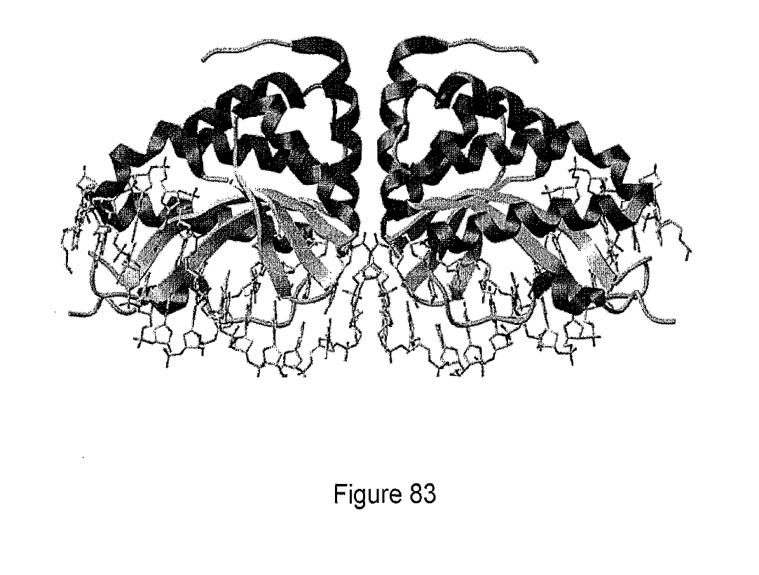

FIG. 83: shows a structural representation of a LAGLIDADG enzyme in combination with its DNA target.

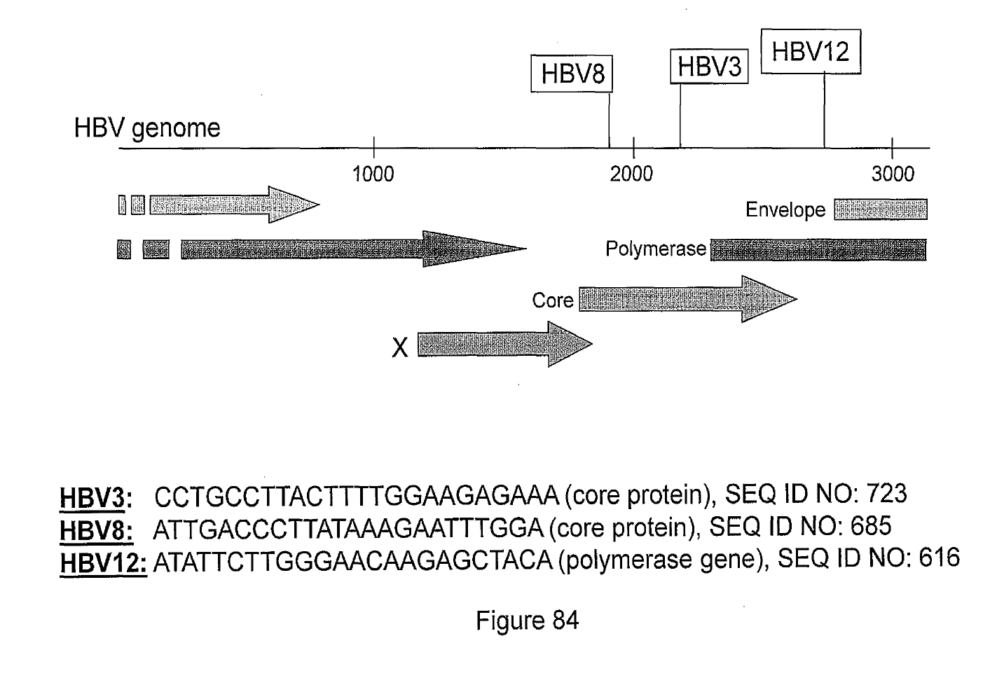

FIG. 84: shows a schematic representation of the coding sequences present in the HBV genome and the HBV3, 8 and 12 targets identified in the HBV genome for which meganuclease variants according to the present invention have been made.

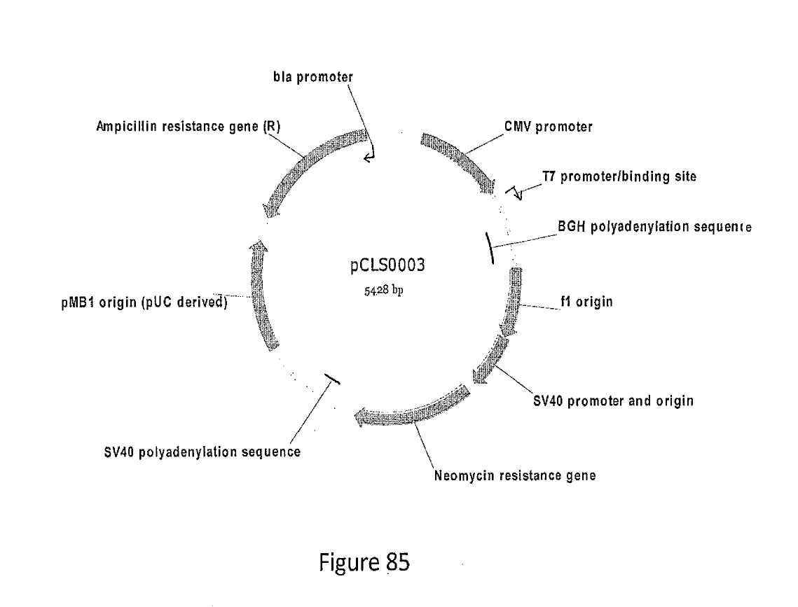

FIG. 85: pCLS0003 plasmid map.

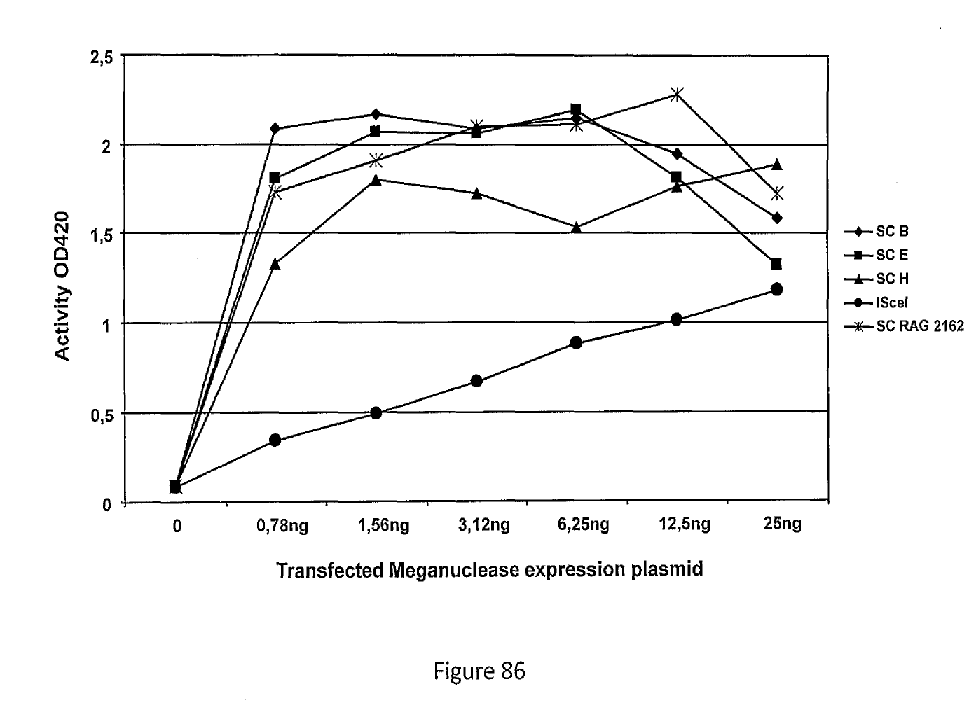

FIG. 86: Cleavage activity in CHO cells of single chain obligate heterodimer SCOH-HBV12-B1 (pCLS2862), SCOH-HBV2-B2 (pCLS2865), SCOH-HBV12-B2 (pCLS2868) meganucleases as well as I-SceI (pCLS1090) and SCOH-RAG-CLS (pCLS2162) meganucleases as positive controls.

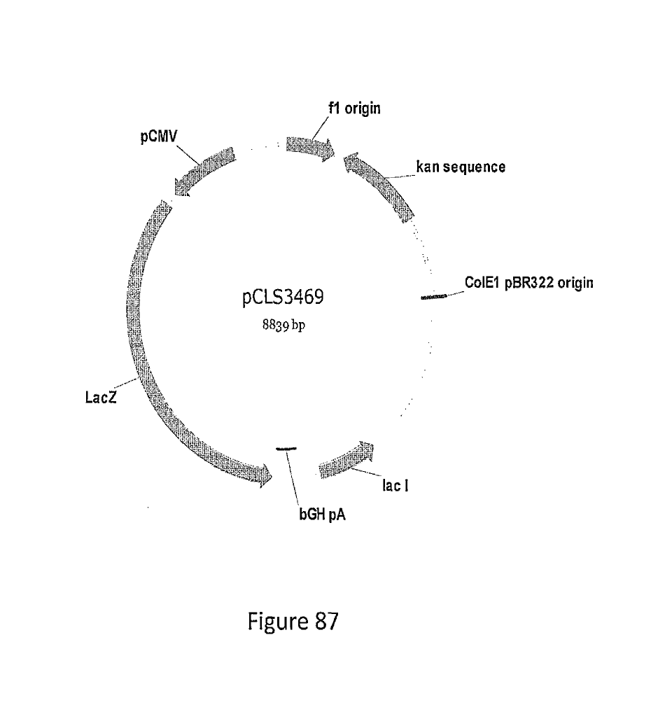

FIG. 87: pCLS3469 plasmid map.

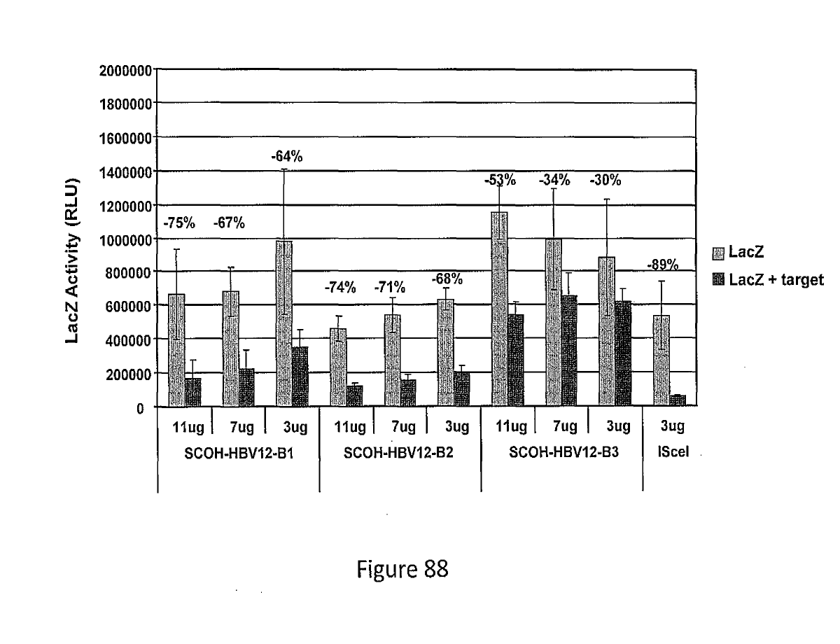

FIG. 88: Cleavage activity in HepG2 cells of single chain obligate heterodimer SCOH-HBV12-B1 (pCLS2862), SCOH-HBV12-B2 (pCLS2865), SCOH-HBV12-B2 (pCLS2868) meganucleases as well as I-SceI (pCLS1090). LacZ activities observed after transfection of different quantities (3-11 μg) of an SCOH-HBV12 expression plasmid and a fixed quantity (100 ng) of either a LacZ episomal substrate containing the HBV12 site (LacZ+target) or a LacZ substrate without the target site (LacZ) are depicted. The percent decrease in LacZ activity observed with the target substrate for each condition is indicated.

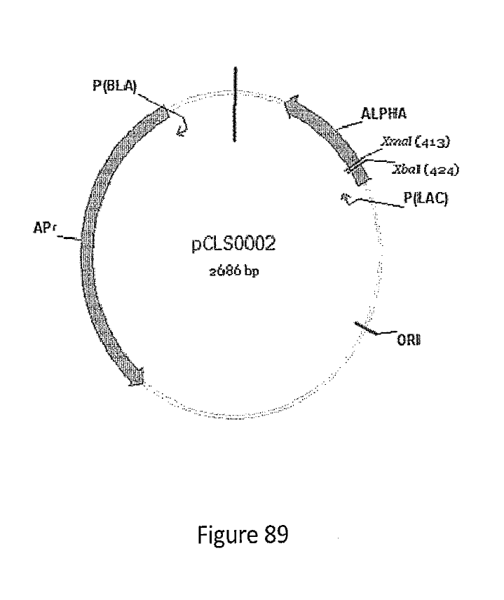

FIG. 89: pCLS0002 plasmid map.

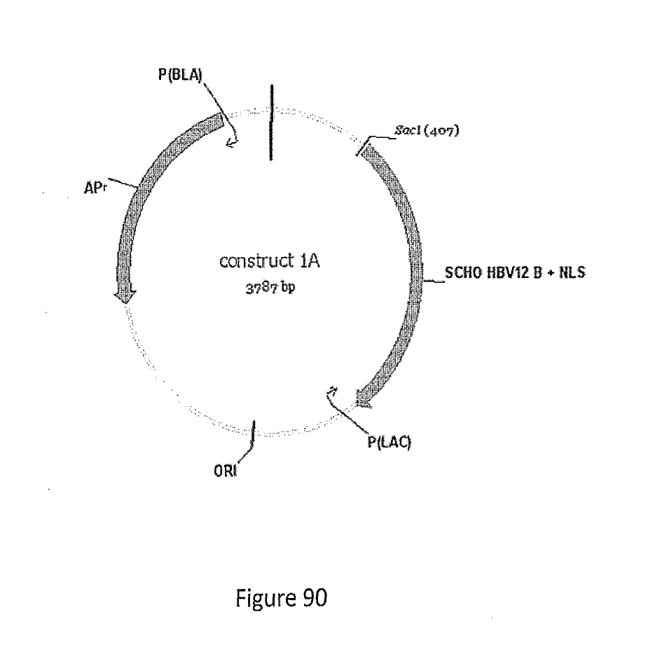

FIG. 90: construct 1A plasmid map.

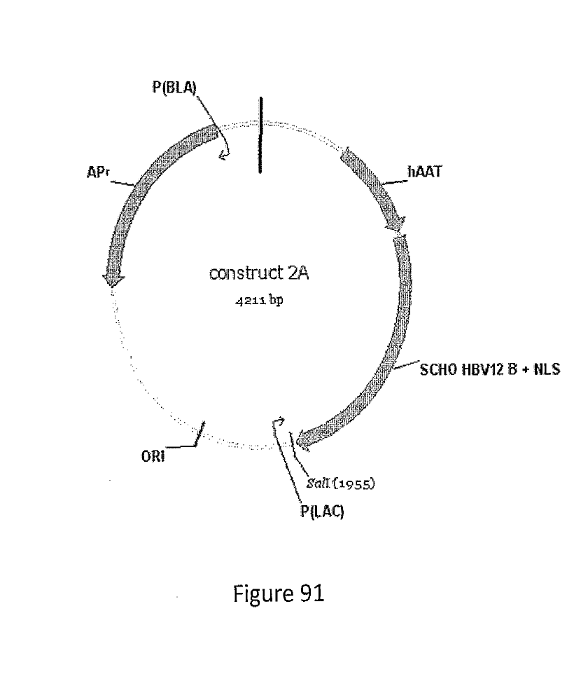

FIG. 91: construct 2A plasmid map.

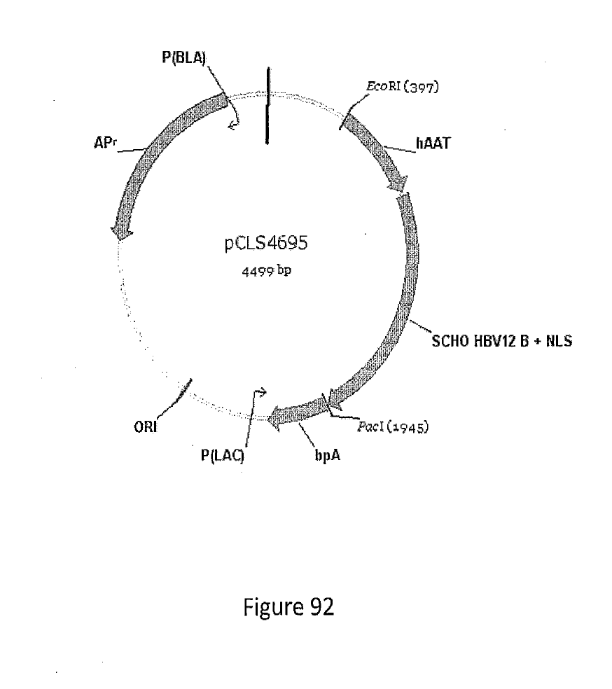

FIG. 92: pCLS4695 plasmid map.

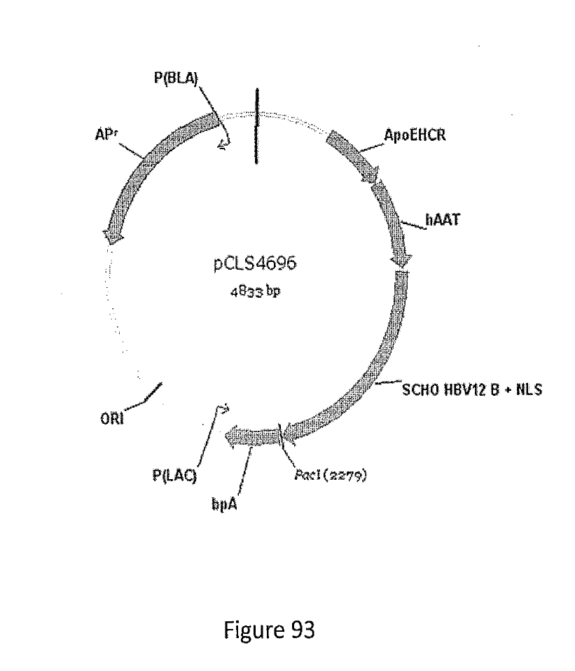

FIG. 93: pCLS4696 plasmid map.

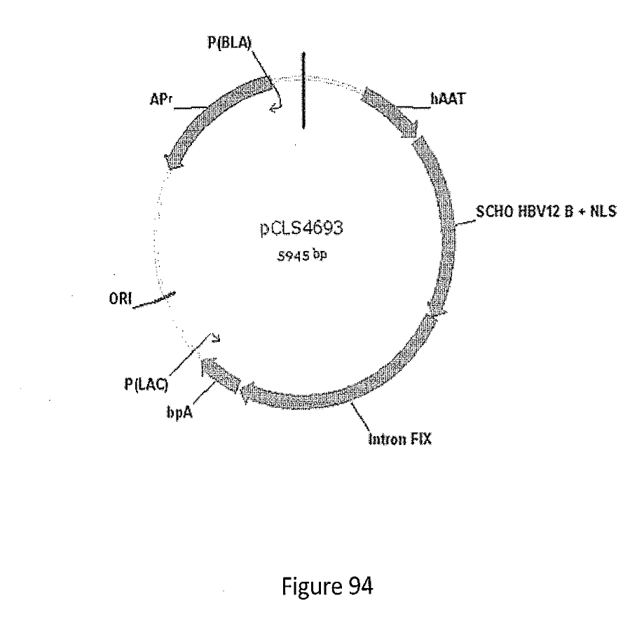

FIG. 94: pCLS4693 plasmid map.

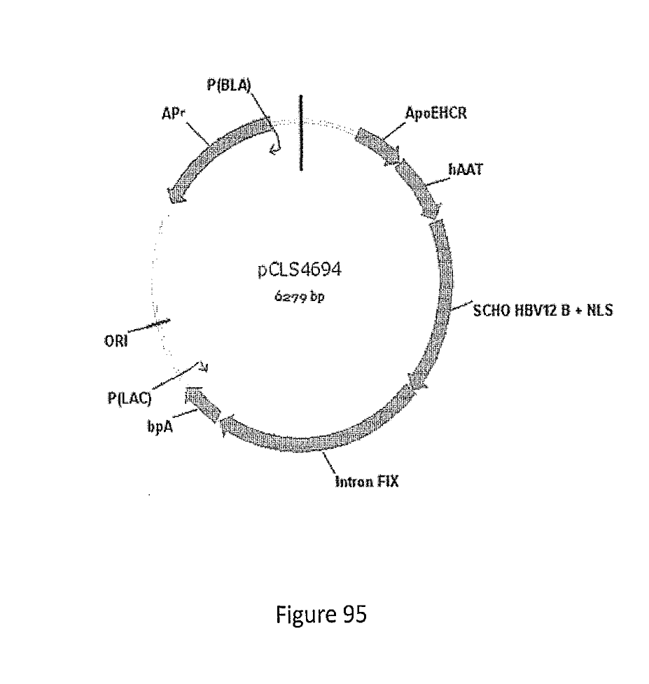

FIG. 95: pCLS4694 plasmid map.

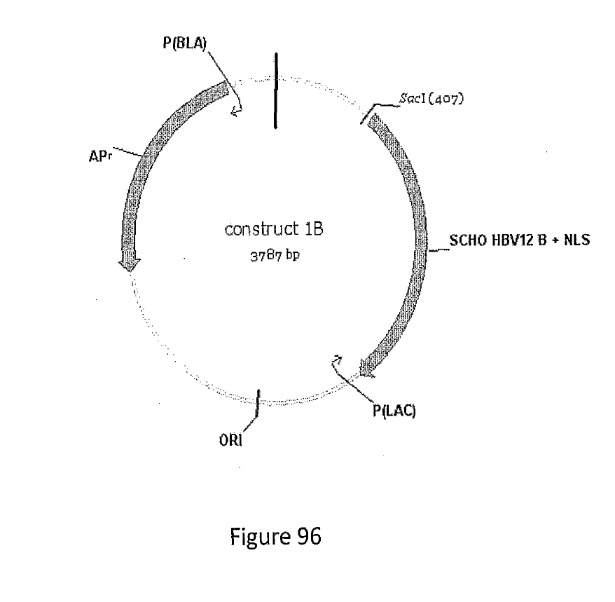

FIG. 96: construct 1B plasmid map.

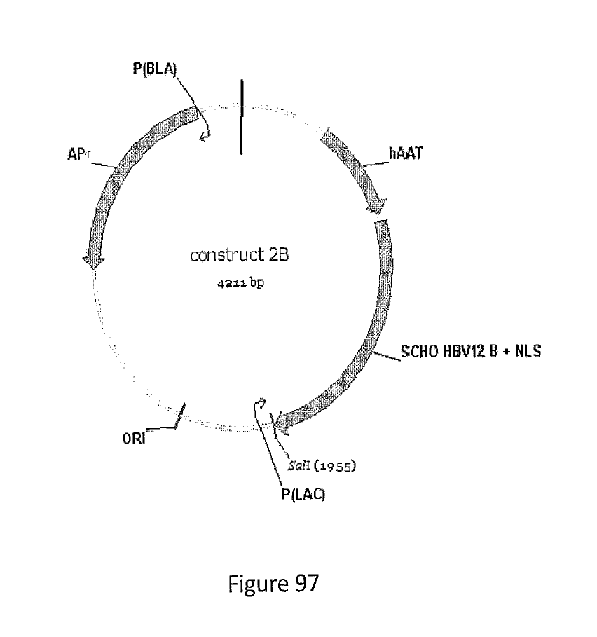

FIG. 97: construct 2B plasmid map.

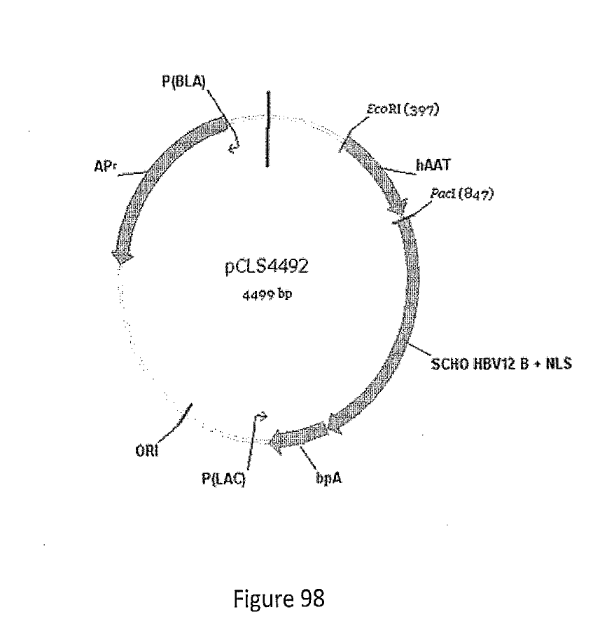

FIG. 98: pCLS4492 plasmid map.

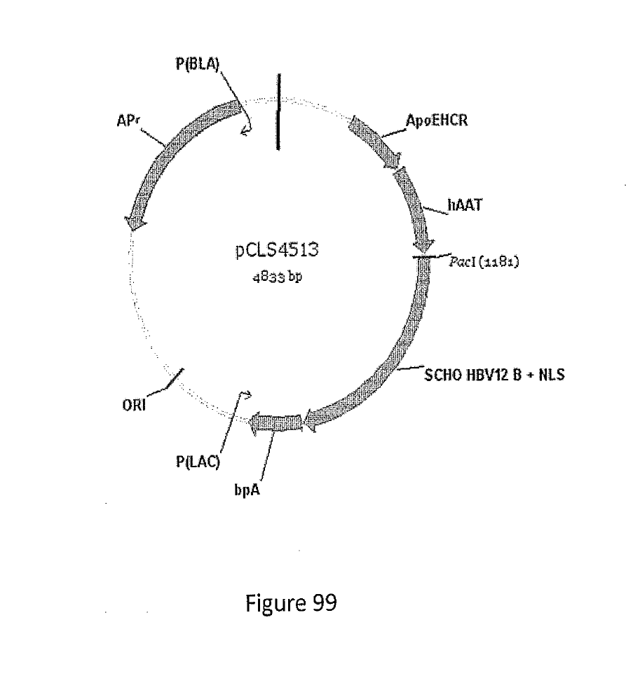

FIG. 99: pCLS4513 plasmid map.

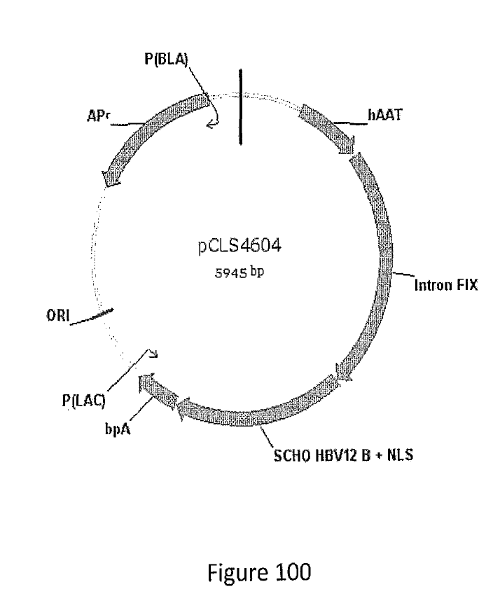

FIG. 100: pCLS4604 plasmid map.

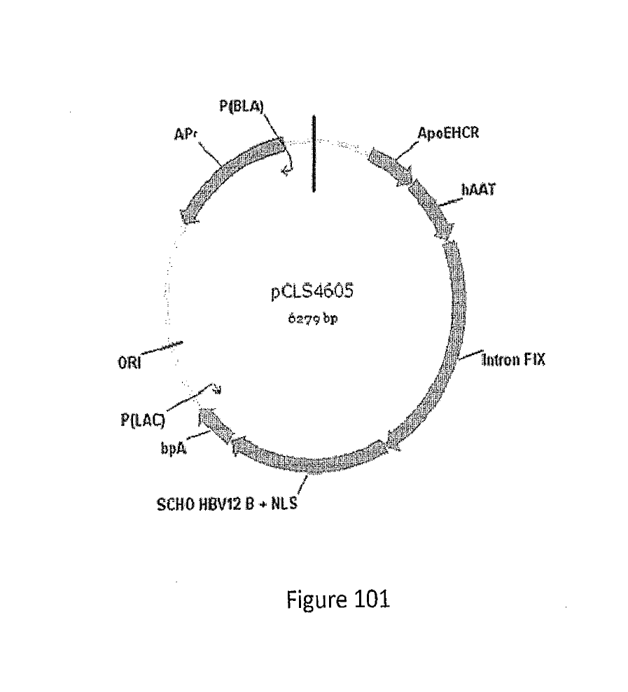

FIG. 101: pCLS4605 plasmid map.

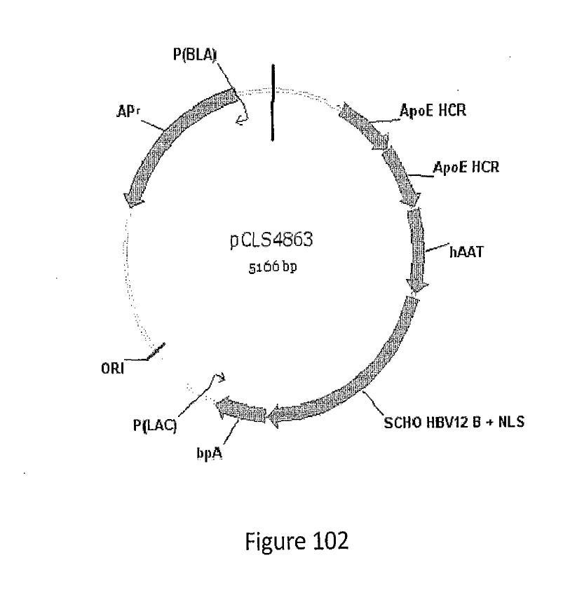

FIG. 102: pCLS4863 plasmid map.

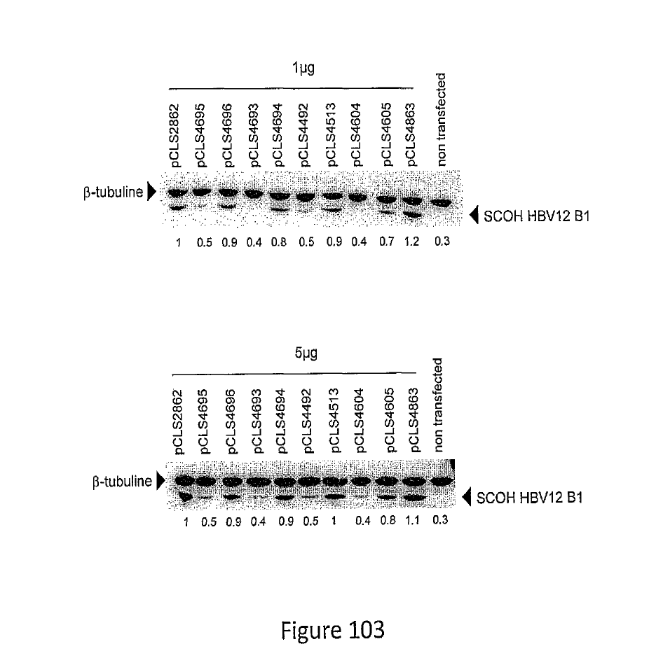

FIG. 103: Expression of single chain obligatory heterodimer SCOH-HBV12-B1 in HepG2 cells. Meganuclease expression levels were analyzed in HepG2 cells by western blotting at 48 h after transfection with various amounts of plasmid (1 and 5 μg). Antibody against β-tubulin was used for the loading control. Indicated are signal intensities as compared to the positive control pCLS2862.

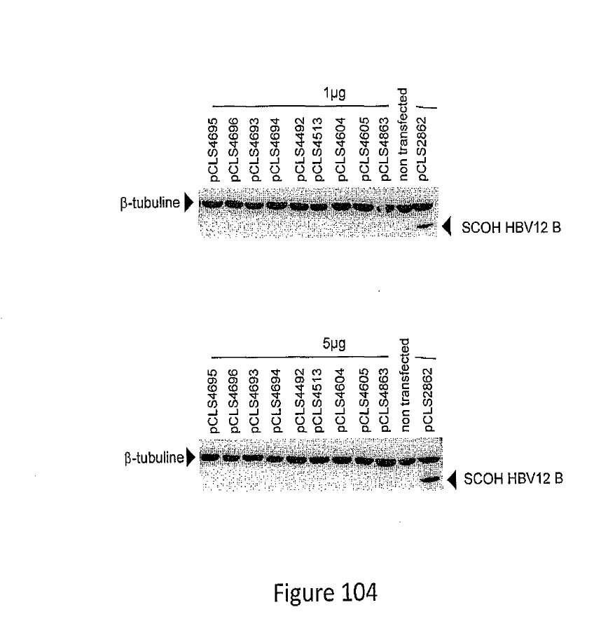

FIG. 104: Expression of single chain obligatory heterodimer SCOH-HBV12-R1 in 293H cells. Meganuclease expression levels were analyzed in 293H cells by western blotting at 48 h after transfection with various amounts of plasmid (1 and 5 μg). Antibody against β-tubulin was used for the loading control.

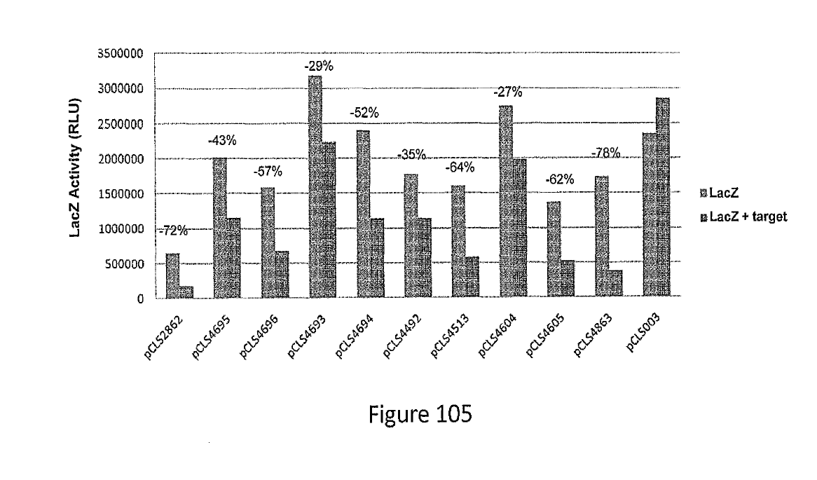

FIG. 105: Cleavage activity of hepatocyte-specific SCOH-HBV12-B1 expression constructs pCLS4695, pCLS4696, pCLS4693, pCLS4694, pCLS4492, pCLS4513, pCLS4604, pCLS4605, pCLS4863 as well as the positive control pCLS2862 and the negative control pCLS003, Indicated are LacZ activities observed after transfection of 11 μg of an SCOH-HBV12 expression plasmid and a fixed quantity (100 ng) of either a LacZ episomal substrate containing the HBV12 site (LacZ+target) or a LacZ substrate without the target site (LacZ). The percent decrease in LacZ activity observed with the target substrate for each condition is indicated.

### EXAMPLE 1

### Strategy for Engineering Novel Meganucleases Cleaving Target from the UL₁₉ Gene in HSV-1 Genome

HSV2 is a 24 bp (non-palindromic) target (SEQ ID NO: 24) present in the UL19 gene encoding the HSV-1 major capsid protein. This 5.7 kb gene is present in one copy at position 35023 to 40768 of the UL region. The HSV1-major capsid protein is expressed without maturation from an ORF located from 36404 to 40528. The target HSV2 is located from nucleotide 36966 to 36989 (accession number NC_{—}001806; FIG. 2).

The 10AAA_P, 5CAC_P, 10AGG_P, 5GCC_P targets sequences are 24 bp derivatives of C1221, a palindromic sequence cleaved by I-CreI (Arnould et al., precited). However, the structure of I-CreI bound to its DNA target suggests that the two external base pairs of these targets (positions −12 and 12) have no impact on binding and cleavage (Chevalier et al., Nat. Struct. Biol., 2001, 8, 312-316; Chevalier and Stoddard, Nucleic Acids Res., 2001, 29, 3757-3774; Chevalier et al., J. Mol. Biol., 2003, 329, 253-269), and in this study, only positions −11 to 11 were considered. Consequently, the HSV2 series of targets were defined as 22 bp sequences instead of 24 bp. HSV2 differs from C1221 in the 4 bp central region. According to the structure of the I-CreI protein bound to its target, there is no contact between the 4 central base pairs (positions −2 to 2) and the I-CreI protein (Chevalier et al., Nat. Struct. Biol., 2001, 8, 312-316; Chevalier and Stoddard, Nucleic Acids Res., 2001, 29, 3757-3774; Chevalier et al., J. Mol. Biol., 2003, 329, 253-269). Thus, the bases at these positions should not impact the binding efficiency. However, they could affect cleavage, which results from two nicks at the edge of this region. Thus, the ACAC sequence in −2 to 2 was first substituted with the GTAC sequence from C1221, resulting in target HSV2.2 (FIG. 3). Then, two palindromic targets, HSV2.3 and HSV2.4, were derived from HSV2.2 (FIG. 3). Since HSV2.3 and HSV2.4 are palindromic, they should be cleaved by homodimeric proteins. Thus, proteins able to cleave the HSV2.3 and HSV2.4 sequences as homodimers were first designed (Examples 1.1 and 1.2) and then co-expressed to obtain heterodimers cleaving HSV2 (Example 1.3). Heterodimers cleaving the HSV2.2 and HSV2 targets could be identified. In order to improve cleavage activity for the HSV2 target, a series of variants cleaving HSV2.3 and HSV2.4 was chosen, and then refined. The chosen variants were subjected to random mutagenesis, and used to form novel homodimers (Examples 1.4 and 1.5). Several improved mutants were then chosen to form heterodimers that were screened against the HSV2 target (Example 1.6). Heterodimers could be identified with an improved cleavage activity for the HSV2 target. Chosen heterodimers were then cloned into mammalian expression vectors for HSV2 cleavage in CHO cells (Example 1.7). These results were then utilized to design single chain molecules directed against the HSV2 target that were cloned into mammalian expression vectors and tested for HSV2 cleavage in CHO cells (Example 1.8). Strong cleavage activity of the HSV2 target could be observed for these single chain molecules in mammalian cells.

### EXAMPLE 1.1

### Identification of Meganucleases Cleaving HSV2.3 and HSV2.5 Targets

This Example shows that T-CreI variants can cut the HSV2.3 and HSV2.5 DNA target sequences derived from the left part of the HSV2 target in a palindromic form. Target sequences described in this Example are 22 bp palindromic sequences. Therefore, they will be described only by the first 11 nucleotides, followed by the suffix _P (For Example, target HSV2.3 will be noted HSV2.3 TAAACTCACGT_P SEQ ID NO: 10).

HSV2.3 and HSV2.5 are similar to 10AAA_P at positions ±10, +9, ±8 and to 5CAC_P at positions ±5, ±4, ±3. It was hypothesized that positions ±7 and ±11 would have little effect on the binding and cleavage activity. Variants able to cleave 10AAA-5CAC_P target were previously obtained by mutagenesis on I-CreI N75 at positions 24, 44, 68, 70, 75 and 77 as described in Arnould et al., J. Mol. Biol., 2006, 355, 443-458; Smith et al. Nucleic Acids Res., 2006, 34, e149; International PCT Applications WO 2006/097784, WO 2006/097853, WO 2007/060495 and WO 2007/049156. 192 of these variants were stored in our database and ready to be assayed for HSV2.3 and HSV2.5 cleavage.

### A) Material and Methods

### a) Construction of Target Vector

The target was cloned as follows: an oligonucleotide corresponding to the HSV2.3 and HSV2.5 targets sequences flanked by gateway cloning sequences was ordered from PROLIGO: HSV2.3

### 5′TGGCATACAAGTTTATAAACTCACOTACGTGAGTTTATCAATCGTCTGTCA3′ (SEQ ID NO: 38);

### HSV2.5

5′TGGCATACAAGTTTATAAACTCACACACGTGAGTTTATCAATCGTCTGTCA3′ (SEQ ID NO: 39). Double-stranded target DNA, generated by PCR amplification of the single stranded oligonucleotide, was cloned using the Gateway protocol (INVITROGEN) into the yeast reporter vector (pCLS1055, FIG. 4). Yeast reporter vector was transformed into Saccharomyces cerevisiae strain FYBL2-7B (MATα, ura3Δ851, trp1Δ63, leu2Δ1, lys2Δ202), resulting in a reporter strain. (MilleGen)

### b) Mating of Meganuclease Expressing Clones and Screening in Yeast

Screening of variants from our data bank was performed as described previously (Arnould et al., J. Mol. Biol., 2006, 355, 443-458). Mating was performed using a colony gridder (QpixII, GENETIX). Variants were gridded on nylon filters covering YPD plates, using a low gridding density (4-6 spots/cm²). A second gridding process was performed on the same filters to spot a second layer consisting of the reporter-harboring yeast strain. Membranes were placed on solid agar YPD rich medium, and incubated at 30° C. for one night, to allow mating. Next, filters were transferred to synthetic medium, lacking leucine and tryptophan, with galactose (2%) as a carbon source, and incubated for five days at 37° C., to select for diploids carrying the expression and target vectors. After 5 days, filters were placed on solid agarose medium with 0.02% X-Gal in 0.5 M sodium phosphate buffer, pH7.0, 0.1% SDS, 6% dimethyl formamide (DMF), 7 mM β-mercaptoethanol, 1% agarose, and incubated at 37° C., to monitor β-galactosidase activity. Results were analyzed by scanning and quantification was performed using appropriate software.

### B) Results

Examples of variants able to cleave 10AAA-5CAC_P target are displayed in Table VIII. Among 192 unique variants, 156 clones were found on HSV2.3 which correspond to 156 different endonucleases (Table IX), 55 of them where able to cut HSV2.5 as well. Examples of positives are shown in Table IX.

TABLE VIII
Panel of variants extracted from our database

| Amino acids positions and residues | |
|---|---|
| of the I-CreI variants | SEQ ID NO: |
| 44N70S75R77Y/ | 40 |
| 44V68E75N77R/24V80K | 41 |
| 44N68E70S75R77K/ | 42 |
| 44K68Q/ | 43 |
| 44R68T/ | 44 |
| 44N68Y70S75R77V/ | 45 |
| 44A70S75R77Y/24V | 46 |
| 44N70S75R77N/ | 47 |
| 44A70S75R77Y/ | 48 |
| 44N68E70S75R77R/ | 49 |
| 44R68N/ | 50 |
| 44K68A/ | 51 |
| 444N70S75R77N/24V | 52 |
| 44T68E70S75R77R/ | 53 |
| 44A68Y70S75Y77K/ | 54 |
| 44N68Y70S75R77Y/ | 55 |
| 44A70S75R77L/ | 56 |
| 44T68K70S75R77R/ | 57 |
| 44N68Y70S75R/ | 58 |
| 44N68E70S75R77R/24V | 59 |
| 44A68Y70S75R/24V | 60 |
| 44N68Y70S75R77V/24V | 61 |
| 44K68H70S75N/ | 62 |
| 44R68A/ | 63 |
| 44N68A70S75R77Y/ | 64 |
| 44D68A70S75K77R/ | 65 |
| 44R68Y70S75N77T/24V | 66 |
| 44R68Y70S75N/24V | 67 |
| 44A68Y70S75R/ | 68 |
| 44I68E75N77R/24V | 69 |
| 44N70S75R/ | 70 |
| 44T68E70S75R77R/24V | 455 |
| 44R68Y70S75Y77T/24V | 456 |
| 44N68Y70S75R77Q/ | 457 |
| 44N68Y70S75Y77K/24V | 458 |
| 44N68K70S75R77N/ | 459 |
| 44R68Y70S75Y77N/ | 460 |
| 44R68H/ | 461 |
| 44A68Y70S75R77V/24V | 462 |
| 44S68E70S75R77K/24V | 463 |

TABLE IX
I-CreI variants capable of cleaving the HSV2.3 as well as HSV2.5 DNA
targets.

| Amino acids positions and residues | |
|---|---|
| of the I-CreI variants | SEQ ID NO: |
| 44N68Y70S75R77Y/ | 71 |
| 44A68Y70S75R/ | 72 |
| 44N70S75R77Y/ | 73 |
| 44N68Y70S75R/ | 74 |
| 44T68T70S75K77E/ | 75 |
| 24V44T68T70S75K77E/ | 76 |
| 44N70S75R77N/ | 77 |
| 44A70S75R77Y/ | 78 |
| 44R68A/ | 79 |
| 44R68T/ | 80 |
| 24V44N68Y70S75Y77R/ | 81 |
| 44N68A70S75R77Y/ | 82 |
| 44N68Y70S75R77V/ | 83 |
| 44N68T70S75R77Y/ | 84 |
| 44T68Y70S75Y77R/ | 85 |
| 44A68Y70S75R77R/ | 86 |
| 44N68Y70S75Y77R/ | 87 |
| 44K68A/ | 88 |
| 44R68H/ | 89 |
| 44A68T70S75Q77R/ | 90 |

### EXAMPLE 1.2

### Identification of Meganucleases Cleaving HSV2.4 and HSV2.6

This Example shows that I-CreI variants can cleave the HSV2.4 and HSV2.6 DNA target sequences derived from the right part of the HSV2 target in a palindromic form (FIG. 3). All target sequences described in this Example are 22 bp palindromic sequences. Therefore, they will be described only by the first 11 nucleotides, followed by the suffix _P (for Example, HSV2.4 will be called CAGGACGCCGT_P).

### A) Material and Methods

### a) Construction of Target Vector

The experimental procedure is as described in Example 1.1, with the exception that an oligonucleotide corresponding to the HSV2.4 and HSV2.6 target sequences were used: 5′ TGGCATACAAGTTTCCAGGACGCCGTACGGCGTCCTGGCAATCGTCTGTCA 3′ (SEQ ID NO: 91).

and
5′TGGCATACAAGTTTCCAGGACGCCACACGGCGTCCTGGCAATCGTCTGTCA3′ (SEQ ID NO: 92) (resp. HSV2.4 and HSV2.6)

### b) Mating of Meganuclease Expressing Clones and Screening in Yeast

Screening was performed as described previously (Arnould et al., J. Mol. Biol., 2006, 355, 443-458). Mating was performed using a colony gridder (QpixII, GENETIX). Variants were gridded on nylon filters covering YPD plates, using a low gridding density (4-6 spots/cm²). A second gridding process was performed on the same filters to spot a second layer consisting of the reporter-harboring yeast strain. Membranes were placed on solid agar YPD rich medium, and incubated at 30° C. for one night, to allow mating. Next, filters were transferred to synthetic medium, lacking tryptophan, adding G418, with galactose (2%) as a carbon source, and incubated for five days at 37° C., to select for diploids carrying the expression and target vectors. After 5 days, filters were placed on solid agarose medium with 0.02% X-Gal in 0.5 M sodium phosphate buffer, pH7.0, 0.1% SDS, 6% dimethyl formamide (DMF), 7 mM β-mercaptoethanol, 1% agarose, and incubated at 37° C., to monitor β-galactosidase activity. Results were analyzed by scanning and quantification was performed using appropriate software. Positives resulting clones were verified by sequencing (MILLEGEN) as described in Example 1.1.

### B) Results

Examples of variants able to cleave 10AGG-5GCC_P target are displayed in Table X. Among 57 clones, 33 clones were positives on HSV2.4, 3 of them where able to cut HSV2.6 too. Examples of positives are shown in Table XI.

TABLE X
Panel of variants extracted from our data bank

| Amino acids positions and residues | |
|---|---|
| of the I-CreI variants | SEQ ID NO: |
| 28E33R38R40K44K70D75N/ | 93 |
| 28E33R38R40K44K70E75N/ | 94 |
| 28E33R38R40K44K68T70G75N/ | 95 |
| 28E33R38R40K44K68T70S75N/ | 96 |
| 28E38R40K44K68S70S75N/ | 97 |
| 30D33A38H44K70D75N/ | 98 |
| 30D33A38H44K70E75N/ | 99 |
| D33H38K44K70D75N/ | 100 |
| 30D33H38K44K70D75N/87L | 101 |
| 30D33H38K44K70E75N/ | 102 |
| 30D33H38K44K70T75N/ | 103 |
| 30D33R38G44K70D75N/ | 104 |
| 30D33R38G44K70D75N/17A | 105 |
| 30D33R38G44K70E75N/ | 106 |
| 30G38G44K70E75N/ | 107 |
| 30G38G44K70S75N/54I | 108 |
| 30G38H44K68A70G75N/ | 109 |
| 30G38H44K68A70Q75N/ | 110 |
| 30G38H44K68A70S75N/ | 111 |
| 28k30G38H44K68G70A75N/ | 112 |
| 28k30G38H44K68N70A75N/ | 113 |
| 30G38H44K68Q70S75N/ | 114 |
| 30G38H44K70D75N/ | 115 |
| 30G38H44K70E75N/ | 116 |
| 30G38H44K70N75N/ | 117 |
| 30G38H44K70S75N/ | 118 |
| 30G38H44K68S70D75N/ | 119 |
| 30G38H44K68T70S75N/ | 120 |
| 30G38R44K68A70G75N/ | 121 |
| 30G38R44K68A70N75N/ | 122 |
| 30G38R44K68A70Q75N/ | 123 |
| 30G38R44K68G70S75N/ | 124 |
| 30G38R44K68G70S75N/3A | 125 |
| 30G38R44K68H70S75N/ | 126 |
| 30G38R44K68Q70D75N/ | 127 |
| 30G38R44K68Q70S75N/ | 128 |
| 30G38R44K70D75N/ | 129 |
| 30G38R44K70E75N/ | 130 |
| 30G38R44K70N75N/ | 131 |

TABLE XI
I-CreI variants capable of cleaving the HSV2.4 and/or HSV2.6 DNA
targets.

| Amino acids positions and residues | |
|---|---|
| of the I-CreI variants | SEQ ID NO: |
| 28E38R40K44K70E75N/54L81V96R153V160R | 132 |
| 28E38R40K44K68S70S75N/94S132V150T | 133 |
| 30G38G44K70S75N/54I | 134 |
| 28E38R40K44K70E75N/163T | 135 |
| 28E38R40K44K70E75N/ | 136 |
| 28E38R40K44K68S70S75N/ | 137 |
| 30G38H44K70S75N/ | 138 |
| 28E38R40K44K70E75N77T/ | 139 |
| 30G38R44K70S75N/162F | 140 |
| 30G38R44K70D75N/ | 141 |
| 30G38R44K68S70S75N/ | 142 |
| 30G38R44K70S75N/ | 143 |
| 30G38R44K68T70S75N/ | 144 |
| 30G38R44K70S75N/62V | 145 |
| 30G38R44K68Q70S75N/ | 146 |
| 30G38H44K70D75N/ | 147 |
| 30G38R44K70E75N/ | 148 |
| 30G38R44K68T70G75N/ | 149 |
| 30G338R44K68G70S75N/3A | 150 |
| 30G38R44K70N75N/ | 151 |

### EXAMPLE 1.3

### Identification of Meganucleases Cleaving HSV2

I-CreI variants able to cleave each of the palindromic HSV2 derived targets (HSV2.3/2.5 and HSV2.4/2.6) were identified in Example 1.1 and 1.2. Pairs of such variants (one cutting HSV2.3 and one cutting HSV2.4) were co-expressed in yeast. Upon co-expression, there should be three active molecular species, two homodimers, and one heterodimer. It was assayed whether the heterodimers that should be formed, cut the non palindromic HSV2 target.

### A) Materials and Methods

### a) Construction of Target Vector

The experimental procedure is as described in Example 1.2, with the exception that an oligonucleotide corresponding to the HSV2 target sequence: 5′ TGGCATACAAGTTTATAAACTCACACACGGCGTCCTGGCAATCGTCTGTCA 3′ (SEQ ID NO: 152) was used.

### b) Co-Expression of Variants

Yeast DNA was extracted from variants cleaving the HSV2.4 target in the pCLS1107 (FIG. 6) expression vector using standard protocols and was used to transform E. coli. The resulting plasmid DNA was then used to transform yeast strains expressing a variant cutting the HSV2.3 target in the pCLS542 expression vector. Transformants were selected on synthetic medium lacking leucine and containing G418.

### c) Mating of Meganucleases Coexpressing Clones and Screening in Yeast

Mating was performed using a colony gridder (QpixIII, Genetix). Variants were gridded on nylon filters covering YPD plates, using a low gridding density (4-6 spots/cm²). A second gridding process was performed on the same filters to spot a second layer consisting of different reporter-harboring yeast strains for each target. Membranes were placed on solid agar YPD rich medium, and incubated at 30° C. for one night, to allow mating. Next, filters were transferred to synthetic medium, lacking leucine and tryptophan, adding G418, with galactose (2%) as a carbon source, and incubated for five days at 37° C., to select for diploids carrying the expression and target vectors. After 5 days, filters were placed on solid agarose medium with 0.02% X-Gal in 0.5 M sodium phosphate buffer, pH7.0, 0.1% SDS, 6% dimethyl formamide (DMF), 7 mM β-mercaptoethanol, 1% agarose, and incubated at 37° C., to monitor β-galactosidase activity. Results were analyzed by scanning and quantification was performed using appropriate software.

### B) Results

Co-expression of variants cleaving the HSV2.4 target (6 variants chosen among those described in Table XI) and 10 variants cleaving the HSV2.3 target (described in Table IX) resulted in cleavage of the HSV2 target in 14 cases (FIG. 7). Functional combinations are summarized in Table XII.

TABLE XII
Cleavage of the HSV2 target by the heterodimeric variants

| Amino acids positions and residues | | | | | | | | | |
|---|---|---|---|---|---|---|---|---|---|
| of the I-CreI variants cleaving the HSV2.3 target | | | | | | | | | |
| | | | | 44T68T7 | 44N68 | | 24V44T68 | | |
| 44A68Y70S7 | 44N70S7 | 44N70S7 | 44A70S7 | 0S75K77 | Y70S75 | 44N68Y7 | T70S75K7 | | |
| 5R | 5R77N | 5R77Y | 5R77Y | E | R77Y | 0S75R | 7E | 44R68T | 44R68A |
| (SEQ ID | (SEQ ID | (SEQ ID | (SEQ ID | (SEQ ID | (SEQ ID | (SEQ ID | (SEQ ID | (SEQ ID | (SEQ ID |
| NO: 159) | NO: 160) | NO: 161) | NO: 162) | NO: 163) | NO: 164) | NO: 165) | NO: 166) | NO: 167) | NO: 168) |

| | | | | | | | | | |
|---|---|---|---|---|---|---|---|---|---|
| Amino | 28E38R40K | | | + | + | + | + | | |
| acids | 44K68S70S7 | | | | | | | | |
| positions | 5N/94S132 | | | | | | | | |
| and | V150T | | | | | | | | |
| residues | (SEQ ID | | | | | | | | |
| of | NO: 153) | | | | | | | | |
| I-CreI | 28E38R40K | + | | | | | | + | |
| variants | 44K70E75N/ | | | | | | | | |
| cleaving | (SEQ ID | | | | | | | | |
| the | NO: 154) | | | | | | | | |
| HSV2.4 | 28E38R40K | | | | | + | | | + |
| target | 44K70E75N/ | | | | | | | | |
| | 77T | | | | | | | | |
| | (SEQ ID | | | | | | | | |
| | NO: 155) | | | | | | | | |
| | 28E38R40K | | | | | | | | |
| | 44K70E75N/ | | | | | | | | |
| | 163T | | | | | | | | |
| | (SEQ ID | | | | | | | | |
| | NO: 156) | | | | | | | | |
| | 30G38G44K | + | | | | | | | |
| | 70S75N/54I | | | | | | | | |
| | (SEQ ID | | | | | | | | |
| | NO: 157) | | | | | | | | |
| | 28E38R40K | | + | + | + | + | + | | |
| | 44K68S70S7 | | | | | | | | |
| | 5N7 | | | | | | | | |
| | (SEQ ID | | | | | | | | |
| | NO: 158) | | | | | | | | |

+ indicates a functional combination

### EXAMPLE 1A

### Improvement of Meganucleases Cleaving HSV2.5 by Random Mutagenesis

I-CreI variants able to cleave the palindromic HSV2.5 target have been previously identified in Example 1.1. Some of them can cleave the HSV2 target when associated with variants able to cut HSV2.6 (Examples 1.2 and 1.3).

Therefore 6 selected variants cleaving HSV2.5 were mutagenized, and variants were screened for activity improvement on HSV2.5. According to the structure of the I-CreI protein bound to its target, there is no contact between the 4 central base pairs (positions −2 to 2) and the I-CreI protein (Chevalier et al., Nat. Struct. Biol., 2001, 8, 312-316; Chevalier and Stoddard, Nucleic Acids Res., 2001, 29, 3757-3774; Chevalier et al., J. Mol. Biol., 2003, 329, 253-269). Thus, it is difficult to rationally choose a set of positions to mutagenize, and mutagenesis was performed on the whole protein. Random mutagenesis results in high complexity libraries. Therefore, to limit the complexity of the variant libraries to be tested, the two components of the heterodimers cleaving HSV2 were mutagenized and screened in parallel.

### A) Material and Methods

### a) Construction of Libraries by Random Mutagenesis

Random mutagenesis was performed on a pool of chosen variants, by PCR using Mn²⁺. PCR reactions were carried out that amplify the I-CreI coding sequence using the primers preATGCreFor (5′-gcataaattactatacttctatagacacgcaaacacaaatacacagcggccttgccacc-3′; SEQ ID NO: 169) and I-CreIpostRev (5′-ggctcgaggagctcgtctagaggatcgctcgagttatcagtcggccgc-3′; SEQ ID NO: 170), which are common to the pCLS0542 (FIG. 5) and pCLS1107 (FIG. 6) vectors. Approximately 25 ng of the PCR product and 75 ng of vector DNA (pCLS0542) linearized by digestion with NcoI and EagI were used to transform the yeast Saccharomyces cerevisiae strain FYC2-6A (MATα, trp1Δ63, leu2Δ1, his3Δ200) using a high efficiency LiAc transformation protocol (Gietz and Woods, Methods Enzymol., 2002, 350, 87-96). Expression plasmids containing an intact coding sequence for the I-CreI variant were generated by in vivo homologous recombination in yeast.

### b) Target Vector Yeast Strains

The yeast strain FYBL2-7B (MATα, ura3Δ851, trp1Δ63, leu2Δ1, lys2Δ202) containing the HSV2.5 target in the yeast reporter vector (pCLS1055 FIG. 4) was constructed as described in Example 1.1.

### c) Mating of Meganuclease Expressing Clones, Screening in Yeast and Sequencing

Mating HSV2.3 target strain and mutagenized variant clones and screening were performed as described in Example 1.1. One variant from first generation was added as control on filter during screening steps for activity improvement evaluation.

### B) Results

Six variants cleaving HSV2.5, (Table XIII), were pooled, randomly mutagenized and transformed into yeast. 2304 transformed clones were then mated with a yeast strain that contains the HSV2.5 target in a reporter plasmid. After mating with this yeast strain, 761 clones were found to cleave the HSV2.5 target. 93 of them were characterized. 72 of them shown high activity and retain HSV2.5/2.3 specificity. An Example of positives is shown in FIG. 8. Sequencing of these 46 positive clones indicates that 32 distinct variants listed in Table XIV were identified.

TABLE XIII
pool of variants cleaving HSV2.3/2.5 and sequences
used as template for random mutagenesis

| | SEQ |
|---|---|
| Amino acids positions and residues | ID |
| of the I-CreI variants | NO: |
| 44A68Y70S75R/ | 171 |
| 44N70S75R77N/ | 172 |
| 44N70S75R77Y/ | 173 |
| 44A70S75R77Y/ | 174 |
| 44T68T70S75K77E/ | 175 |
| 44N68Y70S75R77Y/ | 176 |

TABLE XIV
Improved variants
displaying strong cleavage activity for HSV2.5

| | SEQ |
|---|---|
| Amino acids positions and residues | ID |
| of the I-CreI variants | NO: |
| 44A70S75R77Y/ | 177 |
| 44D68T70S75R77H/ | 178 |
| 44D68T70S75R77P/37Y | 179 |
| 44D68T70S75R77R/ | 180 |
| 44D68T70S75R77R/100R | 181 |
| 44D68T70S75R77R/12H | 182 |
| 44D68T70S75R77R/156G | 183 |
| 44D68T70S75R77R/2D37Y129A | 184 |
| 44D68T70S75R77R/37C105A | 185 |
| 44D68T70S75R77R/37Y | 186 |
| 44D68T70S75R77R/37Y114T | 187 |
| 44D68T70S75R77R/37Y121R | 188 |
| 44D68T70S75R77R/37Y129A | 189 |
| 44D68T70S75R77R/37Y66H | 190 |
| 44D68T70S75R77R/37Y82R | 191 |
| 44D68T70S75R77R/4R151A | 192 |
| 44D68T70S75R77R/4R37Y151A | 193 |
| 44D68T70S75R77R/57E159R | 194 |
| 44D68T70S75R77R/64A | 195 |
| 44D68T70S75R77R/6S37Y | 196 |
| 44D68T70S75R77R/80K | 197 |
| 44H68T70P75R77R/ | 198 |
| 44N70S75R77Y/ | 199 |
| 44N70S75R77Y/157G | 200 |
| 44N68Y70S75R/ | 201 |
| 44N68Y70S75R/132V | 202 |
| 44N68Y70S75R77Y/ | 203 |
| 44R68A75d/54S | 204 |
| 44R68T/111R | 205 |
| 44R68T/54L121E | 206 |
| 44T68T70S75R77Q/ | 207 |
| 24V44D68T70S75R77R/ | 208 |

### EXAMPLE 1.5

### Improvement of Meganucleases Cleaving HSV2.6 by Random Mutagenesis

I-CreI variants able to cleave the palindromic HSV2.4 target has been previously identified in Example 1.2. Some of them can cleave HSV4 target when associated with variants able to cut HSV2.3 (Examples 1.1 and 1.3).

Six of the selected variants cleaving HSV2.6 and 2.4 were mutagenized, and variants were screened for activity improvement on HSV2.6. As described in Example 1.4, mutagenesis was performed on the whole protein and HSV2.4 variants were screened in parallel to HSV2.3 (Example 1.4).

### A) Material and Methods

### a) Construction of Libraries by Random Mutagenesis

Random mutagenesis was performed as described in Example 1.4, on a pool of chosen variants, by PCR using the same primers and Mn²⁺ conditions (preATGCreFor SEQ ID NO: 169 and I-CreIpostRev SEQ ID NO: 170). Approximately 25 ng of the PCR product and 75 ng of vector DNA pCLS1107) linearized by digestion with NcoI and EagI were used to transform the yeast Saccharomyces cerevisiae strain FYC2-6A (MATα, trp1Δ63, leu2Δ1, his3Δ200) using a high efficiency LiAc transformation protocol (Gietz and Woods, Methods Enzymol., 2002, 350, 87-96). Expression plasmids containing an intact coding sequence for the I-CreI variant were generated by in vivo homologous recombination in yeast.

### b) Target Vector Yeast Strains

The yeast strain FYBL2-7B (MATα, ura3Δ851, trp1Δ63, leu2Δ1, lys2Δ202) containing the HSV2.6 target in the yeast reporter vector (pCLS1055 FIG. 4) was constructed as described in Example 1.2.

### c) Mating of Meganuclease Expressing Clones, Screening in Yeast and Sequencing

Mating HSV2.6 target strain and mutagenized variant clones and screening were performed as described in Example 1.2. One variant from first generation was added as control on filter during screening steps for activity improvement evaluation.

### B) Results

Six chosen variants cleaving HSV2.6 and 2.4, (Table XV), were pooled, randomly mutagenized and transformed into yeast. 2304 transformed clones were then mated with a yeast strain that contains the HSV4.4 target in a reporter plasmid. After mating with this yeast strain, 32 clones were found to cleave the HSV2.6 target. An Example of positives is shown in FIG. 9. Sequencing 32 positive clones indicates that 19 distinct variants listed in Table XVI were identified.

TABLE XV
pool of variants cleaving HSV2.6 and 2.4 and
sequences used as template for random mutagenesis

| SEQ | |
|---|---|
| ID | Amino acids positions and residues |
| NO: | of the I-CreI variants |
| 209 | 28E38R40K44K68S70S75N/94S132V150T |
| 210 | 28E38R40K44K70E75N/ |
| 211 | 28E38R40K44K70E75N/77T |
| 212 | 28E38R40K44K70E75N/163T |
| 213 | 30G38G44K70S75N/54I |
| 214 | 28E38R40K44K68S70S75N/ |

TABLE XVI
Improved variants
displaying cleavage activity for HSV2.6

| SEQ ID | |
|---|---|
| NO: | Amino acids positions and residues of the I-CreI variants |
| 215 | 28E38R40K44K70E75N |
| 216 | 28E38R40K44K70E75N77T/50R |
| 217 | 28E38R40K44K70E75N77T/80K |
| 218 | 28E38R40K44K70E75N77T132V150T/17A |
| 219 | 28E38R40K44K70G75N132V150T/ |
| 220 | 28E38R40K44K68S70S75N/50R |
| 221 | 28E38R40K44K68S70S75N132V |
| 222 | 28E38R40K44K68S70S75N132V163T/2D |
| 223 | 28E38R40K44K68S70S75N132V150T |
| 224 | 28E38R40K44K68S70S75N94S132V |
| 225 | 28E38R40K44K68S70S75N94S132V150T |
| 226 | 28E38R40K44K68S70S75N94S132V150T/50R |
| 227 | 28E38R40K44K68S70S75N94S132V150T/66H |
| 228 | 28E38R40K44K68S70S75N77T132V |
| 229 | 28E38R40K44K70E75N77T94S |
| 230 | 28E38R40K44K70S75N |
| 231 | 28E38R40K44K70S75N150T |
| 232 | 28E38R40K44K54L70E75N94S |
| 233 | 28E38R40K44K54L70E75N77T |

### EXAMPLE 1.6

### Identification of Improved Meganucleases Cleaving HSV2

Improved I-CreI variants able to cleave each of the palindromic HSV2 derived targets (HSV2.3/2.5 and HSV2.4/2.6) were identified in Example 1.4 and Example 1.5. As described in Example 1.3, pairs of such variants (one cutting HSV2.3/2.5 and one cutting HSV2.4/2.6) were co-expressed in yeast. The heterodimers that should be formed were assayed for cutting the non palindromic HSV2 target.

### A) Materials and Methods

### a) Construction of Target Vector

The HSV2 target vector was constructed as described in Example 1.3.

### b) Co-Expression of Variants

Yeast DNA was extracted from variants cleaving the HSV2.6target in the pCLS1107 expression vector using standard protocols and was used to transform E. coli. The resulting plasmid DNA was then used to transform yeast strains expressing a variant cutting the HSV2.5target in the pCLS542 expression vector. Transformants were selected on synthetic medium lacking leucine and containing G418.

### c) Mating of Meganucleases Coexpressing Clones and Screening in Yeast

Mating and screening of meganucleases coexpressing clones were performed as described in Example 1.3. Results were analyzed by scanning and quantification was performed using appropriate software.

### B) Results

Co-expression of improved variants cleaving the HSV2.6 target (6 variants chosen among those described in Table XIV) and 7 improved variants cleaving the HSV2.5 target (described in Table XVI) resulted in cleavage of the HSV2 target in all except one case (FIG. 10). All assayed combinations are summarized in Table XVII.

TABLE XVII
Cleavage of the HSV2 target by the heterodimeric improved variants

| | | Amino acids positions and residues | | | | | | |
|---|---|---|---|---|---|---|---|---|
| | | of the I-CreI variants cleaving the HSV2.3 target | | | | | | |
| | | | | | | 44D68T70S | | |
| | | 44D68T70S75R | 44D68T70S75 | 44R6ST/ | 44D68T70S | 75R77R/15 | 44D68T70S75 | 44D68T70S75 |
| | | 77R/80K | R77R/12H | 54L1 21E | 75R77R | 6G | R77R/100R | R77R/64A |
| | | (SEQ ID | (SEQ ID | (SEQ ID | (SEQ ID | (SEQ ID | (SEQ ID | (SEQ ID |
| | | NO: 240) | NO: 241) | NO: 242) | NO: 243) | NO: 244) | NO: 245) | NO: 246) |
| Amino acids | 28E38R40K44K6 | + | + | + | | + | + | + |
| positions and | 8S7QS75N132V1 | | | | | | | |
| residues of | 50T | | | | | | | |
| I-CreI | (SEQ ID NO: 234) | | | | | | | |
| variants | 28E38R40K44KS | + | + | + | + | + | + | + |
| cleaving | 4L70E75N77T | | | | | | | |
| the HSV2.4 | (SEQ ID NO: 235) | | | | | | | |
| target | 28E30n33R40K4 | + | + | + | + | + | + | + |
| | 4K54l68r70S75N | | | | | | | |
| | 77i94f132i150a1 | | | | | | | |
| | 63p | | | | | | | |
| | (SEQ ID NO: 236) | | | | | | | |
| | 28E38R40K44K6 | + | + | + | + | + | + | + |
| | 8S70S75N/50R | | | | | | | |
| | (SEQ ID NO: 237) | | | | | | | |
| | 28E38R40K44K7 | + | + | + | + | + | + | + |
| | 0E75N77T132V1 | | | | | | | |
| | 50T/17A | | | | | | | |
| | (SEQ ID NO: 233) | | | | | | | |
| | 28E38R40K44K7 | + | + | + | + | + | + | + |
| | 0E75N77T94S | | | | | | | |
| | (SEQ ID NO: 239) | | | | | | | |

+ indicates a functional combination

### EXAMPLE 1.7

### Validation of HSV2 Target Cleavage in an Extrachromosomal Model in CHO Cells

I-CreI variants able to efficiently cleave the HSV2 target in yeast when forming heterodimers were described in Examples 1.3 and 1.7. In order to identify heterodimers displaying maximal cleavage activity for the HSV2 target in CHO cells, the efficiency of chosen combinations of variants to cut the HSV2 target was compared, using an extrachromosomal assay in CHO cells. The screen in CHO cells is a single-strand annealing (SSA) based assay where cleavage of the target by the meganucleases induces homologous recombination and expression of a LagoZ reporter gene (a derivative of the bacterial lacZ gene).

### 1) Materials and Methods

### a) Cloning of HSV2 Target in a Vector for CHO Screen

The target was cloned as follows: oligonucleotide corresponding to the HSV2 target sequence flanked by gateway cloning sequence was ordered from PROLIGO 5′TGGCATACAAGTTTATAAACTCACACACGGCGTCCTGGCAATCGTCTGTCA3′ (SEQ ID NO: 152). Double-stranded target DNA, generated by PCR amplification of the single stranded oligonucleotide, was cloned using the Gateway protocol (INVITROGEN) into CHO reporter vector (pCLS1058, FIG. 11). Cloned target was verified by sequencing (MILLEGEN).

### b) Re-Cloning of Meganucleases

The ORF of I-CreI variants cleaving the HSV2.3 and HSV2.4 targets identified in Examples 1.4 and 1.5 were sub-cloned in pCLS2437 (FIG. 12). ORFs were amplified by PCR on yeast DNA using the AT1CA1F (5′-AAAAAGCAGGCTGGCGCGCCTACACAGCGGCCTTGCCACCATG-3′ SEQ ID NO: 247) and AT2CA2R (5′-AGAAAGCTGGGTGCTAGCGCTCGAGTTATCAGTCGG-3′ SEQ ID NO: 248) primers. PCR products were cloned in the CHO expression vector pCLS2437 (FIG. 12) using the Asc I and Xho I for internal fragment replacement. Selected clones resulting from ligation and E. coli transformation steps were verified by sequencing (MILLEGEN).

### c) Extrachromosomal Assay in Mammalian Cells

CHO K cells were transfected with Polyfect® transfection reagent according to the supplier's protocol (QIAGEN). 72 hours after transfection, culture medium was removed and 150 μl of lysis/revelation buffer for β-galactosidase liquid assay was added (typically 1 liter of buffer contained: 100 ml of lysis buffer (Tris-HCl 10 mM pH7.5, NaCl 150 mM, Triton X100 0.1%, BSA 0.1 mg/ml, protease inhibitors), 10 ml of Mg 100× buffer (MgCl₂ 100 mM, β-mercaptoethanol 35%), 110 ml ONPG 8 mg/ml and 780 ml of sodium phosphate 0.1M pH7.5). After incubation at 37° C., OD was measured at 420 nm. The entire process is performed on an automated Velocity11 BioCel platform.

Per assay, 150 ng of target vector was cotransfected with 12.5 ng of each one of both mutants (12.5 ng of mutant cleaving palindromic HSV2.3 target and 12.5 ng of mutant cleaving palindromic HSV2.4 target).

### 2) Results

2 variants cleaving HSV2.5 and 2 variants cleaving HSV2.6 described in Example 1.4, 1.5 and 1.6 were re-cloned in pCLS2437 (FIG. 12). Then, I-CreI variants cleaving the HSV2.5 or HSV2.6 targets were assayed together as heterodimers against the HSV2 target in the CHO extrachromosomal assay.

Table XVIII shows the functional combinations obtained for 4 heterodimers. Analysis of the efficiencies of cleavage and recombination of the HSV2 sequence demonstrates that 4 combinations of I-CreI variants are able to transpose their cleavage activity from yeast to CHO cells without additional mutation.

TABLE XVIII
Functional heterodimeric combinations cutting the HSV2 target in
CHO cells.

| | | Optimized variants | |
|---|---|---|---|
| | | cleaving HSV2.5 | |
| | | 44D68T70S75 | |
| | | R77R/80K | 44D68T70S75R77 |
| | | (SEQ ID NO: | R/(SEQ ID NO: |
| | | 251) | 252) |
| Optimized | 28E38R40K44K70S7 | + | + |
| variants | 5N (SEQ ID NO: 249) | | |
| cleaving | 28E38R40K44K70E7 | + | + |
| HSV2.6 | 5N77T132V150T/17 | | |
| | A (SEQ ID NO: 250) | | |

+ indicates a functional combination

### EXAMPLE 1.8

### Covalent Assembly as Single Chain and Improvement of Meganucleases Cleaving HSV2 by Site-Directed Mutagenesis

Co-expression of the cutters described in Example 1.5, 1.6, 1.7 leads to a high cleavage activity of the HSV2 target in yeast. Some of them have been validated for HSV2 cleavage in a mammalian expression system. One of them is shown in Table XIX.

TABLE XIX
Example of functional heterodimer cutting the HSV2 target in CHO
cells.

| | |
|---|---|
| HSV2.3-M1 (SEQ ID NO: 33) | 44D 68T 70S 75R 77R 80K |
| HSV2.4-MC (SEQ ID NO: 34) | 28E 38R 40K 44K 54I 70S 75N |

The M1×MC HSV2 heterodimer gives high cleavage activity in yeast. M1 is a HSV2.5 cutter that bears the following mutations in comparison with the I-CreI wild type sequence: 44D 68T 70S 75R 77R 80K. MC is a HSV2.6 cutter that bears the following mutations in comparison with the I-CreI wild type sequence: 28E 38R 40K 44K 54I 70S 75N.

Single chain constructs were engineered using the linker RM2 (AAGGSDKYNQALSKYNQALSKYNQALSGGGGS) (SEQ ID NO: 464) resulting in the production of the single chain molecule: M1-RM2-MC. During this design step, the G19S mutation was introduced in the C-terminal MC mutant. In addition, mutations K7E, K96E were introduced into the M1 mutant and mutations E8K, E61R into the MC mutant to create the single chain molecule: M1(K7E K96E)-RM2-MC(E8K E61R) that is called further SCOH-HSV2-M1-MC.

Four additional amino-acid substitutions have been found in previous studies to enhance the activity of I-CreI derivatives: these mutations correspond to the replacement of Phenylalanine 54 with Leucine (F54L), Glutamic acid 80 with Lysine (E80K), Valine 105 with Alanine (V105A) and Isoleucine 132 with Valine (I132V). Only E80K is already present in HSV2.5 variant (i.e. HSV2.5-M1). Some additional combinations were introduced into the coding sequence of N-terminal and C-terminal protein fragment (an Example is shown in Table XX), and the resulting proteins were tested for their ability to induce cleavage of the HSV2 target. The twelve single chain constructs were then tested in CHO for cleavage of the HSV2 target.

TABLE XX
Single Chain I-Cre I variants for HSV2 cleavage in CHO cells.

| | | Mutations on | Mutations on | |
|---|---|---|---|---|
| | | N-terminal | C-terminal | SEQ ID |
| Construct | Single chain | segment | segment | NO |
| pCLS2456 | SCOH-HSV2- | 7E 44D 68T | 8K 19S 28E | 253 |
| | M1-MC | 70S 75R 77R | 38R 40K 44K | |
| | | 80K 96E | 54I 61R 70S | |
| | | | 75N | |
| pCLS2457 | SCOH-HSV2- | 7E 44D 68T | 8K 19S 28E | 254 |
| | M1-MC-132V | 70S 75R 77R | 38R 40K 44K | |
| | | 80K 96E | 54I 61R 70S | |
| | | | 75N 132V | |
| pCLS2458 | SCOH-HSV2- | 7E 44D 68T | 8K 19S 28E | 255 |
| | M1-MC- | 70S 75R 77R | 38R 40K 44K | |
| | 80K132V | 80K 96E | 54I 61R 70S | |
| | | | 75N 80K | |
| | | | 132V | |
| pCLS2459 | SCOH-HSV2- | 7E 44D 68T | 8K 19S 28E | 256 |
| | M1-MC- | 70S 75R 77R | 38R 40K 44K | |
| | 80K105A132V | 80K 96E | 54I 61R 70S | |
| | | | 75N 80K | |
| | | | 105A 132V | |
| pCLS2460 | SCOH-HSV2- | 7E 44D 68T | 8K 19S 28E | 257 |
| | M1-132V-MC | 70S 75R 77R | 38R 40K 44K | |
| | | 80K 96E 132V | 54I 61R 70S | |
| | | | 75N | |
| pCLS2462 | SCOH-HSV2- | 7E 44D 68T | 8K 19S 28E | 258 |
| | M1-132V- | 70S 75R 77R | 38R 40K 44K | |
| | MC-80K105A | 80K 96E 132V | 54I 61R 70S | |
| | | | 75N 80K | |
| | | | 105A | |
| pCLS2463 | SCOH-HSV2- | 7E 44D 68T | 8K 19S 28E | 259 |
| | M1-132V- | 70S 75R 77R | 38R 40K 44K | |
| | MC-80K132V | 80K 96E 132V | 54I 61R 70S | |
| | | | 75N 80K | |
| | | | 132V | |
| pCLS2464 | SCOH-HSV2- | 7E 44D 68T | 8K 19S 28E | 260 |
| | M1-132V- | 70S 75R 77R | 38R 40K 44K | |
| | MC- | 80K 96E 132V | 54I 61R 70S | |
| | 80K105A132V | | 75N 80K | |
| | | | 105A 132V | |
| pCLS2465 | SCOH-HSV2- | 7E 44D 68T | 8K 19S 28E | 261 |
| | M1- | 70S 75R 77R | 38R 40K 44K | |
| | 105A132V- | 80K 96E 105A | 54I 61R 70S | |
| | MC-132V | 132V | 75N 132V | |
| pCLS4381 | SCOH-HSV2- | 7E 44D 68T | 8K 19S 28E | 532 |
| | M1-MC- | 77R 80K 96E | 38R 40K 44K | |
| | 80K105A132 | | 54I 61R 80K | |
| | VRev1 | | 105A 132V | |
| pCLS4382 | SCOH-HSV2- | 7E 44D 68T | 8K 19S 28E | 533 |
| | M1-MC- | 77R 80K 96E | 38R 40K 44K | |
| | 80K105A132 | | 54I 61R 70S | |
| | VRev1 | | 80K 105A | |
| | | | 132V | |

### 1) Material and Methods

### a) Cloning of the SC OH Single Chain Molecule

A series of synthetic gene assembly was ordered to MWG-EUROFINS. Synthetic genes coding for the different single chain variants targeting HSV2 were cloned in pCLS1853 (FIG. 14) using AscI and XhoI restriction sites.

### b) Extrachromosomal Assay in Mammalian Cells

CHO K1 cells were transfected as described in Example 1.8. 72 hours after transfection, culture medium was removed and 150 μl of lysis/revelation buffer for β-galactosidase liquid assay was added. After incubation at 37° C., OD was measured at 420 nm. The entire process is performed on an automated Velocity11 BioCel platform.

Per assay, 150 ng of target vector was cotransfected with an increasing quantity of variant DNA from 0.75 to 25 ng (25 ng of single chain DNA corresponding to 12.5 ng+12.5 ng of heterodimer DNA). Finally, the transfected DNA variant DNA quantity was 0.78 ng, 1.56 ng, 3.12 ng, 6.25 ng, 12.5 ng and 25 ng. The total amount of transfected DNA was completed to 175 ng (target DNA, variant DNA, carrier DNA) using empty vector (pCLS0001).

### 2) Results

The activity of the SCOH-HSV2 single chain molecules (Table XX) against the HSV2 target was monitored using the previously described CHO assay in comparison to the HSV2.3-M1×HSV2.4-MC heterodimer (pCLS2733×pCLS2735) and our internal control SCOH-RAG and I-Sce I meganucleases. All comparisons were done at 0.78 ng, 1.56 ng, 3.12 ng, 6.25 ng, 12.5 ng, and 25 ng transfected variant DNA (FIGS. 18 and 19).

Variants shared specific behaviour upon assayed dose depending on the mutation profile they bear (FIGS. 18 and 19). For Example, SCOH-HSV2-M1-105A132V-MC-132V (pCLS2465) has a similar profile to our internal standard SCOH-RAG (SEQ ID NO: 468): its activity increases from low quantity to high quantity (FIG. 20). SCOH-HSV2-M1-MC-80K105A132V (pCLS2459) has an activity maximum at low quantity of transfected DNA (1.56 ng) and its activity quickly decreases with dose (FIG. 21). SCOH-HSV2-M1-MC-132V (pCLS2457) shares an intermediate profile between the two previous ones, it has maximum activity at a low dose (3.12 ng) which slowly decreases as the dose increases (FIG. 22). All of these variants could be used for HSV-1 genome targeting depending on the tissue infected.

### EXAMPLE 2

### Strategy for Engineering Novel Meganucleases Cleaving Targets from the ICP₀ Gene in HSV-1 Genome

HSV4 is a 24 bp (non-palindromic) target present in the RL2 gene encoding the ICP0 or a0 protein. This 3.6 kb gene repeated twice in TRL (2086 to 5698) and IRL (120673 to 124285) regions is formed of three exons: position 2261 to 2317, 3083 to 3749, 3886 to 5489 and 120882 to 122485, 122622 to 123288, 124054 to 124110. The target sequence present in exon 2 corresponds to positions 3498 to 3521 and 122850 to 122873 in the two copies of the HSV-1 ICP₀ gene (accession number NC_{—}001806; FIG. 23).

The HSV4 sequence is partly a patchwork of the 10AAG_P, 5GGT_P, 5CAG_P, 10ACT_P targets (FIG. 24).

The 10AAG_P, 5GGT_P, 5CAG_P, 10ACT_P targets sequences are 24 bp derivatives of C1221, a palindromic sequence cleaved by I-CreI (Arnould et al., precited). However, the structure of I-CreI bound to its DNA target suggests that the two external base pairs of these targets (positions −12 and 12) have no impact on binding and cleavage (Chevalier et al., Nat. Struct. Biol., 2001, 8, 312-316; Chevalier and Stoddard, Nucleic Acids Res., 2001, 29, 3757-3774; Chevalier et al., J. Mol. Biol., 2003, 329, 253-269), and in this study, only positions −11 to 11 were considered. Consequently, the HSV4 series of targets were defined as 22 bp sequences instead of 24 bp. HSV4 do not differs from C1221 in the 4 bp central region. According to the structure of the I-CreI protein bound to its target, there is no contact between the 4 central base pairs (positions −2 to 2) and the I-CreI protein (Chevalier et al., Nat. Struct. Biol., 2001, 8, 312-316; Chevalier and Stoddard, Nucleic Acids Res., 2001, 29, 3757-3774; Chevalier et al., J. Mol. Biol., 2003, 329, 253-269). Thus, the bases at these positions should not impact the binding efficiency. However if different, they could have affected cleavage, which results from two nicks at the edge of this region. Thus, the sequence gtac in −2 to 2 was not modified during process.

Two palindromic targets, HSV4.3 and HSV4.4, were derived from HSV4 (FIG. 24). Since HSV4.3 and HSV4.4 are palindromic, they should be cleaved by homodimeric proteins. Thus, proteins able to cleave the HSV4.3 and HSV4.4 sequences as homodimers were first designed (Examples 2.1 and 2.2) and then co-expressed to obtain heterodimers cleaving HSV4 (Example 2.3). Heterodimers cleaving the HSV4 target could be identified. In order to improve cleavage activity for the HSV4 target, a series of variants cleaving HSV4.3 and HSV4.4 was chosen, and then refined. The chosen variants were subjected to random or site-directed mutagenesis, and used to form final heterodimers that were assayed against the HSV4 target (Examples 2.4, 2.5 and 2.6). Heterodimers could be identified with an improved cleavage activity for the HSV4 target. Chosen heterodimers were subsequently cloned into mammalian expression vectors and screened against the HSV4 target in CHO cells (Example 2.7). From positive heterodimer combinations in CHO cells, single chain variants with additional mutations were designed as final constructs for HSV4 targeting in mammalian cells. Strong cleavage activity of the HSV4 target could be observed for these heterodimers and single chain variants (Example 2.8).

### EXAMPLE 2.1

### Identification of Meganucleases Cleaving HSV4.3

This Example shows that I-CreI variants can cut the HSV4.3 DNA target sequence derived from the left part of the HSV4 target in a palindromic form. Target sequences described in this Example are 22 bp palindromic sequences. Therefore, they will be described only by the first 11 nucleotides, followed by the suffix _P (For Example, target HSV4.3 will be noted HSV4.3 CAAGCTGGTGT_P SEQ ID NO: 18).

### A) Material and Methods

### a) Construction of Target Vector

The target was cloned as follows: an oligonucleotide corresponding to the HSV4.3 target sequence flanked by gateway cloning sequences was ordered from (PROLIGO): 5′TGGCATACAAGTTTCCAAGCTGGTGTACACCAGCTTGGCAATCGTCTGTCA3′ (SEQ ID NO: 262). Double-stranded target DNA, generated by PCR amplification of the single stranded oligonucleotide, was cloned using the Gateway protocol (INVITROGEN) into the yeast reporter vector (pCLS1055, FIG. 4). Yeast reporter vector was transformed into Saccharomyces cerevisiae strain FYBL2-7B (MATα, ura3Δ851, trp1Δ63, leu2Δ1, lys2Δ202), resulting in a reporter strain. (MilleGen)

### b) Construction of Combinatorial Mutants

I-CreI variants cleaving 10AAG_P or 5GGT_P were previously identified, as described in Smith et al. Nucleic Acids Res., 2006, 34, e149; International PCT Applications WO 2007/060495 and WO 2007/049156, and Arnould et al., J. Mol. Biol., 2006, 355, 443-458; International PCT Applications WO 2006/097784 and WO 2006/097853, respectively for the 10AAG_P and 5GGT_P targets. In order to generate I-CreI derived coding sequences containing mutations from both series, separate overlapping PCR reactions were carried out that amplify the 5′ end (aa positions 1-43) or the 3′ end (positions 39-167) of the I-CreI coding sequence. For both the 5′ and 3′ end, PCR amplification is carried out using primers For both the 5′ and 3′ end, PCR amplification is carried out using primers (Gal10F 5′-gcaactttagtgctgacacatacagg-3′ (SEQ ID NO: 263) or Gal10R 5′-acaaccttgattggagacttgacc-3′(SEQ ID NO: 264)) specific to the vector (pCLS0542, FIG. 5) and primers (assF 5′-ctannnttgaccttt-3′ (SEQ ID NO: 265) or assR 5′-aaaggtcaannntag-3′(SEQ ID NO: 266)), where nnn codes for residue 40, specific to the I-CreI coding sequence for amino acids 39-43. The PCR fragments resulting from the amplification reaction realized with the same primers and with the same coding sequence for residue 40 were pooled. Then, each pool of PCR fragments resulting from the reaction with primers Gal10F and assR or assF and Gal10R was mixed in an equimolar ratio. Finally, approximately 25 ng of each final pool of the two overlapping PCR fragments and 75 ng of vector DNA (pCLS0542, FIG. 5) linearized by digestion with NcoI and EagI were used to transform the yeast Saccharomyces cerevisiae strain FYC2-6A (MATα, trp1Δ63, leu2Δ1, his3Δ200) using a high efficiency LiAc transformation protocol (Gietz and Woods, Methods Enzymol., 2002, 350, 87-96). An intact coding sequence containing both groups of mutations is generated by in vivo homologous recombination in yeast.

### c) Mating of Meganuclease Expressing Clones and Screening in Yeast

Screening was performed as described previously (Arnould et al., J. Mol. Biol., 2006, 355, 443-458). Mating was performed using a colony gridder (QpixII, GENETIX). Variants were gridded on nylon filters covering YPD plates, using a low gridding density (4-6 spots/cm²). A second gridding process was performed on the same filters to spot a second layer consisting of the reporter-harboring yeast strain. Membranes were placed on solid agar YPD rich medium, and incubated at 30° C. for one night, to allow mating. Next, filters were transferred to synthetic medium, lacking leucine and tryptophan, with galactose (2%) as a carbon source, and incubated for five days at 37° C., to select for diploids carrying the expression and target vectors. After 5 days, filters were placed on solid agarose medium with 0.02% X-Gal in 0.5 M sodium phosphate buffer, pH7.0, 0.1% SDS, 6% dimethyl formamide (DMF), 7 mM β-mercaptoethanol, 1% agarose, and incubated at 37° C., to monitor β-galactosidase activity. Results were analyzed by scanning and quantification was performed using appropriate software.

### d) Sequencing of Variants

To recover the variant expression plasmids, yeast DNA was extracted using standard protocols and used to transform E. coli. Sequencing of variant ORFs was then performed on the plasmids by MILLEGEN SA. Alternatively, ORFs were amplified from yeast DNA by PCR (Akada et al., Biotechniques, 2000, 28, 668-670), and sequencing was performed directly on the PCR product by MILLEGEN SA.

### B) Results

I-CreI combinatorial variants were constructed by associating mutations at positions 44, 68, 70, 75 and 77 from proteins cleaving 5GGT_P with the 28, 30, 32, 33, 38 and 40 mutations from proteins cleaving 10AAG_P on the I-CreI scaffold, resulting in a library of complexity 1680. Examples of combinatorial variants are displayed in Table XXI. This library was transformed into yeast and 3348 clones (2 times the diversity) were screened for cleavage against the HSV4.3 DNA target (CCAAGCTGGTGTACACCAGCTTGG). 9 positive clones were found which after sequencing turned out to correspond to 7 different novel endonuclease variants (Table XXII). Examples of positives are shown in Table XXII. The sequences of three variants identified display non parental combinations at positions 28, 30, 32, 33, 38, 40 or 44, 68, 70, 75, 77. These variants may be I-CreI combined variants resulting from micro-recombination between two original variants during in vivo homologous recombination in yeast. Moreover, two of the selected variants display additional mutations to parental combinations (see Examples Table XXII). Such mutations likely result from PCR artifacts during the combinatorial process.

TABLE XXI
Panel of variants theoretically present in the combinatorial library

| Amino acids at | | | | | | | | | | | |
|---|---|---|---|---|---|---|---|---|---|---|---|
| positions 44, 68, | | | | | | | | | | | |
| 70, 75 and 77 | | | | | | | | | | | |
| (ex: ARNNI | | | | | | | | | | | |
| stands for A44, | Amino acids at positions 28, 30, 32, 33, 38 and 40 | | | | | | | | | | |
| R68, N70, N75 | (ex: KHSSQS stands for K28, H30, S32, S33, Q38 and S40) | | | | | | | | | | |
| and I77) | NTSYDS | RNSAYQ | SNSYQK | KASYQS | KASHQS | KASTQS | KDSRQS | KGSYQS | KGAYQS | KGSYGS | KGSYHS |

ASSDR
ARHDI
DQSYR
DRHDI
DRRNI
HKKDI
IRKNV
KYSNV
KSTDI
LRKNV
LRNNI
MRANI
MRCNI
NKSHF
TRHDI
TRKDI
YRSDI
YRSAT
YRSEI
YRSNI
YRSNV
YRSYQ
YRSYT
QRSNL
*Only 264 out of the 1680 combinations are displayed. None of them were identified in the positive clones.

TABLE XXII
I-CreI variants with and without additional mutations capable of
cleaving the HSV4.3 DNA target.

| Amino acids at positions 28, 30, 32, | |
|---|---|
| 33, 38, 40/44, 68, 70, 75 and 77 | |
| of the I-CreI variants | |
| (ex: KRSRES/TYSNI stands for | SEQ |
| K28, R30, S32, R33, E38, S40/T44, | ID |
| Y68, S70, N75 and 177) | NO: |
| KNSYQS/MRANV/132V | 267 |
| KNSYQS/MRCNI/12H | 268 |
| SNSYQK/MRNNI/80K94L | 269 |
| KNSYQS/LRNNI/80K | 270 |
| KGSYQS/IRKNV/132V | 271 |
| KNSYQS/YRKDI | 272 |
| SNSYQK/LRNNI/80K | 273 |

### EXAMPLE 2.2

### Identification of Meganucleases Cleaving HSV4.4

This Example shows that I-CreI variants can cleave the HSV4.4 DNA target sequence derived from the right part of the HSV4 target in a palindromic form (FIG. 24). All target sequences described in this Example are 22 bp palindromic sequences. Therefore, they will be described only by the first 11 nucleotides, followed by the suffix _P (for Example, HSV4.4 will be called CACTATCAGGT_P).

### A) Material and Methods

### a) Construction of Target Vector

The experimental procedure is as described in Example 2.1, with the exception that an oligonucleotide corresponding to the HSV4.4 target sequence was used:

(SEQ ID NO: 274)

5′TGGCATACAAGTTTCCACTATCAGGTACCTGATAGTGGCAATCGTCTG
TCA3′.

### b) Construction of Combinatorial Variants

I-CreI variants cleaving 10ACT_P or 5CAG_P were previously identified, as described in Smith et al. Nucleic Acids Res., 2006, 34, e149; International PCT Applications WO 2007/060495 and WO 2007/049156, and Arnould et al., J. Mol. Biol., 2006, 355, 443-458; International PCT Applications WO 2006/097784 and WO 2006/097853, respectively for the 10ACT_P and 5CAG_P targets. In order to generate I-CreI derived coding sequences containing mutations from both series, separate overlapping PCR reactions were carried out that amplify the 5′ end (aa positions 1-43) or the 3′ end (positions 39-167) of the I-CreI coding sequence. For both the 5′ and 3′ end, PCR amplification is carried out using primers (Gal10F 5′-gcaactttagtgctgacacatacagg-3′ (SEQ ID NO: 263) or Gal10R 5′-acaaccttgattggagacttgacc-3′ (SEQ ID NO: 264)) specific to the vector (pCLS1107, FIG. 6) and primers (assF 5′-ctannnttgaccttt-3′ (SEQ ID NO: 265) or assR 5′-aaaggtcaannntag-3′(SEQ ID NO: 266)), where nnn codes for residue 40, specific to the I-CreI coding sequence for amino acids 39-43. The PCR fragments resulting from the amplification reaction realized with the same primers and with the same coding sequence for residue 40 were pooled. Then, each pool of PCR fragments resulting from the reaction with primers Gal10F and assR or assF and Gal10R was mixed in an equimolar ratio. Finally, approximately 25 ng of each final pool of the two overlapping PCR fragments and 75 ng of vector DNA (pCLS1107, FIG. 6) linearized by digestion with NcoI and EagI were used to transform the yeast Saccharomyces cerevisiae strain FYC2-6A (MATα, trp1Δ63, leu2Δ1, his3Δ200) using a high efficiency LiAc transformation protocol (Gietz and Woods, Methods Enzymol., 2002, 350, 87-96). An intact coding sequence containing both groups of mutations is generated by in vivo homologous recombination in yeast.

### c) Mating of Meganuclease Expressing Clones and Screening in Yeast

Screening was performed as described previously (Arnould et al., J. Mol. Biol., 2006, 355, 443-458). Mating was performed using a colony gridder (QpixII, GENETIX). Variants were gridded on nylon filters covering YPD plates, using a low gridding density (4-6 spots/cm²). A second gridding process was performed on the same filters to spot a second layer consisting of the reporter-harboring yeast strain. Membranes were placed on solid agar YPD rich medium, and incubated at 30° C. for one night, to allow mating. Next, filters were transferred to synthetic medium, lacking tryptophan, adding G418, with galactose (2%) as a carbon source, and incubated for five days at 37° C., to select for diploids carrying the expression and target vectors. After 5 days, filters were placed on solid agarose medium with 0.02% X-Gal in 0.5 M sodium phosphate buffer, pH7.0, 0.1% SDS, 6% dimethyl formamide (DMF), 7 mM β-mercaptoethanol, 1% agarose, and incubated at 37° C., to monitor β-galactosidase activity. Results were analyzed by scanning and quantification was performed using appropriate software. Positives resulting clones were verified by sequencing (MILLEGEN) as described in Example 2.2.

### B) Results

I-CreI combinatorial variants were constructed by associating mutations at positions 44, 68, 70, 75 and 77 from proteins cleaving 5CAG_P with the 28, 30, 32, 33, 38 and 40 mutations from proteins cleaving 10ACT_P on the I-CreI scaffold, resulting in a library of complexity 1600. Examples of combinatorial variants are displayed in Table XXIII. This library was transformed into yeast and 3348 clones (2.1 times the diversity) were screened for cleavage against the HSV4.4 DNA target (CACTATCAGGT_P). A total of 20 positive clones were found to cleave HSV4.4. Sequencing and validation by secondary screening of these I-CreI variants resulted in the identification of 14 different novel endonucleases. The sequence of 4 of the variants identified display non parental combinations at positions 28, 30, 32, 33, 38, 40 or 44, 68, 70, 75, 77 as well as additional mutations (see Examples in Table XXIV). Such variants likely result from PCR artifacts during the combinatorial process. Alternatively, the variants may be I-CreI combined variants resulting from micro-recombination between two original variants during in vivo homologous recombination in yeast.

TABLE XXIII
Panel of variants theoretically present in the combinatorial library

| Amino acids at | | | | | | | | | | |
|---|---|---|---|---|---|---|---|---|---|---|
| positions 44, 68, | | | | | | | | | | |
| 70, 75 and 77 | | | | | | | | | | |
| (ex: HNRDI | | | | | | | | | | |
| stands for H44, | Amino acids at positions 28, 30, 32, 33, 38 and 40 | | | | | | | | | |
| N68, R70, D75 | (ex: KRGYQS stands for K28, R30, G32, Y33, Q38 and S40) | | | | | | | | | |
| and I77) | KASTQS | KCSCQS | KGSYGS | KHSSQS | KKSAQS | KKSHQS | KKSRQS | KKSSQS | KKSTQS | KNDYYS |

ARGNI
ARSNI
ATNNI
ANNNI
NRNNI
ARNNI
AQRNI
ARHNI
QGGNI
AASYK
ARSYT
RYSEV
DHSYI
RYSEV
DHSYI
RYSDT
ASSYK
SYSYV
NHSYN
ATSDR
AYSYI
RYSYV
ARSDR
RYSYN
* Only 220 out of the 1600 combinations are displayed.
+ indicates that a functional combinatorial variant cleaving the HSV4.4 target was found among the identified positives.

TABLE XXIV
I-CreI variants with and without additional mutations capable of
cleaving the HSV4.4 DNA target.

| Amino acids at positions 28, 30, 32, | |
|---|---|
| 33, 38, 40/44, 68, 70, 75 and 77 | |
| of the I-CreI variants | SEQ |
| (ex: KRGYQS/KYSNI stands for K28, R30, G32, | ID |
| Y33, Q38, S40/K44, Y68, S70, N75 and I77) | NO: |
| KNSRES/NHSYN/80K | 275 |
| KNTYSS/TYSYV/80K | 276 |
| KNTYWS/ARSYY | 277 |
| KNTCQS/AYSYK | 278 |
| KNTYSS/AYSYK | 279 |
| KRDYQS/ACSYI/115V | 280 |
| KRSYES/AYSYK | 281 |
| KNEYYS/NYSYK | 282 |
| KNAYYS/AYSYK | 283 |
| KRDYQS/AYSYK | 284 |
| KNEYYS/AYSYK | 285 |
| KNSRES/AYSYK | 286 |
| KRDYQS/ARNNI | 287 |
| KNDYYS/AYSYK | 288 |

### EXAMPLE 2.3

### Identification of Meganucleases Cleaving HSV4

I-CreI variants able to cleave each of the palindromic HSV4 derived targets (HSV4.3 and HSV4.4) were identified in Example 2.2. Pairs of such variants (one cutting HSV4.3 and one cutting HSV4.4) were co-expressed in yeast. Upon co-expression, there should be three active molecular species, two homodimers, and one heterodimer. It was assayed whether the heterodimers that should be formed, cut the non palindromic HSV4 target.

### A) Materials and Methods

### a) Construction of Target Vector

The experimental procedure is as described in Example 2.2, with the exception that an oligonucleotide corresponding to the HSV4 target sequence: 5′TGGCATACAAGTTTCCAAGCTGGTGTACCTGATAGTGGCAATCGTCTGTC A3′ (SEQ ID NO: 289) was used.

### b) Co-Expression of Variants

Yeast DNA was extracted from variants cleaving the HSV4.4 target in the pCLS1107 expression vector using standard protocols and was used to transform E. coli. The resulting plasmid DNA was then used to transform yeast strains expressing a variant cutting the HSV4.3 target in the pCLS542 expression vector. Transformants were selected on synthetic medium lacking leucine and containing G418.

### c) Mating of Meganucleases Coexpressing Clones and Screening in Yeast

Mating was performed using a colony gridder (QpixII, Genetix). Variants were gridded on nylon filters covering YPD plates, using a low gridding density (4-6 spots/cm²). A second gridding process was performed on the same filters to spot a second layer consisting of different reporter-harboring yeast strains for each target. Membranes were placed on solid agar YPD rich medium, and incubated at 30° C. for one night, to allow mating. Next, filters were transferred to synthetic medium, lacking leucine and tryptophan, adding G418, with galactose (2%) as a carbon source, and incubated for five days at 37° C., to select for diploids carrying the expression and target vectors. After 5 days, filters were placed on solid agarose medium with 0.02% X-Gal in 0.5 M sodium phosphate buffer, pH7.0, 0.1% SDS, 6% dimethyl formamide (DMF), 7 mM β-mercaptoethanol, 1% agarose, and incubated at 37° C., to monitor 1-galactosidase activity. Results were analyzed by scanning and quantification was performed using appropriate software.

### B) Results

Co-expression of variants cleaving the HSV4.4 target (9 variants chosen among those described in Table XXIV) and 6 variants cleaving the HSV4.3 target (described in Table XXII) resulted in cleavage of the HSV4 target in some cases (FIG. 25). Functional combinations are summarized in Table XXV.

TABLE XXV
Cleavage of the HSV4 target by the heterodimeric variants

| | | Amino acids at positions 28, 30, 32, 33, 38, 40/44, 68, 70, 75 and 77 | | | | | |
|---|---|---|---|---|---|---|---|
| | | of I-CreI variants cleaving the HSV4.3 target | | | | | |
| | | (ex: KRSRES/TYSNI stands for K28, R30, S32, R33, E38, S40/T44, Y68, S70, N75 and I77) | | | | | |
| | | SNSYQK/ | SNSYQK/ | KNSYQS/ | KNSYQS/ | KNSYQS/ | KGSYQS/ |
| | | MRNNI/80K94L | LRNNI/80K | MRANV/132V | LRNNI/80K | YRKDI | IRKNV/132V |
| | | (SEQ ID | (SEQ ID | (SEQ ID | (SEQ ID | (SEQ ID | (SEQ ID |
| | | NO: 299) | NO: 300) | NO: 301) | NO: 302) | NO: 303) | NO: 304) |
| Amino acids | KNDYYS/AYSYK | + | + | + | + | + | + |
| at positions | (SEQ ID NO: 290) | | | | | | |
| 28, 30, 32, 33, | KNAYYS/AYSYK | | | | | | |
| 38, 40/44, 68, | (SEQ ID NO: 291) | | | | | | |
| 70, 75 and 77 Of | KRDYQS/ARNNI | + | + | + | + | + | + |
| I-CreI variants | (SEQ ID NO: 292) | | | | | | |
| cleaving the | KNTYSS/AYSYK | + | + | + | + | + | + |
| HSV4.4 target | (SEQ ID NO: 293) | | | | | | |
| (ex: KRGYQS/ | KNEYYS/AYSYK | + | + | + | + | + | + |
| RHRDI stands for | (SEQ ID NO: 294) | | | | | | |
| K28, R30, G32, | KNSRES/AYSYK | | | | | | |
| Y33, Q38, S40/ | (SEQ ID NO: 295) | | | | | | |
| R44, H68, R70, | KNSRES/NHSYN/80K | + | + | + | + | + | + |
| D75 and I77) | (SEQ ID NO: 296) | | | | | | |
| | KNTYWS/ARSYY | + | + | + | + | + | + |
| | (SEQ ID NO: 297) | | | | | | |
| | KRDYQS/ACSYI/115V | + | + | + | + | + | + |
| | (SEQ ID NO: 298) | | | | | | |

+ indicates a functional combination

### EXAMPLE 2.4

### Improvement of Meganucleases Cleaving HSV4.3 by Random Mutagenesis

I-CreI variants able to cleave the palindromic HSV4.3 target has been previously identified in Example 2.1. Some of them can cleave HSV4 target when associated with variants able to cut HSV4.4 (Examples 2.2 and 2.3).

Therefore the 6 combinatorial variants cleaving HSV4.3 were mutagenized, and variants were screened for activity improvement on HSV4.3. According to the structure of the I-CreI protein bound to its target, there is no contact between the 4 central base pairs (positions −2 to 2) and the I-CreI protein (Chevalier et al., Nat. Struct. Biol., 2001, 8, 312-316; Chevalier and Stoddard, Nucleic Acids Res., 2001, 29, 3757-3774; Chevalier et al., J. Mol. Biol., 2003, 329, 253-269). Thus, it is difficult to rationally choose a set of positions to mutagenize, and mutagenesis was performed on the whole protein. Random mutagenesis results in high complexity libraries. Therefore, to limit the complexity of the variant libraries to be tested, the two components of the heterodimers cleaving HSV4 were mutagenized and screened in parallel.

### A) Material and Methods

### a) Construction of Libraries by Random Mutagenesis

Random mutagenesis was performed on a pool of chosen variants, by PCR using Mn²⁺. PCR reactions were carried out that amplify the I-CreI coding sequence using the primers preATGCreFor (5′-gcataaattactatacttctatagacacgcaaacacaaatacacagcggccttgccacc-3′; SEQ ID NO: 169) and I-CreIpostRev (5′-ggctcgaggagctcgtctagaggatcgctcgagttatcagtcggccgc-3′; SEQ ID NO: 170), which are common to the pCLS0542 (FIG. 5) and pCLS1107 (FIG. 6) vectors. Approximately 25 ng of the PCR product and 75 ng of vector DNA (pCLS0542) linearized by digestion with NcoI and EagI were used to transform the yeast Saccharomyces cerevisiae strain FYC2-6A (MATα, trp1Δ63, leu2Δ1, his3Δ200) using a high efficiency LiAc transformation protocol (Gietz and Woods, Methods Enzymol., 2002, 350, 87-96). Expression plasmids containing an intact coding sequence for the I-CreI variant were generated by in vivo homologous recombination in yeast.

### b) Target Vector Yeast Strains

The yeast strain FYBL2-7B (MATα, ura3Δ851, trp1Δ63, leu2Δ1, lys2Δ202) containing the HSV4.3 target in the yeast reporter vector (pCLS1055 FIG. 4) was constructed as described in Example 2.1.

### c) Mating of Meganuclease Expressing Clones, Screening in Yeast and Sequencing

Mating HSV4.3 target strain and mutagenized variant clones and screening were performed as described in Example 2.1. One variant from the first generation was added as control on the filter during screening steps for activity improvement evaluation.

### B) Results

The 6 variants cleaving HSV4.3, (Table XXVI), were pooled, randomly mutagenized and transformed into yeast. 2304 transformed clones were then mated with a yeast strain that contains the HSV4.3 target in a reporter plasmid. After mating with this yeast strain, 86 clones were found to cleave the HSV4.3 target and 46 of them shown higher activity than the best original variant. An Example of positives is shown in FIG. 26. Sequencing of these 46 positive clones indicates that 38 distinct variants listed in Table XXVII were identified.

TABLE XXVI
pool of variants cleaving HSV4.3 and sequences
used as template for random mutagenesis

| SEQ | |
|---|---|
| ID | Amino acids positions and residues |
| NO: | of the I-CreI variants |
| 305 | 28S40K44M70N75N/80K94L |
| 306 | 28S40K44L70N75N/80K |
| 307 | 44M70A75N77V/132V |
| 308 | 44L70N75N/80K |
| 309 | 44Y70K/ |
| 310 | 30G44I70K75N77V/132V |

TABLE XXVII
Improved variants
displaying strong cleavage activity for HSV4.3

| SEQ | |
|---|---|
| ID | Amino acids positions and residues |
| NO: | of the I-CreI variants |
| 311 | 28S30N32S33Y38Q40K44M70N75N77I/80K94L |
| 312 | 28S30N40K44M70N75N77I80K94F132I/56A |
| 313 | 28K30N40S44Y70K75N77I80K94F132V/ |
| 314 | 28S30N40K44M70N75N77I80R94L132I/ |
| 315 | 28S30N40K44M70N75N77I80K94L132V/ |
| 316 | 28K30G40S44M70A75N77V80E94F132V/4N155Q |
| 317 | 28K30N40S44M70N75N77I80K94F132I/82R163S |
| 318 | 28K30N40S44M70A75N77I80K94F132I/100R |
| 319 | 28S30N40K44L70N75N77I80R94F132V/ |
| 320 | 28K30N40S44Y70K75D77I80E94F132V/ |
| 321 | 28S30N40K44M70N75N77I80K94L132I/ |
| 322 | 28S30N40K44M70N75N77I80K94F132I/31R79C128R |
| 323 | 28K30N40S44Y70K75D77I80K94F132I/157D |
| 324 | 28K30N40S44M70N75N77I80K94F132I/3P68H69E |
| 325 | 28S30N40K44L70N75N77I80K94F132V/86D |
| 326 | 28S30N40K44M70N75N77I80K94F132I/ |
| 327 | 28K30N40S44Y70K75D77I80K94F132I/ |
| 328 | 28K30N40S44M70A75N77V80E94F132V/54L |
| 329 | 28S30N40K44M70N75D77I80K94F132V/108T154G |
| 330 | 28S30N40K44L70N75N77I80K94F132V/105D |
| 331 | 28S30N40K44L70N75N77I80K94F132V/43L |
| 332 | 28K30N40S44M70N75N77I80K94F132I/56V151A |
| 333 | 28K30G40S44M70A75N77V80E94F132V/156G |
| 334 | 28S30N40K44L70N75N77T80K94F132I/ |
| 335 | 28K30G40S44M70A75N77V80E94F132V/ |
| 336 | 28K30N40S44M70A75N77I80K94F132V/146K156G |
| 337 | 28S30N40K44M70K75N77V80E94F132V/ |
| 338 | 28K30N40S44M70A75N77I80K94F132I/117V |
| 339 | 28K30G40S44I70K75D77I80E94F132I/160E |
| 340 | 28S30N40K44L70N75N77I80K94F132V/ |
| 341 | 28S30N40K44L70N75N77I80K94F132I/7R79T102V |
| 342 | 28S30N40K44L70N75N77I80K94F132I/54V |
| 343 | 28K30N40S44M70N75N77I80K94F132I/ |
| 344 | 28S30N40K44M70N75N77M80R94F132I/71E105A |
| 345 | 28K30N40S44M70K75N77V80E94F132V/7E |
| 346 | 28S30N40K44M70N75N77I80K94F132I/116R |
| 347 | 28K30N40S44M70K75D77I80E94F132V/2Y |
| 348 | 28K30N40S44Y70K75D77I80E94F132V/79C120A152V |
| 349 | 28S30N40K44L70N75N77I80K94F132I/50R |

* Mutations resulting from random mutagenesis are in bold.

### EXAMPLE 2.5

### Improvement of Meganucleases Cleaving HSV4.4 by Random Mutagenesis

I-CreI variants able to cleave the palindromic HSV4.4 target has been previously identified in Example 2.2. Some of them can cleave HSV4 target when associated with variants able to cut HSV4.4 (Examples 2.1 and 2.3).

Therefore 9 of the 14 combinatorial variants cleaving HSV4.4 were mutagenized, and variants were screened for activity improvement on HSV4.4. As described in Example 2.5, mutagenesis was performed on the whole protein and HSV4.4 variants were screened in parallel of HSV4.3 (Example 2.5).

### A) Material and Methods

### a) Construction of Libraries by Random Mutagenesis

Random mutagenesis was performed as described in Example 2.5, on a pool of chosen variants, by PCR using the same primers and Mn²⁺ conditions (preATGCreFor SEQ ID NO: 169 and I-CreIpostRev SEQ ID NO: 170). Approximately 25 ng of the PCR product and 75 ng of vector DNA pCLS1107) linearized by digestion with NcoI and EagI were used to transform the yeast Saccharomyces cerevisiae strain FYC2-6A (MATα, trp1Δ63, leu2Δ1, his3Δ200) using a high efficiency LiAc transformation protocol (Gietz and Woods, Methods Enzymol., 2002, 350, 87-96). Expression plasmids containing an intact coding sequence for the I-CreI variant were generated by in vivo homologous recombination in yeast.

### b) Target Vector Yeast Strains

The yeast strain FYBL2-7B (MATα, ura3Δ851, trp1Δ63, leu2Δ1, lys2Δ202) containing the HSV4.4 target in the yeast reporter vector (pCLS1055 FIG. 4) was constructed as described in Example 2.2.

### c) Mating of Meganuclease Expressing Clones, Screening in Yeast and Sequencing

Mating HSV4.4 target strain and mutagenized variant clones and screening were performed as described in Example 2.2. One variant from first generation was added as control on filter during screening steps for activity improvement evaluation.

### B) Results

Nine chosen variants cleaving HSV4.4, (Table XXVIII), were pooled, randomly mutagenized and transformed into yeast. 2304 transformed clones were then mated with a yeast strain that contains the HSV4.4 target in a reporter plasmid. After mating with this yeast strain, 262 clones were found to cleave the HSV4.4 target and 17 of them shown higher activity than the best original variant. An Example of positives is shown in FIG. 27. Sequencing 93 of these 262 positive clones indicates that 63 distinct variants listed in Table XXIX were identified. 14 of them shown higher activity than the best original variant.

TABLE XXVIII
pool of variants cleaving HSV4.4 and sequences used as template
for random mutagenesis

| SEQ | |
|---|---|
| ID | Amino acids positions and residues |
| NO: | of the I-CreI variants |
| 350 | 28K30N32D33Y38Y40S44A68Y70S75Y77K/ |
| 351 | 28K30N32S33R38E40S44N68Y70S75Y77K/ |
| 352 | 28K30R32D33Y38Q40S44A68R70N75N77I/ |
| 353 | 28K30N32T33Y38S40S44A68Y70S75Y77K/ |
| 354 | 28K30N32E33Y38Y40S44A68Y70S75Y77K/ |
| 355 | 28K30N32S33R38E40S44N68H70S75Y77N/80K |
| 356 | 28K30N32T33Y38W40S44A68R70875Y77Y/ |
| 357 | 28K30R32D33Y38Q40S44A68C70875Y77I/115V |
| 358 | 28K30N32S33R38E40S44A68Y70S75Y77K/ |

TABLE XXIX
Improved variants displaying strong cleavage activity for HSV4.4

| SEQ ID NO: | Amino acids positions and residues of the I-CreI variants |
|---|---|
| 359 | 32E38Y44A68Y70S75Y77K105A |
| 360 | 24F32E38Y44A68Y70S75Y77K107R |
| 361 | 32E38Y44A68Y70S75Y77K132V |
| 362 | 32D38Y44A68Y70S75Y77K |
| 363 | 32E38Y44A68Y70S75Y77K114Y |
| 364 | 1V32G38Y44A68Y70S75Y77K80K103S |
| 365 | 32D38Y44A68Y69G70S75Y77K80A105A162P |
| 366 | 32E38Y44A50R68Y70S75Y77K160E |
| 367 | 32E38Y44A68Y70S75Y77K |
| 368 | 33R38E44A54L68Y70S75Y77K103I |
| 369 | 32T38S44A68Y70S75Y77K124Q132V |
| 370 | 33R38E44A68Y70S75Y77K129A |
| 371 | 6S32E38Y44A68Y70S75Y77K111R |
| 372 | 32D38Y44A68Y70S75Y77N80K132V |
| 373 | 32D38Y44A68Y70S75Y77K164T |
| 374 | 32E38Y44A68Y70S75Y77K163R |
| 375 | 32D38Y44A68Y70S75Y77K82M110D117G132V163Q |
| 376 | 32E38Y44A68Y70S75Y77K162P |
| 377 | 32T36N38Y44A60G68S70S75Y77K107E110K132V |
| 378 | 32E38Y44A68Y70S75Y77K160N |
| 379 | 32E38Y44A68Y70S75Y77K163Q |
| 380 | 32E38Y43L44A68Y70S75Y77K |
| 381 | 32E38Y44A68Y70S75Y77K107R |
| 382 | 32E38Y44A68Y70S75Y77K151A |
| 383 | 2S32D38Y44A68Y70S75Y77K |
| 384 | 32D38Y44A66H68Y70S75Y77K152Q158E |
| 385 | 6S32D38Y44A68Y70S75Y77K |
| 386 | 32D38Y44A68Y70S75Y77K78I |
| 387 | 33R38Y44A68Y70S75Y77K100R161P |
| 388 | 32E38Y44A64A68Y70S72C75Y77K129A135Q156R |
| 389 | 32D38Y44A60N68Y70S75Y77K |
| 390 | 32E38Y44A68Y70S73I75Y77K87L |
| 391 | 32D38Y40C44A68Y70S75Y77K |
| 392 | 32E38Y44A49A68Y70S75Y77K |
| 393 | 33R38E44A68Y70S75Y77K85R |
| 394 | 32E38Y44A68Y70S75Y77K159R |
| 395 | 32K38Y44A68Y70S75Y77N134T |
| 396 | 32T33R38E44A68Y70S75Y77K |
| 397 | 30K32E38Y44A68Y70S75Y77K |
| 398 | 32E38Y44A68Y70S75Y77K96Q |
| 399 | 3S32E38Y44A68Y70S75Y77K |
| 400 | 32E38Y44A68Y70S75Y77K105G |
| 401 | 33R38E44A68Y70S75Y77K |
| 402 | 7E32D36N38Y44A68Y70S75Y77K |
| 403 | 30R31H32D44A49A68Y70S75Y77K |
| 404 | 32T38S44A68Y70S75Y77K114T |
| 405 | 33R38E44A68Y70S75Y77K163S |
| 406 | 33H38E44A66H68Y70S75Y77K |
| 407 | 32D38Y44A68Y70S72Y75Y77K |
| 408 | 33R38E44A68Y70S75Y77R97V161A |
| 409 | 13M32E38Y44A68Y70S75Y77K |
| 410 | 32E38Y44A68Y70S75Y77K107E |
| 411 | 30R32D44A68Y70S75Y77K100N |
| 412 | 30R32D44A68Y70S75Y77K114T120A |
| 413 | 32E38Y44A64A68Y70S75Y77K82M |
| 414 | 30R32D44A68Y70S75Y77K |
| 415 | 32D38Y44A68Y70S71E75Y77K |
| 416 | 33R38E44A57R68Y70S75Y77K |
| 417 | 32T38W44A70S75Y77Y80K110D |
| 418 | 30R32D44A68Y70S75Y77K107R |
| 419 | 32T38S44A68Y70S75Y77K120G |
| 420 | 33R38E44N68H70S75Y77N80K111H131R132V |
| 421 | 32D38Y44A54L68Y70S75Y77K79G129A156G |

* Mutations resulting from random mutagenesis are in bold.

### EXAMPLE 2.6

### Identification of Improved Meganucleases Cleaving HSV4

Improved I-CreI variants able to cleave each of the palindromic HSV4 derived targets (HSV4.3 and HSV4.4) were identified in Example 2.5 and Example 2.6. As described in Example 2.3, pairs of such variants (one cutting HSV4.3 and one cutting HSV4.4) were co-expressed in yeast. The heterodimers that should be formed were assayed for cutting the non palindromic HSV4 target.

### A) Materials and Methods

### a) Construction of Target Vector

The HSV4 target vector was constructed as described in Example 2.3.

### b) Co-Expression of Variants

Yeast DNA was extracted from variants cleaving the HSV4.4 target in the pCLS1107 expression vector using standard protocols and was used to transform E. coli. The resulting plasmid DNA was then used to transform yeast strains expressing a variant cutting the HSV4.3 target in the pCLS542 expression vector. Transformants were selected on synthetic medium lacking leucine and containing G418.

### c) Mating of Meganucleases Coexpressing Clones and Screening in Yeast

Mating and screening of meganucleases coexpressing clones were performed as described in Example 2.3. Results were analyzed by scanning and quantification was performed using appropriate software.

### B) Results

Co-expression of improved variants cleaving the HSV4.4 target (6 variants chosen among those described in Table XXIX) and 6 improved variants cleaving the HSV4.3 target (described in Table XXVII) resulted in cleavage of the HSV4 target in all of cases (FIG. 28). All assayed combinations are summarized in Table XXX.

TABLE XXX
Cleavage of the HSV4 target by the heterodimeric improved variants

| | | Amino acids at positions 28, 30, 32, 33, 38, 40/44, 68, 70, 75 and 77 | | | | | |
|---|---|---|---|---|---|---|---|
| | | of I-CreI variants cleaving the HSV4.3 target | | | | | |
| | | (ex: KRSRES/TYSNI stands for K28, R30, S32, R33, E38, S40/T44, Y68, S70, N75 and I77) | | | | | |
| | | | KNSYQS/MRANI/ | SNSYQK/LRNNI/ | KNSYQS/ | | KNSYQS/ |
| | | SNSYQK/LRNNI/ | 80K132V146 | 80K132V/43 | MRANV/ | SNSYQK/ | MRKDI/ |
| | | 80R132V | K156G | L | 54L132V | MRNNI/80K | 2Y132V |
| | | (SEQ ID | (SEQ ID | (SEQ ID | (SEQ ID | (SEQ ID | (SEQ ID |
| | | NO: 428) | NO: 429) | NO: 430) | NO: 431) | NO: 432) | NO: 433) |
| Amino acids | KNEYYS/AYSYK/ | + | + | + | + | + | + |
| at positions | 50R160E | | | | | | |
| 28, 30, 32, 33, | (SEQ ID NO: 422) | | | | | | |
| 38, 40/44, 68, | KNEYYS/AYSYK/ | + | + | + | + | + | + |
| 70, 75 and 77 Of | 69G80A105A162P | | | | | | |
| I-CreI variants | (SEQ ID NO: 423) | | | | | | |
| cleaving the | KNEYYS/AYSYK/132V | + | + | + | + | + | + |
| HSV4.4 target | (SEQ ID NO: 424) | | | | | | |
| (ex: KRGYQS/ | KNEYYS/AYSYK/ | + | + | + | + | + | + |
| RHRDI stands for | 24F107R | | | | | | |
| K28, R30, G32, | (SEQ ID NO: 425) | | | | | | |
| Y33, Q38, S40/ | KNGYYS/AYSYK/ | + | + | + | + | + | + |
| R44, H68, R70, | 1V80K103S | | | | | | |
| D75 and I77) | (SEQ ID NO: 426) | | | | | | |
| | KNEYYS/AYSYK/105A | + | + | + | + | + | + |
| | (SEQ ID NO: 427 | | | | | | |

+ indicates a functional combination

### EXAMPLE 2.7

### Validation of HSV4 Target Cleavage in an Extrachromosomal Model in CHO Cells

I-CreI variants able to efficiently cleave the HSV4 target in yeast when forming heterodimers were described in Examples 2.3 and 2.7. In order to identify heterodimers displaying maximal cleavage activity for the HSV4 target in CHO cells, the efficiency of chosen combinations of variants to cut the HSV4 target was compared, using an extrachromosomal assay in CHO cells. The screen in CHO cells is a single-strand annealing (SSA) based assay where cleavage of the target by the meganucleases induces homologous recombination and expression of a LagoZ reporter gene (a derivative of the bacterial lacZ gene).

### 1) Materials and Methods

### a) Cloning of HSV4 Target in a Vector for CHO Screen

The target was cloned as follows: oligonucleotide corresponding to the HSV4 target sequence flanked by gateway cloning sequence was ordered from PROLIGO 5′TGGCATACAAGTTTCCAAGCTGGTGTACCTGATAGTGGCAATCGTCTGTCA3′ (SEQ ID NO: 289). Double-stranded target DNA, generated by PCR amplification of the single stranded oligonucleotide, was cloned using the Gateway protocol (INVITROGEN) into CHO reporter vector (pCLS1058, FIG. 11). Cloned target was verified by sequencing (MILLEGEN).

### b) Re-Cloning of Meganucleases

The ORF of I-CreI variants cleaving the HSV4.3 and HSV.4 targets identified in Examples 2.5 and 2.6 were re-cloned in pCLS1768 (FIG. 29). ORFs were amplified by PCR on yeast DNA using the attB1-ICreIFor (5′-ggggacaagtttgtacaaaaaagcaggcttcgaaggagatagaaccatggccaataccaaatataacaaagagttcc-3′; SEQ ID NO: 434) and attB2-ICreIRev (5′-ggggaccactttgtacaagaaagctgggtttagtcggccgccggggaggatttcttcttctcgc-3′; SEQ ID NO: 435) primers. PCR products were cloned in the CHO expression vector pCLS1768 (FIG. 29) using the Gateway protocol (INVITROGEN). Resulting clones were verified by sequencing (MILLEGEN).

### c) Extrachromosomal Assay in Mammalian Cells

CHO K1 cells were transfected with Polyfect® transfection reagent according to the supplier's protocol (QIAGEN). 72 hours after transfection, culture medium was removed and 150 μl of lysis/revelation buffer for β-galactosidase liquid assay was added (typically 1 liter of buffer contained: 100 ml of lysis buffer (Tris-HCl 10 mM pH7.5, NaCl 150 mM, Triton X100 0.1%, BSA 0.1 mg/ml, protease inhibitors), 10 ml of Mg 100× buffer (MgCl₂ 100 mM, β-mercaptoethanol 35%), 110 ml ONPG 8 mg/ml and 780 ml of sodium phosphate 0.1M pH7.5). After incubation at 37° C., OD was measured at 420 nm. The entire process is performed on an automated Velocity11 BioCel platform.

Per assay, 150 ng of target vector was cotransfected with 12.5 ng of each one of both mutants (12.5 ng of mutant cleaving palindromic HSV4.3 target and 12.5 ng of mutant cleaving palindromic HSV4.4 target).

### 2) Results

6 variants cleaving HSV4.3 and 4 variants cleaving HSV4.4 described in Example 2.5, 2.6 and 2.7 were re-cloned in pCLS1768 (FIG. 29). Then, I-CreI variants cleaving the HSV4.3 or HSV4.4 targets were assayed together as heterodimers against the HSV4 target in the CHO extrachromosomal assay.

Table XXXIII shows the functional combinations obtained for 24 heterodimers. Analysis of the efficiencies of cleavage and recombination of the HSV4 sequence demonstrates that 9 combinations of I-CreI variants are able to transpose their cleavage activity from yeast to CHO cells without additional mutation.

TABLE XXXI
Functional heterodimeric combinations cutting the HSV4 target in CHO cells.

| Optimized variants cleaving HSV4.3 | | | | | |
|---|---|---|---|---|---|
| 28S40K44L | 44M70A80K | 28S40K43L44L | 44M54L70A | 28S40K44M | 2Y44M70K |
| 70N80R132V | 132V146K156G | 70N80K132V | 77V132V | 70N80K | 132V |
| (SEQ ID | (SEQ ID | (SEQ ID | (SEQ ID | (SEQ ID | (SEQ ID |
| NO: 440) | NO: 441) | NO: 442) | NO: 443) | NO: 444) | NO: 445) |

| | | | | | | |
|---|---|---|---|---|---|---|
| Optimized | 32D38Y44A68Y | + | + | + | + | |
| variants | 69G70S75Y77 | | | | | |
| cleaving | K80A105A162P | | | | | |
| HSV4.4 | (SEQ ID NO: 436) | | | | | |
| | 24F32E38Y44A | | | | | |
| | 68Y70S75Y77K | | | | | |
| | 107R | | | | | |
| | (SEQ ID NO: 437) | | | | | |
| | 70S75Y77K | | | | | |
| | 80K103S | | | | | |
| | (SEQ ID NO: 438) | | | | | |
| | 32E38Y44A68Y | + | + | + | + | + |
| | 70S75Y77K105A | | | | | |
| | (SEQ ID NO: 439) | | | | | |

+ indicates a functional combination

### EXAMPLE 2.8

### Covalent Assembly as Single Chain and Improvement of Meganucleases Cleaving HSV4 by Site-Directed Mutagenesis

Coexpression of the cutters described in Example 2.6, 2.7, 2.8 leads to a high cleavage activity of the HSV4 target in yeast. Some of them are able to cleave HSV4 in a mammalian expression system. One of them is shown as Example in Table XXXII.

TABLE XXXII
Functional heterodimer cutting the HSV4 target in CHO cells.

| | |
|---|---|
| HSV4.3-M2 (SEQ ID NO: 35) | 44M 70A 80K 132V 146K 156G |
| HSV4.4-MF (SEQ ID NO: 36) | 32E 38Y 44A 68Y 70S 75Y 77K 105A |

The M2/MF HSV4 heterodimer gives high cleavage activity in yeast and CHO cells. M2 is a HSV4.3 cutter that bears the following mutations in comparison with the I-CreI wild type sequence: 44M, 70A, 80K, 132V, 146K, 156G. MF is a HSV4.4 cutter that bears the following mutations in comparison with the I-CreI wild type sequence: 32E, 38Y, 44A, 68Y, 70S, 75Y, 77K, 105A.

Single chain constructs were engineered using the linker RM2 resulting in the production of the single chain molecule: M2-RM2-MF. During this design step, the G19S mutation was introduced in the C-terminal MF mutant. In addition, mutations K7E, K96E were introduced into the M2 mutant and mutations E8K, E61R into the MF mutant to create the single chain molecule: M2(K7E K96E)RM2-MF(E8K E61R) that is called further SCOH-HSV4-M2-MF.

Four additional amino-acid substitutions have been found in previous studies to enhance the activity of I-CreI derivatives: these mutations correspond to the replacement of Phenylalanine 54 with Leucine (F54L), Glutamic acid 80 with Lysine (E80K), Valine 105 with Alanine (V105A) and Isoleucine 132 with Valine (I132V). Some of these are already present in HSV4.3 and HSV4.4 variants (i.e. HSV4.3-M2 and HSV4.4-MF). Some additional combinations were introduced into the coding sequence of N-terminal and C-terminal protein fragment (Example in Table XXXIII), and the resulting proteins were tested for their ability to induce cleavage of the HSV4 target. Twelve single chain constructs were then tested in CHO for cleavage of the HSV4 target.

TABLE XXXIII
Example of single chain I-CreI variants assayed for HSV4
cleavage in CHO cells.

| | | | | SEQ |
|---|---|---|---|---|
| Construct | Single Chain | Mutations on N-terminal segment | Mutations on C-terminal segment | ID NO |
| pCLS2470 | SCOH-HSV4-M2-MF | 7E 44M 70A 80K 96E 132V 146K | 8K 19S 32E 38Y 44A 61R 68Y 70S | 446 |
| | | 156G | 75Y 77K 105A | |
| pCLS2471 | SCOH-HSV4-M2-MF-54L | 7E 44M 70A 80K 96E 132V 146K | 8K 19S 32E 38Y 44A 61R 68Y 70S | 447 |
| | | 156G | 75Y 77K 105A 132V | |
| pCLS2472 | SCOH-HSV4-M2-MF-132V | 7E 44M 70A 80K 96E 132V 146K | 8K 19S 32E 38Y 44A 54L 61R 68Y | 448 |
| | | 156G | 70S 75Y 77K 105A | |
| pCLS2474 | SCOH-HSV4-M2-54L-MF | 7E 44M 54L 70A 80K 96E 132V | 8K 19S 32E 38Y 44A 61R 68Y 70S | 449 |
| | | 146K 156G | 75Y 77K 105A | |
| pCLS2476 | SCOH-HSV4-M2-54L-MF-132V | 7E 44M 54L 70A 80K 96E 132V | 8K 19S 32E 38Y 44A 54L 61R 68Y | 450 |
| | | 146K 156G | 70S 75Y 77K 105A | |
| pCLS2477 | SCOH-HSV4-M2-54L-MF- | 7E 44M 54L 70A 80K 96E 132V | 8K 19S 32E 38Y 44A 61R 68Y 70S | 451 |
| | 80K132V | 146K 156G | 75Y 77K 80K 105A 132V | |
| pCLS2478 | SCOH-HSV4-M2-105A-MF | 7E 44M 70A 80K 96E 105A 132V | 8K 19S 32E 38Y 44A 61R 68Y 70S | 452 |
| | | 146K 156G | 75Y 77K 105A | |
| pCLS2479 | SCOH-HSV4-M2-105A-MF-132V | 7E 44M 70A 80K 96E 105A 132V | 8K 19S 32E 38Y 44A 61R 68Y 70S | 453 |
| | | 146K 156G | 75Y 77K 105A 132V | |
| pCLS2481 | SCOH-HSV4-M2-105A-MF- | 7E 44M 70A 80K 96E 105A 132V | 8K 19S 32E 38Y 44A 61R 68Y 70S | 454 |
| | 80K132V | 146K 156G | 75Y 77K 80K 105A 132V | |
| pCLS2790 | SCOH-HSV4-M2-105A-MF- | 7E44M70A80K96E105A132V | 8K19S32E38Y44A61R68Y70S75Y77K | 534 |
| | 80K132V | 146K156G | 80K105A132V | |

pCLS2790 bears the same variant than pCLS2481 under the control of pCMV promoter (instead of pEF1alpha).

### 1) Material and Methods

### a) Cloning of the SC OH Single Chain Molecule

A series of synthetic gene assembly was ordered to TOPGENE TECHNOLOGY. Synthetic genes coding for the different single chain variants targeting HSV4 were cloned in pCLS0491 (FIG. 31) using Eco RI and Bam HI restriction sites.

### b) Extrachromosomal Assay in Mammalian Cells

CHO K1 cells were transfected as described in Example 2.8. 72 hours after transfection, culture medium was removed and 150 μl of lysis/revelation buffer for β-galactosidase liquid assay was added. After incubation at 37° C., OD was measured at 420 nm. The entire process is performed on an automated Velocity11 BioCel platform.

Per assay, 150 ng of target vector was cotransfected with an increasing quantity of variant DNA from 0.75 to 50 ng (50 ng of single chain DNA corresponding to 25 ng+25 ng of heterodimer DNA). Finally, the transfected DNA variant DNA quantity was 0.78 ng, 1.56 ng, 3.12 ng, 6.25 ng, 12.5 ng, 25 ng and 50 ng. The total amount of transfected DNA was completed to 200 ng (target DNA, variant DNA, carrier DNA) using empty vector (pCLS0001).

2) Results

The activity of the SCOH-HSV4 single chain molecules (Table XXXIII) against the HSV4 target was monitored using the previously described CHO assay by comparison to the HSV4.3-M2×HSV4.4-MF heterodimer and our internal control SCOH-RAG (SEQ ID NO: 468) and I-SceI (SEQ ID NO: 469) meganucleases. All comparisons were done at 0.78 ng, 1.56 ng, 3.12 ng, 6.25 ng, 12.5 ng, 25 ng and 50 ng transfected variant DNA.

All assayed single chain variants are more active than M2×MF heterodimer and the internal control I-SceI at standard dose (25 ng). Variants shared specific behaviour upon assayed dose depending on the mutation profile they bear (FIGS. 33 and 34). For Example, scOH-HSV4-M2-54L-MF (pCLS2474, SEQ ID NO: 449) has a similar profile to our internal standard SCOH-RAG: its activity increase from low quantity to high quantity (FIG. 35). scOH-HSV4-M2-105A-MF80K132V (pCLS2481, SEQ ID NO: 454) is highly active at low quantities of transfected DNA (3.12 ng) and its apparent activity decreases with dose (FIG. 36). scOH-HSV4-M2-MF-132V (pCLS2472, SEQ ID NO: 448) shares an intermediate profile between the two previous ones (FIG. 37). The profile of scOH-HSV4-M2-MF (pCLS2470, SEQ ID NO: 446), which is a common scaffold to all assayed single chain variants, is an average of individual behaviors at low DNA quantity (max at 6.25 ng) and decreases quickly with DNA dose (the lowest at 50 ng) (FIG. 38). All of these variants could be used for HSV-1 genome targeting depending on the tissue infected.

### EXAMPLE 3

### Inhibition of Viral Replication by I-CreI Variants Cleaving HSV2, HSV4 or HSV12 Target Sequences

Single-chain obligate heterodimer I-CreI variants able to efficiently cleave the HSV2 or HSV4 target sequences in yeast and CHO cells were described in Examples 1 and 2.

Single chain obligate heterodimer constructs were also generated for the I-CreI variants able to cleave the HSV12 target sequences described in Table II. These single chain constructs were engineered using the linker RM2 (AAGGSDKYNQALSKYNQALSKYNQALSGGGGS) (SEQ ID NO: 464). During this design step, mutations K7E, K96E were introduced into the M1 or the M1-80K mutant and mutations E8K, E61R into the ME-132V mutant to create the single chain molecules: M1(K7E K96E)-RM2-ME-132V(E8K E61R) that is called SCOH-HSV12-M1-ME-132V and M1-80K(K7E K96E)-RM2-ME-132V(E8K E61R) that is called SCOH-HSV12-M1-80K-ME-132V (Table XXXIV).

TABLE XXXIV
Example of single chain I-CreI variants for HSV12

| | | Mutations on N-terminal | Mutations on C-terminal | SEQ |
|---|---|---|---|---|
| Construct | Single Chain | segment | segment | ID NO |
| pCLS2633 | SCOH-HSV12-M1-ME-132V | 7E 24V 33C 38S 44I 50R | 8K 19S 30R 33S 44K 61R | 465 |
| | | 70S 75N 77R 96E 132V | 66H 68Y 70S 77T 87I | |
| | | | 132V 139R 163S | |
| pCLS2635 | SCOH-HSV12-M1-80K-ME- | 7E 24V 33C 38S 44I 50R | 8K 19S 30R 33S 44K 61R | 466 |
| | 132V | 70S 75N 77R 80K 96E | 66H 68Y 70S 77T 87I | |
| | | 132V | 132V 139R 163S | |

In order to further validate the cleavage activity of these single chain molecules, the ability of I-CreI variants cleaving HSV2, HSV4 or HSV12 target sequences to inhibit viral replication was examined using a recombinant Herpes Simplex Virus (rHSV-1). rHSV was constructed with a cassette containing a CMV promoter driving the LacZ gene (FIG. 39). An I-SceI target site was inserted between the CMV promoter and the LacZ gene and served as a positive control for inactivation of the virus. This expression cassette was introduced into the major LAT locus of HSV by homologous recombination resulting in LacZ expression during lytic infection of COS-7 cells. Thus to evaluate the inhibition of viral replication, the ability of I-SceI or the I-CreI variants cleaving HSV2, HSV4 or HSV12 target sequences to diminish LacZ expression after infection with rHSV1 was evaluated.

### 1) Material and Methods

### a) Single Chain Obligate Heterodimer (SC OH) Molecules

Single chain obligate heterodimer molecules were generated for the I-CreI variants able to cleave the HSV 12 target sequences described in Table II by custom gene synthesis (MWG-EUROFINS). Synthetic genes coding for the different single chain variants targeting HSV12 were cloned in pCLS1853 (FIG. 14) using AscI and XhoI restriction sites.

### b) Cells and Viruses

Viruses were grown and assayed on COS-7 cells. COS-7 cells were cultured in DMEM supplemented with 2 mM L-glutamine, penicillin (100 IU/ml), streptomycin (100 mg/ml), amphotericin B (Fongizone: 0.25 mg/ml, Invitrogen-Life Science) and 10% FBS. HSV-1 was purchased from the American Type Culture Collection (ATCC). Viruses were propagated at a multiplicity of infection of 0.003 PFU/cell and virus titers were determined by plaque assays.

### c) Construction of Recombinant HSV-1

Recombinant virus was generated in a manner similar to that previously described (Lachmann, R. H., Efstathioun S., 1997, Journal of Virology, 3197-3207). An approximately 4.6 kb PstI-BamHI viral genomic fragment was cloned into pUC19. Based on HSV-1 sequence from the database (GenBank NC_{—}001806) this represents nucleotides 118869-123461 and 7502-2910 in the inverted terminal repeats of the HSV-1 genome. A cassette containing the CMV promoter driving LacZ expression was introduced into a 19 bp SmaI/HpaI deletion. This region is located within the major LAT locus of HSV-1. The I-SceI cleavage site (tagggataacagggtaat SEQ ID NO: 467) was inserted after the CMV promoter and before the ATG of the LacZ gene. This construct (pCLS0126, FIG. 40) was used to generate recombinant viruses. Plasmid was linearized by XmnI digestion and 2 μg of this plasmid was co-transfected with 10g of HSV-1 genomic DNA into COS-7 cells using Lipofectamine 2000 (Invitrogen). After 3 days, infected cells were harvested and sonicated. An aliquot of the lysed cells was used to infect a COS monolayer and cells were overlayed with 1% agarose medium. After 3 days, 300 μg/ml of X-gal (5-bromo-4-chloro-3-indolyl-β-D-galactopyranoside) was added to the overlay. β-galactosidase positive ‘blue’ plaques were picked and subjected to three rounds of plaque purification.

### d) Viral Inhibition

6-well plates were seeded with 2×10⁵ cells per well. The next day COS-7 cells were transfected using lipofectamine 2000 (Invitrogen) with either 1 μg or 5 μg of plasmid expressing I-SceI or the I-CreI variants cleaving HSV2, HSV4 or HSV12 target sequences, the total volume of DNA was completed to 5 μg with empty vector pCLS0001 (FIG. 15). The transfection efficiency was between 50-70% using this method. Twenty-four hours later, subconfluent transfected cells were infected with rHSV1 or a wild type F strain (ATCC, ref VR-733). For infection, virus was diluted in DMEM without serum at a MOI from 10⁻³ to 1 adsorbed onto cells for 2 h at 37° C. and then diluted in complete growth medium to a final volume of 2 ml per well. Cells were harvested 24 h after infection and β-galactosidase activity was assayed on a total of 1.0×10³ (for MOI 10⁻² to 10⁻¹) or 2.5×10⁴ (for MOI 10⁻³) rHSV infected-cells using a luminescent β-galactosidase assay (Beta-Glo assay, Promega). Results are converted to % of reduction of viral infection.

### e) Quantification of Viral DNA

Total DNA (COS-7 and viral genomes) from transfected and infected COS-7 cells was extracted and purified using DNeasy Blood and Tissue Kit (Qiagen, France) according to the manufacturer's instructions. Then, the relative quantity of viral DNA was determined via real-time PCR using primers specific to the HSV genome (gB gene) normalized to COS-7 DNA level using primers specific to the glyceraldehyde-3-phosphate deshydrogenase (GAPDH) gene. Oligonucleotide primers used for PCR corresponding to a part of gB gene from viral DNA are forward primer: 5′-AGAAAGCCCCCATTGOCCAGGTAGT (SEQ ID NO:536) and reverse primer: 5′-ATTCTCTITCCGACGCCATATCCACCAC (SEQ ID NO:537) and those corresponding to a part of GAPDH gene from COS-7 DNA are forward primer: 5′-GGCAGAACCCGGGTTTATAACTGTC (SEQ ID NO:538) and reverse primer: 5′-CCAGTCCTGGATGAGAAAGG (SEQ ID NO:539). The PCR was carried out using SYBR Premix Ex taq (TaKaRa, Japan) and PCR amplification included initial denaturation at 95° C. for 5 min, followed by 40 cycles of 95° C. for 15 seconds, 60° C. for 15 seconds, and 72° C. for 30 seconds. Each PCR assay contained a negative control and a series of plasmid DNA dilutions which can be amplified efficiently, to generate the standard curve. Results are converted to % of reduction of viral DNA level.

### f) Meganuclease Expression

COS-7 cells were harvested 24 or 48 hours after the transfection with 0.3 or 5 μg of plasmid expressing I-CreI variants and directly solubilised in Laemmli buffer (100 μl of buffer for 10⁶ cells). The equivalent of 10⁵ cells was loaded on a SDS-PAGE gel and probed by western blot using a rabbit polyclonal antibody against I-CreI.

g) PCR and Sequencing Analysis of rHSV-1 Genome

Total DNA (COS-7 and rHSV-1 genomes) from transfected and infected COS-7 cells was extracted and purified using DNeasy Blood and Tissue Kit (Qiagen, France) according to the manufacturer's instructions. For each tested sample, we designed a pair of tag-forward and biotin-labeled reverse PCR primers recognising a part of rHSV-1 genome corresponding to the HSV2 and HSV4 target sequences. The DNA polymerase used was Herculase II Fusion DNA Polymerase (Stratagene, France). PCR reactions were run during 30 cycles with an annealing temperature of 67° C. The PCR reaction products were resolved on 0.8% TAE/agarose gel. DNA fragments were purified using NucleoSpin Extract II kit (Qiagen, France) according to the manufacturer's instructions before sending them to GATC Biotech (France) for sequence analysis.

### g) Wild Type HSV-1 Virus and BSR Cells

The wild-type SC16 strain of HSV-1 was grown on BSR cells. BSR cells, cloned from baby hamster kidney cells (BHK-21), were obtained from American Type Culture Collection (ATCC) (Teddington, UK) and maintained in Dulbecco's modified Eagle medium (D-MEM) supplemented with 10% foetal calf serum serum, 100 U/ml penicillin and 100 μg/ml streptomycin sulfate (PAA Laboratories, Austria). Viruses were concentrated before titration and kept frozen at −80° C. A titration in BSR cells was performed after thawing and dilution (i.e. immediately before use). Plaques were counted the following day.

### i) Transfection of BSR Cells and Infection by Wild-Type SC16 Strain of HSV-1

The day before transfection, BSR cells were seeded in 6-well culture dishes (Falcon, Becton Dickinson, Le Pont De Claix, France) at 2×10⁵ cells per well and incubated overnight at 37° C. in complete growth medium. The cultures were about 65% confluent on the day of transfection. Co-transfections with 1.5 μg of plasmid expressing I-CreI variants cleaving HSV2, HSV4 or HSV 12 target sequences and 1.5 μg of plasmid expressing GFP were done using LipofectAMINE 2000 (LF2000, Invitrogen Life Technologies, Carlsbad, Calif.) according to the manufacturer's instructions. A control vector was used to monitor background expression of GFP protein in BSR cells. Forty-eight hours after the co-transfection step, cells were infected with wild-type SC16 strain of HSV-1 at the multiplicity of infection (MOI) varying from 0.1 to 8.

### i) Immunofluorescent Cell Staining and Confocal Microscopy

The BSR transfected cells were cultured on glass coverslips 24 h before fixation with 4% paraformaldehyde (PFA) in PBS at room temperature (RT) for 15 min. The cells then were washed twice with PBS and permeabilized with 0.2% Triton X-100 in PBS for 15 min at RT. Non-specific staining was blocked with 0.5% bovine serum albumin (BSA) in PBS. The coverslips were then incubated with the polyclonal rabbit antibody against I-CreI in 0.5% BSA in PBS for 2 h. The monoclonal mouse antibody against the glycoprotein C (gC) of HSV-1 was used at 1:500 dilution. The coverslips were incubated with rhodamine-conjugated anti-mouse IgG (Immunotech, Marseille, France) at 1:150 dilution. The immunofluorescence was analysed using a Leica DMR confocal microscope with a 40× objective.

### k) FACS Analysis

Following 48 h of transfection, cells were infected for 8 h with SC-16 before being washed twice in PBS, resuspended in 500 μl of ice-cold PBS and fixed with 500 μl of 2% PFA in PBS at RT for 15 min. Fixed cells were counted with a heamocytometer and an aliquot of 2.5×10⁵ cells were placed in a 1.5 ml Eppendorf tube. Cells were incubated with i) 1:500 dilution of anti-amino acids 290 to 300 of glycoprotein C (gC) of HSV-1 rabbit antibodies (Sigma Aldrich, H6030) for 2 h; and ii) with 1:500 dilution of phycoerytrine (PE) conjugated goat anti-rabbit IGg seconder antibodies 1 h (Santa Cruz Biotechnology, Inc), with 3 washes in PBS before each step. Fluorescence-activated cell sorting (FACS) analysis was then performed on a Cytometer EPICS ELITE ESP (Beckman-Coulter) using a 488 nm emitting laser used for detection to detect either EGFP (488 nm) or PE (506 nm) emissions. Non-transfected cells were used as a negative control for GFP emission while non-infected cells stained with primary and secondary antibody were used as a negative control for PE. Thresholds were set-up to include viable cells only, as assessed by forward scatter data, and to include a range of fluorescence representing less than 1% of the fluorescence of control cells.

### 2) Results

a) Meganucleases Prevent the Infection of COS-7 Cells by an rHSV-1 Virus

Three single chain variants cleaving the HSV4 target sequence (pCLS2472, SCOH-HSV4-M2-MF-132V, SEQ ID NO: 448; pCLS2474, SCOH-HSV4-M2-54L-MF, SEQ ID NO: 449 and pCLS2481, SCOH-HSV4-M2-105A-MF80K132V, SEQ ID NO: 454) described in Example 2.8, three single-chain variants cleaving the HSV2 target sequence described in Example 1.8 (pCLS2457, SCOH-HSV2-M1-MC-132V, SEQ ID NO: 254; pCLS2459, SCOH-HSV2-M1-MC80K105A132V, SEQ ID NO: 256 and pCLS2465, SCOH-HSV2-M1-105A132V-MC132V, SEQ ID NO: 261) and two single chain variants cleaving the HSV12 target sequence (pCLS2633, SCOH-HSV12-M1-ME-132V, SEQ ID NO: 465; pCLS2635, SCOH-HSV12-M-80K-ME-132V, SEQ ID NO: 466) described in Table XXXIV were tested for their ability to inhibit viral replication of rHSV-1.

FIG. 41 shows the results obtained for the eight single-chain variants as well as I-SceI compared to cells treated with empty vector only. Transfection of 5 μg I-SceI expression vector before viral infection results in a significant reduction in LacZ activity (greater than 3-fold), the levels of LacZ activity observed are only 31% of those observed following transfection of an empty vector. The single-chain obligate heterodimer variants cleaving the HSV4, HSV2 or HSV 12 target sequences display reductions in LacZ activity similar to that of I-SceI (2- to 4-fold). The level of LacZ activity observed was 25-51% of that observed with an empty vector.

The most efficient I-CreI variants cleaving the HSV2, HSV4 and HSV12 target sequences, SCOH-HSV2-M1-MC-80K105A132V (pCLS2459, SEQ ID NO: 256), SCOH-HSV4-M2-105A-MF-80K132V (pCLS2790, SEQ ID NO:534) and SCOH-HSV12-M1-ME-132V (pCLS2633, SEQ ID NO:465), respectively, were characterized further (FIG. 44). pCLS2790 (SEQ ID NO: 534) differs from pCLS2481 (SEQ ID NO: 454) by the presence of a CMV promoter. These three meganucleases were assayed in four independent experiments, and viral load was estimated with two different methods: measurement of β-galactosidase activity (FIG. 44) and monitoring of viral DNA content by Q-PCR (FIG. 45). Both assays gave reproducible and consistent results, with the HSV2 meganuclease having a significantly higher inhibitory effect. Differences in efficacy can reflect expression level, specific activity of the endonuclease, or accessibility of the target (related to epigenetic modifications of the targeted locus). Western blotting demonstrated that the meganucleases were not expressed at similar levels (FIG. 46). HSV2 expression is higher than other meganucleases in COS-7 cells.

To further characterize the anti-viral potential of the I-CreI variants cleaving the HSV2, HSV4 and HSV12 target sequences, cells were infected at a MOI of 10⁻² and 10⁻¹ and viral load was monitored 24 hours post-infection by Q-PCR (FIG. 53). Efficient inhibition of viral infection was observed with all three meganucleases, HSV2, HSV4 and HSV12, in all conditions, with up to 64% reduction of viral load at a MOI of 10⁻¹ (FIG. 53). These results indicate that at higher levels of infection the HSV meganucleases display an efficient anti-viral activity.

b) Anti-HSV Meganucleases Induce High Rates of Mutations at their Target Site.

The inhibition of HSV-1 infection by the anti-HSV meganucleases is thought to be due to cleavage of viral DNA. Cleavage of an episomal sequence can result in its degradation and loss, but also to its repair by the endogenous maintenance systems of the cell. DNA double strand breaks (DSBs) can be repaired by homologous recombination (HR) or by non homologous end joining (NHEJ), two alternative pathways. Following cleavage by an endonuclease, HR or NHEJ will in most of the cases reseal the break in a seamless manner (although by two totally different mechanisms). However, there is an error prone NHEJ pathway that results mostly in small deletions or insertions (indels) at the cleavage site. Although this process is a very inefficient one, it is the one that precludes re-cleavage of the target site after DNA repair, single indels being sufficient to totally abolish recognition by the endonuclease. Thus, in the cells treated with the meganuclease before infection, the remaining viral genomes should in principle display a detectable level of such indels.

We amplified by PCR the HSV2 and HSV4 DNA regions from cells treated with the HSV2 and HSV4 meganucleases before infection (The cell and genomic DNA samples used for the PCR correspond to those used for Q-PCR, FIG. 45), and used deep sequencing to characterize individual PCR products (FIG. 47). In the absence of meganuclease, indels were absent or barely detectable, with no observed events for tHSV4, and 0.05% for tHSV2. However, mutation frequencies increased up to 2.8% in samples transfected with 5 μg of mHSV4 expressing vectors, and 16% in samples treated with 5 μg of mHSV2 expressing vectors. In both cases, deletions largely outnumbered insertions (2.5% of deletions vs. 0.3% of insertions with mHSV4, 15% of deletions and 1% of insertions with mIISV2) and as shown on FIG. 48 and Table XXXVI, there was a strong bias in favor of small deletions. However, deletions of more than 100 bp were detected with mHSV2. Deletions were also mostly small adducts with HSV4, also several large events (40 bp) were observed with HSV4 (FIG. 47).

TABLE XXXVI
Frequencies of deletion/insertion sizes in rHSV-1 genome after
exposure to HSV2 or HSV4 meganuclease

| | | Number of | |
|---|---|---|---|
| | | analyzed | Number of PCR products |
| Target | | PCR product | with mutations (% InDel) |
| tHSV2 | No treatment | 23527 | 12 (0.05%) |
| | mHSV2 treatment | 10356 | 1683 (16%) |
| | | | (deletion event: 15%, |
| | | | insertion event: 1%) |
| tHSV4 | No treatment | 16961 | 0 |
| | mHSV4 treatment | 12228 | 343 (2.8%) |
| | | | (deletion event: 2.5%, |
| | | | insertion event: 0.3%) |

These results confirm that a very high rate of cleavage occurs at the meganucleases cleavage sites, and are consistent with a mechanism of inhibition based on viral DNA cleavage. It is also no surprise that the most active meganuclease, HSV2, which also gives the highest rates of infection inhibition, is also the one that gives the highest frequency of mutations.

### c) Meganucleases Prevent the Infection of COS-7Cells by a Wt HSV-1 Virus

To further validate the anti-viral potential of the I-CreI variant cleaving the HSV2 target sequence, SCOH-HSV2-M1-MC-80K105A132V (pCLS2459, SEQ ID NO: 256), the ability to prevent infection with a wild type virus was examined. COS-7 cells were infected with wt HSV-1 virus at various MOIs (10⁻³, 10⁻², 10⁻¹, 1) following the same protocol as for rHSV1, and viral load was monitored by Q-PCR 24 hours post-infection (FIG. 54). Very efficient inhibition could be observed up to an MOI of 1. These results indicate that the HSV2 meganuclease displays a strong anti-viral activity that is not limited to a recombinant HSV-1 virus.

### d) Meganucleases Prevent the Infection of BSR Cells by a Wt HSV-1 Virus

As a complement to the previous analysis, the antiviral potential I-CreI variants cleaving the HSV2, HSV4 and HSV12 target sequences was tested with a wild type virus using an alternative approach. 200,000 BSR cells were seeded in 6-well plates, and co-transfected 24 hours later (day 1) with 1.5 μg of meganuclease expressing plasmids and 1.5 μg of a GFP expressing plasmid (pCLS0099). Two days later (day 3), these cells were infected with a wild type virus and viral infection was estimated 8 hours after by immunostaining with an antibody recognizing the gC viral glycoprotein. Since the results obtained with rHSV1 suggested that the HSV2 (SEQ ID NO:256), HSV4 (SEQ ID NO:534) and HSV12 (SEQ ID NO:465) meganucleases had a strong antiviral effect, we raised the virus dose in this experiment, using several MOIs ranging from 0.1 to 8. In order to quantify the antiviral potential of each meganuclease, we monitored the ratio of infected cells among transfected (GFP+) and non transfected (GFP−) cells (FIG. 48). An antiviral index was calculated as the ratio of the infection frequencies in GFP+vs. GFP− cells. In the transfection conditions we used, GFP+ and GFP− cells were both well represented in all experiments, with an average GFP+ cell frequency of 0.63 over 90 transfections (standard deviation: 0.20). Results are summarized in FIG. 48B. A strong inhibitory effect was observed at MOIs of 0.1 and 0.5, with 4 to 7 times less infection among GFP+ cells than among GFP− ones. However, this effect disappeared (compared to negative control) by a MOI of 2. One should note that at low MOI, a small but reproducible effect was observed even with the negative control, suggesting that transfection of a GFP plasmid might have an effect by itself, maybe by transiently disrupting cellular membrane metabolism.

### EXAMPLE 4

### Strategy for Engineering Meganucleases Cleaving Target from the US2 Gene in HSV1 Genome

HSV 1 is a 24 bp (non-palindromic) target (HSV1: AT-GGG-AC-GTC-GTAA-GGG-GG-CCT-GG, SEQ ID NO:23, FIG. 49) present in the US2 gene encoding a possibly HSV-1 envelope-associated protein that interacts with cytokeratin 18. This 1.3 kb gene is present in one copy at position 134053 to 135304 of the US region. The Us2 gene is conserved among alphaherpesviruses, but its function is not known. The Us2 protein is packaged as part of the tegument of mature virions (Clase A C et al, J Virol. 2003 November; 77(22):12285-98). Within the human cytomegalovirus family, the US2 glycoprotein is involved immune evasion (Besold K et al., Virology. 2009 Aug. 15; 391(1):5-19. Epub 2009 Jun. 30). This gene is considered as non essential for virus replication is cell culture but was considered of interest due to its potential role in virus evasion. The target HSV1 is located from nucleotide 134215 to 134238 (accession number NC_{—}001806; FIG. 1).

I-CreI heterodimers capable of cleaving a target sequence (HSV 1: AT-GGG-AC-GTC-GTAA-GGG-GG-CCT-GG, SEQ ID NO:23) were identified using methods derived from those described in Chames et al. (Nucleic Acids Res., 2005, 33, e178), Arnould et al. (J. Mol. Biol., 2006, 355, 443-458), Smith et al. (Nucleic Acids Res., 2006, 34, e149), Arnould et al. (Arnould et al. J Mol Biol. 2007 371:49-65). These results were then utilized to design single-chain meganucleases directed against the target sequence of SEQ ID NO: 23. These single-chain meganucleases were cloned into mammalian expression vectors and tested for HSV1 cleavage in CHO cells. Strong cleavage activity of the HSV 1 target could be observed for these single chain molecules in mammalian cells.

### EXAMPLE 4.1

### Identification of Meganucleases Cleaving HSV1

I-CreI variants potentially cleaving the HSV1 target sequence in heterodimeric form were constructed by genetic engineering. Pairs of such variants were then co-expressed in yeast. Upon co-expression, one obtains three molecular species, namely two homodimers and one heterodimer. It was then determined whether the heterodimers were capable of cutting the HSV 1 target sequence of SEQ ID NO:23.

a) Construction of Variants of the I-CreI Meganuclease Cleaving Palindromic Sequences Derived from the HSV 1 Target Sequence

The HSV1 sequence is partially a combination of the 10GGG_P (SEQ ID NO: 473), 5GTC_P (SEQ ID NO:474), 10AGG_P (SEQ ID NO: 475) and 5CCC_P (SEQ ID NO: 476), target sequences which are shown on FIG. 49. These sequences are cleaved by meganucleases obtained as described in International PCT applications WO 2006/097784 and WO 2006/097853, Arnould et al. (J. Mol. Biol., 2006, 355, 443-458) and Smith et al. (Nucleic Acids Res., 2006). Thus, HSV1 should be cleaved by combinatorial variants resulting from these previously identified meganucleases.

The GTAA sequence in −2 to 2 of HSV1 target was first substituted with the GTAC sequence from C1221 (SEQ ID NO:2), resulting in target HSV1.2 (FIG. 49).

Two palindromic targets, HSV1.3 (and HSV1.5) and HSV1.4 (and HSV1.6), were derived from HSV1 (FIG. 49). Since HSV1.3 and HSV1.4 are palindromic, they should be cleaved by homodimeric proteins. Therefore, homodimeric I-CreI variants cleaving either the HSV1.3 palindromic target sequence of SEQ ID NO:477 or the HSV1.4 palindromic target sequence of SEQ ID NO: 478 were constructed using methods derived from those described in Chames et al. (Nucleic Acids Res., 2005, 33, e178), Arnould et al. (J. Mol. Biol., 2006, 355, 443-458), Smith et al. (Nucleic Acids Res., 2006, 34, e149) and Arnould et al. (Arnould et al. J Mol Biol. 2007 371:49-65).

b) Construction of Target Vector

An oligonucleotide of SEQ ID NO:540, corresponding to the HSV1 target sequence flanked by gateway cloning sequences, was ordered from PROLIGO. This oligo has the following sequence: TGGCATACAAGTTTATGGGACGTCGTAAGGGGGCCTGGCAATCGTCTGTCA. Double-stranded target DNA, generated by PCR amplification of the single stranded oligonucleotide, was cloned into the pCLS1055 yeast reporter vector using the Gateway protocol (INVITROGEN).

Yeast reporter vector was transformed into the FYBL2-7B Saccharomyces cerevisiae strain having the following genotype: MATα, ura3Δ851, trp1Δ63, leu2Δ1, lys2Δ202. The resulting strain corresponds to a reporter strain.

c) Co-Expression of Variants

The open reading frames coding for the variants cleaving the HSV1.6 (and HSV1.4) or the HSV1.5 (and HSV1.3) sequence were cloned in the pCLS542 expression vector and in the pCLS1107 expression vector, respectively. Yeast DNA from these variants was extracted using standard protocols and was used to transform E. coli. The resulting plasmids were then used to co-transform yeast. Transformants were selected on synthetic medium lacking leucine and containing G418.

d) Mating of Meganucleases Coexpressing Clones and Screening in Yeast

Mating was performed using a colony gridder (QpixII, Genetix). Variants were gridded on nylon filters covering YPD plates, using a low gridding density (4-6 spots/cm²). A second gridding process was performed on the same filters to spot a second layer consisting of different reporter-harboring yeast strains for each target. Membranes were placed on solid agar YPD rich medium, and incubated at 30° C. for one night, to allow mating. Next, filters were transferred to synthetic medium, lacking leucine and tryptophan, adding G418, with galactose (2%) as a carbon source, and incubated for five days at 37° C., to select for diploids carrying the expression and target vectors. After 5 days, filters were placed on solid agarose medium with 0.02% X-Gal in 0.5 M sodium phosphate buffer, pH7.0, 0.1% SDS, 6% dimethyl formamide (DMF), 7 mM β-mercaptoethanol, 1% agarose, and incubated at 37° C., to monitor β-galactosidase activity. Results were analyzed by scanning and quantification was performed using an appropriate software.

e) Results

Co-expression of different variants resulted in cleavage of the HSV1 target in 48 tested combinations. Functional combinations are summarized in Table XXXVII herebelow. In this Table, “+” indicates a functional combination on the HSV1 target sequence, i.e., the heterodimer is capable of cleaving the HSV1 target sequence.

TABLE XXXVII

| | | Amino acids positions and residues | | | | | | | |
|---|---|---|---|---|---|---|---|---|---|
| | | of the I-CreI variants cleaving the HSV1.5 (SEQ ID NO: 479) | | | | | | | |
| | | (and.3 (SEQ ID NO: 477)) target | | | | | | | |
| | | | | | | | | 7E 30R | 30R 33G |
| | | 30R 33G | 30R 33G | 28E 33R | | | 30R 33G | 33G 38T | 38T 54L |
| | | 38T 75N | 38T 50R | 38R 40K | 30R 33G | 30R 33G | 38T 54L | 72P 75N | 75N 103S |
| | | 111H | 75N | 75N | 38T 122L | 38T 106P | 75N | 105A | 157G |
| | | (SEQ ID | (SEQ ID | (SEQ ID | (SEQ ID | (SEQ ID | (SEQ ID | (SEQ ID | (SEQ ID |
| | | NO: 541) | NO: 542) | NO: 543) | NO: 544) | NO: 545) | NO: 546) | NO: 547) | NO: 548) |
| Amino acids | 30G 38R 44K | + | + | + | + | + | + | + | + |
| positions and | 70E 75N 96E | | | | | | | | |
| residues of | (SEQ ID NO: 549) | | | | | | | | |
| the I-CreI | 30G 38R 44K | + | + | + | + | + | + | + | + |
| variants | 70D 75N | | | | | | | | |
| cleaving | (SEQ ID NO: 550) | | | | | | | | |
| the HSV1.6 | 29S 30G 38R | + | + | + | + | + | + | + | + |
| (SEQ ID NO: 480) | | | | | | | | | |
| (and.4 | 44K 70D 75N | | | | | | | | |
| (SEQ ID NO: 478)) | (SEQ ID NO: 551) | | | | | | | | |
| target | 9Y 30G 38R | + | + | + | + | + | + | + | + |
| | 44K 70E 75N | | | | | | | | |
| | (SEQ ID NO: 552) | | | | | | | | |
| | 30G 38R 44K | + | + | + | + | + | + | + | + |
| | 70E 75N | | | | | | | | |
| | (SEQ ID NO: 553) | | | | | | | | |
| | 30G 38R 44K | + | + | + | + | + | + | + | + |
| | 57E 70E 75N | | | | | | | | |
| | 108V | | | | | | | | |
| | (SEQ ID NO: 554) | | | | | | | | |

In conclusion, several heterodimeric I-CreI variants, capable of cleaving the HSV1 target sequence in yeast, were identified.

### EXAMPLE 4.2

### Covalent Assembly as Single Chain and Improvement of Meganucleases Cleaving HSV1

I-CreI variants able to efficiently cleave the HSV 1 target in yeast when forming heterodimers are described hereabove in Example 4.1. Among them, a couple has been chosen as scaffold for further single chain meganuclease assembly and activity improvement. The screen and validation in CHO cells is a single-strand annealing (SSA) based assay where cleavage of the target by the meganucleases induces homologous recombination and expression of a LagoZ reporter gene (a derivative of the bacterial lacZ gene).

a) Cloning of HSV1 Target in a Vector for CHO Screen

An oligonucleotide corresponding to the HSV 1 target sequence flanked by gateway cloning sequences, was ordered from PROLIGO (SEQ ID NO:555; TGGCATACAAGTTTATGGGACGTCGTAAGGGGGCCTGGCAATCGTCTGTCA). Double-stranded target DNA, generated by PCR amplification of the single stranded oligonucleotide, was cloned using the Gateway protocol (INVITROGEN) into the pCLS1058 CHO reporter vector. Cloned target was verified by sequencing (MILLEGEN).

b) Gene Synthesis and Cloning of HSV1 Meganucleases

The open-reading frames coding for single chain meganuclease variants listed in Table XXXVII were generated by synthetic gene assembly at TOP Gene Technologies, Inc (Montreal, CANADA) and cloned in into the pCLS1853 expression vector using the AscI and XhoI restriction enzymes for internal fragment replacement.

c) Extrachromosomal Assay in Mammalian Cells

CHO K1 cells were transfected as described in Example 1.2. 72 hours after transfection, culture medium was removed and 150 μl of lysis/revelation buffer for β-galactosidase liquid assay was added. After incubation at 37° C., OD was measured at 420 nm. The entire process is performed on an automated Velocity11 BioCel platform. Per assay, 150 ng of target vector was cotransfected with an increasing quantity of variant DNA from 0 to 25 ng. The total amount of transfected DNA was completed to 175 ng (target DNA, variant DNA, carrier DNA) using an empty vector (pCLS0002).

d) Results

Among the I-CreI variants able to cleave the HSV1 target in yeast when forming heterodimers (in Example 4.1), a couple has been chosen as scaffold for further single chain meganuclease assembly and directed mutagenesis for activity improvement (Table XXXVIII).

TABLE XXXVIII

| HSV1 variant | Amino acids positions and residues of the I-CreI variants |
|---|---|
| HSV1.3-M5 | 30R 33G 38T 106P (SEQ ID NO: 545) |
| HSV1.4-MF | 30G 38R 44K 57E 70E 75N 108V (SEQ ID NO: 554) |

The HSV1.3-M5×HSV1.4-MF HSV1 heterodimer gives high cleavage activity in yeast. HSV1.3-M5 (SEQ ID NO:545) is a HSV1.5 cutter that bears the following mutations in comparison with the I-CreI wild type sequence: 30R 33G 38T 106P. HSV1.4-MF is a HSV1.6 cutter that bears the following mutations in comparison with the I-CreI wild type sequence: 30G 38R 44K 57E 70E 75N 108V.

Single chain constructs were engineered using the linker RM2 of SEQ ID NO:464 (AAGGSDKYNQALSKYNQALSKYNQALSGGGGS), thus resulting in the production of the single chain molecule: MA-linkerRM2-M1. During this design step, the G19S mutation was introduced in the C-terminal MF variant. In addition, mutations K7E, K96E were introduced into the M5 variant and mutations E8K, E61R into the MF variant to create the single chain molecule: MA (K7E K96E)-linkerRM2-M1 (E8K E61R G19S) that is further called SCOH-HSV1-M5-MF (SEQ ID NO: 556) scaffold. Some additional amino-acid substitutions have been found in previous studies to enhance the activity of I-CreI derivatives: some of these mutations correspond to the replacement of Glutamic acid 80 with Lysine (E80K), Valine 105 with Alanine (V105A) and Isoleucine 132 with Valine (I132V). The resulting proteins are shown in Table XXXIX below (SEQ ID NO:557-568). All the single chain molecules were assayed in CHO for cleavage of the HSV1 target.

TABLE XXXIX
Example of Single Chain series designed for strong
cleavage of HSV1 target in CHO cells

| | | | HSV1 | | SEQ |
|---|---|---|---|---|---|
| | | mutations in | cleavage | | ID |
| plasmid | variant | Single Chain | in CHO | Protein sequence | NO: |
| pCLS2528 | SCOH- | 7E30R33G38T9 | + | MANTKYNEEFLLYLAGFVDGDGSIIAQIKPRQSGKFK | 556 |
| | HSV1-M5- | 6E106P_8K19S | | HTLSLTFQVTQKTQRRWFLDKLVDEIGVGYVRDRGS | |
| | MF | 30G38R44K57E | | VSDYILSEIKPLHNFLTQLQPFLELKQKQANLVPKIIEQ | |
| | | 61R70E75N108V | | LPSAKESPDKFLEVCTWVDQIAALNDSKTRKTTSETV | |
| | | | | RAVLDSLSEKKKSSPAAGGSDKYNQALSKYNQALSK | |
| | | | | YNQALSGGGGSNKKFLLYLAGFVDSDGSIIAQIKPGQ | |
| | | | | SYKFKHRLSLTFKVTQKTQRRWFLDELVDRIGVGYV | |
| | | | | RDEGSVSNYILSEIKPLHNFLTQLQPFLKLKQKQANL | |
| | | | | VLKVIEQLPSAKESPDKFLEVCTWVDQIAALNDSKTR | |
| | | | | KTTSETVRAVLDSLSEKKKSSP | |
| pCLS2584 | SCOH- | 7E30R33G38T9 | + | MANTKYNEEFLLYLAGFVDGDGSIIAQIKPRQSGKFK | 557 |
| | HSV1-M5- | 6E106P_8K19S | | HTLSLTFQVTQKTQRRWFLDKLVDEIGVGYVRDRGS | |
| | MF-132V | 30G38R44K57E | | VSDYILSEIKPLHNFLTQLQPFLELKQKQANLVPKIIEQ | |
| | | 61R70E75N108 | | LPSAKESPDKFLEVCTWVDQIAALNDSKTRKTTSETV | |
| | | V132V | | RAVLDSLSEKKKSSPAAGGSDKYNQALSKYNQALSK | |
| | | | | YNQALSGGGGSNKKFLLYLAGFVDSDGSIIAQIKPGQ | |
| | | | | SYKFKHRLSLTFKVTQKTQRRWFLDELVDRIGVGYV | |
| | | | | RDEGSVSNYILSEIKPLHNFLTQLQPFLKLKQKQANL | |
| | | | | VLKVIEQLPSAKESPDKFLEVCTWVDQVAALNDSKT | |
| | | | | RKTTSETVRAVLDSLSEKKKSSP | |
| pCLS2585 | SCOH- | 7E30R33G38T9 | + | MANTKYNEEFLLYLAGFVDGDGSIIAQIKPRQSGKFK | 558 |
| | HSV1-M5- | 6E106P_8K19S | | HTLSLTFQVTQKTQRRWFLDKLVDEIGVGYVRDRGS | |
| | MF- | 30G38R44K57E | | VSDYILSEIKPLHNFLTQLQPFLELKQKQANLVPKIIEQ | |
| | 80K105A | 61R70E75N80K | | LPSAKESPDKFLEVCTWVDQIAALNDSKTRKTTSETV | |
| | | 105A108V | | RAVLDSLSEKKKSSPAAGGSDKYNQALSKYNQALSK | |
| | | | | YNQALSGGGGSNKKFLLYLAGFVDSDGSIIAQIKPGQ | |
| | | | | SYKFKHRLSLTFKVTQKTQRRWFLDELVDRIGVGYV | |
| | | | | RDEGSVSNYILSKIKPLHNFLTQLQPFLKLKQKQANL | |
| | | | | ALKVIEQLPSAKESPDKFLEVCTWVDQIAALNDSKTR | |
| | | | | KTTSETVRAVLDSLSEKKKSSP | |
| pCLS2586 | SCOH- | 7E30R33G38T9 | + | MANTKYNEEFLLYLAGFVDGDGSIIAQIKPRQSGKFK | 559 |
| | HSV1-M5- | 6E106P_8K19S | | HTLSLTFQVTQKTQRRWFLDKLVDEIGVGYVRDRGS | |
| | MF- | 30G38R44K57E | | VSDYILSEIKPLHNFLTQLQPFLELKQKQANLVPKIIEQ | |
| | 80K132V | 61R70E75N80K | | LPSAKESPDKFLEVCTWVDQIAALNDSKTRKTTSETV | |
| | | 108V132V | | RAVLDSLSEKKKSSPAAGGSDKYNQALSKYNQALSK | |
| | | | | YNQALSGGGGSNKKFLLYLAGFVDSDGSIIAQIKPGQ | |
| | | | | SYKFKHRLSLTFKVTQKTQRRWFLDELVDRIGVGYV | |
| | | | | RDEGSVSNYILSKIKPLHNFLTQLQPFLKLKQKQANL | |
| | | | | VLKVIEQLPSAKESPDKFLEVCTWVDQVAALNDSKT | |
| | | | | RKTTSETVRAVLDSLSEKKKSSP | |
| pCLS2587 | SCOH- | 7E30R33G38T9 | + | MANTKYNEEFLLYLAGFVDGDGSIIAQIKPRQSGKFK | 560 |
| | HSV1-M5- | 6E106P_8K19S | | HTLSLTFQVTQKTQRRWFLDKLVDEIGVGYVRDRGS | |
| | MF- | 30G38R44K57E | | VSDYILSEIKPLHNFLTQLQPFLELKQKQANLVPKIIEQ | |
| | 80K105A1 | 61R70E75N80K | | LPSAKESPDKFLEVCTWVDQIAALNDSKTRKTTSETV | |
| | 32V | 105A108V132V | | RAVLDSLSEKKKSSPAAGGSDKYNQALSKYNQALSK | |
| | | | | YNQALSGGGGSNKKFLLYLAGFVDSDGSIIAQIKPGQ | |
| | | | | SYKFKHRLSLTFKVTQKTQRRWFLDELVDRIGVGYV | |
| | | | | RDEGSVSNYILSKIKPLHNFLTQLQPFLKLKQKQANL | |
| | | | | ALKVIEQLPSAKESPDKFLEVCTWVDQVAALNDSKT | |
| | | | | RKTTSETVRAVLDSLSEKKKSSP | |
| pCLS2588 | SCOH- | 7E30R33G38T9 | + | MANTKYNEEFLLYLAGFVDGDGSIIAQIKPRQSGKFK | 561 |
| | HSV1-M5- | 6E106P132V_8 | | HTLSLTFQVTQKTQRRWFLDKLVDEIGVGYVRDRGS | |
| | 132V-MF | K19S30G38R44 | | VSDYILSEIKPLHNFLTQLQPFLELKQKQANLVPKIIEQ | |
| | | K57E61R70E75 | | LPSAKESPDKFLEVCTWVDQVAALNDSKTRKTTSET | |
| | | N108V | | VRAVLDSLSEKKKSSPAAGGSDKYNQALSKYNQALS | |
| | | | | KYNQALSGGGGSNKKFLLYLAGFVDSDGSIIAQIKPG | |
| | | | | QSYKFKHRLSLTFKVTQKTQRRWFLDELVDRIGVGY | |
| | | | | VRDEGSVSNYILSEIKPLHNFLTQLQPFLKLKQKQAN | |
| | | | | LVLKVIEQLPSAKESPDKFLEVCTWVDQIAALNDSKT | |
| | | | | RKTTSETVRAVLDSLSEKKKSSP | |
| pCLS2589 | SCOH- | 7E30R33G38T9 | + | MANTKYNEEFLLYLAGFVDGDGSIIAQIKPRQSGKFK | 562 |
| | HSV1-M5- | 6E106P132V_8 | | HTLSLTFQVTQKTQRRWFLDKLVDEIGVGYVRDRGS | |
| | 132V-MF- | K19S30G38R44 | | VSDYILSEIKPLHNFLTQLQPFLELKQKQANLVPKIIEQ | |
| | 132V | K57E61R70E75 | | LPSAKESPDKFLEVCTWVDQVAALNDSKTRKTTSET | |
| | | N108V132V | | VRAVLDSLSEKKKSSPAAGGSDKYNQALSKYNQALS | |
| | | | | KYNQALSGGGGSNKKFLLYLAGFVDSDGSIIAQIKPG | |
| | | | | QSYKFKHRLSLTFKVTQKTQRRWFLDELVDRIGVGY | |
| | | | | VRDEGSVSNYILSEIKPLHNFLTQLQPFLKLKQKQAN | |
| | | | | LVLKVIEQLPSAKESPDKFLEVCTWVDQVAALNDSKT | |
| | | | | RKTTSETVRAVLDSLSEKKKSSP | |
| pCLS2593 | SCOH- | 7E30R33G38T8 | + | MANTKYNEEFLLYLAGFVDGDGSIIAQIKPRQSGKFK | 563 |
| | HSV1-M5- | 0K96E105A106 | | HTLSLTFQVTQKTQRRWFLDKLVDEIGVGYVRDRGS | |
| | 80K105A- | P_8K19S30G38 | | VSDYILSKIKPLHNFLTQLQPFLELKQKQANLAPKIIEQ | |
| | MF | R44K57E61R70 | | LPSAKESPDKFLEVCTWVDQIAALNDSKTRKTTSETV | |
| | | E75N108V | | RAVLDSLSEKKKSSPAAGGSDKYNQALSKYNQALSK | |
| | | | | YNQALSGGGGSNKKFLLYLAGFVDSDGSIIAQIKPGQ | |
| | | | | SYKFKHRLSLTFKVTQKTQRRWFLDELVDRIGVGYV | |
| | | | | RDEGSVSNYILSEIKPLHNFLTQLQPFLKLKQKQANL | |
| | | | | VLKVIEQLPSAKESPDKFLEVCTWVDQIAALNDSKTR | |
| | | | | KTTSETVRAVLDSLSEKKKSSP | |
| pCLS2597 | SCOH- | 7E30R33G38T8 | + | MANTKYNEEFLLYLAGFVDGDGSIIAQIKPRQSGKFK | 564 |
| | HSV1-M5- | 0K96E106P132 | | HTLSLTFQVTQKTQRRWFLDKLVDEIGVGYVRDRGS | |
| | 80K132V- | V_8K19S30G38 | | VSDYILSKIKPLHNFLTQLQPFLELKQKQANLVPKIIEQ | |
| | MF | R44K57E61R70 | | LPSAKESPDKFLEVCTWVDQVAALNDSKTRKTTSET | |
| | | E75N108V | | VRAVLDSLSEKKKSSPAAGGSDKYNQALSKYNQALS | |
| | | | | KYNQALSGGGGSNKKFLLYLAGFVDSDGSIIAQIKPG | |
| | | | | QSYKFKHRLSLTFKVTQKTQRRWFLDELVDRIGVGY | |
| | | | | VRDEGSVSNYILSEIKPLHNFLTQLQPFLKLKQKQAN | |
| | | | | LVLKVIEQLPSAKESPDKFLEVCTWVDQIAALNDSKT | |
| | | | | RKTTSETVRAVLDSLSEKKKSSP | |
| pCLS2598 | SCOH- | 7E30R33G38T8 | + | MANTKYNEEFLLYLAGFVDGDGSIIAQIKPRQSGKFK | 565 |
| | HSV1-M5- | 0K96E106P132 | | HTLSLTFQVTQKTQRRWFLDKLVDEIGVGYVRDRGS | |
| | 80K132V- | V_8K19S30G38 | | VSDYILSKIKPLHNFLTQLQPFLELKQKQANLVPKIIEQ | |
| | MF-105A | R44K57E61R70 | | LPSAKESPDKFLEVCTWVDQVAALNDSKTRKTTSET | |
| | | E75N105A108V | | VRAVLDSLSEKKKSSPAAGGSDKYNQALSKYNQALS | |
| | | | | KYNQALSGGGGSNKKFLLYLAGFVDSDGSIIAQIKPG | |
| | | | | QSYKFKHRLSLTFKVTQKTQRRWFLDELVDRIGVGY | |
| | | | | VRDEGSVSNYILSEIKPLHNFLTQLQPFLKLKQKQAN | |
| | | | | LALKVIEQLPSAKESPDKFLEVCTWVDQIAALNDSKT | |
| | | | | RKTTSETVRAVLDSLSEKKKSSP | |
| pCLS2599 | SCOH- | 7E30R33G38T8 | + | MANTKYNEEFLLYLAGFVDGDGSIIAQIKPRQSGKFK | 566 |
| | HSV1-M5- | 0K96E106P132 | | HTLSLTFQVTQKTQRRWFLDKLVDEIGVGYVRDRGS | |
| | 80K132V- | V_8K19S30G38 | | VSDYILSKIKPLHNFLTQLQPFLELKQKQANLVPKIIEQ | |
| | MF-132V | R44K57E61R70 | | LPSAKESPDKFLEVCTWVDQVAALNDSKTRKTTSET | |
| | | E75N108V132V | | VRAVLDSLSEKKKSSPAAGGSDKYNQALSKYNQALS | |
| | | | | KYNQALSGGGGSNKKFLLYLAGFVDSDGSIIAQIKPG | |
| | | | | QSYKFKHRLSLTFKVTQKTQRRWFLDELVDRIGVGY | |
| | | | | VRDEGSVSNYILSEIKPLHNFLTQLQPFLKLKQKQAN | |
| | | | | LVLKVIEQLPSAKESPDKFLEVCTWVDQVAALNDSKT | |
| | | | | RKTTSETVRAVLDSLSEKKKSSP | |
| pCLS2600 | SCOH- | 7E30R33G38T8 | + | MANTKYNEEFLLYLAGFVDGDGSIIAQIKPRQSGKFK | 567 |
| | HSV1-M5- | 0K96E106P132 | | HTLSLTFQVTQKTQRRWFLDKLVDEIGVGYVRDRGS | |
| | 80K132V- | V_8K19S30G38 | | VSDYILSKIKPLHNFLTQLQPFLELKQKQANLVPKIIEQ | |
| | MF- | R44K57E61R70 | | LPSAKESPDKFLEVCTWVDQVAALNDSKTRKTTSET | |
| | 80K105A | E75N80K105A1 | | VRAVLDSLSEKKKSSPAAGGSDKYNQALSKYNQALS | |
| | | 08V | | KYNQALSGGGGSNKKFLLYLAGFVDSDGSIIAQIKPG | |
| | | | | QSYKFKHRLSLTFKVTQKTQRRWFLDELVDRIGVGY | |
| | | | | VRDEGSVSNYILSKIKPLHNFLTQLQPFLKLKQKQAN | |
| | | | | LALKVIEQLPSAKESPDKFLEVCTWVDQIAALNDSKT | |
| | | | | RKTTSETVRAVLDSLSEKKKSSP | |
| pCLS4379 | SCOH- | 7E30R33G38T9 | + | MANTKYNEEFLLYLAGFVDGDGSIIAQIKPRQSGKFK | 568 |
| | HSV1-M5- | 6E106P132V_8 | | HTLSLTFQVTQKTQRRWFLDKLVDEIGVGYVRDRGS | |
| | MFrev | K19S30G38R44 | | VSDYILSEIKPLHNFLTQLQPFLELKQKQANLVPKIIEQ | |
| | | K57E61R108V | | LPSAKESPDKFLEVCTWVDQVAALNDSKTRKTTSET | |
| | | | | VRAVLDSLSEKKKSSPAAGGSDKYNQALSKYNQALS | |
| | | | | KYNQALSGGGGSNKKFLLYLAGFVDSDGSIIAQIKPG | |
| | | | | QSYKFKHRLSLTFKVTQKTQRRWFLDELVDRIGVGY | |
| | | | | VRDRGSVSDYILSEIKPLHNFLTQLQPFLKLKQKQAN | |
| | | | | LVLKVIEQLPSAKESPDKFLEVCTWVDQIAALNDSKT | |
| | | | | RKTTSETVRAVLDSLSEKKKSSP | |

d) Results

The activity of the single chain molecules against the HSV1 target (SEQ ID NO:23) was monitored using the previously described CHO assay along with our internal control SCOH-RAG and I-Sce I meganucleases. All comparisons were done upon DNA dose response of transfected variant DNA. All the single molecules displayed HSV1 target cleavage activity in CHO assay as listed in Table XXXIX. Variants shared specific behaviour upon assayed dose depending on the mutation profile they bear. For Example, pCLS2588 expressing the SCOH-HSV1-M5-132V-MF (SEQ ID NO:557) has a similar profile than I-Sce I (FIG. 42). Its activity increases with the quantity of transfected DNA. With pCLS4379, expressing SCOH-HSV1-M5-MFrev (SEQ ID NO:568), the global activity can be increased. All of the variants described are active and can be used for the HSV-1 virus US2 gene targeting and cleavage.

### EXAMPLE 5

### Strategy for Engineering Meganucleases Cleaving Target from the UL30 Gene in HSV1 Genome

I-CreI heterodimers capable of cleaving a target sequence (HSV8: CC-GCT-CT-GTT-TTAC-CGC-GT-CTA-CG, SEQ ID NO:481) were identified using methods derived from those described in Chames et al. (Nucleic Acids Res., 2005, 33, e178), Arnould et al. (J. Mol. Biol., 2006, 355, 443-458), Smith et al. (Nucleic Acids Res., 2006, 34, e149), Arnould et al. (Arnould et al. J Mol Biol. 2007 371:49-65). These results were then utilized to design single-chain meganucleases directed against the target sequence of SEQ ID NO:481. These single-chain meganucleases were cloned into mammalian expression vectors and tested for HSV8 cleavage in CHO cells. Strong cleavage activity of the HSV8 target could be observed for these single chain molecules in mammalian cells.

### EXAMPLE 5.1

### Identification of Meganucleases Cleaving HSV8

I-CreI variants potentially cleaving the HSV8 target sequence in heterodimeric form were constructed by genetic engineering. Pairs of such variants were then co-expressed in yeast. Upon co-expression, one obtains three molecular species, namely two homodimers and one heterodimer. It was then determined whether the heterodimers were capable of cutting the HSV8 target sequence of SEQ ID NO:481.

a) Construction of Variants of the I-CreI Meganuclease Cleaving Palindromic Sequences Derived from the HSV8 Target Sequence

The HSV8 target sequence is partially a combination of the 10GCT_P (SEQ ID NO:483), 5GTT_P (SEQ ID NO:484), 10TAG_P (SEQ ID NO:485), 5GCG_P (SEQ ID NO:486) target sequences which are shown on FIG. 50. These sequences are cleaved by meganucleases obtained as described in International PCT applications WO 2006/097784 and WO 2006/097853, Arnould et al. (J. Mol. Biol., 2006, 355, 443-458) and Smith et al. (Nucleic Acids Res., 2006). Thus, HSV8 should be cleaved by combinatorial variants resulting from these previously identified meganucleases.

The TTAC sequence of HSV8 target in −2 to 2 was first substituted with the GTAC sequence from C1221, resulting in target HSV8.2 (FIG. 50).

Two palindromic targets, HSV8.3 (and HSV8.5) and HSV8.4 (and HSV8.6), were derived from HSV8 (FIG. 50). Since HSV8.3 and HSV8.4 are palindromic, they should be cleaved by homodimeric proteins. Therefore, homodimeric I-CreI variants cleaving either the HSV8.3 palindromic target sequence of SEQ ID NO:487 or the HSV8.4 palindromic target sequence of SEQ ID NO:488 were constructed using methods derived from those described in Chames et al. (Nucleic Acids Res., 2005, 33, e178), Arnould et al. (J. Mol. Biol., 2006, 355, 443-458), Smith et al. (Nucleic Acids Res., 2006, 34, e149) and Arnould et al. (Arnould et al. J Mol Biol. 2007 371:49-65).

b) Construction of Target Vector

An oligonucleotide of SEQ ID NO:569, corresponding to the HSV8 target sequence flanked by gateway cloning sequences, was ordered from PROLIGO. This oligo has the following sequence: TGGCATACAAGTTTCCGCTCTGTTTTACCGCGTCTACGCAATCGTCTGTCA. Double-stranded target DNA, generated by PCR amplification of the single stranded oligonucleotide, was cloned into the pCLS1055 yeast reporter vector using the Gateway protocol (INTVITROGEN).

Yeast reporter vector was transformed into the FYBL2-7B Saccharomyces cerevisiae strain having the following genotype: MATα, ura3Δ851, trp1Δ63, leu2Δ1, lys2Δ202. The resulting strain corresponds to a reporter strain.

c) Co-Expression of Variants

The open reading frames coding for the variants cleaving the HSV8.6 (and HSV8.4) or the HSV8.5 (and HSV8.3) sequence were cloned in the pCLS542 expression vector and in the pCLS1107 expression vector, respectively. Yeast DNA from these variants was extracted using standard protocols and was used to transform E. coli. The resulting plasmids were then used to co-transform yeast. Transformants were selected on synthetic medium lacking leucine and containing G418.
d) Mating of Meganucleases Coexpressing Clones and Screening in Yeast

Mating was performed using a colony gridder (QpixII, Genetix). Variants were gridded on nylon filters covering YPD plates, using a low gridding density (4-6 spots/cm²). A second gridding process was performed on the same filters to spot a second layer consisting of different reporter-harboring yeast strains for each target. Membranes were placed on solid agar YPD rich medium, and incubated at 30° C. for one night, to allow mating. Next, filters were transferred to synthetic medium, lacking leucine and tryptophan, adding G418, with galactose (2%) as a carbon source, and incubated for five days at 37° C., to select for diploids carrying the expression and target vectors. After 5 days, filters were placed on solid agarose medium with 0.02% X-Gal in 0.5 M sodium phosphate buffer, pH7.0, 0.1% SDS, 6% dimethyl formamide (DMF), 7 mM β-mercaptoethanol, 1% agarose, and incubated at 37° C., to monitor β-galactosidase activity. Results were analyzed by scanning and quantification was performed using an appropriate software.

e) Results

Co-expression of different variants resulted in cleavage of the HSV8 target in 28 tested combinations. Functional combinations are summarized in Table XXXX here below. In this Table, “+” indicates a functional combination on the HSV8 target sequence, i.e., the heterodimer is capable of cleaving the HSV8 target sequence. “nd” indicates a lack of yeast transformant after co-transformation of HSV8.5 and HSV8.6 variants.

HSV8 target is recognized and cleaved by the meganucleases shown in Table XXXX below.

TABLE XXXX

| | | Amino acids positions and residues | | | | | |
|---|---|---|---|---|---|---|---|
| | | of the I-CreI variants cleaving the HSV8.5 (SEQ ID NO: 489) | | | | | |
| | | (and.3 (SEQ ID NO: 487)) target | | | | | |
| | | 30K33A7 | 30K33T7 | 33H38Y7 | | | |
| | | 0S75H77 | 0S75H77 | 0S75H77 | 30H33S70S7 | 30K33C44L70 | 30K33R70S7 |
| | | Y | Y | Y | 5H77Y | N75N80K | 5H77Y |
| | | (SEQ ID | (SEQ ID | (SEQ ID | (SEQ ID | (SEQ ID | (SEQ ID |
| | | NO: 511) | NO: 512) | NO: 513) | NO: 514) | NO: 515) | NO: 516) |
| Amino acids | 32H 33C 40A | + | + | + | + | + | + |
| positions and | 70S 75N 77K | | | | | | |
| residues of | (SEQ ID NO: 517) | | | | | | |
| the I-CreI | 32H 33C 44R | + | + | + | + | nd | + |
| variants | 68Y 70S 75Y 77N | | | | | | |
| cleaving | (SEQ ID NO: 518) | | | | | | |
| the HSV8.6 | 32H 33C 40A 44R | + | + | + | + | + | + |
| (SEQ ID NO: 490) | 68Y 70S 75Y 77N | | | | | | |
| (and.4 | (SEQ ID NO: 519) | | | | | | |
| (SEQ ID NO: 488)) | 30H 32T 33C 44R | + | + | + | + | + | + |
| target | 68Y 70S 75Y 77N | | | | | | |
| | (SEQ ID NO: 520) | | | | | | |
| | 32N 33C 70S | + | + | + | + | nd | + |
| | 75N 77K | | | | | | |
| | (SEQ ID NO: 521) | | | | | | |

In conclusion, several heterodimeric I-CreI variants, capable of cleaving the HSV8 target sequence in yeast, were identified.

### EXAMPLE 5.2

### Covalent Assembly as Single Chain and Improvement of Meganucleases Cleaving HSV8

I-CreI variants able to efficiently cleave the HSV8 target in yeast when forming heterodimers are described hereabove in Example 5.1. Among them, three couples have been chosen as scaffold for further single chain meganuclease assembly and activity improvement. The screen and validation in CHO cells is a single-strand annealing (SSA) based assay where cleavage of the target by the meganucleases induces homologous recombination and expression of a LagoZ reporter gene (a derivative of the bacterial lacZ gene).

a) Cloning of HSV8 Target in a Vector for CHO Screen

An oligonucleotide corresponding to the HSV8 target sequence flanked by gateway cloning sequences, was ordered from PROLIGO (SEQ ID NO:570; TGGCATACAAGTTTCCGCTCTGTTTTACCGCGTCTACGCAATCGTCTGTCA). Double-stranded target DNA, generated by PCR amplification of the single stranded oligonucleotide, was cloned using the Gateway protocol (INVITROGEN) into the pCLS1058 CHO reporter vector. Cloned target was verified by sequencing (MILLEGEN).

b) Gene Synthesis and Cloning of HSV8 Meganucleases

The open-reading frames coding for single chain meganuclease variants listed in Table XXXXII were generated by synthetic gene assembly at MWG-EUROFINS (Les Ulis, France) and cloned into the pCLS1853 expression vector using the AscI and XhoI restriction enzymes for internal fragment replacement.

c) Extrachromosomal Assay in Mammalian Cells

CHO K1 cells were transfected as described in Example 1.2. 72 hours after transfection, culture medium was removed and 150 μl of lysis/revelation buffer for β-galactosidase liquid assay was added. After incubation at 37° C., OD was measured at 420 nm. The entire process is performed on an automated Velocity11 BioCel platform. Per assay, 150 ng of target vector was cotransfected with an increasing quantity of variant DNA from 0 to 25 ng. The total amount of transfected DNA was completed to 175 ng (target DNA, variant DNA, carrier DNA) using an empty vector (pCLS0002).

d) Results

Among the I-CreI variants able to cleave the HSV8 target in yeast when forming heterodimers (in Example 5.1), three couples have been chosen as scaffolds for further single chain meganuclease assembly and directed mutagenesis for activity improvement (Table XXXXI).

TABLE XXXXI

| | | Amino acids positions and residues of |
|---|---|---|
| | Amino acids positions and residues of the | the I-CreI variants cleaving HSV8.6 |
| HSV8 couple | I-CreI variants cleaving HSV8.5 (and .3) | (and .4) |
| HSV8 b1 | 33H 38Y 70S 75H 77Y (SEQ ID NO: 513) | 32H 33C 40A 70S 75N 77K (SEQ ID |
| | | NO: 517) |
| HSV8 b56 | 30K 33A 70S 75H 77Y (SEQ ID NO: 511) | 32H 33C 40A 70S 75N 77K (SEQ ID |
| | | NO: 517) |
| HSV8 bu | 30K 33R 70S 75H 77Y (SEQ ID NO: 516) | 32H 33C 40A 44R 68Y 70S 75Y 77N (SEQ |
| | | ID NO: 519) |

The three HSV8 b1, b56, bu heterodimers give high cleavage activity in yeast. HSV8 b1 is a couple of HSV8.5×HSV8.6 cutters that bear the following mutations in comparison with the I-CreI wild type sequence: 33H38Y 70S 75H77Y (HSV8.5) (SEQ ID NO:513)×32H33C 40A 70S 75N 77K (HSV8.6) (SEQ ID NO:517). HSV8 b56 is a couple of HSV8.5×HSV8.6 cutters that bear the following mutations in comparison with the I-CreI wild type sequence: 30K 33A 70S 75H77Y (HSV8.5)×32H33C 40A 70S 75N 77K (HSV8.6). HSV8 bu is a couple of HSV8.5×HSV8.6 cutters that bear the following mutations in comparison with the I-CreI wild type sequence: 30K 33R 70S 75H77Y (HSV8.5)×32H33C 40A 44R 68Y 70S 75Y 77N (HSV8.6).

Single chain constructs were engineered using the linker RM2 of SEQ ID NO 464 (AAGGSDKYNQALSKYNQALSKYNQALSGGGGS), thus resulting in the production of the single chain molecule: HSV8.5 variant-linkerRM2-HSV8.6 variant. During this design step, the G19S mutation was introduced in the C-terminal HSV8.6 variant. In addition, mutations K7E, K96E were introduced into the HSV8.5 variant and mutations E8K, E61R into the HSV8.6 variant to create the single chain molecule: HSV8.5-variant (K7E K96E)-linkerRM2-HSV8.6-variant (E8K E61R G19S) that is further called SCOH-HSV8b1, SCOH-HSV8b56 and SCOH-HSV8bu depending on the HSV8 couple used as scaffold (Tables XXXXI and XXXXII). Some additional amino-acid substitutions have been found in previous studies to enhance the activity of I-CreI derivatives: some of these mutations correspond to the replacement of Glutamic acid 80 with Lysine (E80K), Valine 105 with Alanine (V105A) and Isoleucine 132 with Valine (I132V). The resulting proteins are shown in Table XXXXII below. All the single chain molecules were assayed in CHO for cleavage of the HSV8 target.

TABLE XXXXII
Example of Single Chain series designed for strong
cleavage of HSV8 target in CHO cells

| | | | HSV8 | | SEQ |
|---|---|---|---|---|---|
| | | mutations in | cleavage | | ID |
| plasmid | variant | Single Chain | in CHO | Protein sequence | NO: |
| pCLS3301 | SCOH- | 7E33H38Y70S | + | MANTKYNEEFLLYLAGFVDGDGSIIAQIKPNQSHKFKHY | 571 |
| | HSV8b | 75H77Y96E_8 | | LSLTFQVTQKTQRRWFLDKLVDEIGVGYVRDSGSVSHY | |
| | 1-A | K19S32H33C4 | | YLSEIKPLHNFLTQLQPFLELKQKQANLVLKIIEQLPSAK | |
| | | 0A61R70S75N | | ESPDKFLEVCTWVDQIAALNDSKTRKTTSETVRAVLDSL | |
| | | 77K | | SEKKKSSPAAGGSDKYNQALSKYNQALSKYNQALSGG | |
| | | | | GGSNKKFLLYLAGFVDSDGSIIAQIKPNQHCKFKHQLAL | |
| | | | | TFQVTQKTQRRWFLDKLVDRIGVGYVRDSGSVSNYKL | |
| | | | | SEIKPLHNFLTQLQPFLKLKQKQANLVLKIIEQLPSAKES | |
| | | | | PDKFLEVCTWVDQIAALNDSKTRKTTSETVRAVLDSLSE | |
| | | | | KKKSSP | |
| pCLS3302 | SCOH- | 7E33H38Y70S | + | MANTKYNEEFLLYLAGFVDGDGSIIAQIKPNQSHKFKHY | 572 |
| | HSV8b | 75H77Y96E13 | | LSLTFQVTQKTQRRWFLDKLVDEIGVGYVRDSGSVSHY | |
| | 1-B | 2V_8K19S32H | | YLSEIKPLHNFLTQLQPFLELKQKQANLVLKIIEQLPSAK | |
| | | 33C40A61R70 | | ESPDKFLEVCTWVDQVAALNDSKTRKTTSETVRAVLDS | |
| | | S75N77K132V | | LSEKKKSSPAAGGSDKYNQALSKYNQALSKYNQALSG | |
| | | | | GGGSNKKFLLYLAGFVDSDGSIIAQIKPNQHCKFKHQLA | |
| | | | | LTFQVTQKTQRRWFLDKLVDRIGVGYVRDSGSVSNYKL | |
| | | | | SEIKPLHNFLTQLQPFLKLKQKQANLVLKIIEQLPSAKES | |
| | | | | PDKFLEVCTWVDQVAALNDSKTRKTTSETVRAVLDSLS | |
| | | | | EKKKSSP | |
| pCLS3303 | SCOH- | 7E33H38Y70S | + | MANTKYNEEFLLYLAGFVDGDGSIIAQIKPNQSHKFKHY | 573 |
| | HSV8b | 75H77Y80K96 | | LSLTFQVTQKTQRRWVFLDKLVDEIGVGYVRDSGSVSHY | |
| | 1-C | E132V_8K19S | | YLSKIKPLHNFLTQLQPFLELKQKQANLVLKIIEQLPSAK | |
| | | 32H33C40A61 | | ESPDKFLEVCTWVDQVAALNDSKTRKTTSETVRAVLDS | |
| | | R70S75N77K1 | | LSEKKKSSPAAGGSDKYNQALSKYNQALSKYNQALSG | |
| | | 05A132V | | GGGSNKKFLLYLAGFVDSDGSIIAQIKPNQHCKFKHQLA | |
| | | | | LTFQVTQKTQRRWFLDKLVDRIGVGYVRDSGSVSNYKL | |
| | | | | SEIKPLHNFLTQLQPFLKLKQKQANLALKIIEQLPSAKES | |
| | | | | PDKFLEVCTWVDQVAALNDSKTRKTTSETVRAVLDSLS | |
| | | | | EKKKSSP | |
| pCLS3304 | SCOH- | 7E33H38Y70S | + | MANTKYNEEFLLYLAGFVDGDGSIIAQIKPNQSHKFKHY | 574 |
| | HSV8b | 75H77Y80K96 | | LSLTFQVTQKTQRRWFLDKLVDEIGVGYVRDSGSVSHY | |
| | 1-E | E105A132V_8 | | YLSKIKPLHNFLTQLQPFLELKQKQANLALKIIEQLPSAK | |
| | | K19S32H33C4 | | ESPDKFLEVCTWVDQVAALNDSKTRKTTSETVRAVLDS | |
| | | 0A61R70S75N | | LSEKKKSSPAAGGSDKYNQALSKYNQALSKYNQALSG | |
| | | 77K80K105A1 | | GGGSNKKFLLYLAGFVDSDGSIIAQIKPNQHCKFKHQLA | |
| | | 32V | | LTFQVTQKTQRRWFLDKLVDRIGVGYVRDSGSVSNYKL | |
| | | | | SKIKPLHNFLTQLQPFLKLKQKQANLALKIIEQLPSAKES | |
| | | | | PDKFLEVCTWVDQVAALNDSKTRKTTSETVRAVLDSLS | |
| | | | | EKKKSSP | |
| pCLS3305 | SCOH- | 7E30K33A70S | + | MANTKYNEEFLLYLAGFVDGDGSIIAQIKPKQSAKFKHQ | 575 |
| | HSV8b | 75H77Y96E_8 | | LSLTFQVTQKTQRRWFLDKLVDEIGVGYVRDSGSVSHY | |
| | 562-A | K19S32H33C4 | | YLSEIKPLHNFLTQLQPFLELKQKQANLVLKIIEQLPSAK | |
| | | 0A61R70S75N | | ESPDKFLEVCTWVDQIAALNDSKTRKTTSETVRAVLDSL | |
| | | 77K | | SEKKKSSPAAGGSDKYNQALSKYNQALSKYNQALSGG | |
| | | | | GGSNKKFLLYLAGFVDSDGSIIAQIKPNQHCKFKHQLAL | |
| | | | | TFQVTQKTQRRWFLDKLVDRIGVGYVRDSGSVSNYKL | |
| | | | | SEIKPLHNFLTQLQPFLKLKQKQANLVLKIIEQLPSAKES | |
| | | | | PDKFLEVCTWVDQIAALNDSKTRKTTSETVRAVLDSLSE | |
| | | | | KKKSSP | |
| pCLS3306 | SCOH- | 7E30K33A70S | + | MANTKYNEEFLLYLAGFVDGDGSIIAQIKPKQSAKFKHQ | 576 |
| | HSV8b | 75H77Y96E13 | | LSLTFQVTQKTQRRWFLDKLVDEIGVGYVRDSGSVSHY | |
| | 562-B | 2V_8K19S32H | | YLSEIKPLHNFLTQLQPFLELKQKQANLVLKIIEQLPSAK | |
| | | 33C40A61R70 | | ESPDKFLEVCTWVDQVAALNDSKTRKTTSETVRAVLDS | |
| | | S75N77K132V | | LSEKKKSSPAAGGSDKYNQALSKYNQALSKYNQALSG | |
| | | | | GGGSNKKFLLYLAGFVDSDGSIIAQIKPNQHCKFKHQLA | |
| | | | | LTFQVTQKTQRRWFLDKLVDRIGVGYVRDSGSVSNYKL | |
| | | | | SEIKPLHNFLTQLQPFLKLKQKQANLVLKIIEQLPSAKES | |
| | | | | PDKFLEVCTWVDQVAALNDSKTRKTTSETVRAVLDSLS | |
| | | | | EKKKSSP | |
| pCLS3307 | SCOH- | 7E30K33A70S | + | MANTKYNEEFLLYLAGFVDGDGSIIAQIKPKQSAKFKHQ | 577 |
| | HSV8b | 75H77Y80K96 | | LSLTFQVTQKTQRRWFLDKLVDEIGVGYVRDSGSVSHY | |
| | 562-C | E132V_8K19S | | YLSKIKPLHNFLTQLQPFLELKQKQANLVLKIIEQLPSAK | |
| | | 32H33C40A61 | | ESPDKFLEVCTWVDQVAALNDSKTRKTTSETVRAVLDS | |
| | | R70S75N77K1 | | LSEKKKSSPAAGGSDKYNQALSKYNQALSKYNQALSG | |
| | | 05A132V | | GGGSNKKFLLYLAGFVDSDGSIIAQIKPNQHCKFKHQLA | |
| | | | | LTFQVTQKTQRRWFLDKLVDRIGVGYVRDSGSVSNYKL | |
| | | | | SEIKPLHNFLTQLQPFLKLKQKQANLALKIIEQLPSAKES | |
| | | | | PDKFLEVCTWVDQVAALNDSKTRKTTSETVRAVLDSLS | |
| | | | | EKKKSSP | |
| pCLS3308 | SCOH- | 7E30K33A70S | + | MANTKYNEEFLLYLAGFVDGDGSIIAQIKPKQSAKFKHQ | 578 |
| | HSV8b | 75H77Y80K96 | | LSLTFQVTQKTQRRWFLDKLVDEIGVGYVRDSGSVSHY | |
| | 562-E | E132V_8K19S | | YLSKIKPLHNFLTQLQPFLELKQKQANLVLKIIEQLPSAK | |
| | | 32H33C40A61 | | ESPDKFLEVCTWVDQVAALNDSKTRKTTSETVRAVLDS | |
| | | R70S75N77K1 | | LSEKKKSSPAAGGSDKYNQALSKYNQALSKYNQALSG | |
| | | 05A132V | | GGGSNKKFLLYLAGFVDSDGSIIAQIKPNQHCKFKHQLA | |
| | | | | LTFQVTQKTQRRWFLDKLVDRIGVGYVRDSGSVSNYKL | |
| | | | | SEIKPLHNFLTQLQPFLKLKQKQANLALKIIEQLPSAKES | |
| | | | | PDKFLEVCTWVDQVAALNDSKTRKTTSETVRAVLDSLS | |
| | | | | EKKKSSP | |
| pCLS3309 | SCOH- | 7E30K33R70S | + | MANTKYNEEFLLYLAGFVDGDGSIIAQIKPKQSRKFKHQ | 579 |
| | HSV8b | 75H77Y96E_8 | | LSLTFQVTQKTQRRWFLDKLVDEIGVGYVRDSGSVSHY | |
| | u-A | K19S32H33C4 | | YLSEIKPLHNFLTQLQPFLELKQKQANLVLKIIEQLPSAK | |
| | | 0A44R61R68Y | | ESPDKFLEVCTWVDQIAALNDSKTRKTTSETVRAVLDSL | |
| | | 70S75Y77N | | SEKKKSSPAAGGSDKYNQALSKYNQALSKYNQALSGG | |
| | | | | GGSNKKFLLYLAGFVDSDGSIIAQIKPNQHCKFKHQLAL | |
| | | | | TFRVTQKTQRRWFLDKLVDRIGVGYVYDSGSVSYYNLS | |
| | | | | EIKPLHNFLTQLQPFLKLKQKQANLVLKIIEQLPSAKESP | |
| | | | | DKFLEVCTWVDQIAALNDSKTRKTTSETVRAVLDSLSEK | |
| | | | | KKSSP | |
| pCLS3310 | SCOH- | 7E30K33R70S | + | MANTKYNEEFLLYLAGFVDGDGSIIAQIKPKQSRKFKHQ | 580 |
| | HSV8b | 75H77Y96E13 | | LSLTFQVTQKTQRRWFLDKLVDEIGVGYVRDSGSVSHY | |
| | u-B | 2V_8K19S32H | | YLSEIKPLHNFLTQLQPFLELKQKQANLVLKIIEQLPSAK | |
| | | 33C40A44R61 | | ESPDKFLEVCTWVDQVAALNDSKTRKTTSETVRAVLDS | |
| | | R68Y70S75Y7 | | LSEKKKSSPAAGGSDKYNQALSKYNQALSKYNQALSG | |
| | | 7N132V | | GGGSNKKFLLYLAGFVDSDGSIIAQIKPNQHCKFKHQLA | |
| | | | | LTFRVTQKTQRRWFLDKLVDRIGVGYVYDSGSVSYYNL | |
| | | | | SEIKPLHNFLTQLQPFLKLKQKQANLVLKIIEQLPSAKES | |
| | | | | PDKFLEVCTWVDQVAALNDSKTRKTTSETVRAVLDSLS | |
| | | | | EKKKSSP | |

d) Results

The activity of the single chain molecules against the HSV8 target was monitored using the previously described CHO assay along with our internal control SCOH-RAG (pCLS2222, FIG. 16) and 1-Sce I meganucleases.

All comparisons were done upon DNA dose response of transfected variant DNA. All the single molecules displayed strong cleavage activity of HSV8 target in CHO assay as listed in Table XXXXII.

Variants shared specific behaviour upon assayed dose depending on the mutation profile they bear. For Example, pCLS3306 displays an higher activity than I-SceI control and has a similar profile than SCOH-RAG control. Its activity is high even at low dose (0.2 ng DNA) and reaches a plateau at 6 ng. All of the variants described in Table XXXXII are active and can be used for the HSV-1 virus UL30 gene targeting and cleavage.

### EXAMPLE 6

### Strategy for Engineering Meganucleases Cleaving Target from the UL5 Gene in HSV1 Genome

I-CreI heterodimers capable of cleaving a target sequence (HSV9: GC-AAG-AC-CAC-GTAA-GGC-AG-GGG-GG SEQ ID NO:491) were identified using methods derived from those described in Chames et al. (Nucleic Acids Res., 2005, 33, e178), Arnould et al. (J. Mol. Biol., 2006, 355, 443-458), Smith et al. (Nucleic Acids Res., 2006, 34, e149), Arnould et al. (Arnould et al. J Mol Biol. 2007 371:49-65). These results were then utilized to design single-chain meganucleases directed against the target sequence of SEQ ID NO:491. These single-chain meganucleases were cloned into mammalian expression vectors and tested for HSV9 cleavage in CHO cells. Strong cleavage activity of the HSV9 target could be observed for these single chain molecules in mammalian cells.

### EXAMPLE 6.1

### Identification of Meganucleases Cleaving HSV9

I-CreI variants potentially cleaving the HSV9 target sequence in heterodimeric form were constructed by genetic engineering. Pairs of such variants were then co-expressed in yeast. Upon co-expression, one obtains three molecular species, namely two homodimers and one heterodimer. It was then determined whether the heterodimers were capable of cutting the HSV9 target sequence of SEQ ID NO:491.

a) Construction of Variants of the I-CreI Meganuclease Cleaving Palindromic Sequences Derived from the HSV9 Target Sequence

The HSV9 sequence is partially a combination of the 10AAG_P (SEQ ID NO:493), 5CAC_P (SEQ ID NO:494), 10CCC_P (SEQ ID NO:495), 5GCC_P (SEQ ID NO:496), target sequences which are shown on FIG. 51. These sequences are cleaved by meganucleases obtained as described in International PCT applications WO 2006/097784 and WO 2006/097853, Arnould et al. (J. Mol. Biol., 2006, 355, 443-458) and Smith et al. (Nucleic Acids Res., 2006). Thus, HSV9 should be cleaved by combinatorial variants resulting from these previously identified meganucleases.

The GTAA sequence of the HSV9 target in −2 to 2 was first substituted with the GTAC sequence from C1221, resulting in target HSV9.2 (FIG. 51).

Two palindromic targets, HSV9.3 (HSV9.5) and HSV9.4 (HSV9.6), were derived from HSV9 (FIG. 51). Since HSV9.3 and HSV9.4 are palindromic, they should be cleaved by homodimeric proteins. Therefore, homodimeric I-CreI variants cleaving either the HSV9.3 palindromic target sequence of SEQ ID NO:497 or the HSV9.4 palindromic target sequence of SEQ ID NO:498 were constructed using methods derived from those described in Chames et al. (Nucleic Acids Res., 2005, 33, e178), Arnould et al. (J. Mol. Biol., 2006, 355, 443-458), Smith et al. (Nucleic Acids Res., 2006, 34, e149) and Arnould et al. (Arnould et al. J Mol Biol. 2007 371:49-65).

b) Construction of Target Vector

An oligonucleotide of SEQ ID NO:581, corresponding to the HSV9 target sequence flanked by gateway cloning sequences, was ordered from PROLIGO. This oligo has the following sequence: TGGCATACAAGTTTGCAAGACCACGTA AGGCAGGGGGGCAATCG TCTGTCA. Double-stranded target DNA, generated by PCR amplification of the single stranded oligonucleotide, was cloned into the pCLS1055 yeast reporter vector using the Gateway protocol (INVITROGEN).

Yeast reporter vector was transformed into the FYBL2-7B Saccharomyces cerevisiae strain having the following genotype: MAT a, ura3Δ851, trp1Δ63, leu2Δ1, lys2Δ202. The resulting strain corresponds to a reporter strain.

c) Co-Expression of Variants

The open reading frames coding for the variants cleaving the HSV9.6 (and HSV9.4) or the HSV9.5 (and HSV9.3) sequence were cloned in the pCLS542 expression vector and in the pCLS1107 expression vector, respectively. Yeast DNA from these variants was extracted using standard protocols and was used to transform E. coli. The resulting plasmids were then used to co-transform yeast. Transformants were selected on synthetic medium lacking leucine and containing G418.

d) Mating of Meganucleases Coexpressing Clones and Screening in Yeast

Mating was performed using a colony gridder (QpixII, Genetix). Variants were gridded on nylon filters covering YPD plates, using a low gridding density (4-6 spots/cm²). A second gridding process was performed on the same filters to spot a second layer consisting of different reporter-harboring yeast strains for each target. Membranes were placed on solid agar YPD rich medium, and incubated at 30° C. for one night, to allow mating. Next, filters were transferred to synthetic medium, lacking leucine and tryptophan, adding G418, with galactose (2%) as a carbon source, and incubated for five days at 37° C., to select for diploids carrying the expression and target vectors. After 5 days, filters were placed on solid agarose medium with 0.02% X-Gal in 0.5 M sodium phosphate buffer, pH7.0, 0.1% SDS, 6% dimethyl formamide (DMF), 7 mM β-mercaptoethanol, 1% agarose, and incubated at 37° C., to monitor β-galactosidase activity. Results were analyzed by scanning and quantification was performed using an appropriate software.

e) Results

Co-expression of different variants resulted in cleavage of the HSV9 target in 24 tested combinations. Functional combinations are summarized in Table XXXXIII here below. In this Table, “+” indicates a functional combination on the HSV9 target sequence, i.e., the heterodimer is capable of cleaving the HSV9 target sequence.

HSV9 target is recognized and cleaved by the meganucleases shown in Table XXXXIII.

TABLE XXXXIII

| | | Amino acids positions and residues | | | | | |
|---|---|---|---|---|---|---|---|
| | | of the I-CreI variants cleaving the HSV9.5 (SEQ ID NO: 499) | | | | | |
| | | (and.3 (SEQ ID NO: 497)) target | | | | | |
| | | 30G 38R 44I 68E | 30G 38R 44V | 32T 33R 44V 68E | | 44N 68E 70S | 32G 44V 68E 75N |
| | | 75N 77R 80K | 68E 75N 77R | 75N 77R 80K | 44K | 75R 77R | 77R 80K |
| | | (SEQ ID | (SEQ ID | (SEQ ID | (SEQ ID | (SEQ ID | (SEQ ID |
| | | NO: 522) | NO: 523) | NO: 524) | NO: 525) | NO: 526) | NO: 527) |
| Amino acids | 30R 38E 68Y | + | + | + | + | + | + |
| positions and | 70S 75R 77Q | | | | | | |
| residues of | (SEQ ID NO: 528) | | | | | | |
| the I-CreI | 30R 38E 44K | | | | + | | |
| variants | 61G 68E 70S | | | | | | |
| cleaving | 72T 75N 77R | | | | | | |
| the HSV9.6 | 80K 92R 96R | | | | | | |
| (SEQ ID NO: 500) | 105A 154R | | | | | | |
| (and.4 | (SEQ ID NO: 529) | | | | | | |
| (SEQ ID NO: 498)) | 30R 38E 68Y | + | + | + | + | + | + |
| target | 70S 75N 77R | | | | | | |
| | 82E | | | | | | |
| | (SEQ ID NO: 530) | | | | | | |
| | 30R 38E 68N | + | + | + | + | + | + |
| | 70S 75R 77V | | | | | | |
| | (SEQ ID NO: 531) | | | | | | |

In conclusion, several heterodimeric I-CreI variants, capable of cleaving the HSV9 target sequence in yeast, were identified.

### EXAMPLE 6.2

### Covalent Assembly as Single Chain and Improvement of Meganucleases Cleaving HSV9

I-CreI variants able to efficiently cleave the HSV9 target in yeast when forming heterodimers are described hereabove in Example 6.1. Among them, two couples have been chosen as scaffold for further single chain meganuclease assembly and activity improvement. The screen and validation in CHO cells is a single-strand annealing (SSA) based assay where cleavage of the target by the meganucleases induces homologous recombination and expression of a LagoZ reporter gene (a derivative of the bacterial lacZ gene).

a) Cloning of HSV9 Target in a Vector for CHO Screen

An oligonucleotide corresponding to the HSV9 target sequence flanked by gateway cloning sequences, was ordered from PROLIGO (SEQ ID NO:582; TGGCATACAAGTTTGCAAGACCACGTAAGGCAGGGGGGCAATCGTCTGTCA). Double-stranded target DNA, generated by PCR amplification of the single stranded oligonucleotide, was cloned using the Gateway protocol (INVITROGEN) into the pCLS1058 CHO reporter vector. Cloned target was verified by sequencing (MILLEGEN).

b) Gene Synthesis and Cloning of HSV9 Meganucleases

The open-reading frames coding for single chain meganuclease variants listed in Table XXXXIV were generated by synthetic gene assembly at MWG-EUROFINS (Les Ulis, France) and cloned in into the pCLS1853 expression vector using the AscI and XhoI restriction enzymes for internal fragment replacement.

c) Extrachromosomal Assay in Mammalian Cells

CHO K1 cells were transfected as described in Example 1.2. 72 hours after transfection, culture medium was removed and 1501 of lysis/revelation buffer for β-galactosidase liquid assay was added. After incubation at 37° C., OD was measured at 420 nm. The entire process is performed on an automated Velocity11 BioCel platform. Per assay, 150 ng of target vector was cotransfected with an increasing quantity of variant DNA from 0 to 25 ng. The total amount of transfected DNA was completed to 175 ng (target DNA, variant DNA, carrier DNA) using an empty vector (pCLS0002).

d) Results

Among the I-CreI variants able to cleave the HSV9 target in yeast when forming heterodimers (in Example 6.1), two couples have been chosen as scaffolds for further single chain meganuclease assembly and directed mutagenesis for activity improvement (Table XXXXIV).

TABLE XXXXIV

| | Amino acids positions and residues of | |
|---|---|---|
| | the I-CreI variants cleaving HSV9.5 | Amino acids positions and residues of the |
| HSV9 couple | (and .3) | I-CreI variants cleaving HSV9.6 (and .4) |
| HSV9-b56 | 30G 38R 44I 68E 75N 77R 80K (SEQ ID | 30R 38E 68Y 70S 75R 77Q (SEQ ID |
| | NO: 522) | NO: 528) |
| HSV9-bu | 32T 33R 44V 68E 75N 77R 80K (SEQ ID | 30R 38E 68Y 70S 75R 77Q (SEQ ID |
| | NO: 524) | NO: 528) |

The two HSV9b56 and bu heterodimers give high cleavage activity in yeast. HSV9 b56 is a couple of HSV9.5×HSV9.6 cutters that bear the following mutations in comparison with the I-CreI wild type sequence: 30G 38R 44I 68E 75N 77R 80K (HSV9.5)×30R 38E 68Y 70S 75R 77Q (HSV9.6). HSV9 bu is a couple of HSV9.5×HSV9.6 cutters that bear the following mutations in comparison with the I-CreI wild type sequence: 32T 33R 44V 68E 75N 77R 80K (HSV9.5)×30R 38E 68Y 70S 75R 77Q (HSV9.6).

Single chain constructs were engineered using the linker RM2 of SEQ ID NO 464 (AAGOSDKYNQALSKYNQALSKYNQALSGGGGS), thus resulting in the production of the single chain molecule: HSV9.5-variant-linkerRM2-HSV9.6-variant. During this design step, the 019S mutation was introduced in the C-terminal variant. In addition, mutations K7E, K96E were introduced into the HSV9.5 variant and mutations E8K, E61R into the HSV9.6 variant to create the single chain molecule: HSV9.5-variant (K7E K96E)-linkerRM2-HSV9.6-variant (E8K E61R G19S) that is further called SCOH-HSV9b56 and SCOH-HSV9bu depending on the HSV9 couple used as scaffold (Tables XXXXIV and XXXXV). Some additional amino-acid substitutions have been found in previous studies to enhance the activity of I-CreI derivatives: some of these mutations correspond to the replacement of Glutamic acid 80 with Lysine (E80K), Valine 105 with Alanine (V105A) and Isoleucine 132 with Valine (I132V). The resulting proteins are shown in Table XXXXV below. All the single chain molecules were assayed in CHO for cleavage of the HSV9 target.

TABLE XXXXV
Example of Single Chain series designed for strong
cleavage of HSV9 target in CHO cells

| | | | HSV9 | | SEQ |
|---|---|---|---|---|---|
| | | mutations in | cleavage | | ID |
| plasmid | variant | Single Chain | in CHO | Protein sequence | NO: |
| pCLS3311 | SCOH- | 7E30G38R44I | + | MANTKYNEEFLLYLAGFVDGDGSIIAQIKPGQSYK | 583 |
| | HSV9- | 68E75N77R80 | | FKHRLSLTFIVTQKTQRRWFLDKLVDEIGVGYVE | |
| | b56-A | K96E_8K19S3 | | DRGSVSNYRLSKIKPLHNFLTQLQPFLELKQKQAN | |
| | | 0R38E61R68Y | | LVLKIIEQLPSAKESPDKFLEVCTWVDQIAALNDS | |
| | | 70S75R77Q | | KTRKTTSETVRAVLDSLSEKKKSSPAAGGSDKYN | |
| | | | | QALSKYNQALSKYNQALSGGGGSNKKFLLYLAG | |
| | | | | FVDSDGSIIAQIKPRQSYKFKHELSLTFQVTQKTQR | |
| | | | | RWFLDKLVDRIGVGYVYDSGSVSRYQLSEIKPLH | |
| | | | | NFLTQLQPFLKLKQKQANLVLKIIEQLPSAKESPD | |
| | | | | KFLEVCTWVDQIAALNDSKTRKTTSETVRAVLDS | |
| | | | | LSEKKKSSP | |
| pCLS3312 | SCOH- | 7E30G38R44I | + | MANTKYNEEFLLYLAGFVDGDGSIIAQIKPGQSYK | 584 |
| | HSV9- | 68E75N77R80 | | FKHRLSLTFIVTQKTQRRWFLDKLVDEIGVGYVE | |
| | b56-C | K96E132V_8K | | DRGSVSNYRLSKIKPLHNFLTQLQPFLELKQKQAN | |
| | | 19S30R38E61 | | LVLKIIEQLPSAKESPDKFLEVCTWVDQVAALNDS | |
| | | R68Y70S75R7 | | KTRKTTSETVRAVLDSLSEKKKSSPAAGGSDKYN | |
| | | 7Q132V | | QALSKYNQALSKYNQALSGGGGSNKKFLLYLAG | |
| | | | | FVDSDGSIIAQIKPRQSYKFKHELSLTFQVTQKTQR | |
| | | | | RWFLDKLVDRIGVGYVYDSGSVSRYQLSEIKPLH | |
| | | | | NFLTQLQPFLKLKQKQANLVLKIIEQLPSAKESPD | |
| | | | | KFLEVCTWVDQVAALNDSKTRKFFSETVRAVLDS | |
| | | | | LSEKKKSSP | |
| pCLS3313 | SCOH- | 7E30G38R44I | + | MANTKYNEEFLLYLAGFVDGDGSIIAQIKPGQSYK | 585 |
| | HSV9- | 68E75N77R80 | | FKHRLSLTFIVTQKTQRRWFLDKLVDEIGVGYVE | |
| | b56-E | K96E132V_8K | | DRGSVSNYRLSKIKPLHNFLTQLQPFLELKQKQAN | |
| | | 19S30R38E61 | | LVLKIIEQLPSAKESPDKFLEVCTWVDQVAALNDS | |
| | | R68Y70S75R7 | | KTRKTTSETVRAVLDSLSEKKKSSPAAGGSDKYN | |
| | | 7Q105A132V | | QALSKYNQALSKYNQALSGGGGSNKKFLLYLAG | |
| | | | | FVDSDGSIIAQIKPRQSYKFKHELSLTFQVTQKTQR | |
| | | | | RWFLDKLVDRIGVGYVYDSGSVSRYQLSEIKPLH | |
| | | | | NFLTQLQPFLKLKQKQANLALKIIEQLPSAKESPD | |
| | | | | KFLEVCTWVDQVAALNDSKTRKTTSETVRAVLDS | |
| | | | | LSEKKKSSP | |
| pCLS3314 | SCOH- | 7E30G38R44I | + | MANTKYNEEFLLYLAGFVDGDGSIIAQIKPGQSYK | 586 |
| | HSV9- | 68E75N77R80 | | FKHRLSLTFIVTQKTQRRWFLDKLVDEIGVGYVE | |
| | b56-F | K96E105A132 | | DRGSVSNYRLSKIKPLHNFLTQLQPFLELKQKQAN | |
| | | V_8K19S30R3 | | LALKIIEQLPSAKESPDKFLEVCTWVDQVAALNDS | |
| | | 8E61R68Y70S | | KTRKTTSETVRAVLDSLSEKKKSSPAAGGSDKYN | |
| | | 75R77Q80K10 | | QALSKYNQALSKYNQALSGGGGSNKKFLLYLAG | |
| | | 5A132V | | FVDSDGSIIAQIKPRQSYKFKHELSLTFQVTQKTQR | |
| | | | | RWFLDKLVDRIGVGYVYDSGSVSRYQLSKIKPLH | |
| | | | | NFLTQLQPFLKLKQKQANLALKIIEQLPSAKESPD | |
| | | | | KFLEVCTWVDQVAALNDSKTRKTTSETVRAVLDS | |
| | | | | LSEKKKSSP | |
| pCLS3315 | SCOH- | 7E32T33R44V | + | MANTKYNEEFLLYLAGFVDGDGSIIAQIKPNQTRK | 587 |
| | HSV9- | 68E75N77R80 | | FKHQLSLTFVVTQKTQRRWFLDKLVDEIGVGYVE | |
| | bu-A | K96E_8K19S3 | | DRGSVSNYRLSKIKPLHNFLTQLQPFLELKQKQAN | |
| | | 0R38E61R68Y | | LVLKIIEQLPSAKESPDKFLEVCTWVDQIAALNDS | |
| | | 70S75R77Q | | KTRKTTSETVRAVLDSLSEKKKSSPAAGGSDKYN | |
| | | | | QALSKYNQALSKYNQALSGGGGSNKKFLLYLAG | |
| | | | | FVDSDGSIIAQIKPRQSYKFKHELSLTFQVTQKTQR | |
| | | | | RWFLDKLVDRIGVGYVYDSGSVSRYQLSEIKPLH | |
| | | | | NFLTQLQPFLKLKQKQANLVLKIIEQLPSAKESPD | |
| | | | | KFLEVCTWVDQIAALNDSKTRKTTSETVRAVLDS | |
| | | | | LSEKKKSSP | |
| pCLS3316 | SCOH- | 7E32T33R44V | + | MANTKYNEEFLLYLAGFVDGDGSHAQIKPNQTRK | 588 |
| | HSV9- | 68E75N77R80 | | FKHQLSLTFVVTQKTQRRWFLDKLVDEIGVGYVE | |
| | bu-C | K96E132V_8K | | DRGSVSNYRLSKIKPLHNFLTQLQPFLELKQKQAN | |
| | | 19S30R38E61 | | LVLKIIEQLPSAKESPDKFLEVCTWVDQVAALNDS | |
| | | R68Y70S75R7 | | KTRKTTSETVRAVLDSLSEKKKSSPAAGGSDKYN | |
| | | 7Q132V | | QALSKYNQALSKYNQALSGGGGSNKKFLLYLAG | |
| | | | | FVDSDGSIIAQIKPRQSYKFKHELSLTFQVTQKTQR | |
| | | | | RWFLDKLVDRIGVGYVYDSGSVSRYQLSEIKPLH | |
| | | | | NFLTQLQPFLKLKQKQANLVLKIIEQLPSAKESPD | |
| | | | | KFLEVCTWVDQVAALNDSKTRKTTSETVRAVLDS | |
| | | | | LSEKKKSSP | |
| pCLS3317 | SCOH- | 7E32T33R44V | + | MANTKYNEEFLLYLAGFVDGDGSIIAQIKPNQTRK | 589 |
| | HSV9- | 68E75N77R80 | | FKHQLSLTFVVTQKTQRRWFLDKLVDEIGVGYVE | |
| | bu-E | K96E132V_8K | | DRGSVSNYRLSKIKPLHNFLTQLQPFLELKQKQAN | |
| | | 19S30R38E61 | | LVLKIIEQLPSAKESPDKFLEVCTWVDQVAALNDS | |
| | | R68Y70S75R7 | | KTRKTTSETVRAVLDSLSEKKKSSPAAGGSDKYN | |
| | | 7Q105A132V | | QALSKYNQALSKYNQALSGGGGSNKKFLLYLAG | |
| | | | | FVDSDGSIIAQIKPRQSYKFKHELSLTFQVTQKTQR | |
| | | | | RWFLDKLVDRIGVGYVYDSGSVSRYQLSEIKPLH | |
| | | | | NFLTQLQPFLKLKQKQANLALKIIEQLPSAKESPD | |
| | | | | KFLEVCTWVDQVAALNDSKTRKTTSETVRAVLDS | |
| | | | | LSEKKKSSP | |
| pCLS3318 | SCOH- | 7E32T33R44V | + | MANTKYNEEFLLYLAGFVDGDGSIIAQIKPNQTRK | 590 |
| | HSV9- | 68E75N77R80 | | FKHQLSLTFVVTQKTQRRWFLDKLVDEIGVGYVE | |
| | bu-F | K96E105A132 | | DRGSVSNYRLSKIKPLHNFLTQLQPFLELKQKQAN | |
| | | V_8K19S30R3 | | LALKIIEQLPSAKESPDKFLEVCTWVDQVAALNDS | |
| | | 8E61R68Y70S | | KTRKTTSETVRAVLDSLSEKKKSSPAAGGSDKYN | |
| | | 75R77Q80K10 | | QALSKYNQALSKYNQALSGGGGSNKKELLYLAG | |
| | | 5A132V | | FVDSDGSIIAQIKPRQSYKFKHELSLTFQVTQKTQR | |
| | | | | RWFLDKLVDRIGVGYVYDSGSVSRYQLSKIKPLH | |
| | | | | NFLTQLQPFLKLKQKQANLALKIIEQLPSAKESPD | |
| | | | | KFLEVCTWVDQVAALNDSKTRKTTSETVRAVLDS | |
| | | | | LSEKKKSSP | |

d) Results

The activity of the single chain molecules against the HSV9 target was monitored using the previously described CHO assay along with our internal control SCOH-RAG and I-Sce I meganucleases. All comparisons were done upon DNA dose response of transfected variant DNA. All the single molecules displayed strong cleavage activity of HSV9 target in CHO assay as listed in Table XXXXV.

Variants shared specific behaviour upon assayed dose depending on the mutation profile they bear. For Example, pCLS3318 displays a higher activity than I-Sce I control and SCOH-RAG control (FIG. 42). Its activity has reached the plateau even at the lowest dose (0.2 ng DNA). All of the variants described in Table XXXXV are active and can be used for the HSV-1 virus UL5 gene targeting and cleavage.

### EXAMPLE 7

### Strategy for Engineering Novel Meganucleases Cleaving Targets from the RL2/ICP₀ Gene in HSV-1 Genome

A first series of meganucleases targeting the RL2 gene encoding the ICP0 or a0 protein has been described previously (HSV4 target). In the following lines an alternative sequence for gene targeting and cleavage of RL2 is described (HSV12). The RL2 gene is a 3.6 kb gene repeated twice in TRL (5 to 5698) and IRL (120673 to 124285) regions is formed of three exons: position 2261 to 2317, 3083 to 3749, 3886 to 5489 and 120882 to 122485, 122622 to 123288, 124054 to 124110.

HSV 12 sequence is a 24 bp (non-palindromic) target (HSV 12: CC-TGG-AC-ATG-GAGA-CGG-GG-AAC-AT SEQ ID NO:501) present in the exon 3 which corresponds to positions 5168 to 5191 and 121180 to 121203 in the two copies of the HSV-1 ICP0 gene (accession number NC_{—}001806; FIG. 23).

I-CreI heterodimers capable of cleaving a target sequence HSV12 (SEQ ID NO:501) were identified using methods derived from those described in Chames et al. (Nucleic Acids Res., 2005, 33, e178), Arnould et al. (J. Mol. Biol., 2006, 355, 443-458), Smith et al. (Nucleic Acids Res., 2006, 34, e149), Arnould et al. (Arnould et al. J Mol Biol. 2007 371:49-65). These results were then utilized to design single-chain meganucleases directed against the target sequence of SEQ ID NO:501. These single-chain meganucleases were cloned into mammalian expression vectors and tested for HSV12 cleavage in CHO cells. Strong cleavage activity of the HSV 12 target could be observed for these single chain molecules in mammalian cells.

### EXAMPLE 7.1

### Identification of Meganucleases Cleaving HSV12

I-CreI variants potentially cleaving the HSV12 target sequence in heterodimeric form were constructed by genetic engineering. Pairs of such variants were then co-expressed in yeast. Upon co-expression, one obtains three molecular species, namely two homodimers and one heterodimer. It was then determined whether the heterodimers were capable of cutting the HSV 12 target sequence of SEQ ID NO:501.

a) Construction of Variants of the I-CreI Meganuclease Cleaving Palindromic Sequences Derived from the HSV12 Target Sequence

The HSV12 sequence is partially a combination of the 10TGG_P (SEQ ID NO:503), 5ATG_P (SEQ ID NO:504), 10GTT_P (SEQ ID NO:505), 5CCG_P (SEQ ID NO:506) target sequences which are shown on FIG. 52. These sequences are cleaved by meganucleases obtained as described in International PCT applications WO 2006/097784 and WO 2006/097853, Arnould et al. (J. Mol. Biol., 2006, 355, 443-458) and Smith et al. (Nucleic Acids Res., 2006). Thus, HSV12 should be cleaved by combinatorial variants resulting from these previously identified meganucleases.

The GAGA sequence of HSV 12 target in −2 to 2 was first substituted with the GTAC sequence from C1221, resulting in target HSV 12.2 (FIG. 52).

Two palindromic targets, HSV12.3 (and HSV12.5) and HSV12.4 (and HSV12.6), were derived from HSV12 (FIG. 52). Since HSV12.3 and HSV12.4 are palindromic, they should be cleaved by homodimeric proteins. Therefore, homodimeric I-CreI variants cleaving either the HSV 12.3 palindromic target sequence of SEQ ID NO:507 or the HSV12.4 palindromic target sequence of SEQ ID NO:508 were constructed using methods derived from those described in Chames et al. (Nucleic Acids Res., 2005, 33, e178), Arnould et al. (J. Mol. Biol., 2006, 355, 443-458), Smith et al. (Nucleic Acids Res., 2006, 34, e149) and Arnould et al. (Arnould et al. J Mol Biol. 2007 371:49-65).

b) Construction of Target Vector

An oligonucleotide of SEQ ID NO:591, corresponding to the HSV12 target sequence flanked by gateway cloning sequences, was ordered from PROLIGO. This oligo has the following sequence: TGGCATACAAGTTTCCTGGACATGGAGACGGGGAACATCAATCGTCTGTCA. Double-stranded target DNA, generated by PCR amplification of the single stranded oligonucleotide, was cloned into the pCLS1055 yeast reporter vector using the Gateway protocol (INVITROGEN).

Yeast reporter vector was transformed into the FYBL2-7B Saccharomyces cerevisiae strain having the following genotype: MAT a, ura3Δ851, trp1Δ63, leu2Δ1, lys2Δ202. The resulting strain corresponds to a reporter strain.

c) Co-Expression of Variants

The open reading frames coding for the variants cleaving the HSV12.6 (and HSV12.4) or the HSV12.5 (and HSV 12.3) sequence were cloned in the pCLS542 expression vector and in the pCLS1107 expression vector, respectively. Yeast DNA from these variants was extracted using standard protocols and was used to transform E. coli. The resulting plasmids were then used to co-transform yeast. Transformants were selected on synthetic medium lacking leucine and containing G418.
d) Mating of Meganucleases Coexpressing Clones and Screening in Yeast

Mating was performed using a colony gridder (QpixII, Genetix). Variants were gridded on nylon filters covering YPD plates, using a low gridding density (4-6 spots/cm²). A second gridding process was performed on the same filters to spot a second layer consisting of different reporter-harboring yeast strains for each target. Membranes were placed on solid agar YPD rich medium, and incubated at 30° C. for one night, to allow mating. Next, filters were transferred to synthetic medium, lacking leucine and tryptophan, adding G418, with galactose (2%) as a carbon source, and incubated for five days at 37° C., to select for diploids carrying the expression and target vectors. After 5 days, filters were placed on solid agarose medium with 0.02% X-Gal in 0.5 M sodium phosphate buffer, pH7.0, 0.1% SDS, 6% dimethyl formamide (DMF), 7 mM 1-mercaptoethanol, 1% agarose, and incubated at 37° C., to monitor β-galactosidase activity. Results were analyzed by scanning and quantification was performed using an appropriate software.

e) Results

Co-expression of different variants resulted in cleavage of the HSV 12 target in 54 tested combinations. Functional combinations are summarized in Table XXXXVI here below. In this Table, “+” indicates a functional combination on the HSV12 target sequence, i.e., the heterodimer is capable of cleaving the HSV12 target sequence.

TABLE XXXXVI

| | | Amino acids positions and residues | | | | | | | | |
|---|---|---|---|---|---|---|---|---|---|---|
| | | of the I-CreI variants cleaving the HSV12.5 (and.3) target | | | | | | | | |
| | | 24V 33C | 24V 33C | 11V 24V | 24V 33C | 24V 33C | 24V 33C | 24V 33C | 24V 33C | 24V 33C |
| | | 38S 44I | 38S 44I | 33C 38S | 38S 44I | 38S 44I | 38S 44I | 38S 44I | 38S 44I | 38S 44I |
| | | 50R 70S | 54L 70S | 44I 50R | 54L 70S | 70S 75N | 70S 75N | 50R 70S | 50R 70S | 50R 70S |
| | | 75N 77R | 75N 77R | 70S 75N | 75N 77R | 77R 81T | 77R 80K | 75N 77R | 75N 77R | 75N 77R |
| | | 132V | 132V | 77R 132V | 129A 160R | 132V | 192R 32V | 132V 160R | 79G 132V | 79G 105A |
| | | (SEQ ID | (SEQ ID | (SEQ ID | (SEQ ID | (SEQ ID | (SEQ ID | (SEQ ID | (SEQ ID | (SEQ ID |
| | | NO: 25) | NO: 592) | NO: 593) | NO: 594) | NO: 595) | NO: 596) | NO: 597) | NO: 598) | NO: 599) |
| Amino acids | 30R 33S 44K | + | + | + | + | + | + | + | + | + |
| positions and | 68Y 70S 77T | | | | | | | | | |
| residues of | 105A 139R | | | | | | | | | |
| the I-CreI | 153A | | | | | | | | | |
| variants | (SEQ ID NO: 600) | | | | | | | | | |
| cleaving | 8K 30R 33S | + | + | + | + | + | + | + | + | + |
| the HSV12.6 | 44K 66H 68Y | | | | | | | | | |
| (and.4) | 70S 77T 139R | | | | | | | | | |
| target | 160E | | | | | | | | | |
| | (SEQ ID NO: 601) | | | | | | | | | |
| | 8K 30R 33S | + | + | + | + | + | + | + | + | + |
| | 44K 66H 68Y | | | | | | | | | |
| | 70S 77T 82R | | | | | | | | | |
| | 87I 139R | | | | | | | | | |
| | (SEQ ID NO: 602) | | | | | | | | | |
| | 8K 30R 33S | + | + | + | + | + | + | + | + | + |
| | 44K 66H 68Y | | | | | | | | | |
| | 70S 77T 87I | | | | | | | | | |
| | 139R | | | | | | | | | |
| | (SEQ ID NO: 603) | | | | | | | | | |
| | 8K 30R 33S | + | + | + | + | + | + | + | + | + |
| | 44K 66H 68Y | | | | | | | | | |
| | 70S 77T 87I | | | | | | | | | |
| | 139R 163S | | | | | | | | | |
| | (SEQ ID NO: 604) | | | | | | | | | |
| | 30R 33S 44K | + | + | + | + | + | + | + | + | + |
| | 66N 68Y 70S | | | | | | | | | |
| | 77T 139R | | | | | | | | | |
| | (SEQ ID NO: 605) | | | | | | | | | |

In conclusion, several heterodimeric I-CreI variants, capable of cleaving the HSV 12 target sequence in yeast, were identified.

### EXAMPLE 7.2

### Covalent Assembly as Single Chain and Improvement of Meganucleases Cleaving HSV12

I-CreI variants able to efficiently cleave the HSV12 target in yeast when forming heterodimers are described hereabove in Example 7.1. Among them, one couple has been chosen as scaffold for further single chain meganuclease assembly and activity improvement. The screen and validation in CHO cells is a single-strand annealing (SSA) based assay where cleavage of the target by the meganucleases induces homologous recombination and expression of a LagoZ reporter gene (a derivative of the bacterial lacZ gene).

a) Cloning of HSV12 Target in a Vector for CHO Screen

An oligonucleotide corresponding to the HSV12 target sequence flanked by gateway cloning sequences, was ordered from PROLIGO (SEQ ID NO:606; TGGCATACAAGTTTCCTGGACATGOOAGACGGGGAACATCAATCGTCTGTCA). Double-stranded target DNA, generated by PCR amplification of the single stranded oligonucleotide, was cloned using the Gateway protocol (INVITROGEN) into the pCLS1058 CHO reporter vector. Cloned target was verified by sequencing (MILLEGEN).

b) Gene Synthesis and Cloning of HSV12 Meganucleases

The open-reading frames coding for single chain meganuclease variants listed in Table XXXXVIII were generated by synthetic gene assembly at TOP Gene Technologies, Inc (Montreal, CANADA) and cloned in into the pCLS1853 expression vector using the AscI and XhoI restriction enzymes for internal fragment replacement.

c) Extrachromosomal Assay in Mammalian Cells

CHO K1 cells were transfected as described in Example 1.2. 72 hours after transfection, culture medium was removed and 150 μl of lysis/revelation buffer for β-galactosidase liquid assay was added. After incubation at 37° C., OD was measured at 420 nm. The entire process is performed on an automated Velocity11 BioCel platform. Per assay, 150 ng of target vector was cotransfected with an increasing quantity of variant DNA from 0 to 25 ng. The total amount of transfected DNA was completed to 175 ng (target DNA, variant DNA, carrier DNA) using an empty vector (pCLS0002).

d) Results

Among the I-CreI variants able to cleave the HSV12 target in yeast when forming heterodimers (in Example 7.1), one couple has been chosen as scaffold for further single chain meganuclease assembly and directed mutagenesis for activity improvement (Table XXXXVII).

TABLE XXXXVII

| | Amino acids positions and residues of the I- | Amino acids positions and residues of the I- |
|---|---|---|
| HSV12 couple | CreI variants cleaving HSV12.5 (and .3) | CreI variants cleaving HSV12.6 (and .4) |
| HSV12-M1-ME | 24V 33C 38S 44I 50R 70S 75N 77R 132V | 8K 30R 33S 44K 66H 68Y 70S 77T 87I |
| | (SEQ ID NO: 25) | 139R 163S (SEQ ID NO: 604) |

The HSV12-M1×HSV12-ME heterodimer give high cleavage activity in yeast. HSV12-M1 is a HSV12.5 cutter that bears the following mutations in comparison with the I-CreI wild type sequence: 24V 33C 38S 44I 50R 70S 75N 77R 132V. HSV12-ME is a HSV12.6 cutters that bears the following mutations in comparison with the I-CreI wild type sequence: 8K 30R 33S 44K 66H68Y 70S 77T 87I 139R 163S.

Single chain constructs were engineered using the linker RM2 of SEQ ID NO 464 (AAGGSDKYNQALSKYNQALSKYNQALSGGGGS), thus resulting in the production of the single chain molecule: M1-linkerRM2-ME. During this design step, the G19S mutation was introduced in the C-terminal variant. In addition, mutations K7E, K96E were introduced into the HSV 12.5 variant and mutation E61R (E8K already present) into the HSV12.6 variant to create the single chain molecule: HSV12.5-M1 (K7E K96E)-linkerRM2-HSV12.6-ME (EK E61R G19S) that is further called SCOH-HSV12-M1-ME (SEQ ID NO:607) scaffold. Some additional amino-acid substitutions have been found in previous studies to enhance the activity of I-CreI derivatives: some of these mutations correspond to the replacement of Glutamic acid 80 with Lysine (E80K), Valine 105 with Alanine (V105A) and Isoleucine 132 with Valine (I132V). The resulting proteins are shown in Table XXXXVIII below. All the single chain molecules were assayed in CHO for cleavage of the HSV 12 target.

TABLE XXXXVIII
Example of Single Chain series designed for strong
cleavage of HSV12 target in CHO cells

| | | | HSV12 | | |
|---|---|---|---|---|---|
| | | mutations in Single | cleavage | | SEQ |
| plasmid | variant | Chain | in CHO | protein sequence | ID |
| pCLS2632 | SCOH- | 7E24V33C38S44I50R70 | + | MANTKYNEEFLLYLAGFVDGDGS | 607 |
| | HV12-M1- | S75N77R96E132V_8K1 | | IVAQIKPNQSCKFKHSLSLTFIVTQ | |
| | ME | 9S30R33S44K61R66H6 | | KTRRRWFLDKLVDEIGVGYVRDS | |
| | | 8Y70S77T87I139R163S | | GSVSNYRLSEIKPLHNFLTQLQPFL | |
| | | | | ELKQKQANLVLKIIEQLPSAKESPD | |
| | | | | KFLEVCTWVDQVAALNDSKTRKT | |
| | | | | TSETVRAVLDSLSEKKKSSPAAGG | |
| | | | | SDKYNQALSKYNQALSKYNQALS | |
| | | | | GGGGSNKKFLLYLAGFVDSDGSII | |
| | | | | AQIKPRQSSKFKHQLSLTFKVTQK | |
| | | | | TQRRWFLDKLVDRIGVGHVYDSG | |
| | | | | SVSDYTLSEIKPLHNILTQLQPFLK | |
| | | | | LKQKQANLVLKIIEQLPSAKESPD | |
| | | | | KFLEVCTWVDQIAALNDSRTRKT | |
| | | | | TSETVRAVLDSLSEKKKSSS | |
| pCLS2633 | SCOH- | 7E24V33C38S44I50R70 | + | MANTKYNEEFLLYLAGFVDGDGS | 465 |
| | HV12-M1- | S75N77R96E132V_8K1 | | IVAQIKPNQSCKFKHSLSLTFIVTQ | |
| | ME-132V | 9S30R33S44K61R66H6 | | KTRRRWFLDKLVDEIGVGYVRDS | |
| | | 8Y70S77T87I132V139 | | GSVSNYRLSEIKPLHNFLTQLQPFL | |
| | | R163S | | ELKQKQANLVLKIIEQLPSAKESPD | |
| | | | | KFLEVCTWVDQVAALNDSKTRKT | |
| | | | | TSETVRAVLDSLSEKKKSSPAAGG | |
| | | | | SDKYNQALSKYNQALSKYNQALS | |
| | | | | GGGGSNKKFLLYLAGFVDSDGSII | |
| | | | | AQIKPRQSSKFKHQLSLTFKVTQK | |
| | | | | TQRRWFLDKLVDRIGVGHVYDSG | |
| | | | | SVSDYTLSEIKPLHNILTQLQPFLK | |
| | | | | LKQKQANLVLKIIEQLPSAKESPD | |
| | | | | KFLEVCTWVDQVAALNDSRTRKT | |
| | | | | TSETVRAVLDSLSEKKKSSS | |
| pCLS2634 | SCOH- | 7E24V33C38S44I50R70 | + | MANTKYNEEFLLYLAGFVDGDGS | 608 |
| | HV12-M1- | S75N77R80K96E132V_{—} | | IVAQIKPNQSCKFKHSLSLTFIVTQ | |
| | 80K-ME | 8K19S30R33S44K61R6 | | KTRRRWFLDKLVDEIGVGYVRDS | |
| | | 6H68Y70S77T87I139R | | GSVSNYRLSKIKPLHNFLTQLQPFL | |
| | | 163S | | ELKQKQANLVLKIIEQLPSAKESPD | |
| | | | | KFLEVCTWVDQVAALNDSKTRKT | |
| | | | | TSETVRAVLDSLSEKKKSSPAAGG | |
| | | | | SDKYNQALSKYNQALSKYNQALS | |
| | | | | GGGGSNKKFLLYLAGFVDSDGSII | |
| | | | | AQIKPRQSSKFKHQLSLTFKVTQK | |
| | | | | TQRRWFLDKLVDRIGVGHVYDSG | |
| | | | | SVSDYTLSEIKPLHNILTQLQPFLK | |
| | | | | LKQKQANLVLKIIEQLPSAKESPD | |
| | | | | KFLEVCTWVDQIAALNDSRTRKT | |
| | | | | TSETVRAVLDSLSEKKKSSS | |
| pCLS2635 | SCOH- | 7E24V33C38S44I50R70 | + | MANTKYNEEFLLYLAGFVDGDGS | 466 |
| | HV12-M1- | S75N77R80K96E132V_{—} | | IVAQIKPNQSCKFKHSLSLTFIVTQ | |
| | 80K-ME- | 8K19S30R33S44K61R6 | | KTRRRWFLDKLVDEIGVGYVRDS | |
| | I32V | 6H68Y70S77T87I132V | | GSVSNYRLSKIKPLHNFLTQLQPFL | |
| | | 139R163S | | ELKQKQANLVLKIIEQLPSAKESPD | |
| | | | | KFLEVCTWVDQVAALNDSKTRKT | |
| | | | | TSETVRAVLDSLSEKKKSSPAAGG | |
| | | | | SDKYNQALSKYNQALSKYNQALS | |
| | | | | GGGGSNKKFLLYLAGFVDSDGSII | |
| | | | | AQIKPRQSSKFKHQLSLTFKVTQK | |
| | | | | TQRRWFLDKLVDRIGVGHVYDSG | |
| | | | | SVSDYTLSEIKPLEINELTQLQPFLK | |
| | | | | LKQKQANLVLKIIEQLPSAKESPD | |
| | | | | KFLEVCTWVDQVAALNDSRTRKT | |
| | | | | TSETVRAVLDSLSEKKKSSS | |
| pCLS2636 | SCOH- | 7E24V33C38S44I50R70 | + | MANTKYNEEFLLYLAGFVDGDGS | 609 |
| | HV12-M1- | S75N77R96E105A132V | | IVAQIKPNQSCKFKHSLSLTFIVTQ | |
| | 105A-ME | _8K19S30R33S44K61R | | KTRRRWFLDKLVDEIGVGYVRDS | |
| | | 66H68Y70S77T87I139 | | GSVSNYRLSEIKPLHNFLTQLQPFL | |
| | | R163S | | ELKQKQANLALKIIEQLPSAKESPD | |
| | | | | KFLEVCTWVDQVAALNDSKTRKT | |
| | | | | TSETVRAVLDSLSEKKKSSPAAGG | |
| | | | | SDKYNQALSKYNQALSKYNQALS | |
| | | | | GGGGSNKKFLLYLAGFVDSDGSII | |
| | | | | AQIKPRQSSKFKHQLSLTFKVTQK | |
| | | | | TQRRWFLDKLVDRIGVGHVYDSG | |
| | | | | SVSDYTLSEIKPLHNILTQLQPFLK | |
| | | | | LKQKQANLVLKIIEQLPSAKESPD | |
| | | | | KFLEVCTWVDQIAALNDSRTRKT | |
| | | | | TSETVRAVLDSLSEKKKSSS | |
| pCLS2637 | SCOH- | 7E24V33C38S44I50R70 | | MANTKYNEEFLLYLAGFVDGDGS | 610 |
| | HV12-M1- | S75N77R96E105A132V | | IVAQIKPNQSCKFKHSLSLTFIVTQ | |
| | 105A-ME- | _8K19S30R33S44K61R | | KTRRRWFLDKLVDEIGVGYVRDS | |
| | 132V | 66H68Y70S77T87I132 | | GSVSNYRLSEIKPLHNFLTQLQPFL | |
| | | V139R163S | | ELKQKQANLALKIIEQLPSAKESPD | |
| | | | | KFLEVCTWVDQVAALNDSKTRKT | |
| | | | | TSETVRAVLDSLSEKKKSSPAAGG | |
| | | | | SDKYNQALSKYNQALSKYNQALS | |
| | | | | GGGGSNKKFLLYLAGFVDSDGSII | |
| | | | | AQIKPRQSSKFKHQLSLTFKVTQK | |
| | | | | TQRRWFLDKLVDRIGVGHVYDSG | |
| | | | | SVSDYTLSEIKPLHNILTQLQPFLK | |
| | | | | LKQKQANLVLKIIEQLPSAKESPD | |
| | | | | KFLEVCTWVDQVAALNDSRTRKT | |
| | | | | TSETVRAVLDSLSEKKKSSS | |
| pCLS2638 | SCOH- | 7E24V33C38S44I50R70 | + | MANTKYNEEFLLYLAGFVDGDGS | 611 |
| | HV12-M1- | S75N77R96E105A132V | | IVAQIKPNQSCKFKHSLSLTFIVTQ | |
| | 105A-ME- | _8K19S30R33S44K61R | | KTRRRWFLDKLVDEIGVGYVRDS | |
| | 80K132V | 66H68Y70S77T80K87I | | GSVSNYRLSEIKPLHNFLTQLQPFL | |
| | | 132V139R163S | | ELKQKQANLALKIIEQLPSAKESPD | |
| | | | | KFLEVCTWVDQVAALNDSKTRKT | |
| | | | | TSETVRAVLDSLSEKKKSSPAAGG | |
| | | | | SDKYNQALSKYNQALSKYNQALS | |
| | | | | GGGGSNKKFLLYLAGFVDSDGSII | |
| | | | | AQIKPRQSSKFKHQLSLTFKVTQK | |
| | | | | TQRRWFLDKLVDRIGVGHVYDSG | |
| | | | | SVSDYTLSKIKPLHNILTQLQPFLK | |
| | | | | LKQKQANLVLKIIEQLPSAKESPD | |
| | | | | KFLEVCTWVDQVAALNDSRTRKT | |
| | | | | TSETVRAVLDSLSEKKKSSS | |
| pCLS2639 | SCOH- | 7E24V33C38S44I50R70 | + | MANTKYNEEFLLYLAGFVDGDGS | 612 |
| | HVI2-M1- | S75N77R96E105A132V | | IVAQIKPNQSCKFKHSLSLTFIVTQ | |
| | 105A-ME- | _8K19S30R33S44K61R | | KTRRRWFLDKLVDEIGVGYVRDS | |
| | 105A132V | 66H68Y70S77T87I105 | | GSVSNYRLSEIKPLHNFLTQLQPFL | |
| | | A132V139R163S | | ELKQKQANLALKIIEQLPSAKESPD | |
| | | | | KFLEVCTWVDQVAALNDSKTRKT | |
| | | | | TSETVRAVLDSLSEKKKSSPAAGG | |
| | | | | SDKYNQALSKYNQALSKYNQALS | |
| | | | | GGGGSNKKFLLYLAGFVDSDGSII | |
| | | | | AQIKPRQSSKFKHQLSLTFKVTQK | |
| | | | | TQRRWFLDKLVDRIGVGHVYDSG | |
| | | | | SVSDYTLSEIKPLHNILTQLQPFLK | |
| | | | | LKQKQANLALKIIEQLPSAKESPD | |
| | | | | KFLEVCTWVDQVAALNDSRTRKT | |
| | | | | TSETVRAVLDSLSEKKKSSS | |

d) Results

The activity of the single chain molecules against the HSV12 target was monitored using the previously described CHO assay along with our internal control SCOH-RAG and I-Sce I meganucleases. All comparisons were done upon DNA dose response of transfected variant DNA. All the single molecules displayed strong cleavage activity of HSV 12 target in CHO assay as listed in Table XXXXVIII.

Variants shared specific behaviour upon assayed dose depending on the mutation profile they bear. For example, pCLS2633 displays a similar activity and profile than I-Sce I (FIG. 42). Its activity reaches the plateau even at 12.5 ng DNA dose. All of the variants described in Table XXXXVIII are active and can be used for the HSV-1 virus RL2 gene targeting and cleavage.

### EXAMPLE 8

### Validation of tHSV1, tHSV2, tHSV4, tHSV8, tHSV9 or tHSV12 Target Cleavage in an Extrachromosomal Model in CHO Cells and Toxicity Evaluation

### 1) Materials and Methods

Cell Survival Assay

The CHO cells were used to seed plates at a density of 5000 cells in 96-well plates. The next day, various amounts of meganuclease expression vectors and a constant amount of GFP-encoding plasmid complexed to Polyfect® transfection reagent were used to transfect the cells. GFP levels were monitored on days 1 and 6 after transfection, by flow cytometry. Cell survival is expressed as a percentage and was calculated as a ratio: (meganuclease-transfected cell expressing GFP on day 6/control transfected cell expressing GFP on day 6), corrected for the transfection efficiency determined on day 1.

### 2) Results

The activity of the anti-HSV meganucleases was characterized in the CHO extrachromosomal assay. We used as positive controls the I-SceI and mRag1 meganucleases. mHSV4 (pCLS2790) (SEQ ID NO:534) and mHSV12 (pCLS2633) (SEQ ID NO:465) displayed very similar levels of activity, matching the activity of I-SceI and mRag1, and mHSV1 (pCLS2588) (SEQ ID NO:535 or SEQ ID NO:561) proved slightly less active (FIG. 42). However, mHSV2 (pCLS2459), mHSV8 (pCLS3306) (SEQ ID NO:576) and mHSV9 (pCLS3318) (SEQ ID NO:590) displayed a markedly different profile, with maximal activity being observed at a very low dose (0.39 ng), indicative of an extremely active proteins. For comparison, the I-SceI and Rag1 proteins reached approximately the same maximal activity at a 16 times higher dose (6.25 ng) of plasmid. However, at a higher dose (12.5 ng), the activity of mHSV2 decreased (FIG. 42).

In order to evaluate potential meganuclease toxicity, we used a cell survival assay described by different authors in previous studies. Three meganucleases were used as controls in this assay (FIG. 43): I-SceI, mRag1 and the I-CreI natural endonuclease. We have previously shown that in this type of assay, both I-SceI and mRag1 display little toxicity, an outcome that was confirmed in this experiment. In contrast, a significant toxic effect was observed with I-CreI protein, consistent with what we observed in another assay wherein meganucleases are over-expressed in yeast cells at 37° C. Among the six anti-HSV meganucleases, mHSV1 proved quite innocuous, with its profile mimicking the I-SceI and mRag1 profiles. mHSV2 behaved very similar to I-CreI, while the four other proteins displayed intermediate patterns. These results indicate that mHSV2 can be toxic at high doses (FIG. 43).

### EXAMPLE 9

### Strategy for Engineering Novel Meganucleases Cleaving the HBV12 Target from the Hepatitis B Genome

HBV12 is a 22 bp (non-palindromic) target located in the coding sequence of the RNA dependent DNA polymerase gene in the Hepatitis B genome. The target sequence corresponds to positions 2828-2850 of the Hepatitis B genome (accession number X70185, FIG. 84).

The HBV12 sequence is partly a patchwork of the 10ATT_P, 10TAG_P, 5TGG_P and 5_CTT_P targets (FIG. 55) which are cleaved by previously identified meganucleases, obtained as described in International PCT Applications WO 2006/097784 and WO 2006/097853; Arnould et al., J. Mol. Biol., 2006, 355, 443-458; Smith et al., Nucleic Acids Res., 2006. Thus the inventors set out to determine whether HBV12 could be cleaved by combinatorial variants resulting from these previously identified meganucleases.

The 10ATT_P, 10TAG_P, 5TGG_P and 5_CTT_P target sequences are 24 bp derivatives of C1221, a palindromic sequence cleaved by I-CreI (Arnould et al., precited). However, the structure of I-CreI bound to its DNA target suggests that the two external base pairs of these targets (positions −12 and 12) have no impact on binding and cleavage (Chevalier et al., Nat. Struct. Biol., 2001, 8, 312-316; Chevalier and Stoddard, Nucleic Acids Res., 2001, 29, 3757-3774; Chevalier et al., J. Mol. Biol., 2003, 329, 253-269), and in this study, only positions −11 to 11 were considered. Consequently, the HBV12 series of targets were defined as 22 bp sequences instead of 24 bp. HBV12 differs from C1221 in the 4 bp central region. According to the structure of the I-CreI protein bound to its target, there is no contact between the 4 central base pairs (positions −2 to 2) and the I-CreI protein (Chevalier et al., Nat. Struct. Biol., 2001, 8, 312-316; Chevalier and Stoddard, Nucleic Acids Res., 2001, 29, 3757-3774; Chevalier et al., J. Mol. Biol., 2003, 329, 253-269). Thus, the bases at these positions should not impact the binding efficiency. However, they could affect cleavage, which results from two nicks at the edge of this region. Thus, the gaac sequence in −2 to 2 was first substituted with the gtac sequence from C 1221, resulting in target HBV12.2 (FIG. 55). Then, two palindromic targets, HBV12.3 and HBV12.4, were derived from HBV12.2 (FIG. 55). Since HBV12.3 and HBV12.4 are palindromic, they should be cleaved by homodimeric proteins. Thus, proteins able to cleave the HBV12.3 and HBV12.4 sequences as homodimers were first designed (Examples 10 and 11) and then co-expressed to obtain heterodimers cleaving HBV12 (Example 12). Heterodimers cleaving the HBV12 target could be identified. In order to improve cleavage activity for the HBV12 target, a series of variants cleaving HBV12.3 and HBV12.4 was chosen, and then refined. The chosen variants were subjected to random or site-directed mutagenesis, and used to form novel heterodimers that were screened against the HBV12 target (Examples 13, 14 and 15). Strong cleavage activity of the HBV12 target could be observed for these heterodimers.

### EXAMPLE 10

### Identification of Meganucleases Cleaving HBV12.3

This example shows that I-CreI variants can cut the HBV12.3 DNA target sequence derived from the left part of the HBV12.2 target in a palindromic form (FIG. 55). Target sequences described in this example are 22 bp palindromic sequences. Therefore, they will be described only by the first 11 nucleotides, followed by the suffix _P (For example, target HBV12.3 will be noted tattcttgggt_P).

HBV12.3 is similar to 10ATT_P at positions ±1, ±2, ±8, ±9, and +10 and to 5TGG_P at positions ±1, ±2, ±3, ±4, ±5 and ±10. It was hypothesized that positions ±6, ±7 and ±11 would have little effect on the binding and cleavage activity. Variants able to cleave the 0ATT_P target were obtained by mutagenesis of I-CreI N75 or D75, at positions 28, 30, 32, 33, 38, 40 and 70, as described previously in Smith et al. Nucleic Acids Res., 2006, 34, e149; International PCT Applications WO 2007/060495 and WO 2007/049156. Variants able to cleave 5TGG_P were obtained by mutagenesis on I-CreI N75 at positions 44, 68, 70, 75 and 77 as described in Arnould et al., J. Mol. Biol., 2006, 355, 443-458; Smith et al. Nucleic Acids Res., 2006, 34, e149; International PCT Applications WO 2006/097784, WO 2006/097853, WO 2007/060495 and WO 2007/049156.

Both sets of proteins are mutated at position 70. However, the existence of two separable functional subdomains was hypothesized. This implies that this position has little impact on the specificity at bases 10 to 8 of the target.

Therefore, to check whether combined variants could cleave the HBV12.3 target, mutations at positions 44, 68, 70, 75 and 77 from proteins cleaving 5TGG_P were combined with the 28, 30, 32, 33, 38 and 40 mutations from proteins cleaving 10ATT_P.

A) Material and Methods

a) Construction of Target Vector

The target was cloned as follows: an oligonucleotide corresponding to the HBV12.3 target sequence flanked by gateway cloning sequences was ordered from PROLIGO: 5′ tggcatacaagtttatattcttgggtacccaagaatatcaatcgtctgtca 3′ (SEQ ID NO: 620). Double-stranded target DNA, generated by PCR amplification of the single stranded oligonucleotide, was cloned using the Gateway protocol (INVITROGEN) into the yeast reporter vector (pCLS1055, FIG. 4). Yeast reporter vector was transformed into Saccharomyces cerevisiae strain FYBL2-7B (MAT a, ura3Δ851, trp1Δ63, leu2Δ1, lys2Δ202), resulting in a reporter strain.

b) Mating of Meganuclease Expressing Clones and Screening in Yeast

I-CreI variants cleaving 10ATT_P or 5TGG_P were previously identified, as described in Smith et al. Nucleic Acids Res., 2006, 34, e149; International PCT Applications WO 2007/060495 and WO 2007/049156, and Arnould et al., J. Mol. Biol., 2006, 355, 443-458; International PCT Applications WO 2006/097784 and WO 2006/097853, respectively for the 10ATT_P and 5TGG_P targets. In order to generate I-CreI derived coding sequences containing mutations from both series, separate overlapping PCR reactions were carried out that amplify the 5′ end (aa positions 1-43) or the 3′ end (positions 39-167) of the I-CreI coding sequence. For both the 5′ and 3′ end, PCR amplification is carried out using primers (Gal10F 5′-gcaactttagtgctgacacatacagg-3′ (SEQ ID NO: 263) or Gal10R 5′-acaaccttgattggagacttgacc-3′(SEQ ID NO: 264)) specific to the vector (pCLS0542, FIG. 5) and primers (assF 5′-ctannnttgaccttt-3′ (SEQ ID NO: 265) or assR 5′-aaaggtcaannntag-3′(SEQ ID NO: 266)), where nnn codes for residue 40, specific to the I-CreI coding sequence for amino acids 39-43. The PCR fragments resulting from the amplification reaction realized with the same primers and with the same coding sequence for residue 40 were pooled. Then, each pool of PCR fragments resulting from the reaction with primers Gal10F and assR or assF and Gal10R was mixed in an equimolar ratio. Finally, approximately 25 ng of each final pool of the two overlapping PCR fragments and 75 ng of vector DNA (pCLS0542, FIG. 6) linearized by digestion with NcoI and EagI were used to transform the yeast Saccharomyces cerevisiae strain FYC2-6A (MATα, trp1Δ63, leu2Δ1, his3Δ200) using a high efficiency LiAc transformation protocol (Gietz and Woods, Methods Enzymol., 2002, 350, 87-96). An intact coding sequence containing both groups of mutations is generated by in vive homologous recombination in yeast.

c) Mating of Meganuclease Expressing Clones and Screening in Yeast

Screening was performed as described previously (Arnould et al., J. Mol. Biol., 2006, 355, 443-458). Mating was performed using a colony gridder (QpixII, GENETIX). Variants were gridded on nylon filters covering YPD plates, using a low gridding density (4-6 spots/cm²). A second gridding process was performed on the same filters to spot a second layer consisting of the reporter-harboring yeast strain for the target of interest. Membranes were placed on solid agar YPD rich medium, and incubated at 30° C. for one night, to allow mating. Next, filters were transferred to synthetic medium, lacking leucine and tryptophan, with galactose (2%) as a carbon source, and incubated for five days at 37° C., to select for diploids carrying the expression and target vectors. After 5 days, filters were placed on solid agarose medium with 0.02% X-Gal in 0.5 M sodium phosphate buffer, pH7.0, 0.1% SDS, 6% dimethyl formamide (DMF), 7 mM β-mercaptoethanol, 1% agarose and incubated at 37° C., to monitor β-galactosidase activity. Results were analyzed by scanning and quantification was performed using appropriate software.

d) Sequencing of Variants

To recover the variant expression plasmids, yeast DNA was extracted using standard protocols and used to transform E. coli. Sequencing of variant ORFs was then performed on the plasmids by MILLEGEN SA. Alternatively, ORFs were amplified from yeast DNA by PCR (Akada et al., Biotechniques, 2000, 28, 668-670), and sequencing was performed directly on the PCR product by MILLEGEN SA.

B) Results

I-CreI combinatorial variants were constructed by associating mutations at positions 44, 68, 70, 75 and 77 from proteins cleaving 5TGG_P with the mutations at 28, 30, 32, 33, 38 and 40 from proteins cleaving 10ATT_P on the I-CreI scaffold, resulting in a library of complexity 94. Examples of combinatorial variants are displayed in Table XXXXVIX. This library was transformed into yeast and 2232 clones (23.7 times the diversity) were screened for cleavage against the HBV2.3 DNA target (tattcttgggt_P, SEQ ID NO: 618). Six positive clones were found, which after sequencing turned out to correspond to six different novel endonuclease variants (Table L). Examples of positives are shown in FIG. 56. All six variants display non parental combinations at positions 28, 30, 32, 33, 38, 40 or 44, 68, 70, 75, 77. Such combinations likely result from PCR artifacts during the combinatorial process. Alternatively, the variants may be I-CreI combined variants resulting from micro-recombination between two original variants during in vivo homologous recombination in yeast.

TABLE XXXXVIX
Panel of variants* theoretically present in the combinatorial library

Amino acids at
positions 44, 68,
70, 75 and 77
(ex: DYSSR

| stands for D44, | Amino acids at positions 28, 30, 32, 33, 38 and 40 | | | | | | | | | | |
|---|---|---|---|---|---|---|---|---|---|---|---|
| Y68, S70, S75 | (ex: KDSRQS stands for K28, D30, S32, R33, Q38 and S40) | | | | | | | | | | |
| and R77) | KDSRQS | KSSNQS | KSSCQS | KNNGQS | KNTYAS | KNDYGS | KQSTQS | KNDYCS | KNSNTS | KCSGQS | KNSCAS |

DYSSR
YRSYV
*Only 22 out of the 94 combinations are displayed. None of them were identified in the positive clones.

TABLE L
I-CreI variants capable of cleaving the HBV12.3 DNA target.

| Amino acids at positions 28, 30, 32, | |
|---|---|
| 33, 38, 40/44, 68, 70, 75 and 77 | |
| of the I-CreI variants | |
| (ex: KSRSQS/DYSSR stands for | SEQ |
| K28, S30, R32, S33, Q38, S40/D44, | ID |
| Y68, S70, S75 and R77) | NO: |
| KSRSQS/DYSSR | 621 |
| KNQYCS/DYSSR | 622 |
| KNKTQS/DYSSR | 623 |
| KRYSQS/DYSSR | 624 |
| KSSNQS/DYSSR + 66H | 625 |
| KNAAGS/DYSSR + 89A | 626 |

### EXAMPLE 11

### Identification of Meganucleases Cleaving HBV12.4

This Example shows that I-CreI variants can cleave the HBV12.4 DNA target sequence derived from the right part of the HBV12.2 target in a palindromic form (FIG. 55). All target sequences described in this example are 22 bp palindromic sequences. Therefore, they will be described only by the first 11 nucleotides, followed by the suffix _P (for example, HBV12.4 will be called gtagctcttgt_P).

HBV2.4 is similar to 5CTT_P at positions ±1, ±2, ±3, ±4, ±5 and ±9 and to 10TAG_P at positions ±1, ±2, ±4, ±8, ±9 and ±10. It was hypothesized that positions ±6, ±7 and ±11 would have little effect on the binding and cleavage activity. Variants able to cleave 5CTT_P were obtained by mutagenesis of I-CreI N75 at positions 24, 44, 68, 70, 75 and 77, as described previously (Arnould et al., J. Mol. Biol., 2006, 355, 443-458; Smith et al. Nucleic Acids Res., 2006, 34, e149; International PCT Applications WO 2006/097784, WO 2006/097853, WO 2007/060495 and WO 2007/049156). Variants able to cleave the 10TAG_P target were obtained by mutagenesis of I-CreI N75 or D75, at positions 28, 30, 32, 33, 38 and 40, as described previously in Smith et al. Nucleic Acids Res., 2006, 34, e149; International PCT Applications WO 2007/060495 and WO 2007/049156.

Mutations at positions 24 found in variants cleaving the 5CTT_P target will be lost during the combinatorial process. But it was hypothesized that this will have little impact on the capacity of the combined variants to cleave the HBV12.4 target.

Therefore, to check whether combined variants could cleave the HBV12.4 target, mutations at positions 44, 68, 70, 75 and 77 from proteins cleaving 5CTT_P were combined with the 28, 30, 32, 33, 38 and 40 mutations from proteins cleaving 10TAG_P.

A) Material and Methods

a) Construction of Target Vector

The experimental procedure is as described in Example 10, with the exception that an oligonucleotide corresponding to the HBV12.4 target sequence was used: 5′ tggcatacaagttttgtagctcttgtacaagagctacacaatcgtctgtca 3′ (SEQ ID NO: 803).

b) Construction of Combinatorial Variants

I-CreI variants cleaving 10TAG_P or 5CTT_P were previously identified, as described in Smith et al. Nucleic Acids Res., 2006, 34, e149; International PCT Applications WO 2007/060495 and WO 2007/049156, and Arnould et al., J. Mol. Biol., 2006, 355, 443-458; International PCT Applications WO 2006/097784 and WO 2006/097853, respectively for the 10TAG_P and 5CTT_P targets. In order to generate I-CreI derived coding sequences containing mutations from both series, separate overlapping PCR reactions were carried out that amplify the 5′ end (aa positions 1-43) or the 3′ end (positions 39-167) of the I-CreI coding sequence. For both the 5′ and 3′ end, PCR amplification is carried out using primers (Gal10F 5′-gcaactttagtgctgacacatacagg-3′ (SEQ ID NO: 263) or Gal10R 5′-acaaccttgattggagacttgacc-3′ (SEQ ID NO: 264)) specific to the vector (pCLS1107, FIG. 6) and primers (assF 5′-ctannnttgaccttt-3′ (SEQ ID NO: 265) or assR 5′-aaaggtcaannntag-3′(SEQ ID NO: 266)), where nnn codes for residue 40, specific to the I-CreI coding sequence for amino acids 39-43. The PCR fragments resulting from the amplification reaction realized with the same primers and with the same coding sequence for residue 40 were pooled. Then, each pool of PCR fragments resulting from the reaction with primers Gal10F and assR or assF and Gal10R was mixed in an equimolar ratio. Finally, approximately 25 ng of each final pool of the two overlapping PCR fragments and 75 ng of vector DNA (pCLS1107, FIG. 6) linearized by digestion with DraIII and NgoMIV were used to transform the yeast Saccharomyces cerevisiae strain FYC2-6A (MATα, trp1Δ63, leu2Δ1, his3Δ200) using a high efficiency LiAc transformation protocol (Gietz and Woods, Methods Enzymol., 2002, 350, 87-96). An intact coding sequence containing both groups of mutations is generated by in vivo homologous recombination in yeast.

c) Mating of Meganuclease Expressing Clones and Screening in Yeast

Screening was performed as described previously (Arnould et al., J. Mol. Biol., 2006, 355, 443-458). Mating was performed using a colony gridder (QpixII, GENETIX). Variants were gridded on nylon filters covering YPD plates, using a low gridding density (4-6 spots/cm²). A second gridding process was performed on the same filters to spot a second layer consisting of the reporter-harboring yeast strain for the target of interest. Membranes were placed on solid agar YPD rich medium, and incubated at 30° C. for one night, to allow mating. Next, filters were transferred to synthetic medium, lacking tryptophan, including G418, with galactose (2%) as a carbon source, and incubated for five days at 37° C., to select for diploids carrying the expression and target vectors. After 5 days, filters were placed on solid agarose medium with 0.02% X-Gal in 0.5 M sodium phosphate buffer, pH 7.0, 0.1% SDS, 6% dimethyl formamide (DMF), 7 mM β-mercaptoethanol, 1% agarose, and incubated at 37° C., to monitor β-galactosidase activity. Results were analyzed by scanning and quantification was performed using appropriate software.

d) Sequencing of Variants

Experimental procedure is as described in example 10.

B) Results

I-CreI combinatorial variants were constructed by associating mutations at positions 44, 68, 70, 75 and 77 from proteins cleaving 5CTT_P with the mutations 28, 30, 32, 33, 38 and 40 from proteins cleaving 10TAG_P on the 1-CreI scaffold, resulting in a library of complexity 720. Examples of combinatorial variants are displayed in Table LI. This library was transformed into yeast and 2232 clones (3.1 times the diversity) were screened for cleavage against the HBV12.4 DNA target (gtagctcttgt_P, SEQ ID NO: 619). A total of 664 positive clones were found to cleave HBV12.4. Sequencing and validation by secondary screening of 93 of the I-CreI variants resulted in the identification of 51 different novel endonucleases. Examples of positives are shown in FIG. 58. The sequence of several of the variants identified display non parental combinations at positions 28, 30, 32, 33, 38, 40 or 44, 68, 70, 75, 77 as well as additional mutations (see Examples Table LII). Such variants likely result from PCR artifacts during the combinatorial process. Alternatively, the variants may be I-CreI combined variants resulting from micro-recombination between two original variants during in vivo homologous recombination in yeast.

TABLE LI
Panel of variants* theoretically present in the combinatorial library

| Amino acids at | | | | | | | | | | |
|---|---|---|---|---|---|---|---|---|---|---|
| positions 44, 68, | | | | | | | | | | |
| 70, 75 and 77 | | | | | | | | | | |
| (ex: RYSDN | | | | | | | | | | |
| stands for R44, | Amino acids at positions 28, 30, 32, 33, 38 and 40 | | | | | | | | | |
| Y68, S70, D75 | (ex: KNNCQS stands for K28, N30, N32, C33, Q38 and S40) | | | | | | | | | |
| and N77) | KNNCQS | KNRCQS | KNSGQS | KNSCQS | KNHAQS | KNGPQS | KNTCQS | KNGCQS | KNEAQS | KNHSQS |

| | | | | |
|---|---|---|---|---|
| RYSDN | + | + | + | |
| KTSDR | + | | | |
| RYSYN | + | + | + | |
| KESNR | | | | |
| RYSDQ | | + | + | + |
| QASQR | | | | |
| RNSNN | | | | |
| RYSNN | + | + | + | |
| RYSYI | | | | |
| RNSDR | | | | |
| KASDK | | | | |
| KESDR | | | | |
| RYSNI | | | | |
| KSSDI | | | | |
| RASDR | | | | |
| RTSNN | | | | |
| QNSQR | | | | |
| QESNR | | | | |
| QRSQR | | | | |
| QSSNR | | | | |
| RSSNN | | | + | |
| KASDV | | | | |

*Only 220 out of the 720 combinations are dispayed.
+ indicates that a functional combinatorial variant cleaving the HBV12.4 target was found among the identified positives.

TABLE LII
I-CreI variants with additional mutations capable of cleaving the
HBV12.4 DNA target.

| Amino acids at positions 28, 30, 32, | |
|---|---|
| 33, 38, 40/44, 68, 70, 75 and 77 | |
| of the I-CreI variants | |
| (ex: KRGYQS/KYSNI stands for | SEQ |
| K28, R30, G32, Y33, Q38, S40/ | ID |
| K44, Y68, S70, N75 and I77) | NO: |
| KNHCQS/RYSYN | 628 |
| KNHCQS/RYSNQ + 117K | 629 |
| KNHCQS/RYSNQ | 630 |
| KNHCQS/RYSNN | 631 |
| KNHCQS/RYSDQ + 151A | 632 |
| KNHCQS/RYSDQ | 633 |

### EXAMPLE 12

### Making of Meganucleases Cleaving HBV12

I-CreI variants able to cleave each of the palindromic HBV12.2 derived targets (HBV12.3 and HBV12.4) were identified in Example 10 and Example 11. Pairs of such variants (one cutting HBV12.3 and one cutting HBV12.4) were co-expressed in yeast. Upon co-expression, there should be three active molecular species, two homodimers, and one heterodimer. It was assayed whether the heterodimers that should be formed, cut the non palindromic HBV12 target, which differs from the HBV12.2 sequence by 2 bp at positions 1 and 2.

A) Materials and Methods

a) Construction of Target Vector

The experimental procedure is as described in Example 2, with the exception that an oligonucleotide corresponding to the HBV12 target sequence: 5′ tggcatacaagtttatattcttgggaacaagagctacacaatcgtctgtca 3′ (SEQ ID NO: 633) was used.

b) Co-Expression of Variants

Yeast DNA was extracted from variants cleaving the HBV12.3 target (pCLS542 expression vector) as well as those cleaving the HBV12.4 target (pCLS1107 expression vector) using standard protocols and were used to transform E. coli. Plasmid DNA derived from a HBV12.3 variant and a HBV12.4 variant was then co-transformed into the yeast Saccharomyces cerevisiae strain FYC2-6A (MATα, trp1Δ63, leu2Δ1, his3Δ200) using a high efficiency LiAc transformation protocol (Gietz and Woods, Methods Enzymol., 2002, 350, 87-96). Transformants were selected on synthetic medium lacking leucine and containing G418.

c) Mating of Meganuclease Co-Expressing Clones and Screening in Yeast

Mating was performed using a colony gridder (QpixII, Genetix). Variants were gridded on nylon filters covering YPD plates, using a low gridding density (4-6 spots/cm²). A second gridding process was performed on the same filters to spot a second layer consisting of the reporter-harboring yeast strain for the target of interest. Membranes were placed on solid agar YPD rich medium, and incubated at 30° C. for one night, to allow mating. Next, filters were transferred to synthetic medium, lacking leucine and tryptophan, including G418, with galactose (2%) as a carbon source, and incubated for five days at 37° C., to select for diploids carrying the expression and target vectors. After 5 days, filters were placed on solid agarose medium with 0.02% X-Gal in 0.5 M sodium phosphate buffer, pH7.0, 0.1% SDS, 6% dimethyl formamide (DMF), 7 mM β-mercaptoethanol, 1% agarose, and incubated at 37° C., to monitor β-galactosidase activity. Results were analyzed by scanning and quantification was performed using appropriate software.

B) Results

Co-expression of variants cleaving the HBV12.4 target (7 variants chosen among those described in Table LI and Table LII) and the six variants cleaving the HBV12.3 target (described in Table L) resulted in weak cleavage of the HBV12 target in certain cases (FIG. 58). Functional combinations are summarized in Table LIII.

TABLE LIII
Cleavage of the HBV12 target by the hetrodimeric variants

| Amino acids at positions 28, 30, 32, 33, 38, 40/44, 68, 70, 75 and 77 | | | | | |
|---|---|---|---|---|---|
| of the I-CreI variants cleaving the HBV12.3 target | | | | | |
| (ex. KRYSQS/DYSSR stands for K28, R30, Y32, S33, Q38, S40/D44, Y68, S70, S75 and R77 | | | | | |
| KRYSQS/ | KSRSQS/ | KNAAGS/ | KSSNQS/ | KNQYCS/ | KNTQS/ |
| DYSSR | DYSSR | DYSSR + 89A | DYSSR + 66H | DYSSR | DYSSR |
| (SEQ ID | (SEQ ID | (SEQ ID | (SEQ ID | (SEQ ID | (SEQ ID |
| NO: 624) | NO: 621 | NO: 625 | NO: 624 | NO: 622 | NO: 623 |

| | | | | |
|---|---|---|---|---|
| Amino acids | KNNCQS/RYSYN | +* | +* | +* |
| at positions | (SEQ ID NO: 634) | | | |
| 28, 30, 32, 33, | KNHCQS/RYSYN | +* | +* | +* |
| 38, 40/44, 68, | (SEQ ID NO: 627) | | | |
| 70, 75 and 77 of | KNHCQS/RYSNQ + 117K | | +* | +* |
| I-CreI variants | (SEQ ID NO: 628) | | | |
| cleaving the | KNHCQS/RYSNQ | +* | +* | +* |
| HBV12.4 target | (SEQ ID NO: 629 | | | |
| (ex. KNNCQS/ | KNHCQS/RYSNN | | | |
| RYSYN stands for | (SEQ ID NO: 630 | | | |
| K28, N30, N32, | KNHCQS/RYSDQ + 151A | +* | +* | +* |
| C33, Q38, S40/ | (SEQ ID NO: 631) | | | |
| R44, Y68, S70, | KNHCQS/RYSDQ | | +* | +* |
| Y75 and N77) | (SEQ ID NO: 632) | | | |

+ indicates a functional combination
*indicates that the combination weakly cuts the HBV12 target.

### EXAMPLE 13

### Improvement of Meganucleases Cleaving HBV12 by Random Mutagenesis of Proteins Cleaving HBV12.3 and Assembly with Proteins Cleaving HBV12.4

I-CreI variants able to cleave the HBV12 target by assembly of variants cleaving the palindromic HBV12.3 and HBV12.4 target have been previously identified in Example 12. However, these variants display weak activity with the HBV12 target.

Therefore five combinatorial variants cleaving HBV12.3 were mutagenized, and variants were screened for cleavage activity of HBV12 when co-expressed with a variant cleaving HBV12.4. According to the structure of the I-CreI protein bound to its target, there is no contact between the 4 central base pairs (positions −2 to 2) and the I-CreI protein (Chevalier et al., Nat. Struct. Biol., 2001, 8, 312-316; Chevalier and Stoddard, Nucleic Acids Res., 2001, 29, 3757-3774; Chevalier et al., J. Mol. Biol., 2003, 329, 253-269). Thus, it is difficult to rationally choose a set of positions to mutagenize, and mutagenesis was performed on the whole protein. Random mutagenesis results in high complexity libraries. Therefore, to limit the complexity of the variant libraries to be tested, only one of the two components of the heterodimers cleaving HBV12 was mutagenized.

Thus, in a first step, proteins cleaving HBV12.3 were mutagenized, and in a second step, it was assessed whether they could cleave HBV12 when co-expressed with a protein cleaving HBV12.4.

A) Material and Methods
a) Construction of Libraries by Random Mutagenesis

Random mutagenesis was performed on a pool of chosen variants, by PCR using Mn²⁺. PCR reactions were carried out that amplify the I-CreI coding sequence using the primers preATGCreFor (5′-gcataaattactatacttctatagacacgcaaacacagcggccttgccacc-3′; SEQ ID NO: 169) and I-CreIpostRev (5′-ggctcgaggagctcgtctagaggatcgctcgagttatcagtcggccgc-3′; SEQ ID NO: 170), which are common to the pCLS0542 (FIG. 5) and pCLS1107 (FIG. 6) vectors. Approximately 25 ng of the PCR product and 75 ng of vector DNA (pCLS0542) linearized by digestion with NcoI and EagI were used to transform the yeast Saccharomyces cerevisiae strain FYC2-6A (MATα, trp1Δ63, leu2Δ1, his3Δ200) using a high efficiency LiAc transformation protocol (Gietz and Woods, Methods Enzymol., 2002, 350, 87-96). Expression plasmids containing an intact coding sequence for the I-CreI variant were generated by in vivo homologous recombination in yeast.

b) Variant-Target Yeast Strains. Screening and Sequencing

The yeast strain FYBL2-7B (MAT a, ura3Δ851, trp1Δ63, leu2Δ1, lys2Δ202) containing the HBV12 target in the yeast reporter vector (pCLS1055, FIG. 4) was transformed with variants, in the kanamycin vector (pCLS1107), cutting the HBV12.4 target, using a high efficiency LiAc transformation protocol. Variant-target yeast strains were used as target strains for mating assays as described in Example 12. Positives resulting clones were verified by sequencing (MILLEGEN) as described in Example 10.

B) Results

Five variants cleaving HBV12.3 (I-CreI 30R, 32Y, 33S, 44D, 68Y, 70S, 75S, 77R, I-CreI 30S, 32R, 33S, 44D, 68Y, 70S, 75S, 77R, I-CreI 32A, 33A, 38G, 44D, 68Y, 70S, 75 S, 77R, 89A, I-CreI 32Q, 38C, 44D, 68Y, 70S, 75S, 77R and I-CreI 32K, 33T, 44D, 68Y, 70S, 75S, 77R, also called KRYSQS/DYSSR (SEQ ID NO: 624), KSRSQS/DYSSR (SEQ ID NO: 621), KNAAGS/DYSSR +89A (SEQ ID NO: 626), KNQYCS/DYSSR (SEQ ID NO: 622) and KNKTQS/DYSSR (SEQ ID NO: 623) respectively, according to the nomenclature of Table L, were pooled, randomly mutagenized and transformed into yeast. 2232 transformed clones were then mated with a yeast strain that contains (i) the HBV12 target in a reporter plasmid (ii) an expression plasmid containing a variant that cleaves the HBV12.4 target (I-CreI 32H, 33C, 44R, 68Y, 70S, 75N, 77Q or KNHCQS/RYSNQ (SEQ ID NO: 628) according to the nomenclature of Table LII After mating with this yeast strain, 156 clones were found to cleave the HBV12 target more efficiently than the original variant. Thus, 156 positives contained proteins able to form heterodimers with KNHCQS/RYSNQ with an improved cleavage activity for the HBV12 target. An example of positives is shown in FIG. 59. Sequencing of the strongest 93 positive clones indicates that 29 distinct variants were identified (Examples listed in Table LIV).

TABLE LIV
Functional variant combinations displaying improved cleavage
activity for HBV12.

| | | Optimized* Variants HBV12.3 |
|---|---|---|
| | | (SEQ ID NO: 635 to 647) |
| VARIANT HBV12.4 | I-CreI 28K 30N 32H 33C 38Q | 24F 32Q 38C 44D 68Y 70S 75S 77R |
| | 40S 44R 68Y 70S 75N 77Q | 30R 32Y 33S 44D 64A 68Y 70S 75S 77R |
| | KNHCQS/RYSNQ) (SEQ ID NO: 628) | 30S 32H 33S 44D 68Y 70S 75S 77R |
| | | 30S 32R 33S 44D 68Y 70S 72P 75S 77R 81V |
| | | 7E 30S 32R 33S 44D 68Y 70S 75S 77R |
| | | 30S 32R 33S 44D 68Y 70S 75S 77R 107R |
| | | 30S 32R 33S 44D 68Y 70S 75S 77R 153G |
| | | 30S 32R 33S 44D 68Y 70S 75S 77R 81T 160E |
| | | 30S 32R 33S 44D 68Y 70S 75S 77R 81V 162P |
| | | 30S 32R 33S 44D 68Y 70S 75S 77R 89A |
| | | 30S 32R 33S 44D 68Y 70S 75S 77R 99R |
| | | 32G 38G 44D 64A 68Y 70S 75S 77R 89A |
| | | 32G 38G 44D 68Y 70S 75S 77R |
| | | 32G 38G 44D 68Y 70S 75S 77R 81T |
| | | 32Q 38C 44D 68Y 70S 75S 77R 80A |
| | | 32R 33S 44D 68Y 70S 75S 76F 77R |
| | | 32R 38C 44D 68Y 70S 75S 77R 109V 157K |

*Mutations resulting from random mutagenesis are in bold.

### EXAMPLE 14

### Improvement of Meganucleases Cleaving HBV12 by Site-Directed Mutagenesis of Proteins Cleaving HBV12.3 and Assembly with Proteins Cleaving HBV12.4

The optimized I-CreI variants cleaving HBV12.3 described in Table LIV that resulted from random mutagenesis as described in Example 13 were further mutagenized by introducing selected amino-acid substitutions in the proteins and screening for more efficient variants cleaving HBV12 in combination with a variant cleaving HBV12.4.

Six amino-acid substitutions have been found in previous studies to enhance the activity of I-CreI derivatives: these mutations correspond to the replacement of Glycine 19 with Serine (G19S), Phenylalanine 54 with Leucine (F54L), Glutamic acid 80 with Lysine (E80K), Phenylalanine 87 with Leucine (F87L), Valine 105 with Alanine (V105A) and Isoleucine 132 with Valine (I132V). These mutations were introduced into the coding sequence of proteins cleaving HBV12.3, and the resulting proteins were tested for their ability to induce cleavage of the HBV12 target, upon co-expression with a variant cleaving HBV12.4.

A) Material and Methods

a) Site-Directed Mutagenesis

A site-directed mutagenesis library was created by PCR on a pool of chosen variants. For example, to introduce the G19S substitution into the coding sequence of the variants, two separate overlapping PCR reactions were carried out that amplify the 5′ end (residues 1-24) or the 3′ end (residues 14-167) of the I-CreI coding sequence. For both the 5′ and 3′ end, PCR amplification is carried out using a primer with homology to the vector (Gal10F 5′-gcaactttagtgctgacacatacagg-3′ (SEQ ID NO: 263) or Gal10R 5′-acaaccttgattggagacttgacc-3′(SEQ ID NO: 264)) and a primer specific to the I-CreI coding sequence for amino acids 14-24 that contains the substitution mutation G19S (G19SF 5′-gccggctttgtggactctgacggtagcatcatc-3′ (SEQ ID NO: 653) or G19SR 5′-gatgatgctaccgtcagagtccacaaagccggc-3′(SEQ ID NO: 654)). The resulting PCR products contain 33 bp of homology with each other. The PCR fragments were purified.

The same strategy was used with the following pairs of oligonucleotides to introduce the F54L, E80K, F87L, V105A and I132V substitutions, respectively:

(SEQ ID NO: 655 and 656)

* F54LF: 5′-acccagcgccgttggctgctggacaaactagtg-3′
and
F54LR: 5′-cactagtttgtccagcagccaacggcgctgggt-3′;

(SEQ ID NO: 657 and 658)

* E80KF: 5′-ttaagcaaaatcaagccgctgcacaacttcctg-3′
and
E80KR: 5′-caggaagttgtgcagcggcttgattttgcttaa-3′;

(SEQ ID NO: 659 and 660)

* F87LF: 5′-aagccgctgcacaacctgctgactcaactgcag-3′
and
F87LR: 5′-ctgcagttgagtcagcaggttgtgcagcggctt-3′;

(SEQ ID NO: 661 and 662)

* V105AF: 5′-aaacaggcaaacctggctctgaaaattatcgaa-3′
and
V105AR: 5′-ttcgataattttcagagccaggtttgcctgttt-3′;

(SEQ ID NO: 663 and 664)

* I132VF: 5′-acctgggtggatcaggttgcagctctgaacgat-3′
and
I132VR: 5′-atcgttcagagctgcaacctgatccacccaggt-3′.

The two overlapping PCR fragments for each of the six site-directed mutations were pooled and a total of approximately 25 ng was combined with 75 ng of vector DNA (pCLS0542, FIG. 5) linearized by digestion with NcoI and EagI. The DNA was then used to transform the yeast Saccharomyces cerevisiae strain FYC2-6A (MATα, trp1Δ63, leu2Δ1, his3Δ200) using a high efficiency LiAc transformation protocol (Gietz and Woods, Methods Enzymol., 2002, 350, 87-96). Intact coding sequences containing the one or more of the above described site directed substitutions are generated by in vivo homologous recombination in yeast.

c) Mating of Meganuclease Expressing Clones and Screening in Yeast

The experimental procedure is as described in Example 13.

d) Sequencing of Variants

The experimental procedure is as described in Example 10.

B) Results

A library containing site-directed mutations (G19S, F54L, E80K, F87L, V105A, I132V) was constructed from a pool of 7 variants cleaving HBV12.3 (32Q, 38C, 44D, 68Y, 70S, 75S, 77R, 80A, 24F, 32Q, 38C, 44D, 68Y, 70S, 75S, 77R, 30S, 32R, 33S, 44D, 68Y, 70S, 75S, 77R, 81V, 162P, 30S, 32R, 33S, 44D, 68Y, 70S, 75S, 77R, 153G, 32R, 33S, 44D, 68Y, 70S, 75S, 76F, 77R 7E, 30S, 32R, 33S, 44D, 68Y, 70S, 75S, 77R and 30S, 32H, 33S, 44D, 68Y, 70S, 75S, 77R according to the nomenclature of Table VI). The library was transformed into yeast and 1674 individual clones were picked and mated with a yeast strain that contains (i) the HBV12 target in a reporter plasmid (ii) an expression plasmid containing a variant that cleaves the HBV12.4 target (32H, 33C, 44R, 68Y, 70S, 75N, 77Q or KNHCQS/RYSNQ) according to the nomenclature of Table LII).

After mating with this yeast strain, 122 clones were found to cleave the HBV12 target more efficiently than the original variants. An example of positives is shown in FIG. 60. The sequence of eight of the best I-CreI variants cleaving the HBV12 target when forming a heterodimer with the KNHCQS/RYSNQ variant are listed in Table LV.

TABLE LV
Functional variant combinations displaying strong cleavage activity
for HBV12.

| | | Optimized* Variants HBV12.3 |
|---|---|---|
| | | (SEQ ID NO: 665 to 671) |
| VARIANT HBV12.4 | I-CreI 28K 30N 32H 33C 38Q 40S | 24F 32Q 38C 44D 68Y 70S 75S 77R 87L 153G |
| | 44R 68Y 70S 75N | 24F 32Q 38C 44D 68Y 70S 75S 77R 132V |
| | 77Q(KNHCQS/RYSNQ) (SEQ ID | 19S 32Q 38C 44D 68Y 70S 75S 77R 81V |
| | NO: 630) | 7E 24F 32Q 38C 44D 68Y 70S 75S 77R 80K |
| | | 24F 32Q 38C 44D 68Y 70S 75S 77R 80K |
| | | 24F 32Q 38C 44D 68Y 70S 75S 77R 80K 132V |
| | | 24F 32Q 38C 44D 68Y 70S 75S 77R 105A 132V |
| | | 32Q 38C 44D 68Y 70S 75S 77R 80A 153G |

*Mutations resulting from site-directed mutagenesis are in bold.

### EXAMPLE 15

### Improvement of Meganucleases Cleaving HBV12 by Site-Directed Mutagenesis of Proteins Cleaving HBV12.4 and Assembly with Proteins Cleaving HBV12.3

The initial I-CreI variants cleaving HBV12.4 described in Tables LI and LII were mutagenized by introducing selected amino-acid substitutions in the proteins and screening for more efficient variants cleaving HBV12 in combination with a variant cleaving HBV12.3.

Six amino-acid substitutions have been found in previous studies to enhance the activity of I-CreI derivatives: these mutations correspond to the replacement of Glycine 19 with Serine (G19S), Phenylalanine 54 with Leucine (F54L), Glutamic acid 80 with Lysine (E80K), Phenylalanine 87 with Leucine (F87L), Valine 105 with Alanine (V105A) and Isoleucine 132 with Valine (I132V). These mutations were introduced into the coding sequence of proteins cleaving HBV12.4, and the resulting proteins were tested for their ability to induce cleavage of the HBV12 target, upon co-expression with a variant cleaving HBV12.3.

A) Material and Methods

a) Site-Directed Mutagenesis

A site-directed mutagenesis library was created by PCR on a pool of chosen variants. For example, to introduce the G19S substitution into the coding sequence of the variants, two separate overlapping PCR reactions were carried out that amplify the 5′ end (residues 1-24) or the 3′ end (residues 14-167) of the I-CreI coding sequence. For both the 5′ and 3′ end, PCR amplification is carried out using a primer with homology to the vector (Gal10F 5′-gcaactttagtgctgacacatacagg-3′ (SEQ ID NO: 263) or Gal10R 5′-acaaccttgattggagacttgacc-3′(SEQ ID NO: 264) and a primer specific to the I-CreI coding sequence for amino acids 14-24 that contains the substitution mutation G19S (G(19SF 5′-gccggctttgtggactctgacggtagcatcatc-3′ (SEQ ID NO: 654) or G19SR 5′-gatgatgctaccgtcagagtccacaaagccggc-3′(SEQ ID NO: 655)). The resulting PCR products contain 33 bp of homology with each other. The PCR fragments were purified.

The same strategy was used with the following pairs of oligonucleotides to introduce the F54L, E80K, F87L, V105A and I132V substitutions, respectively:

(SEQ ID NO: 655 and 656)

* F54LF: 5′-acccagcgccgttggctgctggacaaactagtg-3′
and
F54LR: 5′-cactagtttgtccagcagccaacggcgctgggt-3′;

(SEQ ID NO: 657 and 658)

* E80KF: 5′-ttaagcaaaatcaagccgctgcacaacttcctg-3′
and
E80KR: 5′-caggaagttgtgcagcggcttgattttgcttaa-3′;

(SEQ ID NO: 659 and 660)

* F87LF: 5′-aagccgctgcacaacctgctgactcaactgcag-3′
and
F87LR: 5′-ctgcagttgagtcagcaggttgtgcagcggctt-3′;

(SEQ ID NO: 661 and 662)

* V105AF: 5′-aaacaggcaaacctggctctgaaaattatcgaa-3′
and
V105AR: 5′-ttcgataattttcagagccaggatgcctgttt-3′;

(SEQ ID NO: 663 and 664)

* I132VF: 5′-acctgggtggatcaggttgcagctctgaacgat-3′
and
I132VR: 5′-atcgttcagagctgcaacctgatccacccaggt-3′.

The two overlapping PCR fragments for each of the six site-directed mutations were pooled and a total of approximately 25 ng was combined with 75 ng of vector DNA (pCLS1107, FIG. 6) linearized by digestion with DraIII and NgoMIV. The DNA was then used to transform the yeast Saccharomyces cerevisiae strain FYC2-6A (MATα, trp1Δ63, leu2Δ1, his3Δ200) using a high efficiency LiAc transformation protocol (Gietz and Woods, Methods Enzymol., 2002, 350, 87-96). Intact coding sequences containing one or more of the above described site directed substitutions are generated by in vivo homologous recombination in yeast.

c) Mating of Meganuclease Expressing Clones and Screening in Yeast

The experimental procedure is as described in Example 13.

d) Sequencing of Variants

The experimental procedure is as described in Example 10.

B) Results

A library containing site-directed mutations (G19S, F54L, E80K, F87L, V105A, I132V) was constructed from a pool of 7 variants cleaving HBV12.4 (32N, 33C, 44R, 68Y, 70S, 75Y, 77N, 32H, 33C, 44R, 68Y, 70S, 75Y, 77N, 32H, 33C, 44R, 68Y, 70S, 75N, 77Q, 117K, 32H, 33C, 44R, 68Y, 70S, 75N, 77Q, 32H, 33C, 44R, 68Y, 70S, 75N, 77N, 32H, 33C, 44R, 68Y, 70S, 75D, 77Q, 151A and 32H, 33C, 44R, 68Y, 70S, 75D, 77Q, also called KNNCQS/RYSYN (SEQ ID NO: 634), KNHCQS/RYSYN (SEQ ID NO: 628), KNHCQS/RYSNQ +117K (SEQ ID NO: 629), KNHCQS/RYSNQ (SEQ ID NO: 630), KNHCQS/RYSNN (SEQ ID NO: 631), KNHCQS/RYSDQ +151A (SEQ ID NO: 632) and KNHCQS/RYSDQ (SEQ ID NO: 633), respectively, according to the nomenclature of Table LI and Table LII). The library was transformed into yeast and 1116 individual clones were picked and mated with a yeast strain that contains (i) the HBV12 target in a reporter plasmid (ii) an expression plasmid containing a variant that cleaves the HBV12.3 target (30S, 32R, 33S, 44D, 68Y, 70S, 75S, 77R or KSRSQS/DYSSR (SEQ ID NO: 621) according to the nomenclature of Table L).

After mating with this yeast strain, >200 clones were found to cleave the HBV12 target more efficiently than the original variants. An example of positives is shown in FIG. 61. The sequence of seven of the best I-CreI variants cleaving the HBV12 target when forming a heterodimer with the KSRSQS/DYSSR variant are listed in Table LVI.

TABLE LVI
Functional variant combinations displaying strong cleavage activity
for HBV12.

| | | Optimized* Variants HBV12.4 |
|---|---|---|
| | | (SEQ ID NO: 672 to 678) |
| VARIANT HBV12.3 | I-CreI 28K 30S 32R 33S 38Q 40S | 32H 33C 40R 44R 68Y 70S 75N 77Q |
| | 44D 68Y 70S 75S | 19S 32H 33C 44R 68Y 70S 75D 77R |
| | 77R(KSRSQS/DYSSR) | 32H 33C 44R 68Y 70S 75Y 77Q 87L |
| | | 32H 33C 44R 68Y 70S 75D 77N 80K |
| | | 32H 33C 44R 68Y 70S 75D 77Q 87L 105A 151A |
| | | 32H 33C 44R 54L 68Y 70S 75D 77Q |
| | | 32H 33C 44R 68Y 70S 75D 77Q 87L 117K |

*Mutations resulting from site-directed mutagenesis are in bold.

### EXAMPLE 16

### Strategy for Engineering Novel Meganucleases Cleaving the HBV8 Target from the Hepatitis B Genome

HBV8 is a 22 bp (non-palindromic) target located in the coding sequence of the core protein gene in the Hepatitis B genome. The target sequence corresponds to positions 1908-1929 of the Hepatitis B genome (accession number X70185, FIG. 84).

The HBV8 sequence is partly a patchwork of the 10TGA_P, 10CAA_P, 5CTT_P and 5_TCT_P targets (FIG. 62) which are cleaved by previously identified meganucleases, obtained as described in International PCT Applications WO 2006/097784 and WO 2006/097853; Arnould et al., J. Mol. Biol., 2006, 355, 443-458; Smith et al., Nucleic Acids Res., 2006. Thus the inventors set out to determine whether HBV8 could be cleaved by combinatorial variants resulting from these previously identified meganucleases.

The 10TGA_P, 10CAA_P, 5CTT_P and 5_TCT_P target sequences are 24 bp derivatives of C1221, a palindromic sequence cleaved by I-CreI (Arnould et al., precited). However, the structure of I-CreI bound to its DNA target suggests that the two external base pairs of these targets (positions −12 and 12) have no impact on binding and cleavage (Chevalier et al., Nat. Struct. Biol., 2001, 8, 312-316; Chevalier and Stoddard, Nucleic Acids Res., 2001, 29, 3757-3774; Chevalier et al., J. Mol. Biol., 2003, 329, 253-269), and in this study, only positions −11 to 11 were considered. Consequently, the HBV8 series of targets were defined as 22 bp sequences instead of 24 bp. HBV8 differs from C1221 in the 4 bp central region. According to the structure of the I-CreI protein bound to its target, there is no contact between the 4 central base pairs (positions −2 to 2) and the I-CreI protein (Chevalier et al., Nat. Struct. Biol., 2001, 8, 312-316; Chevalier and Stoddard, Nucleic Acids Res., 2001, 29, 3757-3774; Chevalier et al., J. Mol. Biol., 2003, 329, 253-269). Thus, the bases at these positions should not impact the binding efficiency. However, they could affect cleavage, which results from two nicks at the edge of this region. Thus, the ataa sequence in −2 to 2 was first substituted with the gtac sequence from C1221, resulting in target HBV8.2 (FIG. 62). Then, two palindromic targets, HBV8.3 and HBV8.4, were derived from HBV8.2 (FIG. 62). Since HBV8.3 and HBV8.4 are palindromic, they should be cleaved by homodimeric proteins. Thus, proteins able to cleave the HBV8.3 and HBV8.4 sequences as homodimers were first designed (Examples 17, 18 and 19). In order to improve the weak cleavage activity of HBV8.4 variants, a series of variants cleaving HBV8.4 was subjected to random mutagenesis and screened for cleavage activity of the HBV8 target when co-expressed with a protein cleaving HBV8.3 (Example 20). Cleavage activity of the HBV8 target could be observed for these heterodimers. To further improve cleavage activity for the HBV8 target, HBV8.4 variants were optimized by site-directed mutagenesis and used to form novel heterodimers that were screened against the HBV8 target (Example 21). Improved cleavage activity of the HBV8 target could be observed for these heterodimers. Chosen heterodimers were then cloned into mammalian expression vectors and screened against the HBV8 target in CHO cells (Example 22). Strong cleavage activity for the HBV8 target could be observed for these heterodimers in mammalian cells.

### EXAMPLE 17

### Identification of Meganucleases Cleaving HBV8.3

This example shows that I-CreI variants can cut the HBV8.3 DNA target sequence derived from the left part of the HBV8.2 target in a palindromic form (FIG. 62). Target sequences described in this example are 22 bp palindromic sequences. Therefore, they will be described only by the first 11 nucleotides, followed by the suffix _P (For example, target HBV8.3 will be noted ttgacccttgt_P).

HBV8.3 is similar to 10TGA_P at positions ±1, ±2, ±4, ±6, ±8, ±9 and ±10 and to 5CTT_P at positions ±1, ±2, ±3, ±4, ±5, ±6 and ±8. It was hypothesized that positions ±7 and ±11 would have little effect on the binding and cleavage activity. Variants able to cleave the 10TGA_P target were obtained by mutagenesis of I-CreI N75 or D75, at positions 28, 30, 32, 33, 38, 40 and 70, as described previously in Smith et al. Nucleic Acids Res., 2006, 34, e149; International PCT Applications WO 2007/060495 and WO 2007/049156. Variants able to cleave 5CTT_P were obtained by mutagenesis on I-CreI N75 at positions 24, 44, 68, 70, 75 and 77 as described in Arnould et al., J. Mol. Biol., 2006, 355, 443-458; Smith et al. Nucleic Acids Res., 2006, 34, e149; International PCT Applications WO 2006/097784, WO 2006/097853, WO 2007/060495 and WO 2007/049156.

Both sets of proteins are mutated at position 70. However, the existence of two separable functional subdomains was hypothesized. This implies that this position has little impact on the specificity at bases 10 to 8 of the target. Mutations at positions 24 found in variants cleaving the 5CT_P target will be lost during the combinatorial process. But it was hypothesized that this will have little impact on the capacity of the combined variants to cleave the HBV8.3 target.

Therefore, to check whether combined variants could cleave the HBV8.3 target, mutations at positions 44, 68, 70, 75 and 77 from proteins cleaving 5CTT_P were combined with the 28, 30, 32, 33, 38 and 40 mutations from proteins cleaving 10TGA_P.

A) Material and Methods

a) Construction of Target Vector

The target was cloned as follows: an oligonucleotide corresponding to the HBV8.3 target sequence flanked by gateway cloning sequences was ordered from PROLIGO: 5′ tggcatacaagtttattgacccttgtacaagggtcaatcaatcgtctgtca 3′ (SEQ ID NO: 687). Double-stranded target DNA, generated by PCR amplification of the single stranded oligonucleotide, was cloned using the Gateway protocol (INVITROGEN) into the yeast reporter vector (pCLS1055, FIG. 4). Yeast reporter vector was transformed into Saccharomyces cerevisiae strain FYBL2-7B (MATα, ura3a851, trp1Δ63, leu2Δ1, lys2Δ202), resulting in a reporter strain.

b) Mating of Meganuclease Expressing Clones and Screening in Yeast

I-CreI variants cleaving 10TGA_P or 5CTT_P were previously identified, as described in Smith et al. Nucleic Acids Res., 2006, 34, e149; International PCT Applications WO 2007/060495 and WO 2007/049156, and Arnould et al., J. Mol. Biol., 2006, 355, 443-458; International PCT Applications WO 2006/097784 and WO 2006/097853, respectively for the 10TGA_P and 5CTT_P targets. In order to generate I-CreI derived coding sequences containing mutations from both series, separate overlapping PCR reactions were carried out that amplify the 5′ end (aa positions 1-43) or the 3′ end (positions 39-167) of the I-CreI coding sequence. For both the 5′ and 3′ end, PCR amplification is carried out using primers (Gal10F 5′-gcaactttagtgctgacacatacagg-3′ (SEQ ID NO: 263) or Gal10R 5′-acaaccttgattggagacttgacc-3′(SEQ ID NO: 264)) specific to the vector (pCLS0542, FIG. 5) and primers (assF 5′-ctannnttgaccttt-3′ (SEQ ID NO: 265) or assR 5′-aaaggtcaannntag-3′(SEQ ID NO: 266)), where nnn codes for residue 40, specific to the I-CreI coding sequence for amino acids 39-43. The PCR fragments resulting from the amplification reaction realized with the same primers and with the same coding sequence for residue 40 were pooled. Then, each pool of PCR fragments resulting from the reaction with primers Gal10F and assR or assF and Gal10R was mixed in an equimolar ratio. Finally, approximately 25 ng of each final pool of the two overlapping PCR fragments and 75 ng of vector DNA (pCLS0542, FIG. 5) linearized by digestion with NcoI and EagI were used to transform the yeast Saccharomyces cerevisiae strain FYC2-6A (MATα, trp1Δ63, leu2Δ1, his3Δ200) using a high efficiency LiAc transformation protocol (Gietz and Woods, Methods Enzymol., 2002, 350, 87-96). An intact coding sequence containing both groups of mutations is generated by in vivo homologous recombination in yeast.

c) Mating of Meganuclease Expressing Clones and Screening in Yeast

Screening was performed as described previously (Arnould et al., J. Mol. Biol., 2006, 355, 443-458). Mating was performed using a colony gridder (QpixII, GENETIX). Variants were gridded on nylon filters covering YPD plates, using a low gridding density (4-6 spots/cm²). A second gridding process was performed on the same filters to spot a second layer consisting of the reporter-harboring yeast strain for the target of interest. Membranes were placed on solid agar YPD rich medium, and incubated at 30° C. for one night, to allow mating. Next, filters were transferred to synthetic medium, lacking leucine and tryptophan, with galactose (2%) as a carbon source, and incubated for five days at 37° C., to select for diploids carrying the expression and target vectors. After 5 days, filters were placed on solid agarose medium with 0.02% X-Gal in 0.5 M sodium phosphate buffer, pH7.0, 0.1% SDS, 6% dimethyl formamide (DMF), 7 mM β-mercaptoethanol, 1% agarose, and incubated at 37° C., to monitor β-galactosidase activity. Results were analyzed by scanning and quantification was performed using appropriate software.

d) Sequencing of Variants

To recover the variant expression plasmids, yeast DNA was extracted using standard protocols and used to transform E. coli. Sequencing of variant ORFs was then performed on the plasmids by MILLEGEN SA. Alternatively, ORFs were amplified from yeast DNA by PCR (Akada et al., Biotechniques, 2000, 28, 668-670), and sequencing was performed directly on the PCR product by MILLEGEN SA.

B) Results

I-CreI combinatorial variants were constructed by associating mutations at positions 44, 68, 70, 75 and 77 from proteins cleaving 5CTT_P with the mutations at 28, 30, 32, 33, 38 and 40 from proteins cleaving 10TGA_P on the I-CreI scaffold, resulting in a library of complexity 1600. Examples of combinatorial variants are displayed in Table LVII. This library was transformed into yeast and 2304 clones (1.4 times the diversity) were screened for cleavage against the HBV8.3 DNA target (ttgacccttgt_P, SEQ ID NO: 687). A total of 160 positive clones were found to cleave HBV8.3. Sequencing and validation by secondary screening of 79 of the best I-CreI variants resulted in the identification of 55 different novel endonucleases. Examples of positives are shown in FIG. 63. The sequence of several of the variants identified display non parental combinations at positions 28, 30, 32, 33, 38, 40 or 44, 68, 70, 75, 77 as well as additional mutations (see examples Table LVIII). Such combinations likely result from PCR artifacts during the combinatorial process. Alternatively, the variants may be I-CreI combined variants resulting from micro-recombination between two original variants during in vive homologous recombination in yeast.

TABLE LVII
Panel of variants* theoretically present in the combinatorial library

| Amino acids at | | | | | | | | | | |
|---|---|---|---|---|---|---|---|---|---|---|
| positions 44, 68, | | | | | | | | | | |
| 70, 75 and 77 | | | | | | | | | | |
| (ex: RYSDN | | | | | | | | | | |
| stands for R44, | Amino acids at positions 28, 30, 32, 33, 38 and 40 | | | | | | | | | |
| Y68, S70, D75 | (ex: KDSRQS stands for K28, D30, S32, R33, Q38 and S40) | | | | | | | | | |
| and N77) | KNRAQS | KNSCSS | KASTQS | KNSVHS | KHSCQS | KNATQS | KNSGTS | KNDCQS | KNSTSS | KNNGQS |
| RYSDN | + | + | + | + | + | + | + | + | + | |
| RYSDQ | | | | | | | | | | |
| RNSNN | | | | | | | | | | |
| RYSNN | | + | | + | + | | | | | |
| RYSYI | | | | | | | | | | |
| RNSDR | | | | | | | | | | |
| RYSNI | | | | | | | | | | |
| RASDR | | | | | | | | | | |
| RTSNN | | | | + | | | | | | |
| RSSNN | | + | | + | | + | | | | |
| RYSHI | | | | | | | | | | |
| RASNN | | | | | | | | | | |
| QNSQR | | | | | | | | | | |
| KASDV | | | | | | | | | | |
| KTSDR | | | | | | | | | | |
| QASNR | | | | | | | | | | |
| KTSDI | | | | | | | | | | |
| RYSYN | | + | | + | + | | | | | + |
| KESDR | | | | | | | | | | |
| KSSDI | | | | | | | | | | |

*Only 200 out of the 1600 combinations are displayed.
+ indicates that a functional combinatorial variant cleaving the HBV8.3 target was found among the identified positives.

TABLE LVIII
I-CreI variants capable of cleaving the HBV8.3 DNA target.

| Amino acids at positions 28, 30, 32, | |
|---|---|
| 33, 38, 40/44, 68, 70, 75 and 77 | |
| of the I-CreI variants | |
| (ex; KNSARS/RYSDN stands for | SEQ |
| K28, N30, S32, A33, R38, S40/R44, | ID |
| Y68, S70, D75 and N77) | NO: |
| KNSARS/RYSDN | 690 |
| KNSCRS/RYSDN | 691 |
| KHSCHS/RYSYN | 692 |
| KNSATS/RAYSN | 693 |
| KNRAQS/RTSNN + 158E | 694 |
| KNSCSS/RYSNN + 162F | 695 |

### EXAMPLE 18

### Identification of Meganucleases Cleaving HBV8.4

This example shows that I-CreI variants can cleave the HBV8.4 DNA target sequence derived from the right part of the HBV8.2 target in a palindromic form (FIG. 62). All target sequences described in this example are 22 bp palindromic sequences. Therefore, they will be described only by the first 11 nucleotides, followed by the suffix _P (for example, HBV8.4 will be called ccaaattctgt_P).

HBV8.4 is similar to 5TCT_P at positions ±1, ±2, ±3, ±4, ±5, ±7, ±8, ±9 and ±11 and to 10CAA_P at positions ±1, ±2, ±7, ±8, ±9, ±10 and ±11. It was hypothesized that position −6 would have little effect on the binding and cleavage activity. Variants able to cleave 5TCT_P were obtained by mutagenesis of I-CreI N75 at positions 24, 44, 68, 70, 75 and 77, as described previously (Arnould et al., J. Mol. Biol., 2006, 355, 443-458; Smith et al. Nucleic Acids Res., 2006, 34, e149; International PCT Applications WO 2006/097784, WO 2006/097853, WO 2007/060495 and WO 2007/049156). Variants able to cleave the 10CAA_P target were obtained by mutagenesis of I-CreI N75 or D75, at positions 28, 30, 32, 33, 38 and 40, as described previously in Smith et al. Nucleic Acids Res., 2006, 34, e149; International PCT Applications WO 2007/060495 and WO 2007/049156.

Mutations at position 24 found in variants cleaving the 5TCT_P target will be lost during the combinatorial process. But it was hypothesized that this will have little impact on the capacity of the combined variants to cleave the HBV8.4 target.

Therefore, to check whether combined variants could cleave the HBV8.4 target, mutations at positions 44, 68, 70, 75 and 77 from proteins cleaving 5TCT_P were combined with the 28, 30, 32, 33, 38 and 40 mutations from proteins cleaving 10CAA_P.

A) Material and Methods

a) Construction of Target Vector

The experimental procedure is as described in Example 17, with the exception that an oligonucleotide corresponding to the HBV8.4 target sequence was used: 5′ tggcatacaagttttccaaattctgtacagaatttggacaatcgtctgtca 3′ (SEQ ID NO: 696).

b) Construction of Combinatorial Variants

I-CreI variants cleaving 10CAA_P or 5TCT_P were previously identified, as described in Smith et al. Nucleic Acids Res., 2006, 34, e149; International PCT Applications WO 2007/060495 and WO 2007/049156, and Arnould et al., J. Mol. Biol., 2006, 355, 443-458; International PCT Applications WO 2006/097784 and WO 2006/097853, respectively for the 10CAA_P and 5TCT_P targets. In order to generate I-CreI derived coding sequences containing mutations from both series, separate overlapping PCR reactions were carried out that amplify the 5′ end (aa positions 1-43) or the 3′ end (positions 39-167) of the I-CreI coding sequence. For both the 5′ and 3′ end, PCR amplification is carried out using primers (Gal10F 5′-gcaactttagtgctgacacatacagg-3′ (SEQ ID NO: 263) or Gal10R 5′-acaaccttgattggagacttgacc-3′ (SEQ ID NO: 264) specific to the vector (pCLS1107, FIG. 6) and primers (assF 5′-ctannnttgaccttt-3′ (SEQ ID NO: 265) or assR 5′-aaaggtcaannntag-3′(SEQ ID NO: 266), where nnn codes for residue 40, specific to the I-CreI coding sequence for amino acids 39-43. The PCR fragments resulting from the amplification reaction realized with the same primers and with the same coding sequence for residue 40 were pooled. Then, each pool of PCR fragments resulting from the reaction with primers Gal10F and assR or assF and Gal10R was mixed in an equimolar ratio. Finally, approximately 25 ng of each final pool of the two overlapping PCR fragments and 75 ng of vector DNA (pCLS1107, FIG. 6) linearized by digestion with DraIII and NgoMIV were used to transform the yeast Saccharomyces cerevisiae strain FYC2-6A (MATα, trp1Δ63, leu2Δ1, his3Δ200) using a high efficiency LiAc transformation protocol (Gietz and Woods, Methods Enzymol., 2002, 350, 87-96). An intact coding sequence containing both groups of mutations is generated by in vivo homologous recombination in yeast.

c) Mating of Meganuclease Expressing Clones and Screening in Yeast

Screening was performed as described previously (Arnould et al., J. Mol. Biol., 2006, 355, 443-458). Mating was performed using a colony gridder (QpixII, GENETIX). Variants were gridded on nylon filters covering YPD plates, using a low gridding density (4-6 spots/cm²). A second gridding process was performed on the same filters to spot a second layer consisting of the reporter-harboring yeast strain. Membranes were placed on solid agar YPD rich medium, and incubated at 30° C. for one night, to allow mating. Next, filters were transferred to synthetic medium, lacking tryptophan, including G418, with galactose (2%) as a carbon source, and incubated for five days at 37° C., to select for diploids carrying the expression and target vectors. After 5 days, filters were placed on solid agarose medium with 0.02% X-Gal in 0.5 M sodium phosphate buffer, pH7.0, 0.1% SDS, 6% dimethyl formamide (DMF), 7 mM β-mercaptoethanol, 1% agarose, and incubated at 37° C., to monitor β-galactosidase activity. Results were analyzed by scanning and quantification was performed using appropriate software. d) Sequencing of variants

The experimental procedure is as described in Example 17.

B) Results

I-CreI combinatorial variants were constructed by associating mutations at positions 44, 68, 70, 75 and 77 from proteins cleaving 5TCT_P with the mutations 28, 30, 32, 33, 38 and 40 from proteins cleaving 10CAA_P on the I-CreI scaffold, resulting in a library of complexity 1600. Examples of combinatorial variants are displayed in Table LIX. This library was transformed into yeast and 2304 clones (1.4 times the diversity) were screened for cleavage against the HBV8.4 DNA target (ccaaattctgt_P SEQ ID NO: 688). Two positive clones were found, which after sequencing turned out to correspond to two different novel endonuclease variants (Table LIX and Table LX). Examples of positives are shown in FIG. 64. One of these two variants display non parental combinations at positions 28, 30, 32, 33, 38, 40 or 44, 68, 70, 75, 77. Such combinations likely result from PCR artifacts during the combinatorial process. Alternatively, the variants may be I-CreI combined variants resulting from micro-recombination between two original variants during in vivo homologous recombination in yeast.

TABLE LIX
Panel of variants* theoretically present in the combinatorial library

| Amino acids at | | | | | | | | | | |
|---|---|---|---|---|---|---|---|---|---|---|
| positions 44, 68, | | | | | | | | | | |
| 70, 75 and 77 | | | | | | | | | | |
| (ex: KGGNI | | | | | | | | | | |
| stands for K44, | Amino acids at positions 28, 30, 32, 33, 38 and 40 | | | | | | | | | |
| G68, G70, N75 | (ex: KNSGQS stands for K28, N30, S32, G33, Q38 and S40) | | | | | | | | | |
| and I77) | KNSGQS | KRDYQQ | KNKTQS | KNGHQS | KNAHQS | KNEYQS | KNTQQS | KNRDQS | KNSCQQ | KNSHQQ |

| | |
|---|---|
| KGGNI | |
| KNANI | |
| KADNI | |
| KNENI | |
| KGSNI | |
| QRSNK | + |
| KYSNI | |
| PCSYT | |
| KESNR | |
| QASNR | |
| KASNI | |
| QQSNR | |
| QNSNR | |
| QDSRR | |
| HYSNH | |

*Only 150 out of the 1600 combinations are displayed.
+ indicates that a functional combinatorial variant cleaving the HBV8.4 target was found among the identified positives.

TABLE LX
I-CreI variants with additional mutations capable of cleaving the
HBV8.4 DNA target.

| Amino acids at positions 28, 30, 32, | |
|---|---|
| 33, 38, 40/44, 68, 70, 75 and 77 | |
| of the I-CreI variants | |
| (ex: KNSHQQ/QRSNK + 163Q | |
| stands for K28, N30, S32, H33, | SEQ |
| Q38, Q40/Q44, R68, S70, N75 and | ID |
| K77, 163Q) | NO: |
| KNSHQQ/QRSNK + 163Q | 697 |

### EXAMPLE 19

### Identification of Meganucleases Cleaving HBV8.4 Through the Generation of Combinatorial Variants Containing 105a and 132V Substitutions

A combinatorial library containing selected amino-acid substitutions was produced as an alternative approach to generating I-CreI variants that cleave the HBV8.4 DNA target.

Two amino-acid substitutions have been found in previous studies to enhance the activity of I-CreI derivatives and could easily be incorporated into a combinatorial library: these mutations correspond to the replacement of Valine 105 with Alanine (V105A) and Isoleucine 132 with Valine (I132V). Both of these substitutions were introduced into all variants of a combinatorial library containing mutations at positions 44, 68, 70, 75 and 77 from proteins cleaving 5TCT_P combined with the 28, 30, 32, 33, 38 and 40 mutations from proteins cleaving 10CAA_P.

A) Material and Methods

a) Construction of Combinatorial Variants

I-CreI variants cleaving 10CAA_P or 5TCT_P were previously identified, as described in Smith et al. Nucleic Acids Res., 2006, 34, e149; International PCT Applications WO 2007/060495 and WO 2007/049156, and Arnould et al., J. Mol. Biol., 2006, 355, 443-458; International PCT Applications WO 2006/097784 and WO 2006/097853, respectively for the 10CAA_P and 5TCT_P targets. In order to generate I-CreI derived coding sequences containing mutations from both series, separate overlapping PCR reactions were carried out that amplify the 5′ end (aa positions 1-43) or the 3′ end (positions 39-104) of the I-CreI coding sequence. The remaining 3′ sequences of I-CreI containing the 105A and 132V substitutions are present in the vector pCLS1884. For both the 5′ and 3′ end amplifications, PCR is carried out using primers (Gal10F 5′-gcaactttagtgctgacacatacagg-3′ (SEQ ID NO: 263) or CreRevBsgI 5′-caggtttgcctgtttctgtttcagtttcagaaacggctg-3′ (SEQ ID NO: 698)) containing homology to the vector (pCLS1884, FIG. 65) and primers (assF 5′-ctannnttgaccttt-3′ (SEQ ID NO: 265) or assR 5′-aaaggtcaannntag-3′(SEQ ID NO: 266)), where nnn codes for residue 40, specific to the I-CreI coding sequence for amino acids 39-43. The PCR fragments resulting from the amplification reaction realized with the same primers and with the same coding sequence for residue 40 were pooled. Then, each pool of PCR fragments resulting from the reaction with primers Gal10F and assR or assF and CreRevBsgI was mixed in an equimolar ratio. Finally, approximately 25 ng of each final pool of the two overlapping PCR fragments and 75 ng of vector DNA (pCLS1884, FIG. 65) linearized by digestion with NcoI and BsgI were used to transform the yeast Saccharomyces cerevisiae strain FYC2-6A (MATα, trp1Δ63, leu2Δ1, his3Δ200) using a high efficiency LiAc transformation protocol (Gietz and Woods, Methods Enzymol., 2002, 350, 87-96). An intact coding sequence containing both groups of mutations as well as the 105A and 132V substitutions is generated by in vivo homologous recombination in yeast.

b) Mating of Meganuclease Expressing Clones and Screening in Yeast

The experimental procedure is as described in Example 18.

c) Sequencing of Variants

The experimental procedure is as described in Example 17.

B) Results

I-CreI combinatorial variants were constructed by associating mutations at positions 44, 68, 70, 75 and 77 from proteins cleaving 5TCT_P with the mutations 28, 30, 32, 33, 38 and 40 from proteins cleaving 10CAA_P on an I-CreI scaffold containing the amino acid substitutions 105A and 132V, resulting in a library of complexity 1600. This library was transformed into yeast and 2304 clones (1.4 times the diversity) were screened for cleavage against the HBV8.4 DNA target (ccaaattctgt_P). Four positive clones were found, which after sequencing turned out to correspond to four different novel endonuclease variants (Table LXI). Examples of positives are shown in FIG. 66. All four variants contain the 105A and 132V substitutions as well as display non parental combinations at positions 28, 30, 32, 33, 38, 40 or 44, 68, 70, 75, 77. Such combinations likely result from PCR artifacts during the combinatorial process. Alternatively, the variants may be I-CreI combined variants resulting from micro-recombination between two original variants during in vivo homologous recombination in yeast.

TABLE LXI
I-CreI variants with additional mutations capable of cleaving the
HBV8.4 DNA target.

| Amino acids at positions 28, 30, 32, | |
|---|---|
| 33, 38, 40/44, 68, 70, 75 and 77 | |
| of the I-CreI variants | |
| (ex: KNSHQQ/KASNI + | |
| 105A + 132V stands for K28, N30, | SEQ |
| S32, H33, Q38, Q40/K44, A68, | ID |
| S70, N75 and I77, 105A, 132V) | NO: |
| KNSHQQ/KASNI + 105A + 132V | 699 |
| KNSHQQ/KNANI + 105A + 132V | 700 |
| KNEYQS/QASNR + 105A + 132V | 701 |
| KNEYQS/QSSNR + 105A + 132V | 702 |

### EXAMPLE 20

### Improvement of Meganucleases Cleaving HBV8.4 by Random Mutagenesis

I-CreI variants able to cleave the palindromic HBV8.4 target have been previously identified in Examples 18 and 19. However, the HBV8.4 variants display very weak activity with the HBV8.4 target. In this example, it was determined if the activity of the HBV8.4 meganucleases could be increased and at the same time it was tested whether they could cleave HBV8 efficiently when co-expressed with a protein cleaving HBV8.3. The six combinatorial variants cleaving HBV8.4 were mutagenized by random mutagenesis, and in a second step, it was assessed whether they could cleave HBV8 when co-expressed with a protein cleaving HBV8.3.

A) Material and Methods

a) Construction of Target Vector

The experimental procedure is as described in Example 17, with the exception that an oligonucleotide corresponding to the HBV8 target sequence: 5′ tggcatacaagtttattgacccttataaagaatttggacaatcgtctgtca 3′ (SEQ ID NO: 685) was used.

b) Construction of Libraries by Random Mutagenesis

Random mutagenesis was performed on a pool of chosen variants, by PCR using Mn²⁺. PCR reactions were carried out that amplify the I-CreI coding sequence using the primers preATGCreFor (5′-gcataaattactatacttctatagacacgcaaacacaaatacacagcggccttgccacc-3′; SEQ ID NO: 169) and I-CreIpostRev (5′-ggctcgaggagctcgtctagaggatcgctcgagttatcagtcggccgc-3′; SEQ ID NO: 170). Approximately 25 ng of the PCR product and 75 ng of vector DNA (pCLS1107, FIG. 6) linearized by digestion with DraIII and NgoMIV were used to transform the yeast Saccharomyces cerevisiae strain FYC2-6A (MATα, trp1Δ63, leu2Δ1, his3Δ200) using a high efficiency LiAc transformation protocol (Gietz and Woods, Methods Enzymol., 2002, 350, 87-96). Expression plasmids containing an intact coding sequence for the I-CreI variant were generated by in vivo homologous recombination in yeast.
c) Variant-Target Yeast Strains

The yeast strain FYBL2-7B (MAT a, ura3Δ851, trp1Δ63, leu2Δ1, lys2Δ202) containing the HBV8 target in the yeast reporter vector (pCLS1055, FIG. 4) was transformed with variants, in the leucine vector (pCLS0542), cutting the HBV8.3 target, using a high efficiency LiAc transformation protocol.

d) Matins of Meganuclease Expressing Clones and Screening in Yeast

Mating was performed using a colony gridder (QpixII, Genetix). Variants were gridded on nylon filters covering YPD plates, using a low gridding density (about 4 spots/cm²). A second gridding process was performed on the same filters to spot a second layer consisting of a variant-target yeast strain for the target of interest. Membranes were placed on solid agar YPD rich medium, and incubated at 30° C. for one night, to allow mating. Next, filters were transferred to synthetic medium, lacking leucine and tryptophan, including G418, with galactose (2%) as a carbon source, and incubated for five days at 37° C., to select for diploids carrying the expression and target vectors. After 5 days, filters were placed on solid agarose medium with 0.02% X-Gal in 0.5 M sodium phosphate buffer, pH7.0, 0.1% SDS, 6% dimethyl formamide (DMF), 7 mM (β-mercaptoethanol, 1% agarose, and incubated at 37° C., to monitor β-galactosidase activity. Results were analyzed by scanning and quantification was performed using appropriate software.

e) Sequencing of Variants

The experimental procedure is as described in Example 17.

B) Results

Six variants cleaving HBV8.4 (I-CreI 33H, 40Q, 70S, 75N, 77K, I-CreI 33H, 40Q, 70S, 75N, 77K, 163Q, I-CreI 33H, 40Q, 44K, 68A, 70S, 75N, 105A, 132V, I-CreI 33H, 40Q, 44K, 68N, 70A, 75N, 105A, 132V, I-CreI 32E, 68A, 70S, 75N, 77R, 105A, 132V and I-CreI 32E, 68S, 70S, 75N, 77R, 105A, 132V also called KNSHQQ/QRSNK (SEQ ID NO: 704), KNSHQQ/QRSNK+163Q (SEQ ID NO: 697), KNSHQQ/KASNI +105A+132V (SEQ ID NO: 699), KNSHQQ/KNANI+105A132V (SEQ ID NO: 700), KNEYQS/QASNR +105A+132V (SEQ ID NO: 701), and KNEYQS/QSSNR +105A+132V (SEQ ID NO: 702), respectively, according to the nomenclature of Table LXIX, LXX and LXXI) were pooled, randomly mutagenized and transformed into yeast. 2304 transformed clones were then mated with a yeast strain that contains (i) the HBV8 target in a reporter plasmid (ii) an expression plasmid containing a variant that cleaves the HBV8.3 target (I-CreI 33C, 38R, 44R, 68Y, 70S, 75D, 77N or KNSCRS/RYSDN (SEQ ID NO: 691) according to the nomenclature of Table LVIII). After mating with this yeast strain, 379 clones were found to cleave the HBV8 target. Thus, 379 positives contained proteins able to form heterodimers with KNSCRS/RYSDN with cleavage activity for the HBV8 target. An example of positives is shown in FIG. 17. Sequencing of the strongest 186 positive clones indicates that 32 distinct variants were identified (Examples listed in Table LXII).

TABLE LXII
Functional variant combinations displaying cleavage activity for
HBV8.

| | | Optimized Variants HBV8.4 |
|---|---|---|
| | | (SEQ ID NO: 705 to 711) |
| VARIANT HBV8.3 | I-CreI 33C 38R 44R 68Y 70S 75D | 33H 40Q 70S 75N 77K 105A 132V |
| | 77N (KNSCRS/RYSDN) (SEQ ID | 33H 40Q 68A 70S 75N 77R 105A 132V |
| | NO: 691 | 33H 40Q 68A 70S 75N 77R 132V |
| | | 33H 40Q 70S 75N 77K 132V |
| | | 33H 40Q 68S 70S 75N 77R 105A 132V |
| | | 33H 40Q 70S 75N 77K 105A |
| | | 33H 40Q 68A 70S 75N 77R 105A |

*Mutations resulting from mutagenesis are in bold.

### EXAMPLE 21

### Improvement of Meganucleases Cleaving HBV8 by Site-Directed Mutagenesis of Proteins Cleaving HBV8.4 and Assembly with Proteins Cleaving HBV8.3

The I-CreI optimized variants cleaving HBV8.4 described in Example 20 were further mutagenized by introducing selected amino-acid substitutions in the proteins and screening for more efficient variants cleaving HBV8 in combination with a variant cleaving HBV8.3.

Two amino-acid substitutions found in previous studies to enhance the activity of I-CreI derivatives were introduced into HBV8.4 variants: these mutations correspond to the replacement of Glycine 19 with Serine (G19S) and Phenylalanine 54 with Leucine (F54L). These mutations were individually introduced into the coding sequence of proteins cleaving HBV8.4, and the resulting proteins were tested for their ability to induce cleavage of the HBV8 target, upon co-expression with a variant cleaving HBV8.3.

A) Material and Methods

a) Site-Directed Mutagenesis

Site-directed mutagenesis libraries were created by PCR on a pool of chosen variants. For example, to introduce the G19S substitution into the coding sequence of the variants, two separate overlapping PCR reactions were carried out that amplify the 5′ end (residues 1-24) or the 3′ end (residues 14-167) of the I-CreI coding sequence. For both the 5′ and 3′ end, PCR amplification is carried out using a primer with homology to the vector (Gal10F 5′-gcaactttagtgctgacacatacagg-3′ (SEQ ID NO: 263) or Gal10R 5′-acaaccttgattggagacttgacc-3′(SEQ ID NO: 264)) and a primer specific to the I-CreI coding sequence for amino acids 14-24 that contains the substitution mutation G19S (G19SF 5′-gccggctttgtggactctgacggtagcatcatc-3′ (SEQ ID NO: 653) or G19SR 5′-gatgatgctaccgtcagagtccacaaagccggc-3′(SEQ ID NO: 654). The resulting PCR products contain 33 bp of homology with each other. The PCR fragments were purified. Approximately 25 ng of each of the two overlapping PCR fragments and 75 ng of vector DNA (pCLS1107, FIG. 6) linearized by digestion with DraIII and NgoMIV were used to transform the yeast Saccharomyces cerevisiae strain FYC2-6A (MATα, trp1Δ63, leu2Δ1, his3Δ200) using a high efficiency LiAc transformation protocol (Gietz and Woods, Methods Enzymol., 2002, 350, 87-96). Intact coding sequences containing the G19S substitution are generated in vivo homologous recombination in yeast.

The same strategy is used with the following pair of oligonucleotides to create the library containing the F54L substitution:

*F54LF: 5′-acccagcgccgttggctgctggacaaactagtg-3′ and F54LR: 5′-cactagtttgtccagcagccaacggcgctgggt-3′ (SEQ ID NO: 655 and 656);

b) Mating of Meganuclease Expressing Clones and Screening in Yeast

The experimental procedure is as described in Example 20.

c) Sequencing of Variants

The experimental procedure is as described in Example 17.

B) Results

Libraries containing one of two amino-acid substitutions (G19S or F54L) were constructed on a pool of five variants cleaving HBV8.4 (33H, 40Q, 70S, 75N, 77K, 105A, 132V (SEQ ID NO: 705); 33H, 40Q, 68A, 70S, 75N, 77R, 105A, 132V (SEQ ID NO: 706); 33H, 40Q, 68A, 70S, 75N, 77R, 132V (SEQ ID NO: 707); 33H, 40Q, 70S, 75N, 77K, 132V (SEQ ID NO: 708) and 33H, 40Q, 68S, 70S, 75N77R, 105A, 132V (SEQ ID NO: 709), according to the nomenclature of Table LXII). 576 transformed clones for each library were then mated with a yeast strain that contains (i) the HBV8 target in a reporter plasmid (ii) an expression plasmid containing a variant that cleaves the HBV8.3 target (I-CreI 33C, 38R, 44R, 68Y, 70S, 75D, 77N or KNSCRS/RYSDN (SEQ ID NO: 691) according to the nomenclature of Table LVIII).

After mating with this yeast strain, a large number of clones (>100) in the library containing amino-acid substitution Glycine 19 with Serine (G19S), were found to cleave the HBV8 target more efficiently than the original variants. An Example of positives is shown in FIG. 68. The sequence of the four best I-CreI variants cleaving the HBV8 target when forming a heterodimer with the KNSCRS/RYSDN variant are listed in Table LXIII.

TABLE LXIII
Functional variant combinations displaying strong cleavage activity
for HBV8.

| | | Optimized* Variants HBV8.4 |
|---|---|---|
| | | (SEQ ID NO: 712 to 715) |
| VARIANT HBV8.3 | I-CreI 33C 38R 44R | 19S 33H 40Q 43I 70S 75N 77K 105A 132V |
| | 68Y 70S 75D | 19S 33H 40Q 70S 75N 77K 105A 132V |
| | 77N(KNSCRS/RYSDN) | 19S 33H 40Q 70S 75N 77K 105A |
| | (SEQ ID NO: | 19S 33H 40Q 70S 75N 77K 132V |
| | 691) | |

*Mutations resulting from mutagenesis are in bold.

### EXAMPLE 22

### Validation of HBV8 Target Cleavage in an Extrachromosomal Model in CHO Cells

I-CreI variants able to efficiently cleave the HBV8 target in yeast when forming heterodimers were described in Examples 20 and 21. In order to further validate heterodimers displaying strong cleavage activity for the HBV8 target in yeast cells, the efficiency of chosen combinations of variants to cut the HBV8 target was analyzed, using an extrachromosomal assay in CHO cells. The screen in CHO cells is a single-strand annealing (SSA) based assay where cleavage of the target by the meganucleases induces homologous recombination and expression of a LagoZ reporter gene (a derivative of the bacterial lacZ gene).

1) Materials and Methods

a) Cloning of HBV8 Target in a Vector for CHO Screen

The target was cloned as follows: oligonucleotide corresponding to the HBV8 target sequence flanked by gateway cloning sequence was ordered from PROLIGO: 5′ tggcatacaagtttattgacccttataaagaatttggacaatcgtctgtca 3′ (SEQ ID NO: 685). Double-stranded target DNA, generated by PCR amplification of the single stranded oligonucleotide, was cloned using the Gateway protocol (INVITROGEN) into CHO reporter vector (pCLS1058, FIG. 11). Cloned target was verified by sequencing (MILLEGEN).

b) Re-Cloning of Meganucleases

The ORF of 1-CreI variants cleaving the HBV8.3 and HBV8.4 targets identified in Examples 17 and 21 were re-cloned in pCLS1768 (FIG. 29). ORFs were amplified by PCR on yeast DNA using the attB1-ICreIFor (5′-ggggacaagtttgtacaaaaaagcaggcttcgaaggagatagaaccatggccaataccaaatataacaaagagttcc-3′; SEQ ID NO: 716) and attB2-ICreIRev (5′-ggggaccactttgtacaagaaagctgggtttagtcggccgccggggaggatttcttcttctcgc-3′; SEQ ID NO: 717) primers. PCR products were cloned in the CHO expression vector pCLS1768 (FIG. 29) using the Gateway protocol (INVITROGEN). Resulting clones were verified by sequencing (MILLEGEN).

c) Extrachromosomal Assay in Mammalian Cells

CHO cells were transfected with Polyfect® transfection reagent according to the supplier's protocol (QIAGEN). 72 hours after transfection, culture medium was removed and 150 μl of lysis/revelation buffer for β-galactosidase liquid assay was added (typically 1 liter of buffer contained: 100 ml of lysis buffer (Tris-HCl 10 mM pH7.5, NaCl 150 mM, Triton X100 0.1%, BSA 0.1 mg/ml, protease inhibitors), 10 ml of Mg 100× buffer (MgCl₂ 100 mM, β-mercaptoethanol 35%), 110 ml ONPG 8 mg/ml and 780 ml of sodium phosphate 0.1M pII7.5). After incubation at 37° C., OD was measured at 420 nm. The entire process is performed on an automated Velocity11 BioCel platform.

Per assay, 150 ng of target vector was co-transfected with 12.5 ng of each one of both variants (12.5 ng of variant cleaving palindromic HBV8.3 target and 12.5 ng of variant cleaving palindromic HBV8.4 target).

2) Results

One HBV8.3 variant (I-CreI 33C, 38R, 44R, 68Y, 70S, 75D, 77N, SEQ ID NO: 691) and two HBV8.4 variants (I-CreI 19S 33H40Q 43I 70S 75N 77K 105A 132V, SEQ ID NO: 712 and I-CreI 19S 33H40Q 70S 75N 77K 105A 132V, SEQ ID NO:713) described in Examples 17 and 21 were first re-cloned in pCLS1768 (FIG. 29). Then, in order to validate the cleavage activity of the heterodimers with the HBV8 target, the I-CreI variants cleaving the HBV8.3 or HBV8.4 targets were tested together as heterodimers against the HBV8 target in the CHO extrachromosomal assay.

FIG. 69 shows the results obtained for the two heterodimers against the HBV8 target in CHO cells assay, compared to the activity of I-SceI against its target (tagggataacagggtaat, SEQ ID NO: 718). Analysis of the efficiencies of cleavage of the HBV8 sequence demonstrates that both combinations of 1-CreI variants are able to cut the HBV8 target in CHO cells with an activity similar to that of I-SceI against the I-SceI target.

### EXAMPLE 23

### Strategy for Engineering Novel Meganucleases Cleaving the HBV3 Target from the HBV Genome

HBV3 is a 22 bp (non-palindromic) target located in the coding sequence of the core protein gene in the Hepatitis B genome. The target sequence corresponds to positions 2216-2237 of the Hepatitis B genome (accession number M38636, FIG. 84).

The HBV3 sequence is partly a patchwork of the 10TGC_P, 10TCT_P, 5TAC_P and 5TCC_P targets (FIG. 70) which are cleaved by previously identified meganucleases, obtained as described in International PCT Applications WO 2006/097784 and WO 2006/097853; Arnould et al., J. Mol. Biol., 2006, 355, 443-458; Smith et al., Nucleic Acids Res., 2006. Thus the inventors set out to determine whether HBV3 could be cleaved by combinatorial variants resulting from these previously identified meganucleases.

The 10TGC_P, 10TCT_P, 5TAC_P and 5TCC_P target sequences are 24 bp derivatives of C1221, a palindromic sequence cleaved by I-CreI (Arnould et al., precited). However, the structure of I-CreI bound to its DNA target suggests that the two external base pairs of these targets (positions −12 and 12) have no impact on binding and cleavage (Chevalier et al., Nat. Struct. Biol., 2001, 8, 312-316; Chevalier and Stoddard, Nucleic Acids Res., 2001, 29, 3757-3774; Chevalier et al., J. Mol. Biol., 2003, 329, 253-269), and in this study, only positions −11 to 11 were considered. Consequently, the HBV3 series of targets were defined as 22 bp sequences instead of 24 bp. HBV3 differs from C1221 in the 4 bp central region. According to the structure of the I-CreI protein bound to its target, there is no contact between the 4 central base pairs (positions −2 to 2) and the I-CreI protein (Chevalier et al., Nat. Struct. Biol., 2001, 8, 312-316; Chevalier and Stoddard, Nucleic Acids Res., 2001, 29, 3757-3774; Chevalier et al., J. Mol. Biol., 2003, 329, 253-269). Thus, the bases at these positions should not impact the binding efficiency. However, they could affect cleavage, which results from two nicks at the edge of this region. Thus, the tttt sequence in −2 to 2 was first substituted with the gtac sequence from C1221, resulting in target HBV3.2 (FIG. 70). Then, two palindromic targets, HBV3.3 and HBV3.4, were derived from HBV3.2 (FIG. 71). Since HBV3.3 and HBV3.4 are palindromic, they should be cleaved by homodimeric proteins. In addition, to test the influence of the tttt sequence on the activity of homodimeric proteins two pseudo-palindromic targets were created, containing the tttt sequence at positions −2 to 2 (targets HBV3.5 and HBV3.6, FIG. 70). Thus, proteins able to cleave the HBV3.3 and HBV3.4 sequences as homodimers were first designed (Examples 24 and 25) and then co-expressed to obtain heterodimers cleaving HBV3.2 (Example 26). In order to obtain cleavage activity for the HBV3 target, a series of variants cleaving HBV3.3 and HBV3.4 was chosen and refined. The chosen variants were subjected to random mutagenesis, screened for activity with the HBV3.5 and HBV3.6 targets (Examples 27 and 28) and were subsequently used to form novel heterodimers that were screened against the HBV3 target (Example 29). Heterodimers could be identified with cleavage activity for the HBV3 target. To further improve the cleavage activity for the HBV3 target, a series of variants cleaving HBV3.3 and HBV3.4 was chosen, refined, cloned into mammalian expression vectors and screened against the HBV3 target in CHO cells (Examples 30, 31 and 32). Heterodimers could be identified with strong cleavage activity for the HBV3 target in mammalian cells. Finally, a single-chain construct was assembled and screened against the HBV3 target in CHO cells (Example 33). The single-chain construct displayed cleavage activity for the HBV3 target in mammalian cells that was comparable to the HBV3.3/HBV3.4 heterodimer.

### EXAMPLE 24

### Identification of Meganucleases Cleaving HBV33

This example shows that I-CreI variants can cut the HBV3.3 DNA target sequence derived from the left part of the HBV3.2 target in a palindromic form (FIG. 70). Target sequences described in this example are 22 bp palindromic sequences. Therefore, they will be described only by the first 11 nucleotides, followed by the suffix _P (For example, target HBV3.3 will be noted ctgccttacgt_P).

HBV3.3 is similar to 10TGC_P at positions ±1, ±2, ±3, ±8, ±9, ±10 and ±11 and to 5TAC_P at positions ±1, ±2, ±3, ±4, ±5 and ±11. It was hypothesized that positions ±6 and ±7 would have little effect on the binding and cleavage activity. Variants able to cleave the 10 TGC_P target were obtained by mutagenesis of I-CreI N75 or D75, at positions 28, 30, 32, 33, 38 and 40, as described previously in Smith et al. Nucleic Acids Res., 2006, 34, e149; International PCT Applications WO 2007/060495 and WO 2007/049156. Variants able to cleave 5TAC_P were obtained by mutagenesis on I-CreI N75 at positions 24, 44, 68, 70, 75 and 77 as described in Arnould et al., J. Mol. Biol., 2006, 355, 443-458; Smith et al. Nucleic Acids Res., 2006, 34, e149; International PCT Applications WO 2006/097784, WO 2006/097853, WO 2007/060495 and WO 2007/049156.

Mutations at positions 24 found in variants cleaving the 5TAC_P target will be lost during the combinatorial process. But it was hypothesized that this will have little impact on the capacity of the combined variants to cleave the HBV3.3 target.

Therefore, to check whether combined variants could cleave the HBV3.3 target, mutations at positions 44, 68, 70, 75 and 77 from proteins cleaving 5TAC_P were combined with the 28, 30, 32, 33, 38 and 40 mutations from proteins cleaving 10TGC_P.

A) Material and Methods

a) Construction of Target Vector

The target was cloned as follows: an oligonucleotide corresponding to the HBV3.3 target sequence flanked by gateway cloning sequences was ordered from PROLIGO: 5′ tggcatacaagtttcctgccttacgtacgtaaggcaggcaatcgtctgtca 3′ (SEQ ID NO: 729). Double-stranded target DNA, generated by PCR amplification of the single stranded oligonucleotide, was cloned using the Gateway protocol (INVITROGEN) into the yeast reporter vector (pCLS1055, FIG. 4). Yeast reporter vector was transformed into Saccharomyces cerevisiae strain FYBL2-7B (MAT a, ura3Δ851, trp1Δ63, leu2Δ1, lys2Δ202), resulting in a reporter strain.

### b) Construction of Combinatorial Variants

I-CreI variants cleaving 10TGC_P or 5TAC_P were previously identified, as described in Smith et al. Nucleic Acids Res., 2006, 34, e149; International PCT Applications WO 2007/060495 and WO 2007/049156, and Arnould et al., J. Mol. Biol., 2006, 355, 443-458; International PCT Applications WO 2006/097784 and WO 2006/097853, respectively for the 10TGC_P and 5TAC_P targets. In order to generate I-CreI derived coding sequences containing mutations from both series, separate overlapping PCR reactions were carried out that amplify the 5′ end (aa positions 1-43) or the 3′ end (positions 39-167) of the I-CreI coding sequence. For both the 5′ and 3′ end, PCR amplification is carried out using primers (Gal10F 5′-gcaactttagtgctgacacatacagg-3′ (SEQ ID NO: 263) or Gal10R 5′-acaaccttgattggagacttgacc-3′(SEQ ID NO: 264)) specific to the vector (pCLS0542, FIG. 5) and primers (assF 5′-ctannnttgaccttt-3′ (SEQ ID NO: 265) or assR 5′-aaaggtcaannntag-3′(SEQ ID NO: 266)), where nnn codes for residue 40, specific to the I-CreI coding sequence for amino acids 39-43. The PCR fragments resulting from the amplification reaction realized with the same primers and with the same coding sequence for residue 40 were pooled. Then, each pool of PCR fragments resulting from the reaction with primers Gal10F and assR or assF and Gal10R was mixed in an equimolar ratio. Finally, approximately 25 ng of each final pool of the two overlapping PCR fragments and 75 ng of vector DNA (pCLS0542, FIG. 5) linearized by digestion with NcoI and EagI were used to transform the yeast Saccharomyces cerevisiae strain FYC2-6A (MATα, trp1Δ63, leu2Δ1, his3Δ200) using a high efficiency LiAc transformation protocol (Gietz and Woods, Methods Enzymol., 2002, 350, 87-96). An intact coding sequence containing both groups of mutations is generated by in vivo homologous recombination in yeast.

c) Mating of Meganuclease Expressing Clones and Screening in Yeast

Screening was performed as described previously (Arnould et al., J. Mol. Biol., 2006, 355, 443-458). Mating was performed using a colony gridder (QpixII, GENETIX). Variants were gridded on nylon filters covering YPD plates, using a low gridding density (4-6 spots/cm²). A second gridding process was performed on the same filters to spot a second layer consisting of the reporter-harboring yeast strain for the target of interest. Membranes were placed on solid agar YPD rich medium, and incubated at 30° C. for one night, to allow mating. Next, filters were transferred to synthetic medium, lacking leucine and tryptophan, with galactose (2%) as a carbon source, and incubated for five days at 37° C., to select for diploids carrying the expression and target vectors. After 5 days, filters were placed on solid agarose medium with 0.02% X-Gal in 0.5 M sodium phosphate buffer, pH7.0, 0.1% SDS, 6% dimethyl formamide (DMF), 7 mM β-mercaptoethanol, 1% agarose, and incubated at 37° C., to monitor β-galactosidase activity. Results were analyzed by scanning and quantification was performed using appropriate software.

d) Sequencing of Variants

To recover the variant expression plasmids, yeast DNA was extracted using standard protocols and used to transform E. coli. Sequencing of variant ORFs was then performed on the plasmids by MILLEGEN SA. Alternatively, ORFs were amplified from yeast DNA by PCR (Akada et al., Biotechniques, 2000, 28, 668-670), and sequencing was performed directly on the PCR product by MILLEGEN SA.

B) Results

I-CreI combinatorial variants were constructed by associating mutations at positions 44, 68, 70, 75 and 77 from proteins cleaving 5TAC_P with the 28, 30, 32, 33, 38 and 40 mutations from proteins cleaving 10TGC_P on the I-CreI scaffold, resulting in a library of complexity 1150. Examples of combinatorial variants are displayed in Table LXIV. This library was transformed into yeast and 4608 clones (4 times the diversity) were screened for cleavage against the HBV3.3 DNA target (ctgccttacgt_P, SEQ ID NO: 725). A total of 550 positive clones were found to cleave HBV3.3. Sequencing and validation by secondary screening of 186 of the best I-CreI variants resulted in the identification of 120 different novel endonucleases. Examples of positives are shown in FIG. 71. The sequence of several of the variants identified display non parental combinations at positions 28, 30, 32, 33, 38, 40 or 44, 68, 70, 75, 77 as well as additional mutations (see Examples Table LXV). Such variants likely result from PCR artifacts during the combinatorial process. Alternatively, the variants may be I-CreI combined variants resulting from micro-recombination between two original variants during in vivo homologous recombination in yeast.

TABLE LXIV
Panel of variants* theoretically present in the combinatorial library

| Amino acids at | | | | | | | | | | | |
|---|---|---|---|---|---|---|---|---|---|---|---|
| positions 44, 68, | | | | | | | | | | | |
| 70, 75 and 77 | | | | | | | | | | | |
| (ex: AYSRT | | | | | | | | | | | |
| stands for A44, | Amino acids at positions 28, 30, 32, 33, 38 and 40 | | | | | | | | | | |
| Y68, S70, R75 | (ex: KNSSSS stands for K28, N30, S32, S33, S38 and S40) | | | | | | | | | | |
| and T77) | KNSSSS | KNTTQS | KNSTAS | KNSTSS | KHSCQS | KNSCSS | KNSCAS | KNSVHS | KNSCTS | KNTCQS | KNSTTS |
| AYSRT | + | + | | | + | + | | | | | |
| ARNNI | | | | | | | | | | | |
| ATRNI | | | | | | | | | | | |
| AHRNI | | | | | | + | | | | | |
| ARGNI | | | | | | | | | | | |
| NHRNI | | | | | | + | | | | | |
| AKNNI | | | | | | | | | | | |
| NARNI | | | | | | | | | | | |
| NKRNI | | | | | | | | | | | |
| NRSRY | | | | | | | + | | | | |
| AYSRI | | | + | | + | + | + | + | + | + | |
| NRSRS | | | | | | | + | | | | |
| NKSRN | | | | | | + | + | | | | |
| NRSRN | | | | | | + | + | | | | |
| ARSRN | | | | | | + | | | | | |
| ARSRL | | | | | | | | | | | |
| ARSRV | | | | | | | | | | | |
| NASRY | | | | | | | | | | | |
| NASRT | | | | | | | | | | | |
| NYSRV | | | + | | | | | + | | + | |
| NRRNI | | | | | | | | | | | |
| NYSRY | + | + | + | + | + | + | | + | + | + | + |
| NTSRI | | | | | | | | | | | |
| AYSRV | | | + | | | | + | | + | + | |

*Only 264 out of the 1150 combinations are displayed.
+ indicates that a functional combinatorial variant cleaving the HBV3.4 target was found among the identified positives.

TABLE LXV
I-CreI variants capable of cleaving the HBV3.3 DNA target.

| Amino acids at positions 28, 30, 32, | |
|---|---|
| 33, 38, 40/44, 68, 70, 75 and 77 | |
| of the I-CreI variants | |
| (ex: KNSSRH/NYSRY stands for | SEQ |
| K28, N30, S32, S33, R38, H40/ | ID |
| N44, Y68, S70, R75 and Y77) | NO: |
| KNSSRH/NYSRY | 730 |
| KNSSRQ/AYSRI | 731 |
| KNSCRS/AYSRT | 732 |
| KNSSRE/NYSRY | 733 |
| KNSSRQ/NYSRV | 734 |

### EXAMPLE 25

### Making of Meganucleases Cleaving HBV3.4

This example shows that I-CreI variants can cleave the HBV3.4 DNA target sequence derived from the right part of the HBV3.2 target in a palindromic form (FIG. 70). All target sequences described in this example are 22 bp palindromic sequences. Therefore, they will be described only by the first 11 nucleotides, followed by the suffix _P (for example, HBV3.4 will be called ttctcttccgt_P).

HBV3.4 is similar to 5TCC_P at positions ±1, ±2, ±3, ±4, ±5 and to 10TCT_P at positions ±1, ±2, ±3, ±8, ±9 and ±10. It was hypothesized that positions ±6, ±7 and ±11 would have little effect on the binding and cleavage activity. Variants able to cleave 5TCC_P were obtained by mutagenesis of I-CreI N75 at positions 24, 44, 68, 70, 75 and 77, as described previously (Arnould et al., J. Mol. Biol., 2006, 355, 443-458; Smith et al. Nucleic Acids Res., 2006, 34, e149; International PCT Applications WO 2006/097784, WO 2006/097853, WO 2007/060495 and WO 2007/049156). Variants able to cleave the 10TCT_P target were obtained by mutagenesis of I-CreI N75 or D75, at positions 28, 30, 32, 33, 38, 40 and 70, as described previously in Smith et al. Nucleic Acids Res., 2006, 34, e149; International PCT Applications WO 2007/060495 and WO 2007/049156.

Both sets of proteins are mutated at position 70. However, the existence of two separable functional subdomains was hypothesized. This implies that this position has little impact on the specificity at bases 10 to 8 of the target. Mutations at positions 24 found in variants cleaving the 5TCC_P target will be lost during the combinatorial process. But it was hypothesized that this will have little impact on the capacity of the combined variants to cleave the HBV3.4 target.

Therefore, to check whether combined variants could cleave the HBV3.4 target, mutations at positions 44, 68, 70, 75 and 77 from proteins cleaving 5TCC_P were combined with the 28, 30, 32, 33, 38 and 40 mutations from proteins cleaving 10 TCT_P.

A) Material and Methods

a) Construction of Target Vector

The experimental procedure is as described in Example 24, with the exception that an oligonucleotide corresponding to the HBV3.4 target sequence was used: 5′ tggcatacaagttttttctcttccgtacgacgtaaagacaatcgtctgtca 3′ (SEQ ID NO: 729).

b) Construction of Combinatorial Variants

I-CreI variants cleaving 10TCT_P or 5TCC_P were previously identified, as described in Smith et al. Nucleic Acids Res., 2006, 34, e149; International PCT Applications WO 2007/060495 and WO 2007/049156, and Arnould et al., J. Mol. Biol., 2006, 355, 443-458; International PCT Applications WO 2006/097784 and WO 2006/097853, respectively for the 10TCT_P and 5TCC_P targets. In order to generate I-CreI derived coding sequences containing mutations from both series, separate overlapping PCR reactions were carried out that amplify the 5′ end (aa positions 1-43) or the 3′ end (positions 39-167) of the I-CreI coding sequence. For both the 5′ and 3′ end, PCR amplification is carried out using primers (Gal10F 5′-gcaactttagtgctgacacatacagg-3′ (SEQ ID NO: 263) or Gal10R 5′-acaaccttgattggagacttgacc-3′ (SEQ ID NO: 264)) specific to the vector (pCLS1107, FIG. 6) and primers (assF 5′-ctannnttgaccttt-3′ (SEQ ID NO: 265) or assR 5′-aaaggtcaannntag-3′(SEQ ID NO: 266)), where nnn codes for residue 40, specific to the I-CreI coding sequence for amino acids 39-43. The PCR fragments resulting from the amplification reaction realized with the same primers and with the same coding sequence for residue 40 were pooled. Then, each pool of PCR fragments resulting from the reaction with primers Gal10F and assR or assF and Gal10R was mixed in an equimolar ratio. Finally, approximately 25 ng of each final pool of the two overlapping PCR fragments and 75 ng of vector DNA (pCLS1107, FIG. 6) linearized by digestion with DraIII and NgoMIV were used to transform the yeast Saccharomyces cerevisiae strain FYC2-6A (MATα, trp1Δ63, leu2Δ1, his3Δ200) using a high efficiency LiAc transformation protocol (Gietz and Woods, Methods Enzymol., 2002, 350, 87-96). An intact coding sequence containing both groups of mutations is generated by in vivo homologous recombination in yeast.

c) Mating of Meganuclease Expressing Clones and Screening in Yeast

Screening was performed as described previously (Arnould et al., J. Mol. Biol., 2006, 355, 443-458). Mating was performed using a colony gridder (QpixII, GENETIX). Variants were gridded on nylon filters covering YPD plates, using a low gridding density (4-6 spots/cm²). A second gridding process was performed on the same filters to spot a second layer consisting of the reporter-harboring yeast strain for the target of interest. Membranes were placed on solid agar YPD rich medium, and incubated at 30° C. for one night, to allow mating. Next, filters were transferred to synthetic medium, lacking tryptophan, including G418, with galactose (2%) as a carbon source, and incubated for five days at 37° C., to select for diploids carrying the expression and target vectors. After 5 days, filters were placed on solid agarose medium with 0.02% X-Gal in 0.5 M sodium phosphate buffer, pH 7.0, 0.1% SDS, 6% dimethyl formamide (DMF), 7 mM β-mercaptoethanol, 1% agarose, and incubated at 37° C., to monitor β-galactosidase activity. Results were analyzed by scanning and quantification was performed using appropriate software.

d) Sequencing of Variants

The experimental procedure is as described in Example 24.

B) Results

I-CreI combinatorial variants were constructed by associating mutations at positions 44, 68, 70, 75 and 77 from proteins cleaving 5TCC_P with the 28, 30, 32, 33, 38 and 40 mutations from proteins cleaving 10TCT_P on the I-CreI scaffold, resulting in a library of complexity 1196. Examples of combinatorial variants are displayed in Table LXVI. This library was transformed into yeast and 4608 clones (3.8 times the diversity) were screened for cleavage against the HBV3.4 DNA target (ttctcttccgt_P, SEQ ID NO: 726). A total of 257 positive clones were found to cleave HBV3.4. Sequencing and validation by secondary screening of 178 of the best I-CreI variants resulted in the identification of 98 different novel endonucleases. Examples of positives are shown in FIG. 72. The sequence of several of the variants identified display non parental combinations at positions 28, 30, 32, 33, 38, 40 or 44, 68, 70, 75, 77 as well as additional mutations (see examples Table LXVII). Such variants likely result from PCR artifacts during the combinatorial process. Alternatively, the variants may be I-CreI combined variants resulting from micro-recombination between two original variants during in vivo homologous recombination in yeast.

TABLE LXVI
Panel of variants* theoretically present in the combinatorial library

Amino acids at

| positions 44, 68, | | | | | | | | | | |
|---|---|---|---|---|---|---|---|---|---|---|
| 70, 75 and 77 | | | | | | | | | | |
| (ex: KHENI | | | | | | | | | | |
| stands for K44, | Amino acids at positions 28, 30, 32, 33, 38 and 40 | | | | | | | | | |
| H68, E70, N75 | (ex: KNSCYS stands for K28, N30, S32, C33, Y38 and S40) | | | | | | | | | |
| and I77) | KNSCYS | KNSGYS | KNTTQS | KHSSQS | KQSGQS | KNSSWS | KNSTSS | KNSCSS | KNSCCS | KNSGTS |
| KNENI | + | | | | | | | | | |
| KHSNI | | | | | | | | | | |
| KSSNI | | | | | | | | | | |
| KQSNI | | | | | | | | | | |
| NHNNI | | | | | | | | | | |
| KNDNI | | | | | | | | | | |
| KASNT | | | | | | | | | | |
| KYSNQ | + | + | | | | + | | + | + | |
| QASNR | | | | | | | | | | |
| KYSYN | + | + | | | | | | | | |
| KYSNY | + | + | | | | | | | | |
| RYYYA | | | | | | | | | | |
| KYSNV + 45M | + | + | | | | | | | | |
| QDSSR | | | | | | | | | | |
| KNSNI | | + | | | | | | | | |
| QGGNI | | | | | | | | | | |
| KQSNT | | | | | | | | | | |
| KANNI | | | | | | | | | | |
| KRHNI | | | | | | | | | | |
| KRQNI | | | | | | | | | | |
| QRSYR | | | | | | | | | | |
| KYSNV | + | | | | | | | | | |

*Only 220 out of the 1196 combinations are displayed.
+ indicates that a functional combinatorial variant cleaving the HBV3.4 target was found among the identified positives.

TABLE LXVII
I-CreI variants with additional mutations capable of cleaving the
HBV3.4 DNA target.

| Amino acids at positions 28, 30, 32, | |
|---|---|
| 33, 38, 40/44, 68, 70, 75 and 77 | |
| of the I-CreI variants | |
| (ex: KNSSYS/KHNNI stands for | SEQ |
| K28, N30, S32, S33, Y38, S40/K44, | ID |
| H68, N70, N75 and I77) | NO: |
| KNSSYS/KHNNI | 736 |
| KNSSWS/KYSNQ | 737 |
| KQSAQS/QYSYR | 738 |
| KNSCAS/KYSNY | 739 |
| KNSCCS/KYSNI | 740 |

### EXAMPLE 26

### Making of Meganucleases Cleaving HBV3.2

I-CreI variants able to cleave each of the palindromic HBV3.2 derived targets (HBV3.3 and HBV3.4) were identified in Example 24 and Example 25. Pairs of such variants (one cutting HBV3.3 and one cutting HBV3.4) were co-expressed in yeast. Upon co-expression, there should be three active molecular species, two homodimers, and one heterodimer. It was assayed whether the heterodimers that should be formed, cut the HBV3.2 and the non palindromic HBV3 targets.

A) Materials and Methods

a) Construction of Target Vector

The experimental procedure is as described in Example 10, with the exception that an oligonucleotide corresponding to the HBV3.2 target sequence: 5′ tggcatacaagtttcctgccttacgtacggaagagaaacaatcgtctgtca 3′(SEQ ID NO: 741) or the HBV3 target sequence: 5′ tggcatacaagtttcctgccttacttttggaagagaaacaatcgtctgtca 3′ (SEQ ID NO: 742) was used.

b) Co-Expression of Variants

Yeast DNA was extracted from variants cleaving the HBV3.4 target in the pCLS1107 expression vector using standard protocols and was used to transform E. coli. The resulting plasmid DNA was then used to transform yeast strains expressing a variant cutting the HBV3.3 target in the pCLS0542 expression vector. Transformants were selected on synthetic medium lacking leucine and containing G418.

c) Mating of Meganucleases Co-Expressing Clones and Screening in Yeast

Mating was performed using a colony gridder (QpixII, Genetix). Variants were gridded on nylon filters covering YPD plates, using a low gridding density (4-6 spots/cm²). A second gridding process was performed on the same filters to spot a second layer consisting of reporter-harboring yeast strain for the target of interest. Membranes were placed on solid agar YPD rich medium, and incubated at 30° C. for one night, to allow mating. Next, filters were transferred to synthetic medium, lacking leucine and tryptophan, including G418, with galactose (2%) as a carbon source, and incubated for five days at 37° C., to select for diploids carrying the expression and target vectors. After 5 days, filters were placed on solid agarose medium with 0.02% X-Gal in 0.5 M sodium phosphate buffer, pH7.0, 0.1% SDS, 6% dimethyl formamide (DMF), 7 mM β-mercaptoethanol, 1% agarose, and incubated at 37° C., to monitor β-galactosidase activity. Results were analyzed by scanning and quantification was performed using appropriate software.

B) Results

Co-expression of variants cleaving the HBV3.4 target (7 variants chosen among those described in Table LXVI and Table LXVII) and four variants cleaving the HBV3.3 target (described in Table LXIV and Table LXV) resulted in efficient cleavage of the HBV3.2 target in all cases (FIG. 73). However, none of these combinations were able to cut the HBV3 natural target that differs from the HBV3.2 sequence by 2 bp at positions 1 and 2 (FIG. 70). Functional combinations cleaving HBV3.2 are summarized in Table LXVIII.

TABLE LXVIII
Cleavage of the HBV3.2 target by the heterodimeric variants

| Variant HBV3.4, | Variant HBV3.3, | | | |
|---|---|---|---|---|
| amino acids at | amino acids at positions 28, 30, 32, 33, | | | |
| positions 28, 30, | 38, 40/44, 68, 70, 75 and 77 | | | |
| 32, 33, 38, 40/44 | (ex: KNSCSS/NYSRY stands for | | | |
| 68 70 75 77 | K28, N30, S32, C33, S38, | | | |
| (ex KNSGYS/KYSNY: | S40/N44, Y68, S70, R75 and Y77) | | | |
| stands for K28, N30, | KNSCSS/ | KNSCRS/ | KNSCSS/ | KNSSRE/ |
| S32, G33, Y38, S40/ | NYSRY | AYSRT | NRSRN | NYSRY |
| K44, Y68, | (SEQ ID | (SEQ ID | (SEQ ID | (SEQ ID |
| S70, N75 and Y77) | NO: 743) | NO: 732) | NO: 744) | NO: 733) |
| KNSGYS/ | + | + | + | + |
| KYSNY (SEQ ID NO: | | | | |
| 745). | | | | |
| KNSCYS/ | + | + | + | + |
| KYSNV + 45M | | | | |
| (SEQ ID NO: 746) | | | | |
| KNSGYS/ | + | + | + | + |
| KYSNV + 45M | | | | |
| (SEQ ID NO: 747) | | | | |
| KNSSYS/ | + | + | + | + |
| KHNNI | | | | |
| (SEQ ID NO: 736) | | | | |
| KNSCYS/ | + | + | + | + |
| KYSNY | | | | |
| (SEQ ID NO: 748) | | | | |
| KNSGYS/ | + | + | + | + |
| KYSNQ | | | | |
| (SEQ ID NO: 749) | | | | |
| KNSCYS/ | + | + | + | + |
| KYSNQ | | | | |
| (SEQ ID NO: 750) | | | | |

+ indicates a functional combination

### EXAMPLE 27

### Improvement of Meganucleases Cleaving HBV3.3 by Random Mutagenesis

I-CreI variants able to cleave the HBV3.2 target by assembly of variants cleaving the palindromic HBV3.3 and HBV3.4 target have been previously identified in Example 26. However, none of these variants were able to cleave the HBV3 target.

Therefore, four combinatorial variants cleaving HBV3.3 were mutagenized, and variants were screened for cleavage activity of the HBV3.5. HBV3.5 is a pseudo-palindromic target similar to HBHV3.3 except that it contains the tttt sequence at positions −2 to 2 (FIG. 70). The Inventors have previously observed that the association of a variant cleaving a pseudo-palindromic target with a wild-type sequence at positions −2 to 2 with a variant cleaving the other pseudo-palindromic target will increase the probability of cleavage of the target of interest. According to the structure of the I-CreI protein bound to its target, there is no contact between the 4 central base pairs (positions −2 to 2) and the I-CreI protein (Chevalier et al., Nat. Struct. Biol., 2001, 8, 312-316; Chevalier and Stoddard, Nucleic Acids Res., 2001, 29, 3757-3774; Chevalier et al., J. Mol. Biol., 2003, 329, 253-269). Thus, it is difficult to rationally choose a set of positions to mutagenize, and mutagenesis was performed on the whole protein.

Thus, in a first step, proteins cleaving HBV3.3 were mutagenized, and in a second step, it was assessed whether they displayed increased activity with the target HBV3.5.

A) Material and Methods

a) Construction of Target Vector

The experimental procedure is as described in Example 24, with the exception that an oligonucleotide corresponding to the HBV3.5 target sequence was used: 5′ tggcatacaagtttcctgcctacttttgtaaggcaggcaatcgtctgtca 3′ (SEQ ID NO: 751).

b) Construction of Libraries by Random Mutagenesis

Random mutagenesis was performed on a pool of chosen variants, by PCR using Mn²⁺. PCR reactions were carried out that amplify the I-CreI coding sequence using the primers preATGCreFor (5′-gcataaattactatacttctatagacacgcaaacacaaatacacagcggccttgccacc-3′; SEQ ID NO: 169) and I-CreIpostRev (5′-ggctcgaggagctcgtctagaggatcgctcgagttatcagtcggccgc-3′; SEQ ID NO: 170), which are common to the pCLS0542 (FIG. 5) and pCLS1107 (FIG. 6) vectors. Approximately 25 ng of the PCR product and 75 ng of vector DNA (pCLS542, FIG. 5) by digestion with NcoI and EagI were used to transform the yeast Saccharomyces cerevisiae strain FYC2-6A (MATα, trp1Δ63, leu2Δ1, his3Δ200) using a high efficiency LiAc transformation protocol (Gietz and Woods, Methods Enzymol., 2002, 350, 87-96). Expression plasmids containing an intact coding sequence for the I-CreI variant were generated by in vivo homologous recombination in yeast.

c) Mating of Meganuclease Expressing Clones and Screening in Yeast

The experimental procedure is as described in Example 24.

d) Sequencing of Variants

The experimental procedure is as described in Example 24.

B) Results

Four variants cleaving HBV3.3, I-CreI 33C, 38S, 44N, 68Y, 70S, 75R, 77Y; I-CreI 33C, 38S, 44N, 70S, 75R77N; I-CreI 33C, 38R, 44A, 68Y, 70S, 75R, 77T and I-CreI 33S, 38R, 40E, 44N, 68Y, 70S, 75R, 77Y also called KNSCSS/NYSRY (SEQ ID NO: 743), KNSCSS/NRSRN (SEQ ID NO: 744), KNSCRS/AYSRT (SEQ ID NO: 732) and KNSSRE/NYSRY (SEQ ID NO: 733) according to the nomenclature of Table LXIV and Table LXV, were pooled, randomly mutagenized and transformed into yeast. 2304 transformed clones were then screened for cleavage against the HBV3.5 DNA target. 58 clones were found to cleave the HBV3.5 target more efficiently than the original variant. An example of positives is shown in FIG. 74. Sequencing of these positive clones indicates that 28 distinct variants were identified (see examples Table LXIX).

TABLE LXIX
Functional variant combinations displaying strong cleavage
activity for HBV3.5.
Optimized* Variants HBV3.3
(SEQ ID NO: 752 to 759)

26R 33C 38S 44N 68Y 70S 75R 77Y 81T
33C 38R 44A 68Y 70S 75R 77T 132V
33C 38R 44A 68Y 70S 75R 77Y
33C 38R 44A 57N 68Y 70S 75R 77Y 80G
33C 38R 44A 68Y 70S 75R 77T 83S
17A 33C 38R 44A 68Y 70S 75R 77T
33C 38R 44A 62L 68Y 70S 75R 77Y
33C 38R 44N 68Y 70S 72P 75R 77Y
*Mutations resulting from random mutagenesis are in bold.

### EXAMPLE 28

### Improvement of Meganucleases Cleaving HBV3.4 by Random Mutagenesis

As a complement to Example 27 we also decided to perform random mutagenesis with variants that cleave HBV3.4. The mutagenized proteins cleaving HBV3.4 were then tested to determine if they could efficiently cleave the pseudo-palindromic target HBV3.6

A) Material and Methods

a) Construction of Target Vector

The experimental procedure is as described in Example 24, with the exception that an oligonucleotide corresponding to the HBV3.6 target sequence was used: 5′ tggcatacaagttttttccttccttttggaagagaaacaatcgtctgtca 3′ (SEQ ID NO: 760).

b) Construction of Libraries by Random Mutagenesis

Random mutagenesis was performed on a pool of chosen variants, by PCR using Mn²⁺. PCR reactions were carried out that amplify the I-CreI coding sequence using the primers preATGCreFor (5′-gcataaattactatacttctatagacacgcaaacacaaatacacagcggccttgccacc-3′; SEQ ID NO: 169) and I-CreIpostRev (5′-ggctcgaggagctcgtctagaggatcgtlcgagttatcagtcggccgc-3′; SEQ ID NO: 170). Approximately 25 ng of the PCR product and 75 ng of vector DNA (pCLS1107, FIG. 6) linearized by digestion with DraIII and NgoMIV were used to transform the yeast Saccharomyces cerevisiae strain FYC2-6A (MATα, trp1Δ63, leu2Δ1, his3Δ200) using a high efficiency LiAc transformation protocol (Gietz and Woods, Methods Enzymol., 2002, 350, 87-96). Expression plasmids containing an intact coding sequence for the I-CreI variant were generated by in vivo homologous recombination in yeast.

c) Mating of Meganuclease Expressing Clones and Screening in Yeast

The experimental procedure is as described in Example 24.

d) Sequencing of Variants

The experimental procedure is as described in Example 24.

B) Results

Seven variants cleaving HBV3.4 (I-CreI 33C, 38Y, 44K, 45M, 68Y, 70S, 75N, 77V I-CreI 33C, 38Y, 44K, 68Y, 70S, 75N, 77Y, I-CreI 33C, 38Y, 44K, 68Y, 70S, 75N, 77Q, I-CreI 33G, 38Y, 44K, 68Y, 70S, 75N, 77Y, I-CreI 33S, 38Y, 44K, 68H, 70N, 75N, I-CreI 33G, 38Y, 44K, 45M, 68Y, 70S, 75N, 77V, and I-CreI 33C, 38Y, 44K, 68Y, 70S, 75N, 77Q also called KNSCYS/KYSNV +45M (SEQ ID NO: 746), KNSCYS/KYSNY (SEQ ID NO: 748), KNSGYS/KYSNQ (SEQ ID NO: 479), KNSGYS/KYSNY (SEQ ID NO: 745), KNSSYS/KHNNI (SEQ ID NO: 736), KNSGYS/KYSNV +45M (SEQ ID NO: 747), and KNSCYS/KYSNQ (SEQ ID NO: 750), respectively, according to the nomenclature of Table LXVI and Table LXVII) were pooled, randomly mutagenized and transformed into yeast. 2304 transformed clones were then screened for cleavage against the HBV3.6 DNA target. 114 clones were found to cleave the HBV3.6 target more efficiently than the original variant. An example of positives is shown in FIG. 75. Sequencing of these positive clones indicates that 65 distinct variants were identified (see examples Table LXX).

TABLE LXX
Functional variant combinations displaying strong cleavage
activity for HBV3.6.
Optimized Variants HBV3.4
(SEQ ID NO: 761 to 765 and 767 to 771)

2D 33S 38Y 44K 68Y 70S 75N 77Y 140M
33C 38Y 44K 45M 54L 68Y 70S 75N 77Y
33C 38Y 44K 64I 68Y 70S 75N 77Y 85R
33C 38Y 44K 45M 68Y 70S 75N 77V 105A
33C 38Y 44K 45M 59A 68Y 70S 75N 77V
33C 38Y 44K 45M 68Y 70S 75N 77V 85R
33S 38Y 44K 45M 68Y 70S 75N 77V 86T
33S 38Y 44K 68Y 70S 75N 77L
33C 38Y 44K 57E 68Y 70S 75N 77Y
32F 33C 38Y 44K 45M 68Y 70S 75N 77V
* Mutations resulting from random mutagenesis are in bold.

### EXAMPLE 29

### Making of Meganucleases Cleaving HBV3

Optimized I-CreI variants able to cleave each of the pseudo-palindromic targets HBV3.5 and HBV3.6 were identified in Example 27 and Example 28. Pairs of such optimized variants (one cutting HBV3.5 and one cutting HBV3.6) were co-expressed in yeast. Upon co-expression, there should be three active molecular species, two homodimers, and one heterodimer. It was determined whether the heterodimers that should be formed cut the non-palindromic HBV3 target.

A) Materials and Methods

a) Co-Expression of Variants

Yeast DNA was extracted from optimized HBV3.3 variants cleaving the HBV3.5 target (pCLS542 expression vector) as well as those optimized HBV3.4 variants cleaving HBV3.6 (pCLS1107 expression vector) using standard protocols and were used to transform E. coli. Plasmid DNA derived from an optimized HBV3.3 variant and an optimized HBV3.4 variant was then co-transformed into the yeast Saccharomyces cerevisiae strain FYC2-6A (MATα, trp1Δ63, leu2Δ1, his3Δ200) using a high efficiency LiAc transformation protocol (Gietz and Woods, Methods Enzymol., 2002, 350, 87-96). Transformants were selected on synthetic medium lacking leucine and containing G418.

b) Mating of Meganucleases Coexpressing Clones and Screening in Yeast

The experimental procedure is as described in Example 26.

B) Results

Co-expression of an optimized HBV3.3 variants cleaving the HBV3.5 target (7 variants chosen among those described in Example 27) and eleven optimized HBV3.4 variants cleaving the HBV3.6 target (described in Example 28) resulted in efficient cleavage of the HBV3 target in some cases (FIG. 76). Functional combinations cleaving HBV3 are summarized in Table LXXI.

TABLE LXXI
Cleavage of the HBV3 target by the heterodimeric variants

| | Optimized HBV3.3 variant cleaving HBV3.5 | | | | | | |
|---|---|---|---|---|---|---|---|
| | 26R 33C | | | | | | 33C 38R |
| | 38S 44N | 33C 38R | 33C 38R | 33C 38R | 33C 38S | 31H 33C | 44A 57N |
| Optimized | 68Y 70S | 44A 68Y | 44A 68Y | 44A 68Y | 44N 68H | 38R 44A | 68Y 70S |
| HBV3.4 | 75R 77Y | 70S 75R | 70S 75R | 70S 75R | 70S 75R | 68Y 70S | 75R 77Y |
| variants | 81T | 77T 132V | 77T 83S | 77Y 154G | 77N 79R | 75R 77Y | 80G |
| cleaving | (SEQ ID | (SEQ ID | (SEQ ID | (SEQ ID | (SEQ ID | (SEQ ID | (SEQ ID |
| HBV3.6 | NO: 752) | NO: 753) | NO: 756) | NO: 772) | NO: 773) | NO: 774) | NO: 755) |

| | | | | |
|---|---|---|---|---|
| 33S 38Y 44K | + | + | + | + |
| 68Y 70S 75N | | | | |
| 77L | | | | |
| (SEQ ID | | | | |
| NO: 770) | | | | |
| 33C 38Y 44K | + | + | + | + |
| 45M 68Y 70S | | | | |
| 75N 77V 121R | | | | |
| (SEQ ID | | | | |
| NO: 775) | | | | |
| 33C 38Y 44K | + | + | + | + |
| 45M 59A 68Y | | | | |
| 70S 75N 77V | | | | |
| (SEQ ID | | | | |
| NO: 765) | | | | |
| 33C 38Y 44K | + | + | + | + |
| 45M 61V 68Y | | | | |
| 70S 75N 77V | | | | |
| 85Y | | | | |
| (SEQ ID | | | | |
| NO: 776) | | | | |
| 33C 38Y 44K | + | + | + | + |
| 64I 68Y 70S | | | | |
| 7SN 77Y 85R | | | | |
| (SEQ ID | | | | |
| NO: 763) | | | | |
| 33S 38Y 44K | + | + | + | + |
| 45M 68Y 70S | | | | |
| 75N 77V 81T | | | | |
| (SEQ ID | | | | |
| NO: 777) | | | | |
| 31R 33C 38Y | + | + | + | + |
| 44K 68Y 70S | | | | |
| 73A 75N 77V | | | | |
| (SEQ ID | | | | |
| NO: 778) | | | | |
| 2S 33C 38Y | + | + | | + |
| 44K 68Y 70S | | | | |
| 75N 77Y 89A | | | | |
| (SEQ ID | | | | |
| NO: 779) | | | | |
| 33S 38Y 44K | + | + | + | + |
| 45M 68Y 70S | | | | |
| 75N 77V 86T | | | | |
| (SEQ ID | | | | |
| NO: 768) | | | | |
| 33C 38Y 44K | + | + | + | + |
| 45M 68Y 70S | | | | |
| 75N 77V 105A | | | | |
| (SEQ ID | | | | |
| NO: 764) | | | | |
| 2D 33S 38Y | + | + | + | + |
| 44K 6SY 70S | | | | |
| 75N 77Y 140M | | | | |
| (SEQ ID | | | | |
| NO: 761) | | | | |

+ indicates a functional combination

### EXAMPLE 30

### Improvement of Meganucleases Cleaving the HBV3 Target Site by Random Mutagenesis of I-CreI Variants Cleaving the HBV3.4 Target and Assembly with Variants Cleaving HBV3.3 in CHO Cells

I-CreI variants able to cleave the HBV3 target in yeast were previously identified in Example 29 by assembly of optimized variants cleaving HBV3.3 and optimized variants cleaving HBV3.4.

In this example, it was determined if the activity of the meganucleases could be increased and at the same time establish if the meganucleases are active in CHO cells. The variants cleaving HBV3.4 described in Example 28 (Table LXX) were subjected to random mutagenesis and more efficient variants cleaving HBV3 in combination with variants cleaving HBV3.3 (identified in Example 27) were identified in CHO cells. The screen in CHO cells is a single-strand annealing (SSA) based assay where cleavage of the target by the meganucleases induces homologous recombination and expression of a LagoZ reporter gene (a derivative of the bacterial lacZ gene).

1) Materials and Methods

a) Cloning of HBV3 Target in a Vector for CHO Screen

The target was cloned as follow: oligonucleotide corresponding to the HBV3 target sequence flanked by gateway cloning sequence was ordered from PROLIGO: 5′ tggcatacaagttcctgccttacttttggaagagaaacaatcgtctgtca 3′ (SEQ ID NO: 742). Double-stranded target DNA, generated by PCR amplification of the single stranded oligonucleotide, was cloned using the Gateway protocol (INVITROGEN) into CHO reporter vector (pCLS1058, FIG. 11). Cloned target was verified by sequencing (MILLEGEN).

b) Construction of Libraries by Random Mutagenesis

I-CreI variants cleaving HBV3.4 were pooled and randomly mutagenized by PCR in the presence of Mn²⁺. Primers used are attB1-ICreIFor (5′-ggggacaagtttgtacaaaaaagcaggcttcgaaggagatagaaccatggccaataccaaatataacaaagagttcc-3′; SEQ ID NO: 716) and attB2-ICreIRev (5′-ggggaccactttgtacaagaaagctgggtttagtcggccgccggggaggatttcttcttctcgc-3′; SEQ ID NO: 717). PCR products obtained were cloned in pCDNA6.2 from INVITROGEN (pCLS1768, FIG. 29), a vector for expression in CHO cells, using the Gateway protocol (INVITROGEN).

c) Re-Cloning of Meganucleases

The ORF of and I-CreI variants cleaving the HBV3.3 target were re-cloned in pCLS1768 (FIG. 29). ORFs were amplified by PCR on yeast DNA using the above described attB1-ICreIFor and attB2-ICreIRev primers. PCR products were cloned in CHO expression vector pCDNA6.2 from INVITROGEN (pCLS1768, FIG. 29) using the Gateway protocol (INVITROGEN). Resulting clones were verified by sequencing (MILLEGEN).

d) Extrachromosomal Assay in Mammalian Cells

CHO cells were transfected with Polyfect® transfection reagent according to the supplier's protocol (QIAGEN). 72 hours after transfection, culture medium was removed and 150 μl of lysis/revelation buffer for β-galactosidase liquid assay was added (typically 1 liter of buffer contained: 100 ml of lysis buffer (Tris-HCl 10 mM pH7.5, NaCl 150 mM, Triton X100 0.1%, BSA 0.1 mg/ml, protease inhibitors), 10 ml of Mg 100× buffer (MgCl₂ 100 mM, 1-mercaptoethanol 35%), 110 ml ONPG 8 mg/ml and 780 ml of sodium phosphate 0.1M pH7.5). After incubation at 37° C., OD was measured at 420 nm. The entire process is performed on an automated Velocity11 BioCel platform. Positives clones resulting of the screen of libraries were secondary screened and verified by sequencing (MILLEGEN).

Per assay, 150 ng of target vector was cotransfected with 12.5 ng of each one of the variants (12.5 ng of variant cleaving palindromic HBV3.3 target and 12.5 ng of variant cleaving palindromic HBV3.4 target).

2) Results

Four optimized variants cleaving HBV3.4 (33C, 38Y, 44K, 45M, 54L, 68Y, 70S, 75N, 77Y (SEQ ID NO: 762), 33S, 38Y, 44K, 68Y, 70S, 75N, 77L (SEQ ID NO: 769), 33S, 38Y, 44K, 45M, 68Y, 70S, 75N, 77V, 86T (SEQ ID NO: 768), and 2D, 33S, 38Y, 44K, 68Y, 70S, 75N, 77Y, 140M (SEQ ID NO: 761), according to the nomenclature of Table LXX in Example 28) were subjected to another round of optimization. They were pooled, randomly mutagenized and a library of new I-CreI variants was cloned in the pCLS1768 vector (FIG. 29) allowing expression of the variant in CHO cells. 3456 clones were screened using the extrachromosomal assay in CHO cells. The screen is carried out by co-transfection of 3 plasmids in CHO cells: one expressing a variant resulting from random mutagenesis of the variant cleaving HBV3.4, a second expressing a chosen variant cleaving HBV3.3 re-cloned in pCLS1768 (FIG. 29) and a third one containing the HBV3 target cloned in pCLS1058 (FIG. 11). The I-CreI variant cleaving HBV3.3 used for the screen of the library: I-CreI 26R, 33C, 38S, 44N, 68Y, 70S, 75R, 77Y, 81T, SEQ ID NO:752, according to Table LXIX in Example 27.

Six clones were found to trigger cleavage of the HBV3 target in the CHO assay when forming heterodimers with the optimized HBV3.3 variant (I-CreI 26R, 33C, 38S, 44N, 68Y, 70S, 75R, 77Y, 81T SEQ ID NO: 752) in a primary screen. The 6 clones (SEQ ID NO: 780 to 785) were validated in a secondary screen (FIG. 11) and sequenced (Table LXXII). In the secondary screen, the efficiency of the 6 clones was compared to one of the initial HBV3.4 variants (I-CreI 33S, 38Y, 44K, 68Y, 70S, 75N, 77L, SEQ ID NO: 769 according to Table LXX in Example 28) co-expressed with the optimized HBV3.3 variant (I-CreI 26R, 33C, 38S, 44N, 68Y, 70S, 75R, 77Y, 81T SEQ ID NO: 752). All six new refined HBV3.4 variants were able to cleave the HBV3 target with an efficacy superior to that observed with the heterodimer formed by the initial HBV3.4 variant (I-CreI 33S, 38Y, 44K, 68Y, 70S, 75N, 77L).

TABLE LXXII
I-CreI variants displaying improved cleavage activity for HBV3
DNA target when forming heterodimers with HBV3.3
(I-CreI 26R, 33C, 38S, 44N, 68Y,70S, 75R,77Y, 81T).

| | | SEQ ID |
|---|---|---|
| Name | Sequence | NO |
| 3.4_A1 | 19S 33C 38Y 44K 68Y 70S 75N 77Q 96E 140M | 780 |
| 3.4_A5 | 19S 33C 38Y 44K 68Y 70S 75N 77Y | 781 |
| 3.4_A7 | 19S 33C 38Y 44K 68Y 70S 75N 77Q 140M | 782 |
| 3.4_E10 | 19S 33C 38R 44K 68Y 70S 75N 77Q 132M | 783 |
| 3.4_B4 | 19S 33C 38Y 44K 68Y 70S 75N 77Q | 784 |
| 3.4_D3 | 19S 33C 38Y 44K 61G 68Y 70S 75N 77Q 28C | 785 |

### EXAMPLE 31

### Improvement of Meganucleases Cleaving the HBV3 Target Site by Random Mutagenesis of I-CreI Variants Cleaving the HBV3.3 Target and Assembly with Variants Cleaving HBV3.4 in CHO Cells

As a complement to Example 30 we also decided to perform random mutagenesis with variants that cleave HBV3.3. The variants cleaving HBV3.3 described in Example 27 (Table LXIX) were subjected to random mutagenesis and more efficient variants cleaving HBV3 in combination with a variant cleaving HBV3.4 (identified in Example 30) were identified in CHO cells.

1) Materials and Methods

a) Construction of Libraries by Random Mutagenesis

I-CreI variants cleaving HBV3.3 were pooled and randomly mutagenized by PCR in the presence of Mn²⁺. Primers used are attB1-ICreIFor (5′-ggggacaagtttgtacaaaaaagcaggcttcgaaggagatagaaccatggccaataccaaatataacaaagagttcc-3′; SEQ ID NO: 716) and attB2-ICreIRev (5′-ggggaccactttgtacaagaaagctgggtttagtcggccgccggggaggatttcttcttctcgc-3′; SEQ ID NO: 717). PCR products obtained were cloned in pCDNA6.2 from INVITROGEN (pCLS1768, FIG. 29), a vector for expression in CHO cells, using the Gateway protocol (INVITROGEN).

b) Extrachromosomal Assay in Mammalian Cells

Extrachromosomal assay in mammalian cells was performed as described in Example 30.

2) Results

Four optimized variants cleaving HBV3.3 (26R, 33C, 38S, 44N, 68Y, 70S, 75R, 77Y, 81T (SEQ ID NO: 752), 33C, 38R, 44A, 68Y, 70S, 75R, 77T, 132V (SEQ ID NO: 753), 33C, 38R, 44A, 68Y, 70S, 75R, 77T, 83S (SEQ ID NO: 756), and 33C, 38R, 44A, 57N, 68Y, 70S, 75R, 77Y, 80G (SEQ ID NO: 755), according to the nomenclature of Table LXIX in Example 27) were subjected to a round of optimization. They were pooled, randomly mutagenized and a library of new I-CreI variants was cloned in the pCLS1768 vector allowing expression of the variant in CHO cells (FIG. 29). 2976 clones were screened using the extrachromosomal assay in CHO cells. The screen is carried out by co-transfection of 3 plasmids in CHO cells: one expressing a variant resulting from random mutagenesis of the variant cleaving HBV3.3, a second expressing a chosen variant cleaving HBV3.4 re-cloned in pCLS1768 (FIG. 29) and a third one containing the HBV3 target cloned in pCLS1058 (FIG. 11). The optimized I-CreI variant cleaving HBV3.4 (3.4_B4) was used for the screen of the library: 19S, 33C, 38Y, 44K, 68Y, 70S, 75N, 77Q (SEQ ID NO: 784) according to Table LXXII in Example 29.

Six clones were found to trigger cleavage of the HBV3 target in the CHO assay when forming heterodimers with the HBV3.4 variant (I-CreI 19S, 33C, 38Y, 44K, 68Y, 70S, 75N, 77Q, SEQ ID NO: 784) in a primary screen. The 6 clones (SEQ ID NO: 786 to 787 and 789 to 792) were validated in a secondary screen (FIG. 78) and sequenced (Table LXXIII). In the secondary screen, the efficiency of the 6 clones was compared to one of the initial HBV3.3 variants (I-CreI 26R, 33C, 38S, 44N, 68Y, 70S, 75R, 77Y, 81T, SEQ ID NO: 752, according to Table LXXVIII in Example 27) co-expressed with the HBV3.4 variant (I-CreI 19S, 33C, 38Y, 44K, 68Y, 70S, 75N, 77Q, SEQ ID NO: 784). One of the new HBV3.3 variants (I-CreI 26R, 33C, 38S, 44N, 68Y, 70S, 75R, 77Y, 81T, 139R, SEQ ID NO:791) was able to cleave the HBV3 target with an efficacy superior to that observed with the heterodimer formed by the initial HBV3.3 variant (I-CreI 26R, 33C, 38S, 44N, 68Y, 70S, 75R, 77Y, 81T, SEQ ID NO: 752).

TABLE LXXIII
I-CreI variants displaying improved cleavage activity for HBV3
DNA target when forming heterodimers with HBV3.4
(I-CreI 19S, 33C, 38Y, 44K, 68Y, 70S, 77Q).

| | | SEQ ID |
|---|---|---|
| Name | Sequence | NO |
| 3.3_B10 | 23V 26R 33C 38S 44N 68Y 70S 75R 77Y 81T | 786 |
| 3.3_C9 | 33C 34R 38R 44A 68Y 70S 75R 77T 81T 83S | 787 |
| 3.3_D3 | 26R 33C 38S 44N 68Y 70S 75R 77Y 81T 130G | 789 |
| 3.3_E9 | 26R 33C 38S 44N 53R 68Y 70S 75R 77Y 81T 89A | 790 |
| 3.3_F1 | 26R 33C 38S 44N 68Y 70S 75R 77Y 81T 139R | 791 |
| 3.3_G6 | 26R 33C 38S 44N 56G 68Y 70S 75R 77Y 81T | 792 |

### EXAMPLE 32

### Improvement of Meganucleases Cleaving the HBV3 DNA Target by Multiple Site-Directed Mutagenesis of HBV3.3 and HBV3.4 Variants

Optimized HBV3.3 and HBV3.4 variants able to cleave the HBV3 target in CHO cells when forming heterodimers were identified in Examples 30 and 31. However, these variants displayed cleavage activity for the HBV3 target that was inferior to that of the I-SceI meganuclease for its target. To try and further improve that activity of the HBV3 meganuclease, HBV3.3 and HBV3.4 variants were subjected to an additional step of optimization by introducing selected amino-acid substitutions.

Three amino-acid substitutions have been found in previous studies to enhance the activity of I-CreI derivatives: these mutations correspond to the replacement of Phenylalanine 54 with Leucine (F54L), Valine 105 with Alanine (V105A) and Isoleucine 132 with Valine (I132V). One, two or all three of these mutations were introduced into the coding sequence of proteins cleaving HBV3.3 and HBV3.4; and the resulting heterodimers were tested for their ability to induce cleavage of the HBV3 target in an extrachromosomal assay in CHO cells.

1) Materials and Methods

a) Construction of Site Directed Variants (Single Mutations)

Site-directed variants containing a single mutation were created by PCR. For example, to introduce the F54L substitution into the coding sequence of the variant, two separate overlapping PCR reactions were carried out that amplify the 5′ end (amino acid residues 1-59) or the 3′ end (amino acid residues 49-167) of the I-CreI coding sequence. For both the 5′ and 3′ end, PCR amplification is carried out using a primer with homology to the vector attB1-ICreIFor (5′-ggggacaagtttgtacaaaaaagcaggcttcgaaggagatagaaccatggccaataccaaatataacaaagagttcc-3′; SEQ ID NO: 717) and attB2-ICreIRev (5′-ggggaccactttgtacaagaaagctgggtttagtcggccgccggggaggatttcttcttctcgc-3′; SEQ ID NO: 718) and a primer specific to the I-CreI coding sequence for amino acids 49-59 that contain the substitution mutation F54L (F54LF: 5′-acccagcgccgttggctgctggacaaactagtg-3′ (SEQ ID NO: 656) or F54LR: 5′-cactagtttgtccagcagccaacggcgctgggt-3′ (SEQ ID NO: 657)). The resulting PCR products contain 33 bp of homology with each other. An intact I-CreI coding sequence is obtained by assembly PCR and subsequently cloned in pCDNA6.2 from INVITROGEN (pCLS1768, FIG. 29), a vector for expression in CHO cells, using the Gateway protocol (INVITROGEN). Resulting clones were verified by sequencing (MILLEGEN).

The same strategy is used with the following pair of oligonucleotides to create variants containing the V 105A and I132V substitutions, respectively:

V105AF: 5′-aaacaggcaaacctggctctgaaaattatcgaa-3′
and V105AR: 5′-ttcgataattttacagagccaggtttgcctgtt
t-3′ SEQ ID NO: 661 and 662);
I132VF: 5′-acctgggtggatcaggttgcagctctgaacgat-3′
and II32VR: 5′-atcgttcagagctgcaacctgatccacccagg
t-3′ SEQ ID NO: 663 and 664).

b) Construction of Site Directed Variants (Multiple Mutations)

To obtain multiple insertions of the F54L, V105A and I132V substitutions into the coding sequence of HBV3.3 and HBV3.4 variants, 4 groups of separate overlapping PCR reactions were carried out that amplify internal fragments of the I-CreI N75 coding sequence containing the sequences between the different mutations. As an example, for the multiple site directed mutagenesis for the insertion of the mutations F54L and V105A, PCR amplification is carried out using a forward primer specific to the I-CreI coding sequence for amino acids 49-59 either with or without the substitution F54L (F54LF: 5′-acccagcgccgttggctgctggacaaactagtg-3′ (SEQ ID NO: 656) and F54wtF: 5′-acccagcgccgttggtttctggacaaactagtg-3′ (SEQ ID NO: 801)) and a reverse primer specific to the I-CreI coding sequence for amino acids 100-110 either with or without V105A (V105AR 5′-ttcgataattttcagagccaggttgcctgttt-3′ (SEQ ID NO: 662) and V105wtR 5′-ttcgataattttcagaaccaggtttgcctgttt-3′ (SEQ ID NO: 802)), leading to the generation of four different PCR fragments.

The same strategy was used with the following groups of oligonucleotides to create the other internal fragments:

attB1-ICreIFor (5′-ggggacaagtttgtacaaaaaagcaggcttc
gaaggagatagaaccatggccaataccaaatataacaaagagacc-3′;
SEQ ID NO: 716) with F54LR 5′-cactagtttgtccagcagcc
aacggcgctgggt-3′(SEQ ID NO: 657) and F54wtR 5′-
cactagtttgtccagaaaccaacggcgctgggt-3′(SEQ ID NO:
801)
V105AF: 5′-aaacaggcaaacctggctctgaaaattatcgaa-3′
(SEQ ID NO: 661) and V105wtF: 5′-aaacaggcaaacctggt
tctgaaaattatcgaa-3′ (SEQ ID NO: 672) with I132VR
5′-atcgttcagagctgcaacctgatccacccaggt-3′(SEQ ID NO:
664) and I132wtR 5′-atcgttcagagctgcaatctgatccaccca
ggt-3′ (SEQ ID NO: 667)
I132VF: 5′-acctgggtggatcaggttgcagctctgaacgat-3′
(SEQ ID NO: 663) and I132wtF: 5′-acctgggtggatcagat
tgcagctctgaacgat-3′ (SEQ ID NO: 766) with attB2-
ICreIRev (5′-ggggaccactttgtacaagaaagctgggtttagtcgg
ccgccggggaggatttcttcttctcgc-3′; (SEQ ID NO: 717).

The resulting overlapping PCR products contain 15 bp of homology with each other. The PCR fragments corresponding to each internal region were then purified and I-CreI coding sequences containing the mutations F54L, V105A and I132V were generated by PCR assembly. The PCR products are subsequently cloned in pCDNA6.2 from INVITROGEN (pCLS1768, FIG. 29), a vector for expression in CHO cells, using the Gateway protocol (INVITROGEN). Resulting clones were verified by sequencing (MILLEGEN).

c) Extrachromosomal Assay in Mammalian Cells

Extrachromosomal assay in mammalian cells was performed as described in Example 30.

2) Results

All possible combinations of three site directed mutations (F54L, V105A and I132V) were inserted into a HBV3.3 variant, HBV3.3_F1 (I-CreI 26R, 33C, 38S, 44N, 68Y, 70S, 75R, 77Y, 81T, 139R, SEQ ID NO: 791) and an HBV3.4 variant, HBV3.4_A7 (I-CreI 19S, 33C, 38Y, 44K, 68Y, 70S, 75N, 77Q, 140M, SEQ ID NO: 782). Thus, seven site-directed variants were generated for each variant (+54L, +54L 105A, +54L 132V, +105A, +105A 132V, +132V, +54L 105A 132V) and were cloned in the pCLS1768 vector allowing expression of the variant in CHO cells (FIG. 29).

All pair-wise combinations of the HBV3.3 and HBV3.4 variants containing site-directed mutations were screened using the extrachromosomal assay in CHO cells. The screen is carried out by co-transfection of 3 plasmids in CHO cells: one expressing a variant cleaving HBV3.3, a second expressing a variant cleaving HBV3.4 and a third one containing the HBV3 target cloned in pCLS1058 (FIG. 11).

Five different heterodimers were found to trigger improved cleavage of the HBV3 target in a primary screen in CHO cells. These heterodimers consisted of a HBV3.3 variant containing site-directed mutations co-expressed with either the initial HBV3.4 variant or one of four HBV3.4 variants containing site-directed mutations (Table LXXIV). These five heterodimers were validated in a secondary screen (FIG. 79). In the secondary screen, the efficiency of the five heterodimers was compared to the initial heterodimer (HBV3.3_F1 variant I-CreI 26R, 33C, 38S, 44N, 68Y, 70S, 75R, 77Y, 81T, 139R, SEQ ID NO: 791 co-expressed with the HBV3.4_A7 variant I-CreI 19S, 33C, 38Y, 44K, 68Y, 70S, 75N, 77Q, 140M, SEQ ID NO: 782). All five optimized heterodimers were able to cleave the HBV3 target with an efficacy superior to that observed with the initial heterodimer.

TABLE LXXIV
I-CreI variant combinations displaying improved cleavage
efficiency of the HBV3 target in CHO cells.

| Optimized | | |
|---|---|---|
| variant | Optimized variant derived from variants | |
| cleaving | cleaving | SEQ ID |
| HBV3.3 | the HBV3.4 target | NO: |
| 3.3_R5 | 3.4_A7: 19S 33C 38Y 44K 68Y 70S 75N | 794 |
| 26R 33C 38S | 77Q 140M | |
| 44N 68Y 70S | 3.4_R2: 19S 33C 38Y 44K 54L 68Y 70S 75N | 795 |
| 75R 77Y 81T | 77Q 105A 140M | |
| 105A 132V | 3.4_R4: 19S 33C 38Y 44K 68Y 70S 75N | 796 |
| 139R | 77Q 105A 140M | |
| (SEQ ID NO: | 3.4_R5: 19S 33C 38Y 44K 68Y 70S 75N | 797 |
| 793) | 77Q 105A 132V 140M | |
| | 3.4_R6: 19S 33C 38Y 44K 68Y 70S 75N | 798 |
| | 77Q 132V 140M | |

* Mutations resulting from site-directed mutagenesis are in bold.

### EXAMPLE 33

### Single-Chain

The optimized HBV3 heterodimer obtained by co-expression of the two variants HBV3.3_R5 and HBV3.4_R4 efficiently cleaves the HBV3 target but will also cleave the HBV3.3 and HBV3.4 targets because of the presence of the two homodimers. To avoid this unwanted cleavage activity, a single chain molecule composed of the two I-CreI derived variants 3.3_R5 and 3.4_R4 was generated. The single chain construct was engineered using the linker RM2 (AAGGSDKYNQALSYNQALSKKYNQALSGGGGS), resulting in the production of the single chain molecule 3.3_R5-RM2-3.4_R4, also called SC_{—}34. In a second step, mutations K7E, K96E were introduced into the 3.3_R5 variant and mutations E8K, E61R into the 3.4_R4 variant of 3.3_R5-RM2-3.4_R4 to create the single chain molecule: 3.3_R5(K7E K96E)-RM2-3.4_R4(E8K E61R) that is also called SC_OH_{—}34. The resulting single chain constructs were then tested in an extrachromosomal assay in CHO for their ability to cleave the HBV3 target.

1) Materials and Methods

a) Cloning of the Single Chain Molecules

The two single chain molecules 3.3_R5-RM2-3.4_R4 (SEQ ID NO: 799) and 3.3_R5(K7E K96E)-RM2-3.4_R4(E8K E61R) (SEQ ID NO: 800) were synthesized by MWG and cloned into pCLS1768 (FIG. 29).

b) Extrachromosomal Assay in Mammalian Cells

Extrachromosomal assay in mammalian cells was performed as described in Example 30.

2) Results

The activity of the two HBV3 single chain molecules SC_{—}34 and SC_OH_{—}34 was monitored against the HBV3 target using the previously described extrachromosomal assay in CHO cells. The ability of these single-chain molecules to cleave the HBV3 target was compared to the heterodimeric meganuclease (3.3_R5/3.4_R4) as well as the I-SceI meganuclease against its proper target (tagggataacagggtaat: SEQ ID NO: 718).

The results of this screen (FIG. 80), indicate that both single chain molecules, SC_{—}34 and SC_OH_{—}34, display a cleavage activity for the HBV3 target that is similar if not greater that the heterodimeric meganuclease (3.3_R5/3.4_R4). In addition the cleavage activity, observed with the single-chain molecule is as active as I-SceI against its proper target. These results demonstrate that it is possible to improve the specificity of the HBV3 meganuclease by generating a single-chain molecule without affecting its activity toward the DNA target of interest.

### EXAMPLE 34

### Covalent Assembly of Single Chain Molecules and Validation of HBV12 Target Cleavage in an Extrachromosomal Model in CHO Cells

I-CreI variants able to efficiently cleave the HBV12 target in yeast when forming heterodimers were described in Examples 13, 14 and 15. Co-expression of two variants to obtain heterodimers that cleave the HBV12 target will also result in the generation of homodimers that can cleave the HBV12.3 and HBV12.4 targets. To avoid this unwanted cleavage activity, several of these variants, shown in Table LXXV, were selected for covalent assembly as single-chain molecules.

TABLE LXXV
I-CreI variants efficiently cleaving HBV12.3 or HBV12.4 target
sequences

| HBV12 | Amino acids positions and residues of the | |
|---|---|---|
| variant | I-CreI variants | Seq. ID NO |
| HBV12.3-MA | 24F 32Q 38C 44D 68Y 70S 75S 77R 80K | 667 |
| HBV12.3-MB | 24F 32Q 38C 44D 68Y 70S 75S 77R 80K | 669 |
| | 132V | |
| HBV12.3-MC | 30S 32R 33S 44D 68Y 70S 75S 77R | 621 |
| HBV12.4-M1 | 32H 33C 40R 44R 68Y 70S 75N 77Q | 672 |

Single chain constructs were engineered using the linker RM2 (AAGGSDKYNQALSKYNQALSKYNQALSGGGGS), thus resulting in the production of the single chain molecules MA-linker RM2-M1, MB-linker RM2-M1 and MC-linker RM2-M1. During this design step, the G19S mutation was introduced in the C-terminal variant, M1. In addition, mutations K7E, K96E were introduced into the MA, MB and MC variants and mutations E8K, E61R into the M1 variant to create the single chain molecules: MA (K7E K96E)-linkerRM2-M1 (E8K E61R G19S), MB (K7E K96E)-linkerRM2-M1 (E8K E61R G19S) and MC (K7E K96E)-linkerRM2-M1 (E8K E61R G19S) that are referred to as the SCOH-HBV12-B1, SCOH-HBV12-B2 and SCOH-HBV12-B3 scaffolds, respectively (Table LXXVI).

TABLE LXXVI
Single Chain I-Cre I variants for HBV12 cleavage in CHO cells.

| | | Mutations | Mutations | |
|---|---|---|---|---|
| | | on N- | on C- | |
| | | terminal | terminal | |
| Construct | Single chain | segment | segment | SEQ ID NO |
| pCLS2862 | SCOH- | 7E 24F 32Q | 8K 19S 32H | 788 |
| | HBV12-B1 | 38C 44D 68Y | 33C 40R 44R | |
| | | 70S 75S 77R | 61R 68Y 70S | |
| | | 80K 96E | 75N 77Q | |
| pCLS2865 | SCOH- | 7E 24F 32Q | 8K 19S 32H | 804 |
| | HBV12-B2 | 38C 44D 68Y | 33C 40R 44R | |
| | | 70S 75S 77R | 61R 68Y 70S | |
| | | 80K 96E | 75N 77Q | |
| | | 132V | | |
| pCLS2868 | SCOH- | 7E 30S 32R | 8K 19S 32H | 805 |
| | HBV12-B3 | 33S 44D 68Y | 33C 40R 44R | |
| | | 70S 75S 77R | 61R 68Y 70S | |
| | | 96E | 75N 77Q | |

In order to identify single-chain molecules displaying maximal cleavage activity for the HBV12 target in CHO cells, the efficiency of single chain constructs to cleave the HBV2 target was compared, using an extrachromosomal assay in CHO cells. The screen in CHO cells is a single-strand annealing (SSA) based assay where cleavage of the target by the meganucleases induces homologous recombination and expression of a LagoZ reporter gene (a derivative of the bacterial lacZ gene).

### 1) Materials and Methods

### a) Cloning of the Single Chain Molecule

A series of synthetic genes was ordered from MWG-EUROFINS. Synthetic genes coding for the different single chain variants targeting HBV12 were cloned into pCLS1853 (FIG. 14) using AscI and XhoI restriction sites.

### b) Cloning of HBV12 Target in a Vector for CHO Screen

The target was cloned as follow: an oligonucleotide corresponding to the HBV12 target sequence flanked by gateway cloning sequence was ordered from PROLIGO: 5′ tggcatacaagtttatattcttgggaacaagagctacacaatcgtctgtca 3′ (SEQ ID NO: 633). Double-stranded target DNA, generated by PCR amplification of the single stranded oligonucleotide, was cloned using the Gateway protocol (INVITROGEN) into CHO reporter vector (pCLS1058, FIG. 11). Cloned target was verified by sequencing (MILLEGEN).

### c) Extrachromosomal Assay in Mammalian Cells

CHO cells were transfected with Polyfect® transfection reagent according to the supplier's protocol (QIAGEN). 72 hours after transfection, culture medium was removed and 150 μl of lysis/revelation buffer for β-galactosidase liquid assay was added. After incubation at 37° C., OD was measured at 420 nm. The entire process is performed on an automated Velocity11 BioCel platform. Per assay, 150 ng of target vector was cotransfected with an increasing quantity of I-CreI variant DNA ranging from 0.78 to 25 ng. The total amount of transfected DNA was completed to 175 ng (target DNA, variant DNA, carrier DNA) using an empty vector, pCLS0002 (FIG. 85).

### 2) Results

The activity of the three HBV12 single chain molecules SCOH-HBV12-B1, SCOH-HBV12-B2 and SCOH-HBV12-B3 was monitored against the HBV12 target using the previously described CHO assay in comparison to our internal controls, SCOH-RAG and the 1-SceI meganuclease against their proper targets (Rag: ttgttctcaggtacctcagccaga SEQ ID NO: 806 and I-SceI: tagggataacagggtaat: SEQ ID NO: 718). All comparisons were done at 0.78 ng, 1.56 ng, 3.12 ng, 6.25 ng, 12.5 ng and 25 ng of transfected variant DNA.

The results of this screen (FIG. 86) indicate that all three single chain molecules, SCOH-HBV12-B1, SCOH-HBV12-B2 and SCOH-HBV12-B3, display a cleavage activity for the HBV12 target that is significantly greater than I-SceI and similar to SCOH-RAG against their proper targets. High levels of cleavage activity for the HBV12 single-chain molecules were observed even at the lowest doses (0.78 ng). These results demonstrate that it is possible to improve the specificity of the HBV12 meganuclease by generating a single-chain molecule without affecting its activity toward the DNA target of interest.

### EXAMPLE 35

### Validation of HBV12 Target Cleavage in an Extrachromosomal Model in HepG2 Cells

Single-chain I-CreI variants able to efficiently cleave the HBV12 target sequence in CHO cells were described in Example 34. In order to further validate these single chain molecules, they were examined for their ability to cleave an extrachromosomal plasmid substrate in a hepatocyte derived cell line, HepG2. The screen in HepG2 cells utilizes a plasmid containing an HBV12 target site inserted between the CMV promoter and a LacZ reporter gene where cleavage of the target by a meganuclease can result in the inactivation of the LacZ gene. HepG2 cells are transfected with plasmids coding for HBV12 single-chain molecules and either a LacZ expression plasmid containing the HBV12 target site or the LacZ plasmid without the target site. The activity of the HBV12 single-chain molecules is then evaluated by the reduction in LacZ activity observed in cells transfected with the target substrate compared to cells transfected with a plasmid without the target.

### 1) Material and Methods

### a) Cloning of HBV12 Target in a Vector for HepG2 Screen

The target was cloned as follows: a pair of complementary oligonucleotides corresponding to the HBV12 target or the I-SceI target flanked by sequences compatible with an NheI restriction site were ordered from Eurogentec (HBV12-For 5′ctagctgtagctcttgttcccaagaatattg3′, SEQ ID NO: 807; HBV12-Rev 5′ctagcatattcttgggaacaagagctacag3′, SEQ ID NO: 808; SCEI-For 5′ctagcacgctagggataacagggtaatatg3′, SEQ ID NO: 809; SCEI-Rev 5′ctagcatattaccctgttatccctagcgtg3′, SEQ ID NO: 810). Double-stranded target DNA, generated by annealing of the single stranded oligonucleotides, was cloned into the unique NheI site located between the CMV promoter and the LacZ reporter gene in the plasmid pCLS3469 (FIG. 87). Cloned target was verified by sequencing (MILLEGEN).

### b) Extrachromosomal Assay in HepG2 Cells

HepG2 cells were cultured in EMEM supplemented with 2 mM L-glutamine, penicillin (100 IU/ml), streptomycin (100 mg/ml), amphotericin B (Fongizone: 0.25 mg/ml, Invitrogen-Life Science) and 10% FBS. 10 cm plates were seeded with 7.5×10⁵ cells per plate. The next day HepG2 cells were transfected using LipoD293 (Gentaur) with either 3 μg, 7 μg or 11 μg of the I-CreI variants cleaving HBV12 target sequences or 3 μg of plasmid expressing I-SceI and 0.1 μg of LacZ plasmid substrate, the total amount of DNA was completed to 11 μg with empty vector pCLS0003 (FIG. 85). The transfection efficiency was between 30-40% using this method. Cells were harvested four days after transfection and β-galactosidase activity was assayed on a total of 5×10⁴ cells using a luminescent β-galactosidase assay (Beta-Glo assay, Promega).

### 2) Results

Three single chain variants cleaving the HBV12 target sequence (SCOH-HBV12-B1, SCOH-HBV12-B2 and SCOH-HBV12-B3) described in Example 34, were tested for their ability to inactivate a LacZ episomal substrate. As a positive control I-SceI was tested against a plasmid containing an I-SceI site inserted between the CMV promoter and the LacZ gene. FIG. 88 shows the results obtained for the three single-chain variants as well as I-SceI after transfection with either a LacZ expression plasmid containing the relevant target site or a LacZ plasmid without the target site.

All three SCOH-HBV12 variants display a significant decrease in LacZ activity when transfected with a LacZ substrate containing a HBV12 target site compared to the control transfection with a LacZ plasmid without the target site. The SCOH-HBV12-B3 variant displays a decrease of between 30-50% depending on the quantity of expression plasmid transfected. SCOH-HBV2-B1 and SCOH-HBV12-B2 display the strongest activity resulting in a decrease of between 64-75% depending on the quantity of expression plasmid transfected. This level of reduction is similar to that observed with the I-SceI control, a reduction of 89% with 3 μg of expression plasmid. Thus, these results indicate that the single chain variants cleaving the HBV12 target sequence are capable of efficiently cleaving an extrachromosomal substrate and inactivating LacZ expression in the hepatocyte cell line HepG2.

### EXAMPLE 36

### Hepatocyte-Specific SCOH-HBV12-B1 Expression

Hepatocytes are the only confirmed site of HBV replication. We thus decided to restrict SCOH-HBV12-B1 expression to these cells. To identify the best suitable combination of liver-specific promoter and enhancer elements allowing a high level of SCOH-HBV12-B1 expression in hepatocytes while inducing no expression in others cell types, we constructed nine liver-specific SCOH-HBV12-B1 expression cassettes that we tested in HepG2 cells and 293H cells.

### 1) Materials and Methods

a) Construction of the Hepatocyte-Specific SCOH-HBV12-B1 Expression Cassettes Construction of pCLS4695

The SCOH-HBV12-B1 sequence (SEQ ID NO: 788) was PCR amplified and cloned as a XmaI and XbaI fragment into the pCLS002 (FIG. 89) giving rise to construct 1A (FIG. 90).

The human α1-antitrypsin (hAAT) promoter sequence (SEQ ID NO: 811) was synthesized by MWG and cloned as a SacI fragment into the construct 1A (FIG. 90) giving rise to construct 2A (FIG. 91). The bovine growth hormone polyadenylation signal (bpA) sequence (SEQ ID NO: 812) was synthesized by MWG and cloned as a SalI fragment into the construct 2A (FIG. 91) giving rise to pCLS4695 (FIG. 92).

Construction of pCLS 4696

The hepatic locus control region from the apoliproprotein E gene (ApoE-HCR) sequence (SEQ ID NO: 813) was synthesized by MWG and cloned as a EcoRI fragment into the pCLS4695 (FIG. 92) giving rise to pCLS4696 (FIG. 93).

Construction of pCLS 4693

A 1.4 kb truncated factor IX first intron sequence (SEQ ID NO: 814) was synthesized by MWG and cloned as a PacI fragment into the pCLS4695 (FIG. 92) giving rise to pCLS4693 (FIG. 94).

Construction of pCLS 4694

A 1.4 kb truncated factor IX first intron sequence (SEQ ID NO: 814) was synthesized by MWG and cloned as a PacI fragment into the pCLS4696 (FIG. 93) giving rise to pCLS4694 (FIG. 95).

Construction of pCLS 4492

The SCOH-HBV12-B1 sequence (SEQ ID NO: 788) was PCR amplified and cloned as a XmaI and XbaI fragment into the pCLS002 (FIG. 89) giving rise to construct 1B (FIG. 96).

The human α1-antitrypsin (hAAT) promoter sequence (SEQ ID NO: 811) was synthesized by MWG and cloned as a SacI fragment into the construct 1B (FIG. 96) giving rise to construct 2B (FIG. 97). The bovine growth hormone polyadenylation signal (bpA) sequence (SEQ ID NO: 812) was synthesized by MWG and cloned as a SalI fragment into the construct 2B (FIG. 97) giving rise to pCLS4492 (FIG. 98).

Construction of pCLS4513

The hepatic locus control region from the apoliproprotein E gene (ApoE-HCR) sequence (SEQ ID NO: 813) was synthesized by MWG and cloned as a EcoRI fragment into the pCLS4492 (FIG. 98) giving rise to pCLS4513 (FIG. 99).

Construction of pCLS4604

A 1.4 kb truncated factor IX first intron sequence (SEQ ID NO: 814) was synthesized by MWG and cloned as a PacI fragment into the pCLS4492 (FIG. 98) giving rise to pCLS4604 (FIG. 100).

Construction of pCLS4605

A 1.4 kb truncated factor IX first intron sequence (SEQ ID NO: 814) was synthesized by MWG and cloned as a Pact fragment into the pCLS4513 (FIG. 99) giving rise to pCLS4605 (FIG. 101).

Construction of pCLS4863

The hepatic locus control region from the apoliproprotein E gene (ApoE-HCR) sequence (SEQ ID NO: 813) was synthesized by MWG and cloned as an EcoRI fragment into pCLS4492 (FIG. 98) giving rise to pCLS4863 (FIG. 102).

### b) Culture and Transfection of HepG2 Cells

HepG2 cells were cultured in EMEM supplemented with 2 mM L-glutamine, penicillin (100 IU/ml), streptomycin (100 mg/ml), amphotericin B (Fongizone: 0.25 mg/ml, Invitrogen-Life Science) and 10% FBS. 10 cm plates were seeded with 7.5×10⁵ cells per plate. The next day HepG2 cells were transfected using LipoD293 (Gentaur) with either 1 μg or 5 μg of plasmid expressing SCOH-HBV12-B under the control of hepatocyte specific promoters or under the control of the CMV promoter. The total amount of transfected DNA was completed to 5 μg using an empty vector, pCLS0003 (FIG. 85). The transfection efficiency was between 30-35% using this method.

c) Culture and Transfection of 293H Cells 293H cells were cultured in DMEM supplemented with 2 mM L-glutamine, penicillin (100 IU/ml), streptomycin (100 mg/ml), amphotericin B (Fongizone: 0.25 mg/ml, Invitrogen-Life Science) and 10% FBS. 10 cm plates were seeded with 1×10⁶ cells per plate. The next day 293H cells were transfected using Lipofectamine2000 (Invitrogen) with either 1 μg or 5 μg of plasmid expressing SCOH-HBV12-B1 under the control of hepatocyte specific promoters or under the control of the CMV promoter. The total amount of transfected DNA was completed to 511g using an empty vector, pCLS0003 (FIG. 85). The transfection efficiency was between 80-85% using this method.

### d) Expression Analysis in HepG2 and 293H Cells

Transfected cells were harvested two days after transfection and lysed in RIPA lysis buffer (Santa Cruz) supplemented with PMSF (Santa Cruz Biotechnology), sodium orthovanadate (Santa Cruz Biotechnology) and antiprotease (Santa Cruz Biotechnology). Proteins were quantified using a Bio-Rad protein assay (Bio-Rad). Forty micrograms of proteins were electrophoresed and Western blotted with a polyclonal rabbit anti-IcreI followed by HRP conjugated goat anti rabbit IgG. Labeled antibodies were detected using an enhanced chemoluminescence kit (Santa Cruz Biotechnology). Signals were quantified using image J (National Institutes of Health, Bethesda, Md.).

### 2) Results

The nine expression cassettes were evaluated for their ability to induce SCOH-HBV12-B1 expression in HepG2 cells. As a positive control the plasmid pCLS2862 in which the SCOH-HBV12-B1 expression is under the control of the cytomegalovirus (CMV) promoter was used. FIG. 103 shows that plasmids containing either the hAAT promoter and the bpA (pCLS4695 and pCLS4492), or the hAAT promoter, the bpA and the truncated factor IX first intron (pCLS4693 and pCLS4604), induce an expression of SCOH-HBV12-B1 that is almost undetectable (FIG. 103). Plasmids containing either the ApoE-HCR, the hAAT promoter, and the bpA (pCLS4696 and pCLS4513), or the ApoE-HCR, the hAAT promoter, the truncated factor IX first intron and the bpA (pCLS4694 and pCLS4605) induce an expression of SCOH-HBV12-B1 that is slightly weaker than that observed with the CMV control plasmid pCLS2862 (FIG. 103). The plasmid pCLS4863 containing two ApoE-HCRs, the hAAT promoter and the bpA induces an expression of SCOH-HBV12-B1 that is as high as the control plasmid pCLS2862 (FIG. 103).

To determine if these promoters were hepatocyte specific, the nine expression cassettes were evaluated for their ability to induce SCOH-HBV12-B1 expression in 293H cells, an embryonic human kidney cell line. As a positive control the plasmid pCLS2862 in which the SCOH-HBV12-B1 expression is under the control of the CMV promoter was used. As seen in FIG. 105, none of the hepatocyte specific promoters tested induce the expression of SCOH-HBV12-B1 in 293H cells. In contrast, the CMV promoter construction results in a strong expression of SCOH-HBV12-B1 in these cells (FIG. 104).

These results indicate that the best suitable combination of known liver-specific promoter and enhancer elements for the expression of SCOH-HBV12-B1 in hepatocytes corresponds to the cassette containing two ApoE-HCRs, a hAAT promoter and a bpA. This cassette induces a strong expression of SCOH-HBV12-B1 in HepG2 cells and no expression in 293H cells. Thus this cassette fulfils the requirements of a liver-specific expression cassette.

### EXAMPLE 37

### Validation of Cleavage Activity of Hepatocyte-Specific SCOH-HBV12-B1 Expression Constructs

Expression plasmids able to induce hepatocyte-specific expression of SCOH-HBV12-B1 were described in Example 36. To further validate these expression plasmids, they were examined for their ability to cleave an extrachromosomal plasmid substrate in a hepatocyte derived cell line, HepG2. The screen in HepG2 cells utilizes a plasmid containing an HBV12 target site inserted between the CMV promoter and a LacZ reporter gene where cleavage of the target by a meganuclease can result in the inactivation of the LacZ gene. HepG2 cells are transfected with one of the HBV12 single-chain expression plasmids described in Example 36 and either a LacZ expression plasmid containing the HBV12 target site or the LacZ plasmid without the target site. The cleavage activity is then evaluated by the reduction in LacZ activity observed in cells transfected with the target substrate compared to cells transfected with a plasmid without the target.

### 1) Material and Methods

### a) Hepatocyte Specific SCOH-HBV12-B1 Expression Plasmids

### See Example 36.

### b) Extrachromosomal Assay in HeGp2 Cells

### See Example 35.

### 2) Results

The nine hepatocyte-specific SCOH-HBV12-B1 expression plasmids described in Example 36, were tested for their ability to inactivate a LacZ episomal substrate in HepG2 cells. pCLS2862 in which SCOH-HBV12-B1 expression is under the control of the CMV promoter, and pCLS003 that does not code for SCOH-HBV12-B1 were used as a positive and negative controls, respectively. The results presented in FIG. 105 indicate that transfection with either the plasmids containing the hAAT promoter and bpA (pCLS4695 and pCLS4492), or the plasmids containing the hAAT promoter, the bpA and the truncated factor IX first intron (pCLS4693 and pCLS4604), results in a decrease of LacZ activity of between 27% and 43%. The SCOH-HBV12-B1 expression plasmids containing the ApoE-HCR, the hAAT promoter, and the bpA (pCLS4696 and pCLS4513), or the plasmids containing the ApoE-HCR, the hAAT promoter, the truncated factor IX first intron and the bpA (pCLS4694 and pCLS4605), display a decrease of between 52% and 64% (FIG. 105). pCLS4863 containing two ApoE-HCRs, the hAAT promoter and the bpA results in the largest reduction, a 78% decrease of LacZ activity (FIG. 105). These results indicate that the level of cleavage activity observed with the nine hepatocyte-specific SCOH-HBV12-B1 expression plasmids correlates with their expression level (see Example 36, FIG. 104). Thus, the best suitable combination of known liver-specific promoter and enhancer elements for SCOH-HBV12-B1 in terms of expression and cleavage activity corresponds to the cassette containing two ApoE-HCRs, a hAAT promoter and a bpA.

1. An I-CreI variant which cleaves a DNA target in the genome of a pathogenic non-integrating virus (NIV), for use in treating an infection of said NIV.

2-17. (canceled)
